SEQUENCE LISTING (187 sequences)

SEQ ID NO: 1 - protein, 9 aa, Homo sapiens
ALNEQIARL

SEQ ID NO: 2 - protein, 122 aa, Artificial Sequence
  Description of Artificial Sequence Synthetic polypeptide
EVQLVQSGAEVKKPGESLKISCEASGYSFSSNWIAWVRQRPGKGLEWMGIIYPGDSDTRYSPSFQGQVTMSADMSISTAYLQWSSLKASDTAMYYCARFAGPGMWSYGFDYWGQGTLVTVSS

SEQ ID NO: 3 - protein, 118 aa, Artificial Sequence
  Description of Artificial Sequence Synthetic polypeptide
QVQLVQSGAEVKKPGSSVKVSCKASGGTFSSYAISWVRQAPGQGLEWMGWISAYNGNTNYAQKLQGRVTMTTDTSTSTAYMELRSLRSDDTAVYYCARKGFYSGFDSWGQGTLVTVSS

SEQ ID NO: 4 - protein, 114 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGVPYDYWGQGTLVTVSS

SEQ ID NO: 5 - protein, 121 aa, Artificial Sequence
  Description of Artificial Sequence Synthetic polypeptide
QLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARFIYGSSSSQTDTWGQGTLVTVSS

SEQ ID NO: 6 - protein, 116 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLEGSYYDVWGQGTLVTVSS

SEQ ID NO: 7 - protein, 118 aa, Artificial Sequence
  Description of Artificial Sequence Synthetic polypeptide
QVQLQQSGAEVKKPGATVKVSCKASGYTFTSYGISWVRQAPGQGLEWMGWISAYNGNTNYAQKLQGRVTMTTDTSTSTAYMELRSLRSDDTAVYYCARMSMSEYIDYWGQGTLVTVSS

SEQ ID NO: 8 - protein, 118 aa, Artificial Sequence
  Description of Artificial Sequence Synthetic polypeptide
QLQLQESGPGLVKPSETLSLTCTVSGDSISGYYWSWIRQPPGKGLEWIGEINHSGSTNYNPSLKSRVTISVDTSKNQFSLKLSSVTAADTAVYYCARYYSGFYSSDGWGQGTLVTVSS

SEQ ID NO: 9 - protein, 117 aa, Artificial Sequence
  Description of Artificial Sequence Synthetic polypeptide
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYYCARYEQGSHDSWGQGTLVTVSS

SEQ ID NO: 10 - protein, 117 aa, Artificial Sequence
  Description of Artificial Sequence Synthetic polypeptide
QVQLQESGPGLVKPPGTLSLTCAVSGGSISSSNWWSWVRQPPGKGLEWIGEIYHSGSTNYNPSLNSRVIISDDTSKNQFSLTLTSVTAADTAVYYCARYYNSYYDSWGQGTLVTVSS

SEQ ID NO: 11 - protein, 119 aa, Artificial Sequence
  Description of Artificial Sequence Synthetic polypeptide
QVQLVQSGAEVRKPGASVKVSCKASGYSFTSNGITWVRQAPGQGLEWMGWISGYNANTRYAQEFQARVTMTTDTSASTAYMELRSLRSDDTAVYYCARHAYWGGDSDYWGQGTLVTVSS

SEQ ID NO: 12 - protein, 117 aa, Artificial Sequence
  Description of Artificial Sequence Synthetic polypeptide
QVQLQESGPGLVKPSGTLSLTCAVSGGSISSSNWWSWVRQAPGKGLEWIGEIYHGGSTNFNPSLKSRVTISVDKSKNQFSLNLTSVTAADTAVYYCARYASSSHDFWGQGTLVTVSS

SEQ ID NO: 13 - protein, 119 aa, Artificial Sequence
  Description of Artificial Sequence Synthetic polypeptide
QVQLQESGPGLVKPSGTLSLTCVVSGDSISSNTWWTWVRQPPGKGLDWIGEINHSGSTNYNPSLKSRVTISVDTSKNQFSLNLSSVTAADTAVYYCARWGYGSAYSDAWGQGTLVTVSS

SEQ ID NO: 14 - protein, 117 aa, Artificial Sequence
  Description of Artificial Sequence Synthetic polypeptide
QLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYFGQKYDYWGQGTLVTVSS

SEQ ID NO: 15 - protein, 117 aa, Artificial Sequence
  Description of Artificial Sequence Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYPGFVWDLWGQGTLVTVSS

SEQ ID NO: 16 - protein, 122 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARRGWLSWGYYGMDVWGQGTTVTVSS

SEQ ID NO: 17 - protein, 117 aa, Artificial Sequence
  Description of Artificial Sequence Synthetic polypeptide
QVQLVQSGAEVKKPGASVKVSCKASGSTFINYGITWVRQAPGQGLEWMGWVSGYNGNTDYAQKFQGRVTMTADTSTSTAYMELRSLRSDDTAVYYCARYYYPPYDYWGSRYSGDRLL

SEQ ID NO: 18 - protein, 119 aa, Artificial Sequence
  Description of Artificial Sequence Synthetic polypeptide
QMQLVQSGAEVKKPGSSVKVSCKASGGTFSSYAISWVRQAPGQGLEWMGGIIPIFGTANYAQKFQGRVTITADESTSTAYMELSSLRSDDTAVYYCARGFSSWPGIDQWGQGTLVTVSS

SEQ ID NO: 19 - protein, 122 aa, Artificial Sequence
  Description of Artificial Sequence Synthetic polypeptide
EVQLVQSGAEVKKPGESLKISCKGSGYSFTTYWIGWVRQMPGKGLEWMGIIYPGDSDTTYSPSFQGQVTISADKSLSIAYLQWSSLKASDTAMYYCARYGGQYFWSDSFDSWGQGTLVTVSS

SEQ ID NO: 20 - protein, 108 aa, Artificial Sequence
  Description of Artificial Sequence Synthetic polypeptide
DIQLTQSPSSLSASVGDRVTITCRASQGIRNDLGWYQQKPGKAPKLMIYAASSLQSGVPSRFSGSGSGTDFTLTISSLQPEDFATYYCLQDYDYPLTFGGGTKLEIKR

SEQ ID NO: 21 - protein, 111 aa, Artificial Sequence
  Description of Artificial Sequence Synthetic polypeptide
QSVLTQPPSVSAAPGQKVTISCSGSSSNIGSNYVSWYQQLPGTAPKLLIYDNTKRPSGIPDRFSGSKSGSSATLGITGLQTGDEADYYCETLDSSLSAYVFGTGTKVTVLG

SEQ ID NO: 22 - protein, 111 aa, Artificial Sequence
  Description of Artificial Sequence Synthetic polypeptide
QSVLTQPPSVSAAAGQKVTISCSGSRSNIGSNFVSWYQLLPGTAPRLLIYDNNVRPSGIPDRFSGSKSGSSATLGITGLQTGDEADYYCGTWDSSLIAGVFGGGTKLTVLG

SEQ ID NO: 23 - protein, 111 aa, Artificial Sequence
  Description of Artificial Sequence Synthetic polypeptide
QSVLTQPPSVSAAPGQKVTISCSGSSSNIGSNYIYWYQQFPGTAPKLLIYDDNKRPSGIPDRFSGSRSGTSATLGITGLQTGDEADYYCGTWDSPLVAWVFGGGTKLTVLG

SEQ ID NO: 24 - protein, 111 aa, Artificial Sequence
  Description of Artificial Sequence Synthetic polypeptide
QSVLTQPPSVSAAPGQKVTVSCSGTTSDIGSNYVTWYQQLPGTTPKLIIYDNNKRPSGIPDRFSGSKSGTSGTLGITGLQTGDEADYYCGTWDSSLNTFVFGTGTKVTVLG

SEQ ID NO: 25 - protein, 112 aa, Artificial Sequence
  Description of Artificial Sequence Synthetic polypeptide
QSVVTQPPSVSGAPGQRITISCTGSSSNIGAGYDVHWYQQLPGTAPKLLIFGNVNRPSGVPDRFSGSKSGTSASLAITGLQAEDEADYYCQSYDSSLSGWVFGGGTKLTVLG

SEQ ID NO: 26 - protein, 111 aa, Artificial Sequence
  Description of Artificial Sequence Synthetic polypeptide
QSVLTQPPSVSAAPRQKVTISCSGSSSNIGSNYVSWYQQLPGTAPKLLIYDNNKRPSGIPDRFSGSRSGTSATLGITGLQTGDEADYYCGSWDSSLSVYVFGTGTKVTVLG

SEQ ID NO: 27 - protein, 111 aa, Artificial Sequence
  Description of Artificial Sequence Synthetic polypeptide
QSVLTQPPSVSAAPGQKVTISCSGSSSNIGSNYISWYQQLPGTAPKLLIYDNSKRPSGIPDRFSGFKSGTSATLGITGLQTGDEADYYCGTWDSSLSAYVFGSGTKVTVLG

SEQ ID NO: 28 - protein, 111 aa, Artificial Sequence
  Description of Artificial Sequence Synthetic polypeptide
QSVLTQPPSVSAAPGQKVTISCSGSSSNIGSNYVSWYQQLPGTAPKLLIYDNNKRPSGIPDRFSGSKSGTSATLGITGLQTGDEADYYCGTWDSSLSAYVFGTGTKVTVLG

SEQ ID NO: 29 - protein, 111 aa, Artificial Sequence
  Description of Artificial Sequence Synthetic polypeptide
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSSNVVFGGGTKLTVLG

SEQ ID NO: 30 - protein, 111 aa, Artificial Sequence
  Description of Artificial Sequence Synthetic polypeptide
QSVLTQPPSVSAAPGQRVTIYCSGSRSNIVSNYVSWYQQLPGTAPKLLIYDNNKRPSGIPDRFSGSKSGTSATLGITGLQTGDEADYYCGTWDSSLSAYVFGTGTKVAVLG

SEQ ID NO: 31 - protein, 111 aa, Artificial Sequence
  Description of Artificial Sequence Synthetic polypeptide
QSVLTQPPSVSAAPGQKVTISCSGSSSNIGSNYVSWYQQLPGTVPKLLIYDNNKRPSGIPDRFSGSKSGTSATLGITGLQTGDEADYYCGIWDSSLSAYVFGTGTKVTVLG

SEQ ID NO: 32 - protein, 111 aa, Artificial Sequence
  Description of Artificial Sequence Synthetic polypeptide
QSVLTQPPSVSAAPGQRVTISCSGSTSNIGSNYVSWYQQFPGTAPKLLIYDSDKRISGIPDRFSGSKSGTSATLGITGLQTGDEADYYCGTWDSSLTVGVFGGGTKVTVLG

SEQ ID NO: 33 - protein, 111 aa, Artificial Sequence
  Description of Artificial Sequence Synthetic polypeptide
QSVLTQPPSVSAAPGQKVTISCSGSSSNIGSNYVSWYQQLPGTAPKLLIYDNNKRPSGIPDRFSGSKSGTSAILGITGLQTGDEADYYCGTWDSSLTAGVFGTGTKVTVLG

SEQ ID NO: 34 - protein, 108 aa, Artificial Sequence
  Description of Artificial Sequence Synthetic polypeptide
LPVLTQPPSVSVSPGQTASITCSGDKLGDKYACWYQQKPGQSPVLVIYQDSKRPSGIPERFSGSNSGNTATLTISGTQAMDEADYYCQAWDSSTAYVFGTGTKVTVLG

SEQ ID NO: 35 - protein, 107 aa, Artificial Sequence
  Description of Artificial Sequence Synthetic polypeptide
SSELTQDPAVSVALGQTVRITCQGDTLRSYYANWYQQKPGQAPVLVIYGRNNRPSGIPDRFSGSDSGNTASLTITGAQAEDEADYYCQSFDYSYSVFGGGTKLTVLG

SEQ ID NO: 36 - protein, 111 aa, Artificial Sequence
  Description of Artificial Sequence Synthetic polypeptide
QSVLTQPPSVSVAPGQKVTISCSGSSSNIGNNYVSWYQQLPGTAPKLLIYDNNKRPSGIPDRFSGSKSGTSATLGITGLQTGDEADYYCQSYDVYNMTSVFGGGTKLTVLG

SEQ ID NO: 37 - protein, 112 aa, Artificial Sequence
  Description of Artificial Sequence Synthetic polypeptide
NFMLTQPHSVSESPGKTVTISCVRSSGSVASEFVQWYQQRPGHAPTLVIYNDFQRPSGVPDRFSGSIDKSSNSASLTISGLKAEDEADYYCQSYDQSSSIVFGGGTKLTVLG

SEQ ID NO: 38 - protein, 251 aa, Artificial Sequence
  Description of Artificial Sequence Synthetic polypeptide
DIQLTQSPSSLSASVGDRVTITCRASQGIRNDLGWYQQKPGKAPKLMIYAASSLQSGVPSRFSGSGSGTDFTLTISSLQPEDFATYYCLQDYDYPLTFGGGTKLEIKRSRGGGGSGGGGSGGGGSLEMAEVQLVQSGAEVKKPGESLKISCEASGYSFSSNWIAWVRQRPGKGLEWMGIIYPGDSDTRYSPSFQGQVTMSADMSISTAYLQWSSLKASDTAMYYCARFAGPGMWSYGFDYWGQGTLVTVSS

SEQ ID NO: 39 - protein, 251 aa, Artificial Sequence
  Description of Artificial Sequence Synthetic polypeptide
QSVVTQPPSVSGAPGQRITISCTGSSSNIGAGYDVHWYQQLPGTAPKLLIFGNVNRPSGVPDRFSGSKSGTSASLAITGLQAEDEADYYCQSYDSSLSGWVFGGGTKLTVLGSRGGGGSGGGGSGGGGSLEMAQVQLQQSGAEVKKPGATVKVSCKASGYTFTSYGISWVRQAPGQGLEWMGWISAYNGNTNYAQKLQGRVTMTTDTSTSTAYMELRSLRSDDTAVYYCARMSMSEYIDYWGQGTLVTVSS

SEQ ID NO: 40 - protein, 251 aa, Artificial Sequence
  Description of Artificial Sequence Synthetic polypeptide
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSSNVVFGGGTKLTVLGSRGGGGSGGGGSGGGGSLEMAQVQLVQSGAEVRKPGASVKVSCKASGYSFTSNGITWVRQAPGQGLEWMGWISGYNANTRYAQEFQARVTMTTDTSASTAYMELRSLRSDDTAVYYCARHAYWGGDSDYWGQGTLVTVSS

SEQ ID NO: 41 - protein, 249 aa, Artificial Sequence
  Description of Artificial Sequence Synthetic polypeptide
QSVLTQPPSVSAAPGQRVTISCSGSTSNIGSNYVSWYQQFPGTAPKLLIYDSDKRISGIPDRFSGSKSGTSATLGITGLQTGDEADYYCGTWDSSLTVGVFGGGTKVTVLGSRGGGGSGGGGSGGGGSLEMAQLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYFGQKYDYWGQGTLVTVSS

SEQ ID NO: 42 - protein, 251 aa, Artificial Sequence
  Description of Artificial Sequence Synthetic polypeptide
QSVLTQPPSVSVAPGQKVTISCSGSSSNIGNNYVSWYQQLPGTAPKLLIYDNNKRPSGIPDRFSGSKSGTSATLGITGLQTGDEADYYCQSYDVYNMTSVFGGGTKLTVLGSRGGGGSGGGGSGGGGSLEMAQMQLVQSGAEVKKPGSSVKVSCKASGGTFSSYAISWVRQAPGQGLEWMGGIIPIFGTANYAQKFQGRVTITADESTSTAYMELSSLRSDDTAVYYCARGFSSWPGIDQWGQGTLVTVSS

SEQ ID NO: 43 - protein, 255 aa, Artificial Sequence
  Description of Artificial Sequence Synthetic polypeptide
NFMLTQPHSVSESPGKTVTISCVRSSGSVASEFVQWYQQRPGHAPTLVIYNDFQRPSGVPDRFSGSIDKSSNSASLTISGLKAEDEADYYCQSYDQSSSIVFGGGTKLTVLGSRGGGGSGGGGSGGGGSLEMAEVQLVQSGAEVKKPGESLKISCKGSGYSFTTYWIGWVRQMPGKGLEWMGIIYPGDSDTTYSPSFQGQVTISADKSLSIAYLQWSSLKASDTAMYYCARYGGQYFWSDSFDSWGQGTLVTVSS

SEQ ID NO: 44 - protein, 8 aa, Artificial Sequence
  Description of Artificial Sequence Synthetic peptide
GYSFSSNW

SEQ ID NO: 45 - protein, 8 aa, Artificial Sequence
  Description of Artificial Sequence Synthetic peptide
IYPGDSDT

SEQ ID NO: 46 - protein, 15 aa, Artificial Sequence
  Description of Artificial Sequence Synthetic peptide
ARFAGPGMWSYGFDY

SEQ ID NO: 47 - protein, 8 aa, Artificial Sequence
  Description of Artificial Sequence Synthetic peptide
GGTFSSYA

SEQ ID NO: 48 - protein, 8 aa, Artificial Sequence
  Description of Artificial Sequence Synthetic peptide
ISAYNGNT

SEQ ID NO: 49 - protein, 11 aa, Artificial Sequence
  Description of Artificial Sequence Synthetic peptide
ARKGFYSGFDS

SEQ ID NO: 50 - protein, 8 aa, Artificial Sequence
  Description of Artificial Sequence Synthetic peptide
GGSFSGYY

SEQ ID NO: 51 - protein, 7 aa, Artificial Sequence
  Description of Artificial Sequence Synthetic peptide
INHSGST

SEQ ID NO: 52 - protein, 8 aa, Artificial Sequence
  Description of Artificial Sequence Synthetic peptide
ARGVPYDY

SEQ ID NO: 53 - protein, 9 aa, Artificial Sequence
  Description of Artificial Sequence Synthetic peptide
GGSISSSNW

SEQ ID NO: 54 - protein, 7 aa, Artificial Sequence
  Description of Artificial Sequence Synthetic peptide
IYHSGST

SEQ ID NO: 55 - protein, 14 aa, Artificial Sequence
  Description of Artificial Sequence Synthetic peptide
ARFIYGSSSSQTDT

SEQ ID NO: 56 - protein, 8 aa, Artificial Sequence
  Description of Artificial Sequence Synthetic peptide
GGSFSGYY

SEQ ID NO: 57 - protein, 7 aa, Artificial Sequence
  Description of Artificial Sequence Synthetic peptide
INHSGST

SEQ ID NO: 58 - protein, 10 aa, Artificial Sequence
  Description of Artificial Sequence Synthetic peptide
ARLEGSYYDV

SEQ ID NO: 59 - protein, 8 aa, Artificial Sequence
  Description of Artificial Sequence Synthetic peptide
GYTFTSYG

SEQ ID NO: 60 - protein, 8 aa, Artificial Sequence
  Description of Artificial Sequence Synthetic peptide
ISAYNGNT

SEQ ID NO: 61 - protein, 11 aa, Artificial Sequence
  Description of Artificial Sequence Synthetic peptide
ARMSMSEYIDY

SEQ ID NO: 62 - protein, 8 aa, Artificial Sequence
  Description of Artificial Sequence Synthetic peptide
GDSISGYY

SEQ ID NO: 63 - protein, 7 aa, Artificial Sequence
  Description of Artificial Sequence Synthetic peptide
INHSGST

SEQ ID NO: 64 - protein, 12 aa, Artificial Sequence
  Description of Artificial Sequence Synthetic peptide
ARYYSGFYSSDG

SEQ ID NO: 65 - protein, 9 aa, Artificial Sequence
  Description of Artificial Sequence Synthetic peptide
GGSISSSNW

SEQ ID NO: 66 - protein, 7 aa, Artificial Sequence
  Description of Artificial Sequence Synthetic peptide
IYHSGST

SEQ ID NO: 67 - protein, 10 aa, Artificial Sequence
  Description of Artificial Sequence Synthetic peptide
ARYEQGSHDS

SEQ ID NO: 68 - protein, 9 aa, Artificial Sequence
  Description of Artificial Sequence Synthetic peptide
GGSISSSNW

SEQ ID NO: 69 - protein, 7 aa, Artificial Sequence
  Description of Artificial Sequence Synthetic peptide
IYHSGST

SEQ ID NO: 70 - protein, 10 aa, Artificial Sequence
  Description of Artificial Sequence Synthetic peptide
ARYYNSYYDS

SEQ ID NO: 71 - protein, 8 aa, Artificial Sequence
  Description of Artificial Sequence Synthetic peptide
GYSFTSNG

SEQ ID NO: 72 - protein, 8 aa, Artificial Sequence
  Description of Artificial Sequence Synthetic peptide
ISGYNANT

SEQ ID NO: 73 - protein, 12 aa, Artificial Sequence
  Description of Artificial Sequence Synthetic peptide
ARHAYWGGDSDY

SEQ ID NO: 74 - protein, 9 aa, Artificial Sequence
  Description of Artificial Sequence Synthetic peptide
GGSISSSNW

SEQ ID NO: 75 - protein, 7 aa, Artificial Sequence
  Description of Artificial Sequence Synthetic peptide
IYHGGST

SEQ ID NO: 76 - protein, 10 aa, Artificial Sequence
  Description of Artificial Sequence Synthetic peptide
ARYASSSHDF

SEQ ID NO: 77 - protein, 9 aa, Artificial Sequence
  Description of Artificial Sequence Synthetic peptide
GDSISSNTW

SEQ ID NO: 78 - protein, 7 aa, Artificial Sequence
  Description of Artificial Sequence Synthetic peptide
INHSGST

SEQ ID NO: 79 - protein, 12 aa, Artificial Sequence
  Description of Artificial Sequence Synthetic peptide
ARWGYGSAYSDA

SEQ ID NO: 80 - protein, 9 aa, Artificial Sequence
  Description of Artificial Sequence Synthetic peptide
GGSISSSNW

SEQ ID NO: 81 - protein, 7 aa, Artificial Sequence
  Description of Artificial Sequence Synthetic peptide
IYHSGST

SEQ ID NO: 82 - protein, 10 aa, Artificial Sequence
  Description of Artificial Sequence Synthetic peptide
ARYFGQKYDY

SEQ ID NO: 83 - protein, 9 aa, Artificial Sequence
  Description of Artificial Sequence Synthetic peptide
GGSISSSNW

SEQ ID NO: 84 - protein, 7 aa, Artificial Sequence
  Description of Artificial Sequence Synthetic peptide
IYHSGST

SEQ ID NO: 85 - protein, 10 aa, Artificial Sequence
  Description of Artificial Sequence Synthetic peptide
ARYPGFVWDL

SEQ ID NO: 86 - protein, 8 aa, Artificial Sequence
  Description of Artificial Sequence Synthetic peptide
GYTFTSYY

SEQ ID NO: 87 - protein, 8 aa, Artificial Sequence
  Description of Artificial Sequence Synthetic peptide
INPSGGST

SEQ ID NO: 88 - protein, 15 aa, Artificial Sequence
  Description of Artificial Sequence Synthetic peptide
ARRGWLSWGYYGMDV

SEQ ID NO: 89 - protein, 8 aa, Artificial Sequence
  Description of Artificial Sequence Synthetic peptide
GSTFINYG

SEQ ID NO: 90 - protein, 8 aa, Artificial Sequence
  Description of Artificial Sequence Synthetic peptide
VSGYNGNT

SEQ ID NO: 91 - protein, 10 aa, Artificial Sequence
  Description of Artificial Sequence Synthetic peptide
ARYYYPPYDY

SEQ ID NO: 92 - protein, 8 aa, Artificial Sequence
  Description of Artificial Sequence Synthetic peptide
GGTFSSYA

SEQ ID NO: 93 - protein, 8 aa, Artificial Sequence
  Description of Artificial Sequence Synthetic peptide
IIPIFGTA

SEQ ID NO: 94 - protein, 12 aa, Artificial Sequence
  Description of Artificial Sequence Synthetic peptide
ARGFSSWPGIDQ

SEQ ID NO: 95 - protein, 8 aa, Artificial Sequence
  Description of Artificial Sequence Synthetic peptide
GYSFTTYW

SEQ ID NO: 96 - protein, 8 aa, Artificial Sequence
  Description of Artificial Sequence Synthetic peptide
IYPGDSDT

SEQ ID NO: 97 - protein, 15 aa, Artificial Sequence
  Description of Artificial Sequence Synthetic peptide
ARYGGQYFWSDSFDS

SEQ ID NO: 98 - protein, 6 aa, Artificial Sequence
  Description of Artificial Sequence Synthetic peptide
QGIRND

SEQ ID NO: 99 - protein, 3 aa, Artificial Sequence
  Description of Artificial Sequence Synthetic peptide
AAS

SEQ ID NO: 100 - protein, 9 aa, Artificial Sequence
  Description of Artificial Sequence Synthetic peptide
LQDYDYPLT

SEQ ID NO: 101 - protein, 8 aa, Artificial Sequence
  Description of Artificial Sequence Synthetic peptide
SSNIGSNY

SEQ ID NO: 102 - protein, 3 aa, Artificial Sequence
  Description of Artificial Sequence Synthetic peptide
DNT

SEQ ID NO: 103 - protein, 11 aa, Artificial Sequence
  Description of Artificial Sequence Synthetic peptide
ETLDSSLSAYV

SEQ ID NO: 104 - protein, 8 aa, Artificial Sequence
  Description of Artificial Sequence Synthetic peptide
RSNIGSNF

SEQ ID NO: 105 - protein, 3 aa, Artificial Sequence
  Description of Artificial Sequence Synthetic peptide
DNN

SEQ ID NO: 106 - protein, 11 aa, Artificial Sequence
  Description of Artificial Sequence Synthetic peptide
GTWDSSLIAGV

SEQ ID NO: 107 - protein, 8 aa, Artificial Sequence
  Description of Artificial Sequence Synthetic peptide
SSNIGSNY

SEQ ID NO: 108 - protein, 3 aa, Artificial Sequence
  Description of Artificial Sequence Synthetic peptide
DDN

SEQ ID NO: 109 - protein, 9 aa, Artificial Sequence
  Description of Artificial Sequence Synthetic peptide
GTWDSPLVA

SEQ ID NO: 110 - protein, 8 aa, Artificial Sequence
  Description of Artificial Sequence Synthetic peptide
TSDIGSNY

SEQ ID NO: 111 - protein, 3 aa, Artificial Sequence
  Description of Artificial Sequence Synthetic peptide
DNN

SEQ ID NO: 112 - protein, 11 aa, Artificial Sequence
  Description of Artificial Sequence Synthetic peptide
GTWDSSLNTFV

SEQ ID NO: 113 - protein, 9 aa, Artificial Sequence
  Description of Artificial Sequence Synthetic peptide
SSNIGAGYD

SEQ ID NO: 114 - protein, 3 aa, Artificial Sequence
  Description of Artificial Sequence Synthetic peptide
GNV

SEQ ID NO: 115 - protein, 11 aa, Artificial Sequence
  Description of Artificial Sequence Synthetic peptide
QSYDSSLSGWV

SEQ ID NO: 116 - protein, 8 aa, Artificial Sequence
  Description of Artificial Sequence Synthetic peptide
SSNIGSNY

SEQ ID NO: 117 - protein, 3 aa, Artificial Sequence
  Description of Artificial Sequence Synthetic peptide
DNN

SEQ ID NO: 118 - protein, 11 aa, Artificial Sequence
  Description of Artificial Sequence Synthetic peptide
GSWDSSLSVYV

SEQ ID NO: 119 - protein, 8 aa, Artificial Sequence
  Description of Artificial Sequence Synthetic peptide
SSNIGSNY

SEQ ID NO: 120 - protein, 3 aa, Artificial Sequence
  Description of Artificial Sequence Synthetic peptide
DNS

SEQ ID NO: 121 - protein, 11 aa, Artificial Sequence
  Description of Artificial Sequence Synthetic peptide
GTWDSSLSAYV

SEQ ID NO: 122 - protein, 8 aa, Artificial Sequence
  Description of Artificial Sequence Synthetic peptide
SSNIGSNY

SEQ ID NO: 123 - protein, 3 aa, Artificial Sequence
  Description of Artificial Sequence Synthetic peptide
DNN

SEQ ID NO: 124 - protein, 11 aa, Artificial Sequence
  Description of Artificial Sequence Synthetic peptide
GTWDSSLSAYV

SEQ ID NO: 125 - protein, 8 aa, Artificial Sequence
  Description of Artificial Sequence Synthetic peptide
SGSIASNY

SEQ ID NO: 126 - protein, 3 aa, Artificial Sequence
  Description of Artificial Sequence Synthetic peptide
EDN

SEQ ID NO: 127 - protein, 9 aa, Artificial Sequence
  Description of Artificial Sequence Synthetic peptide
QSYDSSNVV

SEQ ID NO: 128 - protein, 8 aa, Artificial Sequence
  Description of Artificial Sequence Synthetic peptide
RSNIVSNY

SEQ ID NO: 129 - protein, 3 aa, Artificial Sequence
  Description of Artificial Sequence Synthetic peptide
DNN

SEQ ID NO: 130 - protein, 11 aa, Artificial Sequence
  Description of Artificial Sequence Synthetic peptide
GTWDSSLSAYV

SEQ ID NO: 131 - protein, 8 aa, Artificial Sequence
  Description of Artificial Sequence Synthetic peptide
SSNIGSNY

SEQ ID NO: 132 - protein, 3 aa, Artificial Sequence
  Description of Artificial Sequence Synthetic peptide
DNN

SEQ ID NO: 133 - protein, 11 aa, Artificial Sequence
  Description of Artificial Sequence Synthetic peptide
GIWDSSLSAYV

SEQ ID NO: 134 - protein, 8 aa, Artificial Sequence
  Description of Artificial Sequence Synthetic peptide
TSNIGSNY

SEQ ID NO: 135 - protein, 3 aa, Artificial Sequence
  Description of Artificial Sequence Synthetic peptide
DSD

SEQ ID NO: 136 - protein, 11 aa, Artificial Sequence
  Description of Artificial Sequence Synthetic peptide
GTWDSSLTVGV

SEQ ID NO: 137 - protein, 8 aa, Artificial Sequence
  Description of Artificial Sequence Synthetic peptide
SSNIGSNY

SEQ ID NO: 138 - protein, 3 aa, Artificial Sequence
  Description of Artificial Sequence Synthetic peptide
DNN

SEQ ID NO: 139 - protein, 11 aa, Artificial Sequence
  Description of Artificial Sequence Synthetic peptide
GTWDSSLTAGV

SEQ ID NO: 140 - protein, 6 aa, Artificial Sequence
  Description of Artificial Sequence Synthetic peptide
KLGDKY

SEQ ID NO: 141 - protein, 3 aa, Artificial Sequence
  Description of Artificial Sequence Synthetic peptide
QDS

SEQ ID NO: 142 - protein, 10 aa, Artificial Sequence
  Description of Artificial Sequence Synthetic peptide
QAWDSSTAYV

SEQ ID NO: 143 - protein, 6 aa, Artificial Sequence
  Description of Artificial Sequence Synthetic peptide
TLRSYY

SEQ ID NO: 144 - protein, 3 aa, Artificial Sequence
  Description of Artificial Sequence Synthetic peptide
GRN

SEQ ID NO: 145 - protein, 9 aa, Artificial Sequence
  Description of Artificial Sequence Synthetic peptide
QSFDYSYSV

SEQ ID NO: 146 - protein, 8 aa, Artificial Sequence
  Description of Artificial Sequence Synthetic peptide
SSNIGNNY

SEQ ID NO: 147 - protein, 3 aa, Artificial Sequence
  Description of Artificial Sequence Synthetic peptide
DNN

SEQ ID NO: 148 - protein, 11 aa, Artificial Sequence
  Description of Artificial Sequence Synthetic peptide
QSYDVYNMTSV

SEQ ID NO: 149 - protein, 8 aa, Artificial Sequence
  Description of Artificial Sequence Synthetic peptide
SGSVASEF

SEQ ID NO: 150 - protein, 3 aa, Artificial Sequence
  Description of Artificial Sequence Synthetic peptide
NDF

SEQ ID NO: 151 - protein, 10 aa, Artificial Sequence
  Description of Artificial Sequence Synthetic peptide
QSYDQSSSIV

SEQ ID NO: 152 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 153 - protein, 330 aa, Homo sapiens [sequence omitted]

SEQ ID NO: 154 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 155 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 156 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 157 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 159 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 160 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 162 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 163 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 164 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 165 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 166 - protein, 9 aa, Homo sapiens
ALNEKLVNL

SEQ ID NO: 167 - protein, 9 aa, Homo sapiens
ALNELLQHV

SEQ ID NO: 168 - protein, 9 aa, Homo sapiens
MLANDIARL

SEQ ID NO: 169 - protein, 9 aa, Homo sapiens
TLADIIARL

SEQ ID NO: 170 - protein, 9 aa, Influenza A virus
GILGFVFTL

SEQ ID NO: 171 - protein, 9 aa, Homo sapiens
GLNEEIARV

SEQ ID NO: 172 - protein, 9 aa, Homo sapiens
HLEEQIAKV

SEQ ID NO: 173 - protein, 9 aa, Homo sapiens
KVLERVNAV

SEQ ID NO: 174 - protein, 9 aa, Homo sapiens
RLAEAHAKV

SEQ ID NO: 175 - protein, 11 aa, Homo sapiens
ALNEEAGRLLL

SEQ ID NO: 176 - protein, 9 aa, Homo sapiens
YLDEYIARM

SEQ ID NO: 177 - protein, 9 aa, Homo sapiens
LLFEGIARI

SEQ ID NO: 178 - protein, 9 aa, Unknown
  Description of Unknown Ewing sarcoma-derived peptide sequence
QLQNPSYDK

SEQ ID NO: 179 - protein, 9 aa, Artificial Sequence
  Description of Artificial Sequence Synthetic peptide
GLNEQIARL

SEQ ID NO: 180 - protein, 9 aa, Artificial Sequence
  Description of Artificial Sequence Synthetic peptide
AANEQIARL

SEQ ID NO: 181 - protein, 9 aa, Artificial Sequence
  Description of Artificial Sequence Synthetic peptide
ALAEQIARL

SEQ ID NO: 182 - protein, 9 aa, Artificial Sequence
  Description of Artificial Sequence Synthetic peptide
ALNAQIARL

SEQ ID NO: 183 - protein, 9 aa, Artificial Sequence
  Description of Artificial Sequence Synthetic peptide
ALNEAIARL

SEQ ID NO: 184 - protein, 9 aa, Artificial Sequence
  Description of Artificial Sequence Synthetic peptide
ALNEQAARL

SEQ ID NO: 185 - protein, 9 aa, Artificial Sequence
  Description of Artificial Sequence Synthetic peptide
ALNEQIGRL

SEQ ID NO: 186 - protein, 9 aa, Artificial Sequence
  Description of Artificial Sequence Synthetic peptide
ALNEQIAAL

SEQ ID NO: 187 - protein, 9 aa, Artificial Sequence
  Description of Artificial Sequence Synthetic peptide
ALNEQIARA

(19) United States
(12) Patent Application Publication
(10) Pub. No.: US 2023/0374140 A1
(43) Pub. Date: Nov. 23, 2023

(54) COMPOSITIONS TARGETING NDC80/MHC COMPLEXES AND USES THEREOF

(71) Applicants: MEMORIAL SLOAN KETTERING CANCER CENTER, New York, NY (US); EUREKA THERAPEUTICS INC., Emeryville, CA (US)

(72) Inventors: Martin KLATT, New York, NY (US); Tao DAO, New York, NY (US); David A. SCHEINBERG, New York, NY (US); Zhiyuan YANG, Emeryville, CA (US); Jianying LIU, Emeryville, CA (US)

(21) Appl. No.: 18/247,864

(22) PCT Filed: Oct. 8, 2021
(86) PCT No.: PCT/US2021/054123

§ 371 Date: Apr. 4, 2023

Related U.S. Application Data
(60) Provisional application No. 63/089,754, filed on Oct. 9, 2020.
(60) Provisional application No. 63/188,766, filed on May 14, 2021.

(51) Int. Cl. C07K 16/28

(52) U.S. Cl. CPC C07K 16/2833; C07K 2317/32; C07K 2317/34; C07K 2317/622; A61K 2039/505

(57) ABSTRACT

The present technology relates generally to compositions that specifically recognize and bind to a NDC80 peptide complexed with a major histocompatibility antigen (e.g., HLA-A*02). The compositions of the present technology are useful in methods for treating NDC80-associated diseases (e.g., cancers) in a subject in need thereof.

### CROSS-REFERENCE TO RELATED APPLICATIONS

This application is a U.S. National Stage Application under 35 U.S.C. § 371 of International Patent Application No. PCT/US2021/054123, filed Oct. 8, 2021, which claims the benefit of and priority to U.S. Provisional Patent Application No. 63/089,754, filed Oct. 9, 2020, and U.S. Provisional Patent Application No. 63/188,766, filed May 14, 2021, the entire contents of which are incorporated herein by reference.

### STATEMENT OF GOVERNMENT INTEREST

This invention was made with government support under CA055349, awarded by the National Institutes of Health. The government has certain rights in the invention.

### TECHNICAL FIELD

The present disclosure provides compositions that specifically bind to kinetochore NDC80 protein homolog (NDC80) peptide complexed with MHC, including antibodies such as human, humanized, or chimeric antibodies, antibody fragments, chimeric antibody-T cell receptors (caTCRs), chimeric antigen receptors (CARs), fusion proteins, and conjugates thereof. The compositions of the present technology bind to HLA-A*02-restricted NDC80 peptides and are useful for the treatment of NDC80-associated diseases, including but not limited to cancers.

### SEQUENCE LISTING

The instant application contains a Sequence Listing which has been submitted electronically in ASCII format and is hereby incorporated by reference in its entirety. Said ASCII copy, created on Nov. 24, 2021, is named 115872-2313_SL.txt and is 113,568 bytes in size.

### BACKGROUND

The following description of the background of the present technology is provided simply as an aid in understanding the present technology and is not admitted to describe or constitute prior art to the present technology.

Chimeric antigen receptor (CAR) T cells represent a class of FDA-approved drugs with high efficacy against refractory B cell derived malignancies and potentially other cancer types. However, target selection for CAR T cell therapy remains challenging as cell surface proteins are mostly not cancer-specific and therefore often not adaptable for CAR T cell therapy. In contrast, many intracellular proteins are highly tumor specific and therefore are better targets for cancer immunotherapies. Intracellular proteins are targetable after proteasomal degradation, resulting in short peptides, and presentation on MHC (whose human form being human leukocyte antigen, or HLA) complexes.

The peptide-MHC complexes are displayed at the cell surface where they provide targets for T cell recognition via a peptide-MHC (pMHC)-T cell receptor (TCR) interaction in natural circumstances. Antibodies against such targets (i.e., TCR-mimic antibodies) may be desirable, especially when such targets do not have effective TCR therapies against them. Soluble TCRs have been shown to be difficult to engineer in vitro, and they inherently have lower affinities than antibodies, which may limit their use as therapies. See He et al., Journal of Hematology & Oncology (2019) 12:99. TCR-mimic antibodies, if identified, may be useful in CAR T cell therapies or other cellular immunotherapies against diseases that do not have good cell-surface antigens, including most solid tumors and viral infections.

In addition, cancer antigens can be targeted with monoclonal antibody therapies or other humoral immunotherapies. Monoclonal antibody (mAb) therapies have been shown to exert powerful antitumor effects by multiple mechanisms, including complement-dependent cytotoxicity (CDC), antibody-dependent cellular cytotoxicity (ADCC) and direct cell inhibition or apoptosis-inducing effects on tumor cells that over-express the target molecules. Furthermore, mAb can be used as carriers to specifically deliver a cytotoxic moiety such as a radionuclide, cytotoxic drug or toxin to the tumor cells. Other humoral immunotherapies or immunotherapy candidates include bispecific antibodies that are composed of a cancer-antigen-binding antibody moiety and a T-cell-recruiting antibody moiety.

The outer kinetochore Ndc80 complex, is essential for both microtubule binding and spindle assembly checkpoint signaling (a cell cycle surveillance pathway that delays exit from mitosis). The intracellular protein NDC80 is one of four components of the NDC80 tetrameric complex, and is highly expressed in a variety of human cancers. Overexpression of NDC80 was determined to be associated with poor clinical prognosis in breast cancer and other cancers (van't Veer L J et al., Nature 415:530-536 (2002); Glinsky et al., J Clin Invest. 115:1503-1521 (2005)). These observations highlight a crucial role of NDC80 in tumorigenesis and emerge as a potential mitotic target for cancer intervention. Accordingly, compositions that target NDC80/MHC complexes, for example, a NDC80 peptide/HLA-A*02 complex, would be useful as an effective therapeutic agent alone or as a vehicle capable of delivering potent anti-cancer reagents, such as drugs, toxins and radioactive elements.

### SUMMARY OF THE PRESENT TECHNOLOGY

The present disclosure identifies and characterizes immunoglobulin-related compositions (e.g., antibodies including human, humanized, or chimeric antibodies, antibody fragments, chimeric antibody-T cell receptors (caTCRs), chimeric antigen receptors (CARs), fusion proteins, and conjugates thereof) that are able to target cytosolic/intracellular proteins, for example, NDC80. The disclosed immunoglobulin-related compositions target a peptide/MHC complex as it would typically appear on the surface of a cell following antigen processing of NDC80 protein and presentation by the cell. In that regard, the immunoglobulin-related compositions mimic T-cell receptors in that the immunoglobulin-related compositions have the ability to specifically recognize and bind to a peptide in an MHC-restricted fashion, that is, when the peptide is bound to an MHC antigen. The peptide/MHC complex recapitulates the antigen as it would typically appear on the surface of a cell following antigen processing of the NDC80 protein, which in turn is presented to a T-cell. The immunoglobulin-related compositions disclosed herein specifically recognize and bind to epitopes of a peptide/HLA-A*02 complex, particularly a NDC80/HLA-A*02 complex. Examples of peptides that are recognized by the immunoglobulin-related compositions of the present disclosure as part of an HLA-peptide complex include, for example, a peptide with the amino acid sequence ALNEQIARL (SEQ ID NO: 1).

In one aspect, the present disclosure provides compositions such as antigen binding proteins or immunoglobulin-related compositions comprising antibody moieties that specifically bind to NDC80 peptide/MHC complexes (also called “anti-NDC80 peptide/MHC” or “anti-NDC80/MHC” herein). Such compositions can comprise, consist essentially of, or consist of, e.g., anti-NDC80 peptide/MHC antibodies or antigen binding fragments thereof, chimeric antibody-T cell receptors (caTCRs), chimeric antigen receptors (CARs), fusion proteins, and conjugates. The antibody moieties can comprise: (a) a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 2, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 20, (b) a V_{H} comprising a V_{H}-CDR1 sequence, a V_{H}-CDR2 sequence, and a V_{H}-CDR3 sequence of the V_{H} sequence of SEQ ID NO: 3, and a V_{L} comprising a V_{L}-CDR1 sequence, a V_{L}-CDR2 sequence, and a V_{L}-CDR3 sequence of the V_{L} sequence SEQ ID NO: 21, (c) a V_{H} comprising a V_{H}-CDR1 sequence, a V_{H}-CDR2 sequence, and a V_{H}-CDR3 sequence of the V_{H} sequence of SEQ ID NO: 4, and a V_{L} comprising a V_{L}-CDR1 sequence, a V_{L}-CDR2 sequence, and a V_{L}-CDR3 sequence of the V_{L} sequence SEQ ID NO: 22, (d) a V_{H} comprising a V_{H}-CDR1 sequence, a V_{H}-CDR2 sequence, and a V_{H}-CDR3 sequence of the V_{H} sequence of SEQ ID NO: 5, and a V_{L} comprising a V_{L}-CDR1 sequence, a V_{L}-CDR2 sequence, and a V_{L}-CDR3 sequence of the V_{L} sequence SEQ ID NO: 23, (e) a V_{H} comprising a V_{H}-CDR1 sequence, a V_{H}-CDR2 sequence, and a V_{H}-CDR3 sequence of the V_{H} sequence of SEQ ID NO: 6, and a V_{L} comprising a V_{L}-CDR1 sequence, a V_{L}-CDR2 sequence, and a V_{L}-CDR3 sequence of the V_{L} sequence SEQ ID NO: 24, (f) a V_{H} comprising a V_{H}-CDR1 sequence, a V_{H}-CDR2 sequence, and a V_{H}-CDR3 sequence of the V_{H} sequence of SEQ ID NO: 7, and a V_{L} comprising a V_{L}-CDR1 sequence, a V_{L}-CDR2 sequence, and a V_{L}-CDR3 sequence of the V_{L} sequence SEQ ID NO: 25, (g) a V_{H} comprising a V_{H}-CDR1 sequence, a V_{H}-CDR2 sequence, and a V_{H}-CDR3 sequence of the V_{H} sequence of SEQ ID NO: 8, and a V_{L} comprising a V_{L}-CDR1 sequence, a V_{L}-CDR2 sequence, and a V_{L}-CDR3 sequence of the V_{L} sequence SEQ ID NO: 26, (h) a V_{H} comprising a V_{H}-CDR1 sequence, a V_{H}-CDR2 sequence, and a V_{H}-CDR3 sequence of the V_{H} sequence of SEQ ID NO: 9, and a V_{L} comprising a V_{L}-CDR1 sequence, a V_{L}-CDR2 sequence, and a V_{L}-CDR3 sequence of the V_{L} sequence SEQ ID NO: 27, (i) a V_{H} comprising a V_{H}-CDR1 sequence, a V_{H}-CDR2 sequence, and a V_{H}-CDR3 sequence of the V_{H} sequence of SEQ ID NO: 10, and a V_{L} comprising a V_{L}-CDR1 sequence, a V_{L}-CDR2 sequence, and a V_{L}-CDR3 sequence of the V_{L} sequence SEQ ID NO: 28, (j) a V_{H} comprising a V_{H}-CDR1 sequence, a V_{H}-CDR2 sequence, and a V_{H}-CDR3 sequence of the V_{H} sequence of SEQ ID NO: 11, and a V_{L} comprising a V_{L}-CDR1 sequence, a V_{L}-CDR2 sequence, and a V_{L}-CDR3 sequence of the V_{L} sequence SEQ ID NO: 29, (k) a V_{H} comprising a V_{H}-CDR1 sequence, a V_{H}-CDR2 sequence, and a V_{H}-CDR3 sequence of the V_{H} sequence of SEQ ID NO: 12, and a V_{L} comprising a V_{L}-CDR1 sequence, a V_{L}-CDR2 sequence, and a V_{L}-CDR3 sequence of the V_{L} sequence SEQ ID NO: 30, (1) a V_{H} comprising a V_{H}-CDR1 sequence, a V_{H}-CDR2 sequence, and a V_{H}-CDR3 sequence of the V_{H} sequence of SEQ ID NO: 13, and a V_{L} comprising a V_{L}-CDR1 sequence, a V_{L}-CDR2 sequence, and a V_{L}-CDR3 sequence of the V_{L} sequence SEQ ID NO: 31, (m) a V_{H} comprising a V_{H}-CDR1 sequence, a V_{H}-CDR2 sequence, and a V_{H}-CDR3 sequence of the V_{H} sequence of SEQ ID NO: 14, and a V_{L} comprising a V_{L}-CDR1 sequence, a V_{L}-CDR2 sequence, and a V_{L}-CDR3 sequence of the V_{L} sequence SEQ ID NO: 32, (n) a V_{H} comprising a V_{H}-CDR1 sequence, a V_{H}-CDR2 sequence, and a V_{H}-CDR3 sequence of the V_{H} sequence of SEQ ID NO: 15, and a V_{L} comprising a V_{L}-CDR1 sequence, a V_{L}-CDR2 sequence, and a V_{L}-CDR3 sequence of the V_{L} sequence SEQ ID NO: 33, (o) a V_{H} comprising a V_{H}-CDR1 sequence, a V_{H}-CDR2 sequence, and a V_{H}-CDR3 sequence of the V_{H} sequence of SEQ ID NO: 16, and a V_{L} comprising a V_{L}-CDR1 sequence, a V_{L}-CDR2 sequence, and a V_{L}-CDR3 sequence of the V_{L} sequence SEQ ID NO: 34, (p) a V_{H} comprising a V_{H}-CDR1 sequence, a V_{H}-CDR2 sequence, and a V_{H}-CDR3 sequence of the V_{H} sequence of SEQ ID NO: 17, and a V_{L} comprising a V_{L}-CDR1 sequence, a V_{L}-CDR2 sequence, and a V_{L}-CDR3 sequence of the V_{L} sequence SEQ ID NO: 35, (q) a V_{H} comprising a V_{H}-CDR1 sequence, a V_{H}-CDR2 sequence, and a V_{H}-CDR3 sequence of the V_{H} sequence of SEQ ID NO: 18, and a V_{L} comprising a V_{L}-CDR1 sequence, a V_{L}-CDR2 sequence, and a V_{L}-CDR3 sequence of the V_{L} sequence SEQ ID NO: 36, or (r) a V_{H} comprising a V_{H}-CDR1 sequence, a V_{H}-CDR2 sequence, and a V_{H}-CDR3 sequence of the V_{H} sequence of SEQ ID NO: 19, and a V_{L} comprising a V_{L}-CDR1 sequence, a V_{L}-CDR2 sequence, and a V_{L}-CDR3 sequence of the V_{L} sequence SEQ ID NO: 37. The antibody moiety may be a full-length antibody, a Fab, a F(ab′)₂, a Fab′, a Fᵥ, or a single chain Fv (scFv).

In some embodiments of the antigen binding proteins or immunoglobulin-related compositions disclosed herein, the (a) the V_{H}-CDR1 sequence comprises the sequence of SEQ ID NO: 44, the V_{H}-CDR2 sequence comprises the sequence of SEQ ID NO: 45, the V_{H}-CDR3 sequence comprises the sequence of SEQ ID NO: 46, the V_{L}-CDR1 sequence comprises the sequence of SEQ ID NO: 98, the V_{L}-CDR2 sequence comprises the sequence of SEQ ID NO: 99, and/or the V_{L}-CDR3 sequence comprises the sequence of SEQ ID NO: 100; (b) the V_{H}-CDR1 sequence comprises the sequence of SEQ ID NO: 47, the V_{H}-CDR2 sequence comprises the sequence of SEQ ID NO: 48, the V_{H}-CDR3 sequence comprises the sequence of SEQ ID NO: 49, the V_{L}-CDR1 sequence comprises the sequence of SEQ ID NO: 101, the V_{L}-CDR2 sequence comprises the sequence of SEQ ID NO: 102, and/or the V_{L}-CDR3 sequence comprises the sequence of SEQ ID NO: 103; (c) the V_{H}-CDR1 sequence comprises the sequence of SEQ ID NO: 50, the V_{H}-CDR2 sequence comprises the sequence of SEQ ID NO: 51, the V_{H}-CDR3 sequence comprises the sequence of SEQ ID NO: 52, the V_{L}-CDR1 sequence comprises the sequence of SEQ ID NO: 104, the V_{L}-CDR2 sequence comprises the sequence of SEQ ID NO: 105, and/or the V_{L}-CDR3 sequence comprises the sequence of SEQ ID NO: 106; (d) the V_{H}-CDR1 sequence comprises the sequence of SEQ ID NO: 53, the V_{H}-CDR2 sequence comprises the sequence of SEQ ID NO: 54, the V_{H}-CDR3 sequence comprises the sequence of SEQ ID NO: 55, the V_{L}-CDR1 sequence comprises the sequence of SEQ ID NO: 107, the V_{L}-CDR2 sequence comprises the sequence of SEQ ID NO: 108, and/or the V_{L}-CDR3 sequence comprises the sequence of SEQ ID NO: 109; (e) the V_{H}-CDR1 sequence comprises the sequence of SEQ ID NO: 56, the V_{H}-CDR2 sequence comprises the sequence of SEQ ID NO: 57, the V_{H}-CDR3 sequence comprises the sequence of SEQ ID NO: 58, the V_{L}-CDR1 sequence comprises the sequence of SEQ ID NO: 110, the V_{L}-CDR2 sequence comprises the sequence of SEQ ID NO: 111, and/or the V_{L}-CDR3 sequence comprises the sequence of SEQ ID NO: 112; (f) the V_{H}-CDR1 sequence comprises the sequence of SEQ ID NO: 59, the V_{H}-CDR2 sequence comprises the sequence of SEQ ID NO: 60, the V_{H}-CDR3 sequence comprises the sequence of SEQ ID NO: 61, the V_{L}-CDR1 sequence comprises the sequence of SEQ ID NO: 113, the V_{L}-CDR2 sequence comprises the sequence of SEQ ID NO: 114, and/or the V_{L}-CDR3 sequence comprises the sequence of SEQ ID NO: 115; (g) the V_{H}-CDR1 sequence comprises the sequence of SEQ ID NO: 62, the V_{H}-CDR2 sequence comprises the sequence of SEQ ID NO: 63, the V_{H}-CDR3 sequence comprises the sequence of SEQ ID NO: 64, the V_{L}-CDR1 sequence comprises the sequence of SEQ ID NO: 116, the V_{L}-CDR2 sequence comprises the sequence of SEQ ID NO: 117, and/or the V_{L}-CDR3 sequence comprises the sequence of SEQ ID NO: 118; (h) the V_{H}-CDR1 sequence comprises the sequence of SEQ ID NO: 65, the V_{H}-CDR2 sequence comprises the sequence of SEQ ID NO: 66, the V_{H}-CDR3 sequence comprises the sequence of SEQ ID NO: 67, the V_{L}-CDR1 sequence comprises the sequence of SEQ ID NO: 119, the V_{L}-CDR2 sequence comprises the sequence of SEQ ID NO: 120, and/or the V_{L}-CDR3 sequence comprises the sequence of SEQ ID NO: 121; (i) the V_{H}-CDR1 sequence comprises the sequence of SEQ ID NO: 68, the V_{H}-CDR2 sequence comprises the sequence of SEQ ID NO: 69, the V_{H}-CDR3 sequence comprises the sequence of SEQ ID NO: 70, the V_{L}-CDR1 sequence comprises the sequence of SEQ ID NO: 122, the V_{L}-CDR2 sequence comprises the sequence of SEQ ID NO: 123, and/or the V_{L}-CDR3 sequence comprises the sequence of SEQ ID NO: 124; (j) the V_{H}-CDR1 sequence comprises the sequence of SEQ ID NO: 71, the V_{H}-CDR2 sequence comprises the sequence of SEQ ID NO: 72, the V_{H}-CDR3 sequence comprises the sequence of SEQ ID NO: 73, the V_{L}-CDR1 sequence comprises the sequence of SEQ ID NO: 125, the V_{L}-CDR2 sequence comprises the sequence of SEQ ID NO: 126, and/or the V_{L}-CDR3 sequence comprises the sequence of SEQ ID NO: 127; (k) the V_{H}-CDR1 sequence comprises the sequence of SEQ ID NO: 74, the V_{H}-CDR2 sequence comprises the sequence of SEQ ID NO: 75, the V_{H}-CDR3 sequence comprises the sequence of SEQ ID NO: 76, the V_{L}-CDR1 sequence comprises the sequence of SEQ ID NO: 128, the V_{L}-CDR2 sequence comprises the sequence of SEQ ID NO: 129, and/or the V_{L}-CDR3 sequence comprises the sequence of SEQ ID NO: 130; (1) the V_{H}-CDR1 sequence comprises the sequence of SEQ ID NO: 77, the V_{H}-CDR2 sequence comprises the sequence of SEQ ID NO: 78, the V_{H}-CDR3 sequence comprises the sequence of SEQ ID NO: 79, the V_{L}-CDR1 sequence comprises the sequence of SEQ ID NO: 131, the V_{L}-CDR2 sequence comprises the sequence of SEQ ID NO: 132, and/or the V_{L}-CDR3 sequence comprises the sequence of SEQ ID NO: 133; (m) the V_{H}-CDR1 sequence comprises the sequence of SEQ ID NO: 80, the V_{H}-CDR2 sequence comprises the sequence of SEQ ID NO: 81, the V_{H}-CDR3 sequence comprises the sequence of SEQ ID NO: 82, the V_{L}-CDR1 sequence comprises the sequence of SEQ ID NO: 134, the V_{L}-CDR2 sequence comprises the sequence of SEQ ID NO: 135, and/or the V_{L}-CDR3 sequence comprises the sequence of SEQ ID NO: 136; (n) the V_{H}-CDR1 sequence comprises the sequence of SEQ ID NO: 83, the V_{H}-CDR2 sequence comprises the sequence of SEQ ID NO: 84, the V_{H}-CDR3 sequence comprises the sequence of SEQ ID NO: 85, the V_{L}-CDR1 sequence comprises the sequence of SEQ ID NO: 137, the V_{L}-CDR2 sequence comprises the sequence of SEQ ID NO: 138, and/or the V_{L}-CDR3 sequence comprises the sequence of SEQ ID NO: 139; (o) the V_{H}-CDR1 sequence comprises the sequence of SEQ ID NO: 86, the V_{H}-CDR2 sequence comprises the sequence of SEQ ID NO: 87, the V_{H}-CDR3 sequence comprises the sequence of SEQ ID NO: 88, the V_{L}-CDR1 sequence comprises the sequence of SEQ ID NO: 140, the V_{L}-CDR2 sequence comprises the sequence of SEQ ID NO: 141, and/or the V_{L}-CDR3 sequence comprises the sequence of SEQ ID NO: 142; (p) the V_{H}-CDR1 sequence comprises the sequence of SEQ ID NO: 89, the V_{H}-CDR2 sequence comprises the sequence of SEQ ID NO: 90, the V_{H}-CDR3 sequence comprises the sequence of SEQ ID NO: 91, the V_{L}-CDR1 sequence comprises the sequence of SEQ ID NO: 143, the V_{L}-CDR2 sequence comprises the sequence of SEQ ID NO: 144, and/or the V_{L}-CDR3 sequence comprises the sequence of SEQ ID NO: 145; (q) the V_{H}-CDR1 sequence comprises the sequence of SEQ ID NO: 92, the V_{H}-CDR2 sequence comprises the sequence of SEQ ID NO: 93, the V_{H}-CDR3 sequence comprises the sequence of SEQ ID NO: 94, the V_{L}-CDR1 sequence comprises the sequence of SEQ ID NO: 146, the V_{L}-CDR2 sequence comprises the sequence of SEQ ID NO: 147, and/or the V_{L}-CDR3 sequence comprises the sequence of SEQ ID NO: 148; or (r) the V_{H}-CDR1 sequence comprises the sequence of SEQ ID NO: 95, the V_{H}-CDR2 sequence comprises the sequence of SEQ ID NO: 96, the V_{H}-CDR3 sequence comprises the sequence of SEQ ID NO: 97, the V_{L}-CDR1 sequence comprises the sequence of SEQ ID NO: 149, the V_{L}-CDR2 sequence comprises the sequence of SEQ ID NO: 150, and/or the V_{L}-CDR3 sequence comprises the sequence of SEQ ID NO: 151.

Additionally or alternatively, in some embodiments of the antigen binding proteins or immunoglobulin-related compositions disclosed herein, (a) the V_{H} comprises an amino acid sequence having at least 90% identity to a sequence selected from the group consisting of: SEQ ID NOs: 2-19, and/or (b) the V_{L} comprises an amino acid sequence having at least 90% identity to a sequence selected from the group consisting of: SEQ ID NOs: 20-37. In certain embodiments, (a) the V_{H} comprises an amino acid sequence selected from the group consisting of: SEQ ID NOs: 2-19 or a variant thereof having one or more conservative amino acid substitutions; and/or (b) the V_{L} comprises an amino acid sequence selected from the group consisting of: SEQ ID NOs: 20-37 or a variant thereof having one or more conservative amino acid substitutions.

Additionally or alternatively, in some embodiments, the antigen binding proteins or immunoglobulin-related compositions of the present technology comprise a V_{H} amino acid sequence and a V_{L} amino acid sequence selected from the group consisting of: SEQ ID NO: 2 and SEQ ID NO: 20; SEQ ID NO: 3 and SEQ ID NO: 21; SEQ ID NO: 4 and SEQ ID NO: 22; SEQ ID NO: 5 and SEQ ID NO: 23; SEQ ID NO: 6 and SEQ ID NO: 24; SEQ ID NO: 7 and SEQ ID NO: 25; SEQ ID NO: 8 and SEQ ID NO: 26; SEQ ID NO: 9 and SEQ ID NO: 27; SEQ ID NO: 10 and SEQ ID NO: 28; SEQ ID NO: 11 and SEQ ID NO: 29; SEQ ID NO: 12 and SEQ ID NO: 30; SEQ ID NO: 13 and SEQ ID NO: 31; SEQ ID NO: 14 and SEQ ID NO: 32; SEQ ID NO: 15 and SEQ ID NO: 33; SEQ ID NO: 16 and SEQ ID NO: 34; SEQ ID NO: 17 and SEQ ID NO: 35; SEQ ID NO: 18 and SEQ ID NO: 36; and SEQ ID NO: 19 and SEQ ID NO: 37.

Additionally or alternatively, in some embodiments, the antigen binding proteins or immunoglobulin-related compositions of the present technology comprise an amino acid sequence having at least 90% identity to a sequence selected from the group consisting of: SEQ ID NOs: 38-43. In certain embodiments, the antigen binding proteins or immunoglobulin-related compositions of the present technology comprise an amino acid sequence selected from the group consisting of: SEQ ID NOs: 38-43.

In any of the preceding embodiments, the antigen binding proteins or immunoglobulin-related compositions of the present technology further comprise a Fc domain of an isotype selected from the group consisting of IgG1, IgG2, IgG3, IgG4, IgA1, IgA2, IgM, IgD, and IgE.

Additionally or alternatively, in some embodiments, the antigen binding proteins or immunoglobulin-related compositions of the present technology are chimeric antibody-T cell receptors (caTCR) and/or comprise at least a fragment of a T cell receptor (TCR) chain. In some embodiments, the fragment of TCR chain comprises the transmembrane domain of the TCR chain. In certain embodiments, the fragment of TCR chain does not comprise any CDR sequence of the TCR chain. Additionally or alternatively, in some embodiments, the antigen binding proteins or immunoglobulin-related compositions of the present technology are chimeric antigen receptors (CARs).

In any and all of the preceding embodiments, the antigen binding proteins or immunoglobulin-related compositions of the present technology may be monospecific, multispecific, or bispecific. In some embodiments, the antigen binding proteins or immunoglobulin-related compositions of the present technology comprise a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. Additionally or alternatively, in some embodiments, the antigen binding proteins or immunoglobulin-related compositions of the present technology comprise a tandem scFv with at least one peptide linker between two scFvs. In certain embodiments, the antigen binding proteins or immunoglobulin-related compositions of the present technology comprise a second antibody moiety that specifically binds to a second antigen. The second antigen may be a disease-specific antigen that is not NDC80/MHC, or an antigen on the surface of a T cell, a natural killer cell, a neutrophil, a monocyte, a macrophage, or a dendritic cell.

In any and all of the preceding embodiments, the antigen binding proteins or immunoglobulin-related compositions of the present technology may be a monoclonal antibody, a chimeric antibody, a humanized antibody, or a human antibody. In some embodiments, the antigen binding protein or immunoglobulin-related composition of the present technology is a fully human antibody. Additionally or alternatively, in some embodiments, the antigen binding proteins or immunoglobulin-related compositions of the present technology may be an immunoglobulin polypeptide, or an immunoglobulin-like polypeptide.

Additionally or alternatively, in some embodiments, the antigen binding proteins or immunoglobulin-related compositions of the present technology specifically bind to a NDC80 peptide complexed with HLA-A*02. The HLA-A*02 may be HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50. In certain embodiments, the NDC80 peptide comprises the amino acid sequence ALNEQIARL (SEQ ID NO: 1).

In one aspect, the present disclosure provides an anti-NDC80/MHC composition comprising an antibody moiety that competes with the any of the antigen binding proteins or immunoglobulin-related compositions of the present technology for specific binding to a NDC80/MHC complex.

In one aspect, the present disclosure provides recombinant nucleic acids or a set of recombinant nucleic acids encoding any and all embodiments of the antigen binding proteins or immunoglobulin-related compositions described herein, with all components of the composition encoded by one nucleic acid or by the set of nucleic acids. In another aspect, the present disclosure provides a vector comprising said recombinant nucleic acids, as well as a set of vectors comprising said set of recombinant nucleic acids. Also disclosed herein are cells comprising any of the recombinant nucleic acids, set of recombinant nucleic acids, vectors, or set of vectors disclosed herein, as well as cells that display on its surface or secrete any of the antigen binding proteins or immunoglobulin-related compositions of the present technology. The cells may be a T cell, a NK cell, a B cell, or a monocyte/macrophage.

In one aspect, the present disclosure provides a pharmaceutical compositions comprising the antigen binding proteins or immunoglobulin-related compositions of the present technology, as well as any of the recombinant nucleic acids, set of recombinant nucleic acids, vectors, set of vectors, or cells disclosed herein, together with a pharmaceutically acceptable carrier.

Additionally or alternatively, in some embodiments, the antigen binding proteins or immunoglobulin-related compositions of the present technology are conjugated to an agent selected from the group consisting of detectable label, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In one aspect, the present disclosure provides a method for detecting NDC80 expression levels in a biological sample comprising (a) contacting the biological sample with any of the antigen binding proteins or immunoglobulin-related compositions of the present technology; and (b) detecting binding to a NDC80 peptide-HLA-A*02 complex in the biological sample. In some embodiments, the NDC80 peptide comprises the amino acid sequence ALNEQIARL (SEQ ID NO: 1).

In another aspect, the present disclosure provides methods for treating a NDC80-associated disease in a subject in need thereof, comprising administering to the subject an effective amount of any of the antigen binding proteins or immunoglobulin-related compositions disclosed herein, or any of the recombinant nucleic acids, set of recombinant nucleic acids, vectors, set of vectors, or cells disclosed herein, or any of the pharmaceutical compositions disclosed herein.

In one aspect, the present disclosure provides a method for treating a NDC80-associated disease in a subject in need thereof, comprising administering to the subject an effective amount of a composition comprising an antibody moiety that specifically binds to a NDC80 peptide/HLA-A*02 complex.

In another aspect, the present disclosure provides a method for treating a NDC80-associated disease in a subject in need thereof, comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

In yet another aspect, the present disclosure provides a method for treating a NDC80-associated disease in a subject in need thereof, comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a). In certain embodiments, the cell of (c) or (d) is a T cell, a NK cell, a B cell, or a monocyte/macrophage.

Additionally or alternatively, in some embodiments of the methods disclosed herein, the NDC80 peptide comprises the amino acid sequence ALNEQIARL (SEQ ID NO: 1).

In any and all embodiments of the methods disclosed herein, said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Additionally or alternatively, in some embodiments of the methods disclosed herein, the NDC80-associated disease is a cancer. Examples of cancer include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02.

In one aspect, the present disclosure provides a method for mitigating immunotherapy-related toxicity in a subject in need thereof comprising administering to the subject an effective amount of any of the antigen binding proteins or immunoglobulin-related compositions disclosed herein, or any of the recombinant nucleic acids, set of recombinant nucleic acids, vectors, set of vectors, or cells disclosed herein, or any of the pharmaceutical compositions disclosed herein.

In one aspect, the present disclosure provides a method for mitigating immunotherapy-related toxicity in a subject in need thereof comprising administering to the subject an effective amount of a composition comprising an antibody moiety that specifically binds to a NDC80 peptide/HLA-A*02 complex.

In another aspect, the present disclosure provides a method for mitigating immunotherapy-related toxicity in a subject in need thereof comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

In yet another aspect, the present disclosure provides a method for mitigating immunotherapy-related toxicity in a subject in need thereof comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a). In certain embodiments, the cell of (c) or (d) is a T cell, a NK cell, a B cell, or a monocyte/macrophage.

Additionally or alternatively, in some embodiments of the methods disclosed herein, the NDC80 peptide comprises the amino acid sequence ALNEQIARL (SEQ ID NO: 1). In any and all embodiments of the methods disclosed herein, said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Additionally or alternatively, in some embodiments of the methods disclosed herein, the subject has been diagnosed with or is suffering from a NDC80-associated disease, such as cancer. Examples of cancer include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02.

Additionally or alternatively, in some embodiments of the methods disclosed herein, the immunotherapy-related toxicity is selected from the group consisting of T-cell fratricide, hematopoietic stem cell toxicity, peripheral blood mononuclear cell (PBMC) toxicity, cardiomyocyte toxicity, cardiac fibroblast toxicity, and thymic fibroblast toxicity.

Additionally or alternatively, in some embodiments, the methods of the present technology further comprise separately, sequentially or simultaneously administering at least one additional therapeutic agent to the subject. Examples of additional therapeutic agents include, but are not limited to alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, immune checkpoint inhibitors, monoclonal antibodies that specifically target tumor antigens, T-cell therapy, immune activating agents, oncolytic virus therapy and cancer vaccines.

Also disclosed herein are kits comprising any of the antigen binding proteins or immunoglobulin-related compositions of the present technology and instructions for use. In some embodiments of the kits of the present technology, the antigen binding proteins or immunoglobulin-related compositions are coupled to at least one detectable label selected from the group consisting of a radioactive label, a fluorescent label, and a chromogenic label. Additionally or alternatively, in some embodiments, the kits further comprise a secondary antibody that specifically binds to the antigen binding proteins or immunoglobulin-related compositions of the present technology.

### BRIEF DESCRIPTION OF THE DRAWINGS

FIGS. 1A-1F show identification of the NDC80 derived ALNEQIARL (SEQ ID NO: 1) peptide as a tumor-associated HLA ligand. FIG. 1A shows the experimental strategy. FIG. 1A discloses SEQ ID NOS 1, 171-172 and 1, respectively, in order of appearance. FIG. 1B shows mean NDC80 expression levels in different cancer cell lines. Only cancer types with at least 5 data points were considered. Whiskers indicate min to max. FIG. 1C shows mean NDC80 expression of healthy tissues and corresponding cancer cell lines. ****p<0.0001 (Wilcoxon matched-pairs signed rank test). Error bars denote SD. FIG. 1D shows mean NDC80 expression of adjacent healthy tissues and corresponding primary cancer tissues. ****p<0.0001 (Wilcoxon matched-pairs signed rank test). Error bars denote SD. FIG. 1E shows ELISpot results from three healthy donors using two biological replicates per donor. Data were normalized to results from CD14 positive cells alone. EW served as control peptide. PHA is an immune-intolerant control. ***p<0.001 (Mann Whitney test). Error bars denote SD. FIG. 1F shows an overview of cell lines where HLA-A*02:01:ALNEQIARL (SEQ ID NO: 1) complex has been identified using mass spectrometry-based analysis.

FIG. 2 shows the transduction efficiency of 28z-myc-tagged lentiviral CAR T cell constructs using flow cytometry (Clones #1, #7, #11, #14, #18, #19, and a Foxp3 chimeric receptor as a transduction efficiency control).

FIGS. 3A-3G show the results of peptide-pulsed T2 cells using an LDH release assay with CAR T cells expressing Clones #1, #7, #11, #14, #18, and #19.

FIGS. 4A-4G show the 18 hour LDH release assay results with CAR T cells expressing Clones #1, #7, #11, #14, #18, and #19 in BV173, MCF7 and Raji cell lines. Clones 1 and 11 were selected for engineering into mouse IgG formats based on this data.

FIGS. 5A-5K show the 18 hour LDH release assay results with CAR T cells expressing Clones #1 and #11 in various indicated hematological cancer cell lines. HL60 is a negative control cell line lacking A02; target Peptide A was not detectable in SUDHL4 cells by mass spectrometry, most likely due to low A02 levels.

FIGS. 6A-6G show the 18 hour LDH release assay results using CAR T cells expressing Clones #1 and #11 in various indicated adherent cell lines from solid cancers such as Melanoma (SKMEL5), Thyroid cancer (TPC1), Mesothelioma (JMN), breast cancer (MDA-MB231), and pancreatic cancer (PANC-1). T47D is a negative control cell line lacking A02; HCT116 is negative for target Peptide A by mass spectrometry.

FIGS. 7A-7C show alanine screening results measuring the binding of CAR T cell-expressing clones of the present technology to peptide-pulsed T2 cells (FIG. 7A) and cell lysis (FIGS. 7B and 7C) as a readout. FIG. 7A discloses SEQ ID NOS 1 and 179-187, respectively, in order of appearance. FIG. 7D shows A*02 stabilization assay after T2 cells were pulsed with different peptides for alanine screens.

FIGS. 8A-8B show alanine screening results measuring the binding of TCR mimic antibodies of the present technology (2 μg/ml) to peptide-pulsed T2 cells by flow cytometry.

FIGS. 9A-9C show binding specificity of PE-conjugated TCR mimic antibodies of the present technology (2 μg/ml) using peptides having the amino acid sequences ALNEQIARL (SEQ ID NO: 1), ALNEKLVNL (SEQ ID NO: 166), and MLANDIARL (SEQ ID NO: 168) by flow cytometry. Flu peptide GILGFVFTL (SEQ ID NO: 170) is used as a negative control.

FIGS. 10A-10I show binding specificity of PE-conjugated TCR mimic antibodies of the present technology at several high concentrations (2 μg/ml, 10 μg/ml, and 50 μg/ml) to peptides having the amino acid sequence of ALNEQIARL (SEQ ID NO: 1), ALNEKLVNL (SEQ ID NO: 166), and MLANDIARL (SEQ ID NO: 168) measured by flow cytometry.

FIGS. 11A-11G show results of direct antibody staining by PE-conjugated TCR mimic antibodies of the present technology (2 μg/ml) in hematological cancer cell lines and controls as measured by flow cytometry. FIG. 11H shows A*02 density for different cell lines. A*02 molecules per cell were quantitated by flow cytometry and divided by mean FSC. Error bars indicate SD. HL60 serves as hematopoetic negative control, JMN is representative for all non-hematopoetic cell lines.

FIGS. 12A-12C show results of direct antibody staining by PE-conjugated TCR mimic antibodies (2 μg/ml) to adherent cancer cell lines as measured by flow cytometry.

FIGS. 13A-13E show that TCR mimic antibody binding can be enhanced through pre-treatment of target cells with the microtubule stabilizing drug docetaxel for a period of 72 hours measured by flow cytometry.

FIGS. 14A-14D show a lack of PBMC binding activity by the TCR mimic antibodies of the present technology as measured by flow cytometry.

FIGS. 15A-15C show that NDC80 knockdown by siRNA abrogates CAR T cell killing in a dose-dependent manner as shown by Western blot and cell killing assays.

FIG. 16 shows a mass spectrometry pulldown experiment with TCR mimic antibodies from clone 1. FIG. 16 discloses SEQ ID NOS 166, 1, 174, 173 and 168, respectively, in order of appearance.

FIGS. 17A-17B show exemplary FACS screening of phage clones using NDC80 peptide pulsed T2 cells stained with a biotinylated mouse anti-M13 mAb and a PE conjugated streptavidin as compared to T2 cells pulsed with negative control endogenous peptide mixture P20 (T2-P20). Geometric means are indicated within parentheses.

FIG. 18 FACS analysis shows that clone 1 had high specificity of NDC80 Peptide A/MHC I binding with no cross-reactivity to any of the four homologous peptides NC1-NC4.

FIG. 19 FACS analysis shows that clone 2 had medium specificity of NDC80 Peptide A/MHC I binding with some low-level cross-reactivity to at least one of the four homologous peptides NC1-NC4.

FIGS. 20A-20S FACS analysis shows that clones 1-2, and 4-19 exhibited specific binding to NDC80 Peptide A/HLA-A*02:01 complex, but low to no binding to T2 cells loaded with the mixture of NC1-NC4 peptides.

FIGS. 21A-21F FACS analysis shows that clones 1, 7, 11, 14, 18, and 19 exhibit highly specific binding to NDC80 Peptide A and no binding to any of the four homologous peptides (SEQ ID NOs: 166-169). The tables indicating the geometric means correspond to T2 cells loaded with NC4, NC3, NC2, NC1, P20, NC1-4 mixture, and 024P (target NDC80 peptide A) and T2 cells only from top to bottom order. FIG. 21G shows the results of the negative control.

FIG. 22 shows a summary of the results of the screening methods described in Example 1.

FIG. 23 shows the heavy chain variable domain (V_{H}) CDR 1-3 and light chain variable domain (V_{L}) CDR 1-3 amino acid sequences of the NDC80-specific clones of the present technology. FIG. 23 discloses SEQ ID NOS 44, 47, 50, 53, 56, 59, 62, 65, 68, 71, 74, 77, 80, 83, 86, 89, 92, 95, 45, 48, 51, 54, 57, 60, 63, 66, 69, 72, 75, 78, 81, 84, 87, 90, 93, 96, 46, 49, 52, 55, 58, 61, 64, 67, 70, 73, 76, 79, 82, 85, 88, 91, 94, 97, 98, 101, 104, 107, 110, 113, 116, 119, 122, 125, 128, 131, 134, 137, 140, 143, 146, 149, 99, 102, 105, 108, 111, 114, 117, 120, 123, 126, 129, 132, 135, 138, 141, 144, 147, 150, 100, 103, 106, 109, 112, 115, 118, 121, 124, 127, 130, 133, 136, 139, 142, 145, 148 and 151, respectively, in order of columns.

FIGS. 24A-24E show that clone 1 (labeled as Clone #1 in FIG. 24A) CAR T cells do not mediate toxicity towards healthy leukocytes, activated lymphocytes or hematopoetic stem cells. FIG. 24A shows the results of flow cytometry of clone 1 or mIgG1 isotype binding of healthy A*02 positive CD3, CD19, CD33 and CD15 positive cells. Data are representative of three donors. FIG. 24B shows the results of LDH killing assay with clone 1 CAR T cells and MACS sorted CD3, CD14 and CD19 positive cells. CD13 and CD19 positive cells were also tested after 48h of activations. BV173 cells served as positive control. Error bars denote SD. FIG. 24C shows the results of Zebra plot after co-culture of clone 1 CAR T cells produced from cells of A*02 positive and negative blood donors. Flow cytometry was performed after 18 hours co-culture at 1:1 ratio. Plot is representative of 3 biological replicates. FIG. 24D shows cell proliferation capacity of stimulated, but untransduced T cells, HLA-A*02 positive and negative clone 1 CAR T cells. Error bars denote SD. FIG. 24E shows the results of colony forming unit assays of cord blood isolated CD34 positive HSCs, OCI-AML02 cell line as positive control and N3 primary AML cells. Cells were plated after 18 hours 1:1 co-culture of CART cells and target cells. MUC16 specific 4H11 CAR T cells served as control. *p<0.05 (unpaired t test). Error bars denote SD.

FIGS. 25A-25H show that clone 1 CAR T cells control tumor growth and prolong survival in leukemia and mesothelioma mouse models. FIG. 25A shows experimental design for BV173 i.v. leukemia model. FIG. 25B shows mean tumor burden as defined by percentage of A*02 positive blast cells in mouse blood. 5 mice per group. **p<0.01 (Mann Whitney test). Error bars denote SD. FIG. 25C shows overall survival in BV173 leukemia model. *p<0.05, **p<0.01 (Log rank test). FIG. 25D shows experimental design for JMN i.p. mesothelioma model. FIG. 25E shows spaghetti plot depicting individual tumor burden relative to day 3 (D3). FIG. 25F shows geometric mean of average tumor burden Spaghetti plot depicting individual tumor burden relative to Day 3. 5 mice per group. *p<0.05, **p<0.01 (Mann Whitney test). Error bars denote 95% CI. FIG. 25G shows bioluminescence imaging using luciferase pre and post CAR T cell treatment. FIG. 25H shows overall survival in JMN mesothelioma model. **p<0.01 (Log rank test).

FIGS. 26A-26E show that NDC80 expression in healthy and cancer cells. FIG. 26A shows NDC80 expression in various cancer cell lines (CCLE data) compared to healthy tissues (GTEX consortium data). FIG. 26B shows NDC80 expression in cancer tissues and corresponding adjacent healthy tissues (both PCAWG data). FIG. 26C shows fold changes of NDC80 expression for different cancer types based on cancer cell line data from FIG. 26A. FIG. 26D shows fold changes of NDC80 expression for different cancer types based on primary tissue data from FIG. 26B. FIG. 26E shows NDC80 expression of cancer cell lines compared to Primary tissues.

FIG. 27 shows relative quantitation of mass spectrometry identified target and potential off-target HLA ligands identified via clone 1 immunoprecipitation. Quantitation is done by peak areas of precursor ion and three isotope variants using skyline. FIG. 27 discloses SEQ ID NOS 1, 168, 174, 166, 173, 1, 166, 168 and 173-174, respectively, in order of appearance.

FIGS. 28A-28D show the results of lineage specific flow cytometry for cord blood isolated HSCs and LDH assay with clone 1 CAR T cells. FIG. 28A shows cell counts of colony forming cells of A02 positive and negative HSCs acer co-culture with clone 1 CAR T cells or MUC16 specific control CAR T cells for 18 hours. FIGS. 28B-28C shows lineage specific flow cytometry for conditions described in FIG. 28A. A myeloid panel (PE-CD13, FITC-CD14, APC-CD33, Pacific Blue-Mac1) and erythroid panel (FITC-CD71, PE-GlycophorinA) were used. FIG. 28D shows killing as determined by LDH assay for conditions described in FIG. 28A. OCI-AML02 served as positive control. For all graphs error bars indicate SD.

FIGS. 29A-29B show in vitro killing of GFP-Luc transduced JMN cells and individual tumor burdens in BV173 leukemia model. FIG. 29A shows LDH assay for clone 1 CAR T cells with either transduced and untransduced JMN cells. FIG. 29B shows individual tumor burden defined by percentage of HLA-A*02 positive blasts in mouse peripheral blood after cheek bleeds identified by flow cytometry.

FIG. 30 shows the binding of clone 1 in normal human cardiomyocytes (HCM), cardiac fibroblast (HCF) and thymic fibroblasts (HTyF) as determined by flow cytometry. The AML-14 leukemia cell line was used as a positive control. BB7 mAb is used to determine HLA-A2 expression on targets.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

To identify a tumor specific target that is presented as a peptide in conjunction with the highly prevalent HLA allele A*02:01, peptide/MHC complexes were immunopurified from various cancer cell lines of different origins, HLA ligands were separated from complexes and their peptide sequences identified via mass spectrometry. Network analysis of the resulting HLA ligand datasets identified shared biological processes among the tumor cell lines that were not present in network analyses of published datasets of healthy human tissue HLA ligandomes. Through this filtering process, an HLA ligand derived from kinetochore NDC80 protein homolog (NDC80) was selected as a target. NDC80 has been shown to be differentially expressed in malignant tissues compared to adjacent non-malignant tissues, and is associated with poor prognosis in many cancer types.

The NDC80 derived peptide ALNEQIARL (SEQ ID NO: 1) was detected in over 90% of the A*02 positive cell lines tested, and never reported to be present in HLA ligand datasets of healthy human tissues. These results were unexpected in view of prior studies that reported no T cell reactivity with the ALNEQIARL (SEQ ID NO: 1) peptide in both healthy individuals and AML patients. Dissertation of Anne Claudia Berlin, Kartierung des HLA-Ligandoms der akuten myeloischen Leukämie zur Entwicklung einer therapeutischen Multipeptidvakzine (2018). Likewise, initial T cell peptide stimulation studies against the ALNEQIARL (SEQ ID NO: 1) peptide were also found to be negative for T cell reactivity.

The TCR mimic antibodies disclosed herein showed high specificity for the target HLA:peptide complex in antibody and CAR T cell formats in vitro, and demonstrated binding primarily to the central region of the HLA ligand as determined by alanine screening assays. The specificity of the anti-NDC80/MHC TCR mimic antibodies was further illustrated by NDC80 knockdown experiments as well as successful immunopurification of the target peptide together with no relevant off-targets from BV173 ALL cells in mass spectrometry assays. Given the high specificity, sensitivity was assessed primarily in a potent CAR T cell format. Multiple tumor cell lines of different origin (e.g. ALL, AML, lymphoma, melanoma, mesothelioma, pancreatic and thyroid cancer) were successfully killed in vitro by CAR T cells that target NDC80/HLA complexes. No toxicity towards A*02:01 positive CAR T cells, healthy PBMCs or NDC80 target negative cell lines was observed. Anti-NDC80/MHC CAR T cells demonstrated highest efficacy in hematological malignancies most likely correlating with elevated expression of antigen presentation machinery and rapid cell division which leads to strong surface presentation of NDC80 peptides. In summary, CAR T cells directed against peptide/HLA-A*02 derived from the NDC80 protein effectively kill multiple cancer cell lines in vitro without significant off-target killing. The more effective killing especially against ALL, AML and lymphomas highlights the potential of these CAR T cells to preferentially eliminate cancer cells with high proliferative capacity.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the terms “clone #1,” “clone #2,” “clone #3,” “clone #4,” “clone #5,” “clone #6,” “clone #7,” “clone #8,” “clone #9,” “clone #10,” “clone #11,” “clone #12,” “clone #13,” “clone #14,” “clone #15,” “clone #16,” “clone #17,” “clone #18” and “clone #19” are interchangeable with the terms “clone 1,” “clone 2,” “clone 3,” “clone 4,” “clone 5,” “clone 6,” “clone 7,” “clone 8,” “clone 9,” “clone 10,” “clone 11,” “clone 12,” “clone 13,” “clone 14,” “clone 15,” “clone 16,” “clone 17,” “clone 18,” and “clone 19” respectively.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

An “antigen-binding protein” is a protein or polypeptide that comprises an antigen-binding region or antigen-binding portion, that has a strong affinity to another molecule to which it binds. Antigen-binding proteins encompass antibodies, antibody fragments, chimeric antibody-T cell receptors (caTCRs), chimeric antigen receptors (CARs) and fusion proteins, and conjugates thereof.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³M⁻¹ greater, at least 10⁴M⁻¹ greater or at least 10⁵M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, an antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds NDC80/MHC complex will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). An antibody or antigen binding fragment thereof specifically binds to an antigen.

As used herein, the term “antibody moiety” encompasses full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)2 fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi specific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an immunoglobulin-related composition (e.g., antibody or antigen binding fragment thereof) can selectively bind. The target antigen may be a protein, carbohydrate, nucleic acid, lipid, hapten, or other naturally occurring or synthetic compound. In some embodiments, the target antigen may be a peptide/MHC complex (e.g., a NDC80 peptide/MHC complex, also called “NDC80/MHC complex”, “NDC80 peptide-MHC complex”, “NDC80-MHC complex”, or “NDC80 peptide complexed with MHC” as used herein). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an immunoglobulin-related composition) and its binding partner (e.g., an antigen). The affinity of a molecule X for its partner Y can generally be represented by the dissociation constant (K_{D}). Affinity can be measured by standard methods known in the art, including those described herein. A low-affinity complex contains an immunoglobulin-related composition that generally tends to dissociate readily from the antigen, whereas a high-affinity complex contains an immunoglobulin-related composition that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “chimeric antibody-T cell receptors (caTCRs)” refers to a functional polypeptide complex typically comprising at least two separate polypeptide chains. The caTCR complex comprises an antigen-binding module that specifically binds to a target antigen and a T cell receptor module (TCRM). The TCRM typically comprises a first TCR domain (TCRD) on one of the two polypeptide chains, comprising a first TCR transmembrane domain (TCR-TM), and a second TCRD on the other polypeptide chain, comprising a second TCR-TM, wherein the TCRM facilitates recruitment of at least one TCR-associated signaling molecule, e.g., CD3ζ, upon specific binding of the antigen-binding module to its target antigen. The antigen-binding module of a caTCR complex is derived from an antibody, and caTCR complexes typically do not comprise an antigen-binding TCR variable domain or TCR CDR sequences. The caTCR complex itself does not comprise a functional primary immune cell signaling sequence, such as a functional signaling sequence comprising an ITAM, e.g., the intracellular domain of CD3ζ, CD3γ, CD3δ, CD3ε, FcRγ, FcRβ, CD5, CD22, CD79a, CD79b, or CD66d. In some embodiments, the caTCR complex itself does not comprise any primary immune cell signaling sequence,

In some embodiments, the antigen-binding module in caTCR comprises the anti-NDC80/MHC antibody moiety described herein. In some embodiments, the antigen-binding module comprises a Fab, a Fab′, a F(ab′)2, an Fv, or a single chain Fv (scFv). In some embodiments, the antigen-binding module comprises at least one scFv on one of the two polypeptide chains. In some embodiments, the antigen-binding module comprises at least one scFv on each of the two polypeptide chains. In some embodiments, the antigen-binding module comprises at least one Fab composed of an antibody heavy chain variable region (V_{H}) on one of the two polypeptide chains and an antibody light chain variable region (V_{L}) on the other polypeptide chain.

One of the TCR-TMs or TCRDs can be derived from or is a fragment of a TCR chain selected from TCRα, TCRβ, TCRγ, and TCRδ, while the other TCR-TM or TCRD can be derived from or is a fragment of another TCR chain selected from TCRα, TCRβ, TCRγ, and TCR which pairs with the first TCR fragment, e.g., TCRα-TCRβ pairing or TCRγ-TCRδ pairing.

In some embodiments, one of the two polypeptide chains comprises a V_{H} fused directly or indirectly to one of the TCRD (which can be called “the first TCRD”), while the other polypeptide chain comprises a V_{L} fused directly or indirectly to the other TCRD (which can be called “the second TCRD”). In some embodiments, there is a linker between V_{H} and the first TCRD and/or between V_{L} and the second TCRD. In some embodiments, the linker between V_{H} and the first TCRD can be CH1, CH2, CH3, CH4, or a TCR constant domain (which can be called “the first TCR constant domain), while the linker between V_{L} and the second TCRD can be CL (which pairs with CH1), CH2 (which pairs with CH2), CH3 (which pairs with CH₃), CH4 (which pairs with CH4), or a TCR constant region that pairs with the first TCR constant domain, respectively. In some embodiments, the two polypeptide chains in a caTCR complex are linked by at least one disulfide bond.

In some embodiments, the caTCR does not include a functional co-stimulatory signaling sequences (e.g., the intracellular domain of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, or the like). In some embodiments, the caTCR does not include any co-stimulatory signaling sequences.

The terms “caTCR” and “antibody-TCR chimeric molecule or construct (abTCR)” are used interchangeably. Further descriptions and examples of caTCR and abTCR may be found in, e.g., WO2017/070608, filed Oct. 21, 2016 and WO2018/200582, filed Apr. 24, 2018, which are incorporated by reference herein in its entirety.

Chimeric antigen receptors (CARs): Compositions of the present technology, including single chain variable fragments (scFv), may be used for the preparation of chimeric antigen receptors, the preparation and use of which is generally known in the art. A chimeric antigen receptor (CAR) is an artificially constructed hybrid single-chain protein or single-chain polypeptide containing a single-chain variable fragment (scFv) as a part of the extracellular antigen-binding domain, linked to a transmembrane domain (e.g., the transmembrane domain of a co-stimulatory molecule such as CD28 or CD8), which is in turn linked to an intracellular immune cell (e.g., T cell or NK cell) signaling domain which comprises at least a functional primary immune cell signaling sequence. Primary immune cell signaling sequences act in a stimulatory manner and contain signaling motifs which are known as immunoreceptor tyrosine-based activation motifs or ITAMs. Examples of ITAM-containing primary immune cell signaling sequences include those derived from CD3ζ (a.k.a. TCR), FcRγ, FcRβ, CD3γ, CD3δ, CD3ε, CD5, CD22, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprise fragments or variants of primary immune cell signaling sequences, are unable to transduce an immune cell activation signal.

There are currently three generations of CARs. The “first generation” CARs are typically single-chain polypeptides composed of a scFv as the antigen-binding domain fused to a transmembrane domain fused to cytoplasmic/intracellular domain of the T cell receptor (TCR) chain. The “first generation” CARs typically have the intracellular domain from the CD3ζ chain, which is the primary transmitter of signals from endogenous TCRs through TCR complexes which comprise TCRs and CD3 molecules. The “first generation” CARs can provide de novo antigen recognition and cause activation of both CD4+ and CD8+ T cells through their CD3ζ chain signaling domain in a single fusion molecule, independent of HLA-mediated antigen presentation.

The “second generation” CARs add intracellular domains from various co-stimulatory molecules (e.g., CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, or the like) to the cytoplasmic tail of the CAR to provide additional signals to the T cell. “Second generation” CARs comprise those that provide both co-stimulation (e.g., CD28 or 4-IBB) and activation (e.g., CD3ζ). Preclinical studies have indicated that the “second generation” CARs can improve the antitumor activity of T cells. For example, robust efficacy of the “second generation” CAR modified T cells was demonstrated in clinical trials targeting the CD19 molecule in patients with chronic lymphoblastic leukemia (CLL) and acute lymphoblastic leukemia (ALL).

The “third generation” CARs have multiple intracellular domains from various co-stimulatory molecules (e.g., from both CD28 and 4-1BB).

Benefits of caTCRs and CARs include their abilities to redirect immune cell (e.g., T cell or NK cell) specificity and reactivity toward a selected target in either MHC-restricted (in case of TCR-mimic antibodies) or non-MHC-restricted (in case of antibodies against cell surface proteins) manners, exploiting the antigen-binding properties of monoclonal antibodies.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means an antigenic determinant capable of specific binding to an immunoglobulin-related composition such as an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” is a region of the target antigen to which the anti-NDC80/MHC immunoglobulin-related compositions of the present technology specifically bind. In some embodiments, the epitope is a conformational epitope or a non-conformational epitope. To screen for anti-NDC80/MHC immunoglobulin-related compositions which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-NDC80/MHC antibody binds the same site or epitope as an anti-NDC80/MHC antibody of the present technology. Alternatively, or additionally, epitope mapping can be performed by methods known in the art. For example, the antibody sequence can be mutagenized such as by alanine scanning, to identify contact residues. In a different method, MHC complexes including peptides corresponding to different regions of NDC80 protein can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

The term “HLA-A2”, as used herein, representatively refers to the subtypes, examples of which include, but are not limited to, HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28 and HLA-A*02:50.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR.” As used herein, the term “CDR” or “complementarity determining region” of an antibody (or immunoglobulin) is intended to mean the non-contiguous antigen combining sites found within the variable region of both heavy and light chain polypeptides. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The exemplary CDR sequences of the anti-NDC80/MHC-specific antibody clones disclosed herein were predicted using the IMGT algorithm which is based on Lefranc et al., supra.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an immunoglobulin-related composition described herein or amino acid sequence of an immunoglobulin-related composition described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an immunoglobulin-related composition) which recognizes and binds another molecule (e.g., an antigen), but that does not substantially recognize and bind other molecules. The terms “specific binding,” “specifically binds to,” or is “specific for” a particular molecule, as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an immunoglobulin-related composition) binds to a particular target molecule or complex (e.g., a NDC80 peptide/MHC complex), without substantially binding to any other molecule or complex.

As used herein, the term “simultaneous” therapeutic use refers to the administration of at least two active ingredients by the same route and at the same time or at substantially the same time.

The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a TCR constant domain (C) that is anchored to cell membrane by a connecting peptide and a transmembrane (TM) region. The TM region associates with the invariant subunits of the CD3 signaling apparatus. Each of the V domains has three TCR CDRs. These TCR CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

The term “TCR-associated signaling molecule” refers to a molecule having a cytoplasmic immunoreceptor tyrosine-based activation motif (ITAM) that is part of the TCR-CD3 complex. TCR-associated signaling molecules include CD3γε, CD3ε, and CD3ζ (also known as ζζ, CD3ζζ, or TCRζ).

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-NDC80/MHC immunoglobulin-related compositions described herein are contemplated. For example, it may be desirable to improve the binding affinity and/or other biological properties of an immunoglobulin-related composition disclosed herein. Amino acid sequence variants of an anti-NDC80/MHC immunoglobulin-related composition are prepared by introducing appropriate nucleotide changes into its nucleic acid, or by peptide synthesis. Such modifications include, for example, deletions from, and/or insertions into and/or substitutions of, residues within the amino acid sequences of the immunoglobulin-related composition. Any combination of deletion, insertion, and substitution is made to obtain the immunoglobulin-related composition of interest, as long as the obtained immunoglobulin-related composition possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 2
Amino Acid Substitutions

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

### Immunoglobulin-Related Compositions of the Present Technology

“Immunoglobulin-related compositions” as used herein, refers to antibodies (including monoclonal antibodies, polyclonal antibodies, humanized antibodies, chimeric antibodies, human antibodies, recombinant antibodies, multispecific antibodies, bispecific antibodies, etc.,), antibody fragments, chimeric antibody-T cell receptors (caTCRs), chimeric antigen receptors (CARs), fusion proteins, and conjugates thereof.

The present disclosure relates to immunoglobulin-related compositions that specifically recognize epitopes of a complex of a peptide/protein fragment derived from an intracellular NDC80 protein, and an MHC class I molecule, for example, as the complex might be displayed at the cell surface during antigen presentation.

Traditionally, the MHC-peptide complex could only be recognized by a T-cell receptor (TCR), limiting the ability to detect an epitope of interest using T cell-based readout assays. The present technology describes methods and compositions for the generation and use of anti-NDC80/MHC immunoglobulin-related compositions. In the present disclosure, immunoglobulin-related compositions, including antibodies, having an antigen-binding region based on scFvs that are selected from human scFv phage display libraries using recombinant HLA-peptide complexes are described. These molecules demonstrated specificity, for example as shown with anti-NDC80/MHC antibodies that recognize only HLA-A*02-ALNEQIARL (SEQ ID NO: 1) complexes. Accordingly, the immunoglobulin-related compositions of the present disclosure operate as “TCR mimic” antigen binding proteins. In addition, the molecules were also unable to bind HLA-complexes containing other peptides, further demonstrating their TCR-like specificity.

The anti-NDC80/MHC immunoglobulin-related compositions of the present disclosure may be useful in the diagnosis, or treatment of NDC80-associated diseases (e.g., cancers). Anti-NDC80/MHC immunoglobulin-related compositions within the scope of the present technology include, e.g., but are not limited to, monoclonal antibodies, polyclonal antibodies, humanized antibodies, human antibodies (e.g., fully human antibodies), chimeric antibodies, recombinant antibodies, multispecific antibodies, bispecific antibodies, diabodies, antibody fragments (e.g., Fab, F(ab)′2, Fab′, scFᵥ, and Fᵥ), chimeric antibody-T cell receptors (caTCRs), chimeric antigen receptors (CARs), fusion proteins, and conjugates that specifically bind the target antigen, a homolog, derivative or a fragment thereof. Additionally or alternatively, in some embodiments, the immunoglobulin-related compositions of the present technology may be an immunoglobulin polypeptide, or an immunoglobulin-like polypeptide.

Without wishing to be bound by theory, it is anticipated that the TCR mimic immunoglobulin-related compositions of the present disclosure simulate the function of a TCR in a T cell because like a TCR, the immunoglobulin-related compositions only recognize the target peptide ALNEQIARL (SEQ ID NO: 1) when complexed with HLA-A*02.

FIG. 23 and Table 3 below provides V_{H} CDR sequences of the immunoglobulin-related compositions of present technology:

TABLE 3

| Clone | V_{H} CDR1 | V_{H} CDR2 | V_{H} CDR3 |
|---|---|---|---|
| 1 | GYSFSSNW | IYPGDSDT | ARFAGPGMWSYGFDY |
| | (SEQ ID | (SEQ ID | (SEQ ID |
| | NO: 44) | NO: 45) | NO: 46) |
| 2 | GGTFSSYA | ISAYNGNT | ARKGFYSGFDS |
| | (SEQ ID | (SEQ ID | (SEQ ID |
| | NO: 47) | NO: 48) | NO: 49) |
| 4 | GGSFSGYY | INHSGST | ARGVPYDY |
| | (SEQ ID | (SEQ ID | (SEQ ID |
| | NO: 50) | NO: 51) | NO: 52) |
| 5 | GGSISSSNW | TYHSGST | ARFIYGSSSSQTDT |
| | (SEQ ID | (SEQ ID | (SEQ ID |
| | NO: 53) | NO: 54) | NO: 55) |
| 6 | GGSFSGYY | INHSGST | ARLEGSYYDV |
| | (SEQ ID | (SEQ ID | (SEQ ID |
| | NO: 56) | NO: 57) | NO: 58) |
| 7 | GYTFTSYG | ISAYNGNT | ARMSMSEYIDY |
| | (SEQ ID | (SEQ ID | (SEQ ID |
| | NO: 59) | NO: 60) | NO: 61) |
| 8 | GDSISGYY | INHSGST | ARYYSGFYSSDG |
| | (SEQ ID | (SEQ ID | (SEQ ID |
| | NO: 62) | NO: 63) | NO: 64) |
| 9 | GGSISSSNW | IYHSGST | ARYEQGSHDS |
| | (SEQ ID | (SEQ ID | (SEQ ID |
| | NO: 65) | NO: 66) | NO: 67) |
| 10 | GGSISSSNW | TYHSGST | ARYYNSYYDS |
| | (SEQ ID | (SEQ ID | (SEQ ID |
| | NO: 68) | NO: 69) | NO: 70) |
| 11 | GYSFTSNG | ISGYNANT | ARHAYWGGDSDY |
| | (SEQ ID | (SEQ ID | (SEQ ID |
| | NO: 71) | NO: 72) | NO: 73) |
| 12 | GGSISSSNW | IYHGGST | ARYASSSHDF |
| | (SEQ ID | (SEQ ID | (SEQ ID |
| | NO: 74) | NO: 75) | NO: 76) |
| 13 | GDSISSNTW | INHSGST | ARWGYGSAYSDA |
| | (SEQ ID | (SEQ ID | (SEQ ID |
| | NO: 77) | NO: 78) | NO: 79) |
| 14 | GGSISSSNW | IYHSGST | ARYFGQKYDY |
| | (SEQ ID | (SEQ ID | (SEQ ID |
| | NO: 80) | NO: 81) | NO: 82) |
| 15 | GGSISSSNW | TYHSGST | ARYPGFVWDL |
| | (SEQ ID | (SEQ ID | (SEQ ID |
| | NO: 83) | NO: 84) | NO: 85) |
| 16 | GYTFTSYY | INPSGGST | ARRGWLSWGYYGMDV |
| | (SEQ ID | (SEQ ID | (SEQ ID |
| | NO: 86) | NO: 87) | NO: 88) |
| 17 | GSTFINYG | VSGYNGNT | ARYYYPPYDY |
| | (SEQ ID | (SEQ ID | (SEQ ID |
| | NO: 89) | NO: 90) | NO: 91) |
| 18 | GGTFSSYA | IIPIFGTA | ARGFSSWPGIDQ |
| | (SEQ ID | (SEQ ID | (SEQ ID |
| | NO: 92) | NO: 93) | NO: 94) |
| 19 | GYSFTTYW | IYPGDSDT | ARYGGQYFWSDSFDS |
| | (SEQ ID | (SEQ ID | (SEQ ID |
| | NO: 95) | NO: 96) | NO: 97) |

FIG. 23 and Table 4 below provides V_{L} CDR sequences of the immunoglobulin-related compositions of present technology:

TABLE 4

| Clone | V_{L} CDR1 | V_{L} CDR2 | V_{L}CDR3 |
|---|---|---|---|
| 1 | QGIRND | AAS | LODYDYPLT |
| | (SEQ ID | (SEQ ID | (SEQ ID |
| | NO: 98) | NO: 99) | NO: 100) |
| 2 | SSNIGSNY | DNT | ETLDSSLSAYV |
| | (SEQ ID | (SEQ ID | (SEQ ID |
| | NO: 101) | NO: 102) | NO: 103) |
| 4 | RSNIGSNF | DNN | GTWDSSLIAGV |
| | (SEQ ID | (SEQ ID | (SEQ ID |
| | NO: 104) | NO: 105) | NO: 106) |
| 5 | SSNIGSNY | DDN | GTWDSPLVA |
| | (SEQ ID | (SEQ ID | (SEQ ID |
| | NO: 107) | NO: 108) | NO: 109) |
| 6 | TSDIGSNY | DNN | GTWDSSLNTFV |
| | (SEQ ID | (SEQ ID | (SEQ ID |
| | NO: 110) | NO: 111) | NO: 112) |
| 7 | SSNIGAGYD | GNV | QSYDSSLSGWV |
| | (SEQ ID | (SEQ ID | (SEQ ID |
| | NO: 113) | NO: 114) | NO: 115) |
| 8 | SSNIGSNY | DNN | GSWDSSLSVYV |
| | (SEQ ID | (SEQ ID | (SEQ ID |
| | NO: 116) | NO: 117) | NO: 118) |
| 9 | SSNIGSNY | DNS | GTWDSSLSAYV |
| | (SEQ ID | (SEQ ID | (SEQ ID |
| | NO: 119) | NO: 120) | NO: 121) |
| 10 | SSNIGSNY | DNN | GTWDSSLSAYV |
| | (SEQ ID | (SEQ ID | (SEQ ID |
| | NO: 122) | NO: 123) | NO: 124) |
| 11 | SGSIASNY | EDN | QSYDSSNVV |
| | (SEQ ID | (SEQ ID | (SEQ ID |
| | NO: 125) | NO: 126) | NO: 127) |
| 12 | RSNIVSNY | DNN | GTWDSSLSAYV |
| | (SEQ ID | (SEQ ID | (SEQ ID |
| | NO: 128) | NO: 129) | NO: 130) |
| 13 | SSNIGSNY | DNN | GIWDSSLSAYV |
| | (SEQ ID | (SEQ ID | (SEQ ID |
| | NO: 131) | NO: 132) | NO: 133) |
| 14 | TSNIGSNY | DSD | GTWDSSLTVGV |
| | (SEQ ID | (SEQ ID | (SEQ ID |
| | NO: 134) | NO: 135) | NO: 136) |
| 15 | SSNIGSNY | DNN | GTWDSSLTAGV |
| | (SEQ ID | (SEQ ID | (SEQ ID |
| | NO: 137) | NO: 138) | NO: 139) |
| 16 | KLGDKY | QDS | QAWDSSTAYV |
| | (SEQ ID | (SEQ ID | (SEQ ID |
| | NO: 140) | NO: 141) | NO: 142) |
| 17 | TLRSYY | GRN | QSFDYSYSV |
| | (SEQ ID | (SEQ ID | (SEQ ID |
| | NO: 143) | NO: 144) | NO: 145) |
| 18 | SSNIGNNY | DNN | QSYDVYNMTSV |
| | (SEQ ID | (SEQ ID | (SEQ ID |
| | NO: 146) | NO: 147) | NO: 148) |
| 19 | SGSVASEF | NDF | QSYDQSSSIV |
| | (SEQ ID | (SEQ ID | (SEQ ID |
| | NO: 149) | NO: 150) | NO: 151) |

The V_{H} amino acid sequences of the NDC80-specific clones of the present technology (SEQ ID NOs: 2-19) are provided below. The V_{H} CDR 1-3 sequences are underlined.

Clone 1 heavy chain variable domain, protein
(SEQ ID NO: 2)
EVQLVQSGAEVKKPGESLKISCEASGYSFSSNWIAWVRQRPGKGLEWMGIIYPGDSDTRYSPSFQGQVTMS
ADMSISTAYLQWSSLKASDTAMYYCARFAGPGMWSYGFDYWGQGTLVTVSS
Clone 2 heavy chain variable domain, protein
(SEQ ID NO: 3)
QVQLVQSGAEVKKPGSSVKVSCKASGGTFSSYAISWVRQAPGQGLEWMGWISAYNGNTNYAQKLQGRV
TMTTDTSTSTAYMELRSLRSDDTAVYYCARKGFYSGFDSWGQGTLVTVSS
Clone 4 heavy chain variable domain, protein
(SEQ ID NO: 4)
QVQLQQWGAGLLKPSETLSLTCAVYGGSFSGYYWSWIRQPPGKGLEWIGEINHSGSTNYNPSLKSRVTISV
DTSKNQFSLKLSSVTAADTAVYYCARGVPYDYWGQGTLVTVSS
Clone 5 heavy chain variable domain, protein
(SEQ ID NO: 5)
QLQLQESGPGLVKPSGTLSLTCAVSGGSISSSNWWSWVRQPPGKGLEWIGEIYHSGSTNYNPSLKSRVTISV
DKSKNQFSLKLSSVTAADTAVYYCARFIYGSSSSQTDTWGQGTLVTVSS
Clone 6 heavy chain variable domain, protein
(SEQ ID NO: 6)
QVQLQQWGAGLLKPSETLSLTCAVYGGSFSGYYWSWIRQPPGKGLEWIGEINHSGSTNYNPSLKSRVTISV
DTSKNQFSLKLSSVTAADTAVYYCARLEGSYYDVWGQGTLVTVSS
Clone 7 heavy chain variable domain, protein
(SEQ ID NO: 7)
QVQLQQSGAEVKKPGATVKVSCKASGYTFTSYGISWVRQAPGQGLEWMGWISAYNGNTNYAQKLQGRV
TMTTDTSTSTAYMELRSLRSDDTAVYYCARMSMSEYIDYWGQGTLVTVSS
Clone 8 heavy chain variable domain, protein
(SEQ ID NO: 8)
QLQLQESGPGLVKPSETLSLTCTVSGDSISGYYWSWIRQPPGKGLEWIGEINHSGSTNYNPSLKSRVTISVDT
SKNQFSLKLSSVTAADTAVYYCARYYSGFYSSDGWGQGTLVTVSS
Clone 9 heavy chain variable domain, protein
(SEQ ID NO: 9)
QVQLQESGPGLVKPPGTLSLTCAVSGGSISSSNWWSWVRQPPGKGLEWIGEIYHSGSTNYNPSLKSRVTISV
DKSKNQFSLKLSSVTAADTAVYYCARYEQGSHDSWGQGTLVTVSS
Clone 10 heavy chain variable domain, protein
(SEQ ID NO: 10)
QVQLQESGPGLVKPPGTLSLTCAVSGGSISSSNWWSWVRQPPGKGLEWIGEIYHSGSTNYNPSLNSRVIISD
DTSKNQFSLTLTSVTAADTAVYYCARYYNSYYDSWGQGTLVTVSS
Clone 11 heavy chain variable domain, protein
(SEQ ID NO: 11)
QVQLVQSGAEVRKPGASVKVSCKASGYSFTSNGITWVRQAPGQGLEWMGWISGYNANTRYAQEFQARVT
MTTDTSASTAYMELRSLRSDDTAVYYCARHAYWGGDSDYWGQGTLVTVSS
Clone 12 heavy chain variable domain, protein
(SEQ ID NO: 12)
QVQLQESGPGLVKPSGTLSLTCAVSGGSISSSNWWSWVRQAPGKGLEWIGEIYHGGSTNFNPSLKSRVTISV
DKSKNQFSLNLTSVTAADTAVYYCARYASSSHDFWGQGTLVTVSS
Clone 13 heavy chain variable domain, protein
(SEQ ID NO: 13)
QVQLQESGPGLVKPSGTLSLTCVVSGDSISSNTWWTWVRQPPGKGLDWIGEINHSGSTNYNPSLKSRVTISV
DTSKNQFSLNLSSVTAADTAVYYCARWGYGSAYSDAWGQGTLVTVSS
Clone 14 heavy chain variable domain, protein
(SEQ ID NO: 14)
QLQLQESGPGLVKPSGTLSLTCAVSGGSISSSNWWSWVRQPPGKGLEWIGEIYHSGSTNYNPSLKSRVTISV
DKSKNQFSLKLSSVTAADTAVYYCARYFGQKYDYWGQGTLVTVSS
Clone 15 heavy chain variable domain, protein
(SEQ ID NO: 15)
QVQLQESGPGLVKPSGTLSLTCAVSGGSISSSNWWSWVRQPPGKGLEWIGEIYHSGSTNYNPSLKSRVTISV
DKSKNQFSLKLSSVTAADTAVYYCARYPGFVWDLWGQGTLVTVSS
Clone 16 heavy chain variable domain, protein
(SEQ ID NO: 16)
QVQLVQSGAEVKKPGASVKVSCKASGYTFTSYYMHWVRQAPGQGLEWMGIINPSGGSTSYAQKFQGRVT
MTRDTSTSTVYMELSSLRSEDTAVYYCARRGWLSWGYYGMDVWGQGTTVTVSS
Clone 17 heavy chain variable domain, protein
(SEQ ID NO: 17)
QVQLVQSGAEVKKPGASVKVSCKASGSTFINYGITWVRQAPGQGLEWMGWVSGYNGNTDYAQKFQGRV
TMTADTSTSTAYMELRSLRSDDTAVYYCARYYYPPYDYWGSRYSGDRLL
Clone 18 heavy chain variable domain, protein
(SEQ ID NO: 18)
QMQLVQSGAEVKKPGSSVKVSCKASGGTFSSYAISWVRQAPGQGLEWMGGIIPIFGTANYAQKFQGRVTIT
ADESTSTAYMELSSLRSDDTAVYYCARGFSSWPGIDQWGQGTLVTVSS
Clone 19 heavy chain variable domain, protein
(SEQ ID NO: 19)
EVQLVQSGAEVKKPGESLKISCKGSGYSFTTYWIGWVRQMPGKGLEWMGIIYPGDSDTTYSPSFQGQVTIS
ADKSLSIAYLQWSSLKASDTAMYYCARYGGQYFWSDSFDSWGQGTLVTVSS
The VL amino acid sequences of the NDC80-specific clones of the present technology
(SEQ ID NOs: 20-37) are provided below. The V_{L} CDR 1-3 sequences are underlined.
Clone 1 light chain variable domain, protein
(SEQ ID NO: 20)
DIQLTQSPSSLSASVGDRVTITCRASQGIRNDLGWYQQKPGKAPKLMIYAASSLQSGVPSRFSGSGSGTDFT
LTISSLQPEDFATYYCLQDYDYPLTFGGGTKLEIKR
Clone 2 light chain variable domain, protein
(SEQ ID NO: 21)
QSVLTQPPSVSAAPGQKVTISCSGSSSNIGSNYVSWYQQLPGTAPKLLIYDNTKRPSGIPDRFSGSKSGSSAT
LGITGLQTGDEADYYCETLDSSLSAYVFGTGTKVTVLG
Clone 4 light chain variable domain, protein
(SEQ ID NO: 22)
QSVLTQPPSVSAAAGQKVTISCSGSRSNIGSNFVSWYQLLPGTAPRLLIYDNNVRPSGIPDRFSGSKSGSSAT
LGITGLQTGDEADYYCGTWDSSLIAGVFGGGTKLTVLG
Clone 5 light chain variable domain, protein
(SEQ ID NO: 23)
QSVLTQPPSVSAAPGQKVTISCSGSSSNIGSNYIYWYQQFPGTAPKLLIYDDNKRPSGIPDRFSGSRSGTSATL
GITGLQTGDEADYYCGTWDSPLVAWVFGGGTKLTVLG
Clone 6 light chain variable domain, protein
(SEQ ID NO: 24)
QSVLTQPPSVSAAPGQKVTVSCSGTTSDIGSNYVTWYQQLPGTTPKLIIYDNNKRPSGIPDRFSGSKSGTSGT
LGITGLQTGDEADYYCGTWDSSLNTFVFGTGTKVTVLG
Clone 7 light chain variable domain, protein
(SEQ ID NO: 25)
QSVVTQPPSVSGAPGQRITISCTGSSSNIGAGYDVHWYQQLPGTAPKLLIFGNVNRPSGVPDRFSGSKSGTSA
SLAITGLQAEDEADYYCQSYDSSLSGWVFGGGTKLTVLG
Clone 8 light chain variable domain, protein
(SEQ ID NO: 26)
QSVLTQPPSVSAAPRQKVTISCSGSSSNIGSNYVSWYQQLPGTAPKLLIYDNNKRPSGIPDRFSGSRSGTSAT
LGITGLQTGDEADYYCGSWDSSLSVYVFGTGTKVTVLG
Clone 9 light chain variable domain, protein
(SEQ ID NO: 27)
QSVLTQPPSVSAAPGQKVTISCSGSSSNIGSNYISWYQQLPGTAPKLLIYDNSKRPSGIPDRFSGFKSGTSATL
GITGLQTGDEADYYCGTWDSSLSAYVFGSGTKVTVLG
Clone 10 light chain variable domain, protein
(SEQ ID NO: 28)
QSVLTQPPSVSAAPGQKVTISCSGSSSNIGSNYVSWYQQLPGTAPKLLIYDNNKRPSGIPDRFSGSKSGTSAT
LGITGLQTGDEADYYCGTWDSSLSAYVFGTGTKVTVLG
Clone 11 light chain variable domain, protein
(SEQ ID NO: 29)
NFMLTQPHSVSESPGKTVTISCTRSSGSIASNYVQWYQQRPGSAPTTVIYEDNQRPSGVPDRFSGSIDSSSNS
ASLTISGLKTEDEADYYCQSYDSSNVVFGGGTKLTVLG
Clone 12 light chain variable domain, protein
(SEQ ID NO: 30)
QSVLTQPPSVSAAPGQRVTIYCSGSRSNIVSNYVSWYQQLPGTAPKLLIYDNNKRPSGIPDRFSGSKSGTSAT
LGITGLQTGDEADYYCGTWDSSLSAYVFGTGTKVAVLG
Clone 13 light chain variable domain, protein
(SEQ ID NO: 31)
QSVLTQPPSVSAAPGQKVTISCSGSSSNIGSNYVSWYQQLPGTVPKLLIYDNNKRPSGIPDRFSGSKSGTSAT
LGITGLQTGDEADYYCGIWDSSLSAYVFGTGTKVTVLG
Clone 14 light chain variable domain, protein
(SEQ ID NO: 32)
QSVLTQPPSVSAAPGQRVTISCSGSTSNIGSNYVSWYQQFPGTAPKLLIYDSDKRISGIPDRFSGSKSGTSATL
GITGLQTGDEADYYCGTWDSSLTVGVFGGGTKVTVLG
Clone 15 light chain variable domain, protein
(SEQ ID NO: 33)
QSVLTQPPSVSAAPGQKVTISCSGSSSNIGSNYVSWYQQLPGTAPKLLIYDNNKRPSGIPDRFSGSKSGTSAIL
GITGLQTGDEADYYCGTWDSSLTAGVFGTGTKVTVLG
Clone 16 light chain variable domain, protein
(SEQ ID NO: 34)
LPVLTQPPSVSVSPGQTASITCSGDKLGDKYACWYQQKPGQSPVLVIYQDSKRPSGIPERFSGSNSGNTATL
TISGTQAMDEADYYCQAWDSSTAYVFGTGTKVTVLG
Clone 17 light chain variable domain, protein
(SEQ ID NO: 35)
SSELTQDPAVSVALGQTVRITCQGDTLRSYYANWYQQKPGQAPVLVIYGRNNRPSGIPDRFSGSDSGNTAS
LTITGAQAEDEADYYCQSFDYSYSVFGGGTKLTVLG
Clone 18 light chain variable domain, protein
(SEQ ID NO: 36)
QSVLTQPPSVSVAPGQKVTISCSGSSSNIGNNYVSWYQQLPGTAPKLLIYDNNKRPSGIPDRFSGSKSGTSAT
LGITGLQTGDEADYYCQSYDVYNMTSVFGGGTKLTVLG
Clone 19 light chain variable domain, protein
(SEQ ID NO: 37)
NFMLTQPHSVSESPGKTVTISCVRSSGSVASEFVQWYQQRPGHAPTLVIYNDFQRPSGVPDRFSGSIDKSSNS
ASLTISGLKAEDEADYYCQSYDQSSSIVFGGGTKLTVLG

In some embodiments, the present disclosure includes anti-NDC80/MHC immunoglobulin-related compositions that have a scFv sequence fused to one or more constant domains of a heavy chain to form an antibody with an Fc region of a human immunoglobulin to yield a bivalent protein, increasing the overall avidity and stability of the antibody. The results presented here highlight the specificity, sensitivity and utility of the immunoglobulin-related compositions of the present disclosure in targeting MHC-peptide complexes.

The molecules of the present disclosure are based on the identification and selection of single chain variable fragments (scFv) using phage display, the amino acid sequence of which confers the molecules' specificity for the MHC restricted peptide of interest and forms the basis of all immunoglobulin-related compositions of the disclosure. The scFv, therefore, can be used to design a diverse array of “antibody” molecules, including, for example, full length antibodies, fragments thereof, such as Fab and F(ab′)2, minibodies, fusion proteins, including scFv-Fc fusions, multivalent antibodies, that is, antibodies that have more than one specificity for the same antigen or different antigens, for example, bispecific T-cell engaging antibodies (BiTe), tribodies, etc. (see Cuesta et al., Multivalent antibodies: when design surpasses evolution. Trends in Biotechnology 28:355-362 2010).

In one aspect, the present disclosure provides immunoglobulin-related compositions comprising antibody moieties that specifically bind to NDC80 peptide/MHC complexes (also called “anti-NDC80 peptide/MHC” or “anti-NDC80/MHC” herein). Such compositions can comprise, consist essentially of, or consist of, e.g., anti-NDC80 peptide/MHC antibodies or antigen binding fragments thereof, chimeric antibody-T cell receptors (caTCRs), chimeric antigen receptors (CARs), fusion proteins, and conjugates. The antibody moieties can comprise: (a) a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 2, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 20, (b) a V_{H} comprising a V_{H}-CDR1 sequence, a V_{H}-CDR2 sequence, and a V_{H}-CDR3 sequence of the V_{H} sequence of SEQ ID NO: 3, and a V_{L} comprising a V_{L}-CDR1 sequence, a V_{L}-CDR2 sequence, and a V_{L}-CDR3 sequence of the V_{L} sequence SEQ ID NO: 21, (c) a V_{H} comprising a V_{H}-CDR1 sequence, a V_{H}-CDR2 sequence, and a V_{H}-CDR3 sequence of the V_{H} sequence of SEQ ID NO: 4, and a V_{L} comprising a V_{L}-CDR1 sequence, a V_{L}-CDR2 sequence, and a V_{L}-CDR3 sequence of the V_{L} sequence SEQ ID NO: 22, (d) a V_{H} comprising a V_{H}-CDR1 sequence, a V_{H}-CDR2 sequence, and a V_{H}-CDR3 sequence of the V_{H} sequence of SEQ ID NO: 5, and a V_{L} comprising a V_{L}-CDR1 sequence, a V_{L}-CDR2 sequence, and a V_{L}-CDR3 sequence of the V_{L} sequence SEQ ID NO: 23, (e) a V_{H} comprising a V_{H}-CDR1 sequence, a V_{H}-CDR2 sequence, and a V_{H}-CDR3 sequence of the V_{H} sequence of SEQ ID NO: 6, and a V_{L} comprising a V_{L}-CDR1 sequence, a V_{L}-CDR2 sequence, and a V_{L}-CDR3 sequence of the V_{L} sequence SEQ ID NO: 24, (f) a V_{H} comprising a V_{H}-CDR1 sequence, a V_{H}-CDR2 sequence, and a V_{H}-CDR3 sequence of the V_{H} sequence of SEQ ID NO: 7, and a V_{L} comprising a V_{L}-CDR1 sequence, a V_{L}-CDR2 sequence, and a V_{L}-CDR3 sequence of the V_{L} sequence SEQ ID NO: 25, (g) a V_{H} comprising a V_{H}-CDR1 sequence, a V_{H}-CDR2 sequence, and a V_{H}-CDR3 sequence of the V_{H} sequence of SEQ ID NO: 8, and a V_{L} comprising a V_{L}-CDR1 sequence, a V_{L}-CDR2 sequence, and a V_{L}-CDR3 sequence of the V_{L} sequence SEQ ID NO: 26, (h) a V_{H} comprising a V_{H}-CDR1 sequence, a V_{H}-CDR2 sequence, and a V_{H}-CDR3 sequence of the V_{H} sequence of SEQ ID NO: 9, and a V_{L} comprising a V_{L}-CDR1 sequence, a V_{L}-CDR2 sequence, and a V_{L}-CDR3 sequence of the V_{L} sequence SEQ ID NO: 27, (i) a V_{H} comprising a V_{H}-CDR1 sequence, a V_{H}-CDR2 sequence, and a V_{H}-CDR3 sequence of the V_{H} sequence of SEQ ID NO: 10, and a V_{L} comprising a V_{L}-CDR1 sequence, a V_{L}-CDR2 sequence, and a V_{L}-CDR3 sequence of the V_{L} sequence SEQ ID NO: 28, (j) a V_{H} comprising a V_{H}-CDR1 sequence, a V_{H}-CDR2 sequence, and a V_{H}-CDR3 sequence of the V_{H} sequence of SEQ ID NO: 11, and a V_{L} comprising a V_{L}-CDR1 sequence, a V_{L}-CDR2 sequence, and a V_{L}-CDR3 sequence of the V_{L} sequence SEQ ID NO: 29, (k) a V_{H} comprising a V_{H}-CDR1 sequence, a V_{H}-CDR2 sequence, and a V_{H}-CDR3 sequence of the V_{H} sequence of SEQ ID NO: 12, and a V_{L} comprising a V_{L}-CDR1 sequence, a V_{L}-CDR2 sequence, and a V_{L}-CDR3 sequence of the V_{L} sequence SEQ ID NO: 30, (l) a V_{H} comprising a V_{H}-CDR1 sequence, a V_{H}-CDR2 sequence, and a V_{H}-CDR3 sequence of the V_{H} sequence of SEQ ID NO: 13, and a V_{L} comprising a V_{L}-CDR1 sequence, a V_{L}-CDR2 sequence, and a V_{L}-CDR3 sequence of the V_{L} sequence SEQ ID NO: 31, (m) a V_{H} comprising a V_{H}-CDR1 sequence, a V_{H}-CDR2 sequence, and a V_{H}-CDR3 sequence of the V_{H} sequence of SEQ ID NO: 14, and a V_{L} comprising a V_{L}-CDR1 sequence, a V_{L}-CDR2 sequence, and a V_{L}-CDR3 sequence of the V_{L} sequence SEQ ID NO: 32, (n) a V_{H} comprising a V_{H}-CDR1 sequence, a V_{H}-CDR2 sequence, and a V_{H}-CDR3 sequence of the V_{H} sequence of SEQ ID NO: 15, and a V_{L} comprising a V_{L}-CDR1 sequence, a V_{L}-CDR2 sequence, and a V_{L}-CDR3 sequence of the V_{L} sequence SEQ ID NO: 33, (o) a V_{H} comprising a V_{H}-CDR1 sequence, a V_{H}-CDR2 sequence, and a V_{H}-CDR3 sequence of the V_{H} sequence of SEQ ID NO: 16, and a V_{L} comprising a V_{L}-CDR1 sequence, a V_{L}-CDR2 sequence, and a V_{L}-CDR3 sequence of the V_{L} sequence SEQ ID NO: 34, (p) a V_{H} comprising a V_{H}-CDR1 sequence, a V_{H}-CDR2 sequence, and a V_{H}-CDR3 sequence of the V_{H} sequence of SEQ ID NO: 17, and a V_{L} comprising a V_{L}-CDR1 sequence, a V_{L}-CDR2 sequence, and a V_{L}-CDR3 sequence of the V_{L} sequence SEQ ID NO: 35, (q) a V_{H} comprising a V_{H}-CDR1 sequence, a V_{H}-CDR2 sequence, and a V_{H}-CDR3 sequence of the V_{H} sequence of SEQ ID NO: 18, and a V_{L} comprising a V_{L}-CDR1 sequence, a V_{L}-CDR2 sequence, and a V_{L}-CDR3 sequence of the V_{L} sequence SEQ ID NO: 36, or (r) a V_{H} comprising a V_{H}-CDR1 sequence, a V_{H}-CDR2 sequence, and a V_{H}-CDR3 sequence of the V_{H} sequence of SEQ ID NO: 19, and a V_{L} comprising a V_{L}-CDR1 sequence, a V_{L}-CDR2 sequence, and a V_{L}-CDR3 sequence of the V_{L} sequence SEQ ID NO: 37.

In one aspect, the present disclosure provides immunoglobulin-related compositions (e.g., antibodies or antigen binding fragments thereof, chimeric antibody-T cell receptors (caTCRs), chimeric antigen receptors (CARs), fusion proteins, and conjugates) comprising a heavy chain immunoglobulin variable domain (V_{H}) and a light chain immunoglobulin variable domain (V_{L}), wherein (a) the V_{H} comprises a V_{H}-CDR1 sequence selected from the group consisting of GYSFSSNW (SEQ ID NO: 44), GGTFSSYA (SEQ ID NO: 47), GGSFSGYY (SEQ ID NO: 50), GGSISSSNW (SEQ ID NO: 53), GGSFSGYY (SEQ ID NO: 56), GYTFTSYG (SEQ ID NO: 59), GDSISGYY (SEQ ID NO: 62), GGSISSSNW (SEQ ID NO: 65), GGSISSSNW (SEQ ID NO: 68), GYSFTSNG (SEQ ID NO: 71), GGSISSSNW (SEQ ID NO: 74), GDSISSNTW (SEQ ID NO: 77), GGSISSSNW (SEQ ID NO: 80), GGSISSSNW (SEQ ID NO: 83), GYTFTSYY (SEQ ID NO: 86), GSTFINYG (SEQ ID NO: 89), GGTFSSYA (SEQ ID NO: 92), and GYSFTTYW (SEQ ID NO: 95), a V_{H}-CDR2 sequence selected from the group consisting of IYPGDSDT (SEQ ID NO: 45), ISAYNGNT (SEQ ID NO: 48), INHSGST (SEQ ID NO: 51), IYHSGST (SEQ ID NO: 54), INHSGST (SEQ ID NO: 57), ISAYNGNT (SEQ ID NO: 60), INHSGST (SEQ ID NO: 63), IYHSGST (SEQ ID NO: 66), IYHSGST (SEQ ID NO: 69), ISGYNANT (SEQ ID NO: 72), IYHGGST (SEQ ID NO: 75), INHSGST (SEQ ID NO: 78), IYHSGST (SEQ ID NO: 81), IYHSGST (SEQ ID NO: 84), INPSGGST (SEQ ID NO: 87), VSGYNGNT (SEQ ID NO: 90), IIPIFGTA (SEQ ID NO: 93) and IYPGDSDT (SEQ ID NO: 96), and a V_{H}-CDR3 sequence selected from the group consisting of ARFAGPGMWSYGFDY (SEQ ID NO: 46), ARKGFYSGFDS (SEQ ID NO: 49), ARGVPYDY (SEQ ID NO: 52), ARFIYGSSSSQTDT (SEQ ID NO: 55), ARLEGSYYDV (SEQ ID NO: 58), ARMSMSEYIDY (SEQ ID NO: 61), ARYYSGFYSSDG (SEQ ID NO: 64), ARYEQGSHDS (SEQ ID NO: 67), ARYYNSYYDS (SEQ ID NO: 70), ARHAYWGGDSDY (SEQ ID NO: 73), ARYASSSHDF (SEQ ID NO: 76), ARWGYGSAYSDA (SEQ ID NO: 79), ARYFGQKYDY (SEQ ID NO: 82), ARYPGFVWDL (SEQ ID NO: 85), ARRGWLSWGYYGMDV (SEQ ID NO: 88), ARYYYPPYDY (SEQ ID NO: 91), ARGFSSWPGIDQ (SEQ ID NO: 94), and ARYGGQYFWSDSFDS (SEQ ID NO: 97), and/or (b) the V_{L} comprises a V_{L}-CDR1 sequence selected from the group consisting of QGIRND (SEQ ID NO: 98), SSNIGSNY (SEQ ID NO: 101), RSNIGSNF (SEQ ID NO: 104), SSNIGSNY (SEQ ID NO: 107), TSDIGSNY (SEQ ID NO: 110), SSNIGAGYD (SEQ ID NO: 113), SSNIGSNY (SEQ ID NO: 116), SSNIGSNY (SEQ ID NO: 119), SSNIGSNY (SEQ ID NO: 122), SGSIASNY (SEQ ID NO: 125), RSNIVSNY (SEQ ID NO: 128), SSNIGSNY (SEQ ID NO: 131), TSNIGSNY (SEQ ID NO: 134), SSNIGSNY (SEQ ID NO: 137), KLGDKY (SEQ ID NO: 140), TLRSYY (SEQ ID NO: 143), SSNIGNNY (SEQ ID NO: 146), and SGSVASEF (SEQ ID NO: 149), a V_{L}-CDR2 sequence selected from the group consisting of AAS (SEQ ID NO: 99), DNT (SEQ ID NO: 102), DNN (SEQ ID NO: 105), DDN (SEQ ID NO: 108), DNN (SEQ ID NO: 111), GNV (SEQ ID NO: 114), DNN (SEQ ID NO: 117), DNS (SEQ ID NO: 120), DNN (SEQ ID NO: 123), EDN (SEQ ID NO: 126), DNN (SEQ ID NO: 129), DNN (SEQ ID NO: 132), DSD (SEQ ID NO: 135), DNN (SEQ ID NO: 138), QDS (SEQ ID NO: 141), GRN (SEQ ID NO: 144), DNN (SEQ ID NO: 147), and NDF (SEQ ID NO: 150), and a V_{L}-CDR3 sequence selected from the group consisting of LQDYDYPLT (SEQ ID NO: 100), ETLDSSLSAYV (SEQ ID NO: 103), GTWDSSLIAGV (SEQ ID NO: 106), GTWDSPLVA (SEQ ID NO: 109), GTWDSSLNTFV (SEQ ID NO: 112), QSYDSSLSGWV (SEQ ID NO: 115), GSWDSSLSVYV (SEQ ID NO: 118), GTWDSSLSAYV (SEQ ID NO: 121), GTWDSSLSAYV (SEQ ID NO: 124), QSYDSSNVV (SEQ ID NO: 127), GTWDSSLSAYV (SEQ ID NO: 130), GIWDSSLSAYV (SEQ ID NO: 133), GTWDSSLTVGV (SEQ ID NO: 136), GTWDSSLTAGV (SEQ ID NO: 139), QAWDSSTAYV (SEQ ID NO: 142), QSFDYSYSV (SEQ ID NO: 145), QSYDVYNMTSV (SEQ ID NO: 148), and QSYDQSSSIV (SEQ ID NO: 151).

Additionally or alternatively, in some embodiments of the immunoglobulin-related compositions (e.g., antibodies or antigen binding fragments thereof, chimeric antibody-T cell receptors (caTCRs), chimeric antigen receptors (CARs), fusion proteins, and conjugates) described herein, (a) the V_{H}-CDR1 sequence comprises the sequence of SEQ ID NO: 44, the V_{H}-CDR2 sequence comprises the sequence of SEQ ID NO: 45, the V_{H}-CDR3 sequence comprises the sequence of SEQ ID NO: 46, the V_{L}-CDR1 sequence comprises the sequence of SEQ ID NO: 98, the V_{L}-CDR2 sequence comprises the sequence of SEQ ID NO: 99, and/or the V_{L}-CDR3 sequence comprises the sequence of SEQ ID NO: 100; (b) the V_{H}-CDR1 sequence comprises the sequence of SEQ ID NO: 47, the V_{H}-CDR2 sequence comprises the sequence of SEQ ID NO: 48, the V_{H}-CDR3 sequence comprises the sequence of SEQ ID NO: 49, the V_{L}-CDR1 sequence comprises the sequence of SEQ ID NO: 101, the V_{L}-CDR2 sequence comprises the sequence of SEQ ID NO: 102, and/or the V_{L}-CDR3 sequence comprises the sequence of SEQ ID NO: 103; (c) the V_{H}-CDR1 sequence comprises the sequence of SEQ ID NO: 50, the V_{H}-CDR2 sequence comprises the sequence of SEQ ID NO: 51, the V_{H}-CDR3 sequence comprises the sequence of SEQ ID NO: 52, the V_{L}-CDR1 sequence comprises the sequence of SEQ ID NO: 104, the V_{L}-CDR2 sequence comprises the sequence of SEQ ID NO: 105, and/or the V_{L}-CDR3 sequence comprises the sequence of SEQ ID NO: 106; (d) the V_{H}-CDR1 sequence comprises the sequence of SEQ ID NO: 53, the V_{H}-CDR2 sequence comprises the sequence of SEQ ID NO: 54, the V_{H}-CDR3 sequence comprises the sequence of SEQ ID NO: 55, the V_{L}-CDR1 sequence comprises the sequence of SEQ ID NO: 107, the V_{L}-CDR2 sequence comprises the sequence of SEQ ID NO: 108, and/or the V_{L}-CDR3 sequence comprises the sequence of SEQ ID NO: 109; (e) the V_{H}-CDR1 sequence comprises the sequence of SEQ ID NO: 56, the V_{H}-CDR2 sequence comprises the sequence of SEQ ID NO: 57, the V_{H}-CDR3 sequence comprises the sequence of SEQ ID NO: 58, the V_{L}-CDR1 sequence comprises the sequence of SEQ ID NO: 110, the V_{L}-CDR2 sequence comprises the sequence of SEQ ID NO: 111, and/or the V_{L}-CDR3 sequence comprises the sequence of SEQ ID NO: 112; (f) the V_{H}-CDR1 sequence comprises the sequence of SEQ ID NO: 59, the V_{H}-CDR2 sequence comprises the sequence of SEQ ID NO: 60, the V_{H}-CDR3 sequence comprises the sequence of SEQ ID NO: 61, the V_{L}-CDR1 sequence comprises the sequence of SEQ ID NO: 113, the V_{L}-CDR2 sequence comprises the sequence of SEQ ID NO: 114, and/or the V_{L}-CDR3 sequence comprises the sequence of SEQ ID NO: 115; (g) the V_{H}-CDR1 sequence comprises the sequence of SEQ ID NO: 62, the V_{H}-CDR2 sequence comprises the sequence of SEQ ID NO: 63, the V_{H}-CDR3 sequence comprises the sequence of SEQ ID NO: 64, the V_{L}-CDR1 sequence comprises the sequence of SEQ ID NO: 116, the V_{L}-CDR2 sequence comprises the sequence of SEQ ID NO: 117, and/or the V_{L}-CDR3 sequence comprises the sequence of SEQ ID NO: 118; (h) the V_{H}-CDR1 sequence comprises the sequence of SEQ ID NO: 65, the V_{H}-CDR2 sequence comprises the sequence of SEQ ID NO: 66, the V_{H}-CDR3 sequence comprises the sequence of SEQ ID NO: 67, the V_{L}-CDR1 sequence comprises the sequence of SEQ ID NO: 119, the V_{L}-CDR2 sequence comprises the sequence of SEQ ID NO: 120, and/or the V_{L}-CDR3 sequence comprises the sequence of SEQ ID NO: 121; (i) the V_{H}-CDR1 sequence comprises the sequence of SEQ ID NO: 68, the V_{H}-CDR2 sequence comprises the sequence of SEQ ID NO: 69, the V_{H}-CDR3 sequence comprises the sequence of SEQ ID NO: 70, the V_{L}-CDR1 sequence comprises the sequence of SEQ ID NO: 122, the V_{L}-CDR2 sequence comprises the sequence of SEQ ID NO: 123, and/or the V_{L}-CDR3 sequence comprises the sequence of SEQ ID NO: 124; (j) the V_{H}-CDR1 sequence comprises the sequence of SEQ ID NO: 71, the V_{H}-CDR2 sequence comprises the sequence of SEQ ID NO: 72, the V_{H}-CDR3 sequence comprises the sequence of SEQ ID NO: 73, the V_{L}-CDR1 sequence comprises the sequence of SEQ ID NO: 125, the V_{L}-CDR2 sequence comprises the sequence of SEQ ID NO: 126, and/or the V_{L}-CDR3 sequence comprises the sequence of SEQ ID NO: 127; (k) the V_{H}-CDR1 sequence comprises the sequence of SEQ ID NO: 74, the V_{H}-CDR2 sequence comprises the sequence of SEQ ID NO: 75, the V_{H}-CDR3 sequence comprises the sequence of SEQ ID NO: 76, the V_{L}-CDR1 sequence comprises the sequence of SEQ ID NO: 128, the V_{L}-CDR2 sequence comprises the sequence of SEQ ID NO: 129, and/or the V_{L}-CDR3 sequence comprises the sequence of SEQ ID NO: 130; (1) the V_{H}-CDR1 sequence comprises the sequence of SEQ ID NO: 77, the V_{H}-CDR2 sequence comprises the sequence of SEQ ID NO: 78, the V_{H}-CDR3 sequence comprises the sequence of SEQ ID NO: 79, the V_{L}-CDR1 sequence comprises the sequence of SEQ ID NO: 131, the V_{L}-CDR2 sequence comprises the sequence of SEQ ID NO: 132, and/or the V_{L}-CDR3 sequence comprises the sequence of SEQ ID NO: 133; (m) the V_{H}-CDR1 sequence comprises the sequence of SEQ ID NO: 80, the V_{H}-CDR2 sequence comprises the sequence of SEQ ID NO: 81, the V_{H}-CDR3 sequence comprises the sequence of SEQ ID NO: 82, the V_{L}-CDR1 sequence comprises the sequence of SEQ ID NO: 134, the V_{L}-CDR2 sequence comprises the sequence of SEQ ID NO: 135, and/or the V_{L}-CDR3 sequence comprises the sequence of SEQ ID NO: 136; (n) the V_{H}-CDR1 sequence comprises the sequence of SEQ ID NO: 83, the V_{H}-CDR2 sequence comprises the sequence of SEQ ID NO: 84, the V_{H}-CDR3 sequence comprises the sequence of SEQ ID NO: 85, the V_{L}-CDR1 sequence comprises the sequence of SEQ ID NO: 137, the V_{L}-CDR2 sequence comprises the sequence of SEQ ID NO: 138, and/or the V_{L}-CDR3 sequence comprises the sequence of SEQ ID NO: 139; (o) the V_{H}-CDR1 sequence comprises the sequence of SEQ ID NO: 86, the V_{H}-CDR2 sequence comprises the sequence of SEQ ID NO: 87, the V_{H}-CDR3 sequence comprises the sequence of SEQ ID NO: 88, the V_{L}-CDR1 sequence comprises the sequence of SEQ ID NO: 140, the V_{L}-CDR2 sequence comprises the sequence of SEQ ID NO: 141, and/or the V_{L}-CDR3 sequence comprises the sequence of SEQ ID NO: 142; (p) the V_{H}-CDR1 sequence comprises the sequence of SEQ ID NO: 89, the V_{H}-CDR2 sequence comprises the sequence of SEQ ID NO: 90, the V_{H}-CDR3 sequence comprises the sequence of SEQ ID NO: 91, the V_{L}-CDR1 sequence comprises the sequence of SEQ ID NO: 143, the V_{L}-CDR2 sequence comprises the sequence of SEQ ID NO: 144, and/or the V_{L}-CDR3 sequence comprises the sequence of SEQ ID NO: 145; (q) the V_{H}-CDR1 sequence comprises the sequence of SEQ ID NO: 92, the V_{H}-CDR2 sequence comprises the sequence of SEQ ID NO: 93, the V_{H}-CDR3 sequence comprises the sequence of SEQ ID NO: 94, the V_{L}-CDR1 sequence comprises the sequence of SEQ ID NO: 146, the V_{L}-CDR2 sequence comprises the sequence of SEQ ID NO: 147, and/or the V_{L}-CDR3 sequence comprises the sequence of SEQ ID NO: 148; or (r) the V_{H}-CDR1 sequence comprises the sequence of SEQ ID NO: 95, the V_{H}-CDR2 sequence comprises the sequence of SEQ ID NO: 96, the V_{H}-CDR3 sequence comprises the sequence of SEQ ID NO: 97, the V_{L}-CDR1 sequence comprises the sequence of SEQ ID NO: 149, the V_{L}-CDR2 sequence comprises the sequence of SEQ ID NO: 150, and/or the V_{L}-CDR3 sequence comprises the sequence of SEQ ID NO: 151.

In one aspect, the present technology provides immunoglobulin-related compositions (e.g., antibodies or antigen binding fragments thereof, chimeric antibody-T cell receptors (caTCRs), chimeric antigen receptors (CARs), fusion proteins, and conjugates) comprising a heavy chain immunoglobulin variable domain (V_{H}) and a light chain immunoglobulin variable domain (V_{L}), wherein (a) the V_{H} comprises an amino acid sequence selected from the group consisting of: SEQ ID NOs: 2-19, or a variant thereof having one or more conservative amino acid substitutions; and/or (b) the V_{L} comprises an amino acid sequence selected from the group consisting of: SEQ ID NOs: 20-37, or a variant thereof having one or more conservative amino acid substitutions.

Additionally or alternatively, in some embodiments, the immunoglobulin-related compositions of the present technology comprise a V_{H} amino acid sequence and a V_{L} amino acid sequence selected from the group consisting of: SEQ ID NO: 2 and SEQ ID NO: 20; SEQ ID NO: 3 and SEQ ID NO: 21; SEQ ID NO: 4 and SEQ ID NO: 22; SEQ ID NO: 5 and SEQ ID NO: 23; SEQ ID NO: 6 and SEQ ID NO: 24; SEQ ID NO: 7 and SEQ ID NO: 25; SEQ ID NO: 8 and SEQ ID NO: 26; SEQ ID NO: 9 and SEQ ID NO: 27; SEQ ID NO: 10 and SEQ ID NO: 28; SEQ ID NO: 11 and SEQ ID NO: 29; SEQ ID NO: 12 and SEQ ID NO: 30; SEQ ID NO: 13 and SEQ ID NO: 31; SEQ ID NO: 14 and SEQ ID NO: 32; SEQ ID NO: 15 and SEQ ID NO: 33; SEQ ID NO: 16 and SEQ ID NO: 34; SEQ ID NO: 17 and SEQ ID NO: 35; SEQ ID NO: 18 and SEQ ID NO: 36; and SEQ ID NO: 19 and SEQ ID NO: 37.

Additionally or alternatively, in some embodiments, the immunoglobulin-related compositions of the present technology comprise an amino acid sequence having at least 80%, at least 85%, at least 90%, at least 95% or at least 99% identity to a sequence selected from the group consisting of: SEQ ID NOs: 38-43. In certain embodiments, the antigen binding proteins or immunoglobulin-related compositions of the present technology comprise an amino acid sequence selected from the group consisting of: SEQ ID NOs: 38-43.

In any of the above embodiments, the immunoglobulin-related composition further comprises a Fc domain of any isotype, e.g., but are not limited to, IgG (including IgG1, IgG2, IgG3, and IgG4), IgA (including IgA1 and IgA2), IgD, IgE, or IgM, and IgY. Non-limiting examples of constant region sequences include:

Human IgD constant region, Uniprot: P01880
(SEQ ID NO: 152)
APTKAPDVFPIISGCRHPKDNSPVVLACLITGYHPTSVTV
TWYMGTQSQPQRTFPEIQRRDSYYMTSSQLSTPLQQWRQG
EYKCVVQHTASKSKKEIFRWPESPKAQASSVPTAQPQAQD
LWLRDKATFTCFVVGSDLKDAHLTWEVAGKVPTGGVEEGL
LERHSNGSQSQHSRLTLPRSLWNAGTSVTCTLNHPSLPPQ
RLMALREPAAQAPVKLSLNLLASSDPPEAASWLLCEVSGF
SPPNILLMWLEDQREVNTSGFAPARPPPQPGSTTFWAWSV
LRVPAPPSPQPATYTCVVSHEDSRTLLNASRSLEVSYVTD
HGPMK
Human IgG1 constant region, Uniprot: P01857
(SEQ ID NO: 153)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSRDE
LTKNQVSLTCLVKGFYPSDIAVEWESNGQPENNYKTTPPV
LDSDGSFFLYSKLTVDKSRWQQGNVFSCSVMHEALHNHYT
QKSLSLSPGK
Human IgG2 constant region, Uniprot: P01859
(SEQ ID NO: 154)
ASTKGPSVFPLAPCSRSTSESTAALGCLVKDYFPEPVTVS
WNSGALTSGVHTFPAVLQSSGLYSLSSVVTVPSSNFGTQT
YTCNVDHKPSNTKVDKTVERKCCVECPPCPAPPVAGPSVF
LFPPKPKDTLMISRTPEVTCVVVDVSHEDPEVQFNWYVDG
VEVHNAKTKPREEQFNSTFRVVSVLTVVHQDWLNGKEYKC
KVSNKGLPAPIEKTISKTKGQPREPQVYTLPPSREEMTKN
QVSLTCLVKGFYPSDISVEWESNGQPENNYKTTPPMLDSD
GSFFLYSKLTVDKSRWQQGNVFSCSVMHEALHNHYTQKSL
SLSPGK
Human IgG3 constant region, Uniprot: P01860
(SEQ ID NO: 155)
ASTKGPSVFPLAPCSRSTSGGTAALGCLVKDYFPEPVTVS
WNSGALTSGVHTFPAVLQSSGLYSLSSVVTVPSSSLGTQT
YTCNVNHKPSNTKVDKRVELKTPLGDTTHTCPRCPEPKSC
DTPPPCPRCPEPKSCDTPPPCPRCPEPKSCDTPPPCPRCP
APELLGGPSVFLFPPKPKDTLMISRTPEVTCVVVDVSHED
PEVQFKWYVDGVEVHNAKTKPREEQYNSTFRVVSVLTVLH
QDWLNGKEYKCKVSNKALPAPIEKTISKTKGQPREPQVYT
LPPSREEMTKNQVSLTCLVKGFYPSDIAVEWESSGQPENN
YNTTPPMLDSDGSFFLYSKLTVDKSRWQQGNIFSCSVMHE
ALHNRFTQKSLSLSPGK
Human IgM constant region, Uniprot: P01871
(SEQ ID NO: 156)
GSASAPTLFPLVSCENSPSDTSSVAVGCLAQDFLPDSITL
SWKYKNNSDISSTRGFPSVLRGGKYAATSQVLLPSKDVMQ
GTDEHVVCKVQHPNGNKEKNVPLPVIAELPPKVSVFVPPR
DGFFGNPRKSKLICQATGFSPRQIQVSWLREGKQVGSGVT
TDQVQAEAKESGPTTYKVTSTLTIKESDWLGQSMFTCRVD
HRGLTFQQNASSMCVPDQDTAIRVFAIPPSFASIFLTKST
KLTCLVTDLTTYDSVTISWTRQNGEAVKTHTNISESHPNA
TFSAVGEASICEDDWNSGERFTCTVTHTDLPSPLKQTISR
PKGVALHRPDVYLLPPAREQLNLRESATITCLVTGFSPAD
VFVQWMQRGQPLSPEKYVTSAPMPEPQAPGRYFAHSILTV
SEEEWNTGETYTCVAHEALPNRVTERTVDKSTGKPTLYNV
SLVMSDTAGTCY
Human IgG4 constant region, Uniprot: P01861
(SEQ ID NO: 157)
ASTKGPSVFPLAPCSRSTSESTAALGCLVKDYFPEPVTVS
WNSGALTSGVHTFPAVLQSSGLYSLSSVVTVPSSSLGTKT
YTCNVDHKPSNTKVDKRVESKYGPPCPSCPAPEFLGGPSV
FLFPPKPKDTLMISRTPEVTCVVVDVSQEDPEVQFNWYVD
GVEVHNAKTKPREEQFNSTYRVVSVLTVLHQDWLNGKEYK
CKVSNKGLPSSIEKTISKAKGQPREPQVYTLPPSQEEMTK
NQVSLTCLVKGFYPSDIAVEWESNGQPENNYKTTPPVLDS
DGSFFLYSRLTVDKSRWQEGNVFSCSVMHEALHNHYTQKS
LSLSLGK
Human IgA1 constant region, Uniprot: P01876
(SEQ ID NO: 158)
ASPTSPKVFPLSLCSTQPDGNVVIACLVQGFFPQEPLSVT
WSESGQGVTARNFPPSQDASGDLYTTSSQLTLPATQCLAG
KSVTCHVKHYTNPSQDVTVPCPVPSTPPTPSPSTPPTPSP
SCCHPRLSLHRPALEDLLLGSEANLTCTLTGLRDASGVTF
TWTPSSGKSAVQGPPERDLCGCYSVSSVLPGCAEPWNHGK
TFTCTAAYPESKTPLTATLSKSGNTFRPEVHLLPPPSEEL
ALNELVTLTCLARGFSPKDVLVRWLQGSQELPREKYLTWA
SRQEPSQGTTTFAVTSILRVAAEDWKKGDTFSCMVGHEAL
PLAFTQKTIDRLAGKPTHVNVSVVMAEVDGTCY
Human IgA2 constant region, Uniprot: P01877
(SEQ ID NO: 159)
ASPTSPKVFPLSLDSTPQDGNVVVACLVQGFFPQEPLSVT
WSESGQNVTARNFPPSQDASGDLYTTSSQLTLPATQCPDG
KSVTCHVKHYTNPSQDVTVPCPVPPPPPCCHPRLSLHRPA
LEDLLLGSEANLTCTLTGLRDASGATFTWTPSSGKSAVQG
PPERDLCGCYSVSSVLPGCAQPWNHGETFTCTAAHPELKT
PLTANITKSGNTFRPEVHLLPPPSEELALNELVTLTCLAR
GFSPKDVLVRWLQGSQELPREKYLTWASRQEPSQGTTTFA
VTSILRVAAEDWKKGDTFSCMVGHEALPLAFTQKTIDRMA
GKPTHVNVSVVMAEVDGTCY
Human Ig kappa constant region, Uniprot: P01834
(SEQ ID NO: 160)
TVAAPSVFIFPPSDEQLKSGTASVVCLLNNFYPREAKVQW
KVDNALQSGNSQESVTEQDSKDSTYSLSSTLTLSKADYEK
HKVYACEVTHQGLSSPVTKSFNRGEC
Human Ig lambda constant 3, Uniprot: PODOY3
(SEQ ID NO: 161)
GQPKAAPSVTLFPPSSEELQANKATLVCLISDFYPGAVTV
AWKADSSPVKAGVETTTPSKQSNNKYAASSYLSLTPEQWK
SHKSYSCQVTHEGSTVEKTVAPTECS
Human Ig lambda constant 2, Uniprot: PODOY2
(SEQ ID NO: 162)
GQPKAAPSVTLFPPSSEELQANKATLVCLISDFYPGAVTV
AWKADSSPVKAGVETTTPSKQSNNKYAASSYLSLTPEQWK
SHRSYSCQVTHEGSTVEKTVAPTECS
Human Ig lambda constant 7, Uniprot: A0M8Q6
(SEQ ID NO: 163)
GQPKAAPSVTLFPPSSEELQANKATLVCLVSDFNPGAVTV
AWKADGSPVKVGVETTKPSKQSNNKYAASSYLSLTPEQWK
SHRSYSCRVTHEGSTVEKTVAPAECS
Human Ig lambda constant 6, Uniprot: POCF74
(SEQ ID NO: 164)
GQPKAAPSVTLFPPSSEELQANKATLVCLISDFYPGAVKV
AWKADGSPVNTGVETTTPSKQSNNKYAASSYLSLTPEQWK
SHRSYSCQVTHEGSTVEKTVAPAECS
Human Ig lambda constant 1, Uniprot: POCG04
(SEQ ID NO: 165)
GQPKANPTVTLFPPSSEELQANKATLVCLISDFYPGAVTV
AWKADGSPVKAGVETTKPSKQSNNKYAASSYLSLTPEQWK
SHRSYSCQVTHEGSTVEKTVAPTECS

In some embodiments, the immunoglobulin-related compositions of the present technology comprise a heavy chain constant region that is at least 80%, at least 85%, at least 90%, at least 95%, at least 99%, or is 100% identical to SEQ ID NOs: 152-159. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO:160-165. In some embodiments, the epitope is a conformational epitope or non-conformational epitope. Additionally or alternatively, in some embodiments, the immunoglobulin-related compositions of the present technology bind to an epitope comprising the amino acid sequence of SEQ ID NO: 1.

In some embodiments in which the immunoglobulin-related composition is a full length antibody, the heavy and light chains of an antibody of the present disclosure may be full-length (e.g., an antibody can include at least one, or two, complete heavy chains, and at least one, or two, complete light chains) or may include an antigen-binding portion (a Fab, F(ab′)2, Fv or a single chain Fv fragment (“scFv”)). In other embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1). In another embodiment, the antibody light chain constant region is chosen from, e.g., kappa or lambda, particularly human kappa or human lambda. The choice of antibody type will depend on the immune effector function that the antibody is designed to elicit. In constructing a recombinant immunoglobulin, appropriate amino acid sequences for constant regions of various immunoglobulin isotypes and methods for the production of a wide array of antibodies are known to those of skill in the art.

In any of the above embodiments of the immunoglobulin-related compositions, the V_{H} and V_{L} amino acid sequences form an antigen binding site that binds to a peptide:MHC complex (e.g., a NDC80 peptide/MHC complex). In some embodiments, the epitope is a conformational epitope or a non-conformational epitope. In some embodiments, the V_{H} and V_{L} sequences are components of the same polypeptide chain. In other embodiments, the V_{H} and V_{L} sequences are components of different polypeptide chains. In certain embodiments, the immunoglobulin-related composition is a full-length antibody.

In some embodiments, the immunoglobulin-related compositions of the present technology bind specifically to at least one peptide/MHC complex (e.g., a NDC80 peptide/MHC complex). In some embodiments, the immunoglobulin-related compositions of the present technology bind at least one peptide/MHC complex (e.g., a NDC80 peptide/MHC complex) with a dissociation constant (K_{D}) of about 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the immunoglobulin-related compositions are monoclonal antibodies, chimeric antibodies, humanized antibodies, or bispecific antibodies. In some embodiments, the immunoglobulin-related compositions comprise a human antibody framework region.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NOs: 20-37; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2-19. In another aspect, one or more amino acid residues in the immunoglobulin-related compositions provided herein are substituted with another amino acid. The substitution may be a “conservative substitution” as defined herein.

In one aspect, the present disclosure provides an anti-NDC80/MHC composition comprising an antibody moiety that competes with the any of the antigen binding proteins or immunoglobulin-related compositions of the present technology for specific binding to a NDC80/MHC complex.

The immunoglobulin-related compositions of the present technology are intended to encompass bispecific antibodies, including bispecific T-cell engaging antibodies, that is, antibodies comprising two antibody variable domains on a single polypeptide chain that are able to bind two separate antigens. Where a first portion of a bispecific antibody binds an antigen on a tumor cell for example and a second portion of a bispecific antibody recognizes an antigen on the surface of a human immune effector cell, the antibody is capable of recruiting the activity of that effector cell by specifically binding to the effector antigen on the human immune effector cell. In some instances, bispecific antibodies, therefore, are able to form a link between effector cells, for example, T cells and tumor cells, thereby enhancing effector function. In one embodiment, the constant region/framework region is altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site). Furthermore, conjugation of the antibody to a drug, toxin, radioisotope, cytokine, inflammatory peptide or cytotoxic agent is also contemplated.

In some aspects, the anti-NDC80/MHC immunoglobulin-related compositions described herein contain structural modifications to facilitate rapid binding and cell uptake and/or slow release. In some aspects, the anti-NDC80/MHC immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

Additionally or alternatively, in some embodiments, the antigen binding proteins or immunoglobulin-related compositions of the present technology are chimeric antibody-T cell receptors (caTCR) and/or comprise at least a fragment of a T cell receptor (TCR) chain. In some embodiments, the fragment of TCR chain comprises the transmembrane domain of the TCR chain. In certain embodiments, the fragment of TCR chain does not comprise any CDR sequence of the TCR chain. Additionally or alternatively, in some embodiments, the antigen binding proteins or immunoglobulin-related compositions of the present technology are chimeric antigen receptors (CARs).

In any and all of the preceding embodiments, the antigen binding proteins or immunoglobulin-related compositions of the present technology may be monospecific, multispecific, or bispecific. In some embodiments, the antigen binding proteins or immunoglobulin-related compositions of the present technology comprise a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. Additionally or alternatively, in some embodiments, the antigen binding proteins or immunoglobulin-related compositions of the present technology comprise a tandem scFv with at least one peptide linker between two scFvs. In certain embodiments, the antigen binding proteins or immunoglobulin-related compositions of the present technology comprise a second antibody moiety that specifically binds to a second antigen. The second antigen may be a disease-specific antigen that is not NDC80/MHC, or an antigen on the surface of a T cell, a natural killer cell, a neutrophil, a monocyte, a macrophage, or a dendritic cell.

Additionally or alternatively, in some embodiments, the immunoglobulin-related compositions (e.g., antibodies, antigen binding fragments, chimeric antibody-T cell receptors (caTCRs), chimeric antigen receptors (CARs), fusion proteins, and conjugates thereof) specifically bind to a NDC80 peptide complexed with HLA-A*02. The HLA-A*02 may be HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50. In certain embodiments, the NDC80 peptide comprises the amino acid sequence ALNEQIARL (SEQ ID NO: 1).

In one aspect, the present disclosure provides recombinant nucleic acids or a set of recombinant nucleic acids encoding any and all embodiments of the antigen binding proteins or immunoglobulin-related compositions described herein, with all components of the composition encoded by one nucleic acid or by the set of nucleic acids. In another aspect, the present disclosure provides a vector comprising said recombinant nucleic acids, as well as a set of vectors comprising said set of recombinant nucleic acids. Vectors comprising the nucleic acids of the present technology for antibody-based treatment by vectored immunotherapy are also contemplated by the present technology. Vectors include expression vectors which enable the expression and secretion of immunoglobulin-related compositions, such as antibodies, as well as vectors which are directed to cell surface expression of the immunoglobulin-related compositions, such as chimeric antigen receptors.

Also disclosed herein are cells comprising any of the recombinant nucleic acids, set of recombinant nucleic acids, vectors, or set of vectors disclosed herein, as well as cells that display on its surface or secrete any of the antigen binding proteins or immunoglobulin-related compositions of the present technology. The cells may be immune effector cells, such as a T cell, a NK cell, a B cell, or a monocyte/macrophage.

The immunoglobulin-related compositions of the present technology (e.g., an anti-NDC80/MHC antibody) can be monospecific, bispecific, trispecific or of greater multispecificity. Multispecific antibodies can be specific for different epitopes of one or more peptide/MHC complexes (e.g., a NDC80 peptide/MHC complex) or can be specific for both the NDC80 peptide/MHC complexes as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In certain embodiments, the Fc portion allows the direct conjugation of other molecules, including but not limited to fluorescent dyes, cytotoxins, radioisotopes etc. to the immunoglobulin-related composition for example, for use in antigen quantitation studies, to immobilize the immunoglobulin-related composition for affinity measurements, for targeted delivery of a therapeutic agent, to test for Fc-mediated cytotoxicity using immune effector cells and many other applications.

In any of the above embodiments of the immunoglobulin-related compositions of the present technology, the immunoglobulin-related compositions may be optionally conjugated to an agent selected from the group consisting of detectable labels, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. Conjugates of the immunoglobulin-related compositions of the present technology with therapeutic agents, include without limitation, drugs (such as calecheamicin, aureastatin, doxorubicin), or toxins (such as ricin, diphtheria, gelonin) or radioisotopes emitting alpha or beta particles (such as ⁹⁰Y, ¹³¹I, ²²⁵Ac, ²¹³Bi, ²²³Ra and ²²⁷Th), inflammatory peptides (such as IL2, TNF, IFN-γ) are encompassed by the present technology.

For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate·2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

The present technology is based on the identification of antigen-specific binding sequences from which a variety of immunoglobulin-related compositions can be produced. In addition to immunoglobulin-related compositions specific for an antigen that represents a protein fragment (peptide)/MHC complex similar to that typically recognized by a T-cell receptor following antigen processing and presentation of the protein to the T-cell, identification of amino acid and nucleic sequences as disclosed herein for the preparation of antibodies can also be used to generate other antigen-binding molecules including chimeric antigen receptors (CARs), with specificity for the protein fragment (peptide)/MHC complex. These can be incorporated into cells to make them specifically cytotoxic to the antigen expressing cell.

The present technology employs an approach to obtaining therapeutic antibodies to proteins that are inaccessible because they are not expressed on the cell surface. Nearly any intracytoplasmic or intranuclear protein (in addition to cell surface proteins) is a potential target for the approach described herein. This approach is to generate recombinant mAbs that recognize the peptide/MHC complex expressed on the cell surface, with the same specificity as a T-cell receptor (TCR). Such mAbs share functional homology with TCRs regarding target recognition, but confer higher affinity and capabilities of arming with potent cytotoxic agents that antibodies feature. TCR-like mAbs may be generated by conventional hybridoma techniques known to those of skill in the art, to produce human, humanized or chimeric antibodies. Recombinant antibodies with TCR-like specificity represent a valuable tool for research and therapeutic applications in tumor immunology and immunotherapy.

Further, fully-human mAbs may be used for therapeutic applications in humans because murine antibodies cause an immunogenicity reaction, known as the HAMA (human anti-mouse antibodies) response, when administered to humans, causing serious side effects, including anaphylaxis and hypersensitivity reactions. This immunogenicity reaction is triggered by the human immune system recognizing the murine antibodies as foreign because of slightly different amino acid sequences from natural human antibodies. Humanization methods known in the art can be employed to reduce the immunogenicity of murine-derived antibodies.

Recently, the use of phage display libraries has made it possible to select large numbers of Ab repertoires for unique and rare Abs against very defined epitopes (for more details on phage display see McCafferty et al., Phage antibodies: filamentous phage displaying antibody variable domains. Nature, 348: 552-554). The rapid identification of human Fab or single chain Fv (scFv) fragments highly specific for tumor antigen-derived peptide-MHC complex molecules has thus become possible. More recently, immuno-toxins, generated by fusing TCR-like Fab specific for melanoma Ag MART-1 26-35/A2 or gp100 280-288/A2 to a truncated form of Pseudomonas endotoxin, have been shown to inhibit human melanoma growth both in vitro and in vivo. In addition, by engineering full-length mAb using the Fab fragments, it is possible to directly generate a therapeutic human mAb, bypassing months of time-consuming work normally needed for developing therapeutic mAbs. The present technology involves the development of a TCR-like, human mAb that recognizes, for example, the NDC80 peptide/HLA-A*02 complex (ALNEQIARL (SEQ ID NO: 1)) for cancer therapy.

Identification of Peptides with High Predictive Binding to HLA Molecules. In some embodiments, the present technology relates to a method for the generation of antibodies that specifically bind to HLA-restricted peptides, which, when presented as part of a peptide/MHC complex are able to elicit a specific cytotoxic T-cell response. HLA class I molecules present endogenous derived peptides of about 8-12 amino acids in length to CD8⁺ cytotoxic T lymphocytes. Peptides to be used in the method of the present technology are generally about 6-22 amino acids in length, and in some embodiments, between about 9 and 20 amino acids and comprise an amino acid sequence derived from a protein of interest, for example, human NDC80 protein (Uniprot Accession No. O14777-1) or an analog thereof.

Peptides suitable for use in generating antibodies in accordance with the method of the present technology can be determined based on the presence of HLA-A*02-binding motifs and the cleavage sites for proteasomes and immune-proteasomes using computer prediction models known to those of skill in the art. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), SYFPEITHI database (see Schuler et al. SYFPEITHI, Database for Searching and T-Cell Epitope Prediction. in Immunoinformatics Methods in Molecular Biology, vol 409(1): 75-93 2007), and netMHCpan 4.1 (see Birkir Reynisson et al., NetMHCpan-4.1 and NetMHCIIpan-4.0: Improved predictions of MHC antigen presentation by concurrent motif deconvolution and integration of MS MHC eluted ligand data. Nucleic Acids Research, 48(W1):W449-W454 (2020)). HLA-A*02:01 is expressed in 39-46% of all caucasians and therefore, represents a suitable choice of MHC antigen for use in the present method. For preparation of one embodiment of a NDC80 peptide antigen, amino acid sequences and predicted binding of putative CD84⁺ epitopes to HLA-A*02:01 molecules were identified using the predictive algorithm of netMHCpan 4.1.

Once appropriate peptides have been identified, peptide synthesis may be done in accordance with protocols well known to those of skill in the art. Because of their relatively small size, the peptides of the present technology may be directly synthesized in solution or on a solid support in accordance with conventional peptide synthesis techniques. Various automatic synthesizers are commercially available and can be used in accordance with known protocols. The synthesis of peptides in solution phase has become a well-established procedure for large-scale production of synthetic peptides and as such is a suitable alternative method for preparing the peptides of the present technology. See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520 1998).

Each of the peptides used in the protocols described herein may be synthesized using fluorenylmethoxycarbonyl chemistry and solid-phase synthesis and purified by high-pressure liquid chromatography. The quality of the peptides can be assessed by high-performance liquid chromatography analysis, and the expected molecular weight can be observed using matrix-assisted laser desorption mass spectrometry. Peptides are preferably sterile and 70% to 90% pure. The peptides may be dissolved in DMSO and diluted in PBS (pH 7.4) or saline at 5 mg/mL and stored at −80° C.

Subsequent to peptide selection, binding activity of selected peptides is tested using the antigen-processing-deficient T2 cell line, which increases expression of HLA-A when stabilized by a peptide in the antigen-presenting groove. Briefly, T2 cells are pulsed with peptide for a time sufficient to induce HLA-A expression. HLA-A expression of T2 cells is then measured by immunostaining with a fluorescently labeled monoclonal antibody specific for HLA-A (for example, BB7.2) and flow cytometry. Fluorescence index (FI) is calculated as the mean fluorescence intensity (MFI) of HLA-A*02 on T2 cells as determined by fluorescence-activated cell-sorting analysis, using the formula FI=(MFI [T2 cells with peptide]/MFI [T2 cells without peptide]−1.

Fully human T-cell receptor (TCR)-like antibodies to NDC80 are produced using the method disclosed herein. TCR-like anti-NDC80/MHC antibodies generated by phage display technology are specific for a NDC80 peptide/HLA complex similar to that which induces HLA-restricted cytotoxic CD8 T-cells. The NDC80 protein sequence may be screened using the netMHCpan 4.1 algorithm and NDC80 peptides may be identified that had predicted high-affinity binding to multiple HLA molecules that are highly expressed in the Caucasian population.

Heteroclitic peptides can also be designed by conservative amino acid substitutions of MHC-binding residues expected to enhance the affinity toward the MHC class 1 allele, as predicted by the prediction algorithm. Peptides used for alanine mutagenesis of NDC80 are named based on the position where the substitution was made. Examples of NDC80 peptides which may be used include SEQ ID NO: 1 and SEQ ID NOs: 166-169. Once a suitable peptide has been identified, the target antigen to be used for phage display library screening, that is, a peptide/HLA complex (for example, NDC80 peptide/HLA-A*02) is prepared by bringing the peptide and the histocompatibility antigen together in solution to form the complex.

Selecting a High Affinity ScFv Against a NDC80 Peptide. The next step is the selection of phage that bind to the target antigen of interest with high affinity, from phage in a human phage display library that either do not bind or that bind with lower affinity. This is accomplished by iterative binding of phage to the antigen, which is bound to a solid support, for example, beads or mammalian cells followed by removal of non-bound phage and by elution of specifically bound phage. In certain embodiments, antigens are first biotinylated for immobilization to, for example, streptavidin-conjugated Dynabeads M-280. The phage library is incubated with the cells, beads or other solid support and non binding phage is removed by washing. Clones that bind are selected and tested.

Once selected, positive scFv clones are tested for their binding to HLA-A*02/peptide complexes on live T2 cell surfaces by indirect flow cytometry. Briefly, phage clones are incubated with T2 cells that have been pulsed with a NDC80 peptide, or an irrelevant peptide (control). The cells are washed and then with a mouse anti-M13 coat protein mAb. Cells are washed again and labeled with a FITC-goat (Fab)₂ anti-mouse Ig prior to flow cytometry.

In other embodiments, the anti-NDC80/MHC antibodies may comprise one or more framework region amino acid substitutions designed to improve protein stability, antibody binding, expression levels or to introduce a site for conjugation of therapeutic agents. These scFvs are then used to produce recombinant human monoclonal Igs in accordance with methods known to those of skill in the art.

### Uses of the Immunoglobulin-Related Compositions of the Present Technology

Methods for reducing the proliferation of leukemia cells and/or progression of the tumor or pathologic condition are also included, comprising contacting leukemia cells with an immunoglobulin-related composition of the present technology. Progression includes, e.g, the growth, invasiveness, metastases and/or recurrence of the tumor or pathologic condition. In a related aspect, the immunoglobulin-related compositions of the present technology can be used for the prevention or treatment of NDC80-associated diseases (e.g., cancers). Such treatment can be used in patients identified as having pathologically high levels of NDC80 (e.g., those diagnosed by the methods described herein) or in patients diagnosed with a disease known to be associated with such pathological levels.

In one aspect, the present disclosure provides a method for treating a NDC80-associated disease in a subject in need thereof, comprising administering to the subject an effective amount of a composition comprising an antibody moiety that specifically binds to a NDC80 peptide/HLA-A*02 complex.

In another aspect, the present disclosure provides a method for treating a NDC80-associated disease in a subject in need thereof, comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

In yet another aspect, the present disclosure provides a method for treating a NDC80-associated disease in a subject in need thereof, comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a). In certain embodiments, the cell of (c) or (d) is a T cell, a NK cell, a B cell, or a monocyte/macrophage.

In one aspect, the present disclosure provides methods for treating a NDC80-associated disease in a subject in need thereof, comprising administering to the subject an effective amount of any immunoglobulin-related composition of the present technology.

In another aspect, the present disclosure provides methods for treating a NDC80-associated disease in a subject in need thereof, comprising administering to the subject an effective amount of any of the recombinant nucleic acids, set of recombinant nucleic acids, vectors, set of vectors, or cells of the present technology.

In yet another aspect, the present disclosure provides methods for treating a NDC80-associated disease in a subject in need thereof, comprising administering to the subject an effective amount of any of the pharmaceutical compositions disclosed herein.

Additionally or alternatively, in some embodiments of the methods disclosed herein, the NDC80 peptide comprises the amino acid sequence ALNEQIARL (SEQ ID NO: 1). In any and all embodiments of the methods disclosed herein, said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

In any and all embodiments of the methods disclosed herein, the NDC80-associated disease is a cancer. Examples of cancers that can be treated by the immunoglobulin-related compositions of the present technology include any cancer presenting the ALNEQIARL (SEQ ID NO: 1) peptide in complex with HLA-A*02, including but not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, and thyroid cancer.

In one aspect, the present disclosure provides a method for mitigating immunotherapy-related toxicity in a subject in need thereof comprising administering to the subject an effective amount of any of the antigen binding proteins or immunoglobulin-related compositions disclosed herein, or any of the recombinant nucleic acids, set of recombinant nucleic acids, vectors, set of vectors, or cells disclosed herein, or any of the pharmaceutical compositions disclosed herein.

In one aspect, the present disclosure provides a method for mitigating immunotherapy-related toxicity in a subject in need thereof comprising administering to the subject an effective amount of a composition comprising an antibody moiety that specifically binds to a NDC80 peptide/HLA-A*02 complex.

In another aspect, the present disclosure provides a method for mitigating immunotherapy-related toxicity in a subject in need thereof comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

In yet another aspect, the present disclosure provides a method for mitigating immunotherapy-related toxicity in a subject in need thereof comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a). In certain embodiments, the cell of (c) or (d) is a T cell, a NK cell, a B cell, or a monocyte/macrophage.

Additionally or alternatively, in some embodiments of the methods disclosed herein, the subject has been diagnosed with or is suffering from a NDC80-associated disease, such as cancer. Examples of cancer include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02.

Additionally or alternatively, in some embodiments of the methods disclosed herein, the NDC80 peptide comprises the amino acid sequence ALNEQIARL (SEQ ID NO: 1). In any and all embodiments of the methods disclosed herein, said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Additionally or alternatively, in some embodiments of the methods disclosed herein, the immunotherapy-related toxicity is selected from the group consisting of T-cell fratricide, hematopoietic stem cell toxicity, peripheral blood mononuclear cell (PBMC) toxicity, cardiomyocyte toxicity, cardiac fibroblast toxicity, and thymic fibroblast toxicity.

The compositions of the present technology may be employed in conjunction with other therapeutic agents useful in the treatment of NDC80-associated diseases (e.g., cancers). For example, the immunoglobulin-related compositions of the present technology may be separately, sequentially or simultaneously administered with at least one additional therapeutic agent. Examples of additional therapeutic agents include, but are not limited to, antiangiogenic agents, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Other examples of additional therapeutic agents include, but are not limited to, immune checkpoint inhibitors, monoclonal antibodies that specifically target tumor antigens, cell-mediated immunotherapy (e.g., T cell therapy), immune activating agents (e.g., interferons, interleukins, cytokines), oncolytic virus therapy and cancer vaccines. Examples of immune checkpoint inhibitors include immuno-modulating/stimulating antibodies such as an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Specific immuno-modulating/stimulating antibodies include ipilimumab, Nivolumab, Pembrolizumab, Atezolizumab, Avelumab, and Durvalumab. Additionally or alternatively, in some embodiments, the monoclonal antibodies that specifically target tumor antigens bind to one or more targets selected from among CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1). Examples of immune activating agents include, but are not limited to, interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The compositions of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved. A clinician skilled in the art can readily determine, for example, by the use of clinical tests, physical examination and medical/family history, if an individual is a candidate for such treatment.

In accordance with the methods of the present disclosure, at least one NDC80 immunoglobulin-related composition described herein is used to promote a positive therapeutic response to a NDC80-associated disease (e.g., cancer). By “positive therapeutic response” is intended any improvement in the disease conditions associated with the activity of the immunoglobulin-related compositions of the present technology, and/or an improvement in the symptoms associated with the disease. Thus, for example, an improvement in the disease can be characterized as a complete response. By “complete response” is intended an absence of clinically detectable disease with normalization of any previously test results. Such a response can in some cases persist, e.g., for at least one month following treatment according to the methods of the present technology. Alternatively, an improvement in the disease can be categorized as being a partial response.

Clinical response can be assessed using screening techniques such as magnetic resonance imaging (MM) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, ELISPOT, MA, chromatography, and the like. In addition to these positive therapeutic responses, the subject undergoing therapy with the immunoglobulin-related compositions described herein can experience the beneficial effect of an improvement in the symptoms associated with the disease.

In some embodiments, the immunoglobulin-related compositions of the present technology comprise pharmaceutical formulations which may be administered to subjects in need thereof in one or more doses. Dosage regimens can be adjusted to provide the desired response (e.g., a therapeutic response).

Typically, an effective amount of the immunoglobulin-related compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-NDC80/MHC immunoglobulin-related compositions, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of immunoglobulin-related composition ranges from 0.1-10,000 micrograms per kg body weight. In one embodiment, immunoglobulin-related composition concentrations in a carrier range from 0.2 to 2000 micrograms per delivered milliliter. An exemplary treatment regime entails administration once per every two weeks or once a month or once every 3 to 6 months. Anti-NDC80/MHC immunoglobulin-related compositions may be administered on multiple occasions. Intervals between single dosages can be hourly, daily, weekly, monthly or yearly. Intervals can also be irregular as indicated by measuring blood levels of the immunoglobulin-related composition in the subject. In some methods, dosage of the immunoglobulin-related composition is adjusted to achieve a serum concentration in the subject of from about 75 μg/mL to about 125m/mL, 100 μg/mL to about 150m/mL, from about 125m/mL to about 175m/mL, or from about 150 μg/mL to about 200m/mL. Alternatively, anti-NDC80/MHC immunoglobulin-related compositions can be administered as a sustained release formulation, in which case less frequent administration is required. Dosage and frequency vary depending on the half-life of the immunoglobulin-related composition in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In one aspect, the present disclosure provides a method for detecting expression levels of NDC80 in a sample includes (a) contacting said sample with any of the immunoglobulin-related compositions described herein (e.g., antibodies such as human, humanized, or chimeric antibodies, antibody fragments, chimeric antibody-T cell receptors (caTCRs), chimeric antigen receptors (CARs), fusion proteins, and conjugates thereof) and (b) detecting binding to a NDC80 peptide-HLA-A*02 complex in the biological sample. In some embodiments, the NDC80 peptide comprises the amino acid sequence ALNEQIARL (SEQ ID NO: 1). In another aspect, the present disclosure provides a method for detecting NDC80 peptides on the surface of cells or tissues using any of the immunoglobulin-related compositions of the present disclosure (e.g., antibodies such as humanized, chimeric or human antibodies, antibody fragments, chimeric antibody-T cell receptors (caTCRs), chimeric antigen receptors (CARs), fusion proteins, and conjugates thereof). Methods for detecting peptide or protein expression are well known in the art and include, but are not limited to, PCR techniques, immunohistochemistry, flow cytometry, Western blot, ELISA, and the like.

Also provided is the use of NDC80/MHC binding molecules, e.g., humanized, chimeric or fully human antibodies against NDC80, antibody fragments, chimeric antibody-T cell receptors (caTCRs), chimeric antigen receptors (CARs), fusion proteins, and conjugates thereof, for diagnostic monitoring of protein levels (e.g., NDC80 levels) in blood or tissue as part of a clinical testing procedure, e.g., to determine the efficacy of a given treatment regimen. For example, detection can be facilitated by coupling the immunoglobulin-related composition to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, 3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

This disclosure further provides a diagnostic method useful during diagnosis of NDC80-mediated diseases such as cancers that present the ALNEQIARL (SEQ ID NO: 1) peptide in complex with HLA-A*02. Examples of such cancers include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, and thyroid cancer, which involves measuring the expression level of NDC80 in tissue or body fluid from an individual and comparing the measured expression level with a standard NDC80 expression level in normal tissue or body fluid, whereby an increase in the expression level compared to the standard is indicative of a disorder treatable by an immunoglobulin-related composition as provided herein.

The anti-NDC80/MHC immunoglobulin-related compositions provided herein can be used to assay NDC80/MHC levels in a biological sample using classical immunohistological methods known to those of skill in the art (e.g., see Jalkanen, et al, J. Cell. Biol. 101:916-985 (1985); Jalkanen et al, J. Cell Biol. 105:3087-3096 (1987)). Other antibody-based methods useful for detecting NDC80 protein or peptide expression include immunoassays, such as the enzyme linked immunosorbent assay (ELISA), immunoprecipitation, or Western blotting.

By “assaying the expression level of NDC80/MHC” is intended qualitatively or quantitatively measuring or estimating the level of NDC80/MHC complexes in a first biological sample either directly (e.g., by determining or estimating absolute levels) or relatively (e.g., by comparing to the disease associated levels in a second biological sample). The NDC80/MHC complex levels in the first biological sample can be measured or estimated and compared to a standard NDC80/MHC complex level, the standard being taken from a second biological sample obtained from an individual not having the disorder or being determined by averaging levels from a population of individuals not having the disorder. As will be appreciated in the art, once the “standard” NDC80/MHC complex level is known, it can be used repeatedly as a standard for comparison.

Formulations of Pharmaceutical Compositions. According to the methods of the present technology, the immunoglobulin-related compositions of the present technology (e.g., antibodies such as humanized, chimeric or fully human antibodies, antibody fragments, chimeric antibody-T cell receptors (caTCRs), chimeric antigen receptors (CARs), fusion proteins, and conjugates thereof) can be incorporated into pharmaceutical compositions suitable for administration. The pharmaceutical compositions generally comprise a recombinant or substantially purified immunoglobulin-related composition and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the immunoglobulin-related compositions (See, e.g., Remington's Pharmaceutical Sciences, Mack Publishing Co., Easton, PA 18th ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-NDC80/MHC immunoglobulin-related composition, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-NDC80/MHC immunoglobulin-related composition named in this technology can be present in unsalified or unesterified form, or in salified and/or esterified form, and the naming of such anti-NDC80/MHC immunoglobulin-related composition is intended to include both the original (unsalified and unesterified) compound and its pharmaceutically-acceptable salts and esters. Also, certain embodiments of the present technology can be present in more than one stereoisomeric form, and the naming of such anti-NDC80/MHC immunoglobulin-related composition is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the immunoglobulin-related compositions disclosed herein, use thereof in the compositions is contemplated. Supplementary active compounds can also be incorporated into the compositions. Other suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

A pharmaceutical composition of the present technology is formulated to be compatible with its intended route of administration. The anti-NDC80/MHC immunoglobulin-related compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The anti-NDC80/MHC immunoglobulin-related composition can optionally be administered in combination with other agents that are at least partly effective in treating various NDC80-associated diseases (e.g., cancers).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating an anti-NDC80/MHC immunoglobulin-related composition of the present technology in the required amount in an appropriate solvent with one or a combination of ingredients enumerated above, as required, followed by filtered sterilization. Generally, dispersions are prepared by incorporating the anti-NDC80/MHC immunoglobulin-related composition into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The immunoglobulin-related compositions of the present technology can be administered in the form of a depot injection or implant preparation which can be formulated in such a manner as to permit a sustained or pulsatile release of the active ingredient.

Oral compositions generally include an inert diluent or an edible carrier. The immunoglobulin-related compositions of the present disclosure may be stabilized in some form, or protected from digestion, including but not limited to the use of D-amino acids. Oral compositions can be enclosed in gelatin capsules or compressed into tablets. For the purpose of oral therapeutic administration, the anti-NDC80/MHC immunoglobulin-related composition can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-NDC80/MHC immunoglobulin-related composition is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-NDC80/MHC immunoglobulin-related composition is formulated into ointments, salves, gels, or creams as generally known in the art.

The anti-NDC80/MHC immunoglobulin-related composition can also be prepared as pharmaceutical compositions in the form of suppositories (e.g., with conventional suppository bases such as cocoa butter and other glycerides) or retention enemas for rectal delivery.

In one embodiment, the anti-NDC80/MHC immunoglobulin-related composition is prepared with carriers that will protect the anti-NDC80/MHC immunoglobulin-related composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Kits

The present technology provides kits for the detection and/or treatment of NDC80-associated diseases (e.g., cancers), comprising at least one immunoglobulin-related composition of the present technology (e.g., any antibodies (including monoclonal antibodies, polyclonal antibodies, human antibodies, humanized antibodies, chimeric antibodies, recombinant antibodies, multispecific antibodies, bispecific antibodies, etc.,), antibody fragments, chimeric antibody-T cell receptors (caTCRs), chimeric antigen receptors (CARs), fusion proteins, and conjugates thereof described herein), or a functional variant (e.g., substitutional variant) thereof. Optionally, the above described components of the kits of the present technology are packed in suitable containers and labeled for diagnosis and/or treatment of NDC80-associated diseases (e.g., cancers). The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, assay reagents, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive NDC80/MHC complex in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more immunoglobulin-related compositions of the present technology (e.g., antibodies such as humanized, chimeric or human antibodies, antibody fragments, chimeric antibody-T cell receptors (caTCRs), chimeric antigen receptors (CARs), fusion proteins, and conjugates thereof) capable of binding a NDC80/MHC complex in a biological sample; means for determining the amount of the NDC80/MHC complex in the sample; and means for comparing the amount of the immunoreactive NDC80/MHC complex in the sample with a standard. One or more of the immunoglobulin-related compositions may be labeled. The kit components, (e.g., reagents) can be packaged in a suitable container. The kit can further comprise instructions for using the kit to detect the immunoreactive NDC80/MHC complex.

For antibody-based kits, the kit can comprise, e.g., 1) a first immunoglobulin-related composition of the present technology, e.g. a humanized, chimeric or bispecific anti-NDC80/MHC antibody or an antigen binding fragment thereof, attached to a solid support, which binds to a NDC80/MHC complex; and, optionally; 2) a second, different antibody which binds to either the NDC80/MHC complex or to the first antibody, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a NDC80/MHC complex in vitro or in vivo, or for treatment of NDC80-associated diseases (e.g., cancers) in a subject in need thereof. In certain embodiments, the use of the reagents can be according to the methods of the present technology.

### EXAMPLES

The present technology is further illustrated by the following Examples, which should not be construed as limiting in any way.

### Example 1: Materials and Methods

Human Cell Lines. Cell lines were originally obtained from ATCC and frozen as aliquots in liquid nitrogen. The following cell lines were available at MSKCC: AML14, OCI-AML02, SK-Me15, SK-Me137, PANC-1, and SUDHL4. MDA-MB231, T47D, HCT116, SUDHL6 and HL60 were obtained from ATCC. MAC2A cells were obtained from Mads H. Andersen (University of Copenhagen, Copenhagen, Denmark). BV173 was provided by H. J. Stauss (University College London, London, United Kingdom). Cell lines of unknown HLA were HLA typed by American Red Cross. All cell lines were cultured in RPMI 1640 or DMEM supplemented with 10% FCS, 1% penicillin, 1% streptomycin, 2 mM 1-glutamine, and 10 mM HEPES at 37° C. and 5% CO2. Normal human cardiomyocytes (HCM), cardiac fibroblast (HCF) and thymic fibroblasts (HTyF) were purchased from Science Cell Research laboratories (Carlsbad, CA). Cells were cultured in the medium with the respective supplement provided by the vendor, according to the manufacturer's instructions.

NDC80 expression profiles. Datasets on NDC80 expression normalized by TPM in cancer cell lines, healthy tissues, cancer tissues and adjacent healthy tissues were retrieved from following sources: (1) Cancer cell line data were downloaded through the expression atlas from the European Institute of Bioinformatics based on the Cancer Cell Line Encyclopedia dataset (EMTAB2770) (Barretina et al., Nature 483: 603-607 (2012)); (2) Data from healthy tissues were downloaded from the GTEX consortium V8 (Aguet et al., Science 369, 1318-1330 (2020)); (3) Data from cancer tissues and adjacent healthy tissues were accessed through OmicsDI and were generated by the PCAWG initiative (E-MTAB-5200) (Goldman et al., Nat Commun 11: 3400 (2020)).

Immunopurification of HLA class I ligands. Suspension cells were harvested through direct resuspension, adherent cell lines after incubating 15 min with CellStripper™ solution (Corning™, Cat #25056CI). Harvested cells were pelleted and washed three times in ice-cold sterile PBS. For all experiments 50M cells were used. Cells were then lysed in 7.5 ml of 1% CHAPS (Sigma-Aldrich, St. Louis, MO, Cat #C3023) dissolved in PBS, and supplemented with protease inhibitors (cOmplete™, Cat #11836145001). Cell lysis was performed for 1 hour at 4° C., lysates were spun down for 1 hour at 20,000×g at 4° C. and supernatant fluids isolated.

Affinity columns were prepared as follows: 40 mg of Cyanogen bromide-activated-Sepharose® 4B (Sigma-Aldrich, St. Louis, MO, Cat #C9142) were activated with 1 mM hydrochloric acid (Sigma-Aldrich, St. Louis, MO, Cat #320331) for 30 min. Subsequently, 0.5 mg (and for the experiments considering amount of antibody usage either 0 μg, 50 μg, 200 μg, 500 μg or 1000 μg) of W6/32 antibody (BioXCell, Lebanon, NH, Cat. #BE0079), BB7.2 antibody (MSKCC Antibody Core Facility), clone 1 or murine IgG1 were coupled to sepharose in presence of binding buffer (150 mM sodium chloride, 50 mM sodium bicarbonate, pH 8.3; sodium chloride: Sigma-Aldrich, St. Louis, MO, Cat #59888, sodium bicarbonate: Sigma-Aldrich, St. Louis, MO, Cat #56014) for at least 2 hours at room temperature. Sepharose was blocked for 1 h with glycine (Sigma-Aldrich, St. Louis, MO, Cat #410225). Columns were washed twice in PBS and equilibrated for 10 minutes. 1-3×10⁷ cells were harvested and washed three times in ice-cold sterile PBS.

Afterward, cells were lysed in 1 mL 1% CHAPS (Sigma-Aldrich, Cat. #C3023) in PBS, supplemented with 1 tablet of protease inhibitors (Omplete™, Cat. #11836145001) for 1 hour at 4° C. This lysate was spun down for 1 hour at 20,000×g at 4° C. Supernatant fluid was run over the affinity column using peristaltic pumps at 1 mL/minute overnight at 4° C. Affinity columns were washed with PBS for 15 minutes, run dry, and HLA complexes subsequently eluted five times with 200 mL 1% trifluoracetic acid (TFA, Sigma-Aldrich, Cat. #02031).

For separation of HLA ligands and their HLA complexes tC18 columns (Sep-Pak® C18 1 cc Vac Cartridge, 50 mg Sorbent per Cartridge, 37-55 μm particle size, Waters, Milford, Massachusetts, Cat #WAT054955) were prewashed with 80% acetonitrile (ACN, Sigma-Aldrich, St. Louis, MO, Cat #34998) in 0.1% TFA and equilibrated with two washes of 0.1% TFA. Samples were loaded, washed again with 0.1% TFA and eluted in 400 mL 30% ACN in 0.1% TFA followed by 400 mL 40% ACN in 0.1% TFA, then 400 mL 50% ACN in 0.1% TFA.

For separation by size exclusion filters 0.5 ml 3 kDA cut-off filters were used (Millipore Sigma, Burlington, MA, Cat. #UFC5003). Sample volume was reduced by vacuum centrifugation for mass spectrometry analysis.

Solid Phase Extractions (SPE). In house C18 mini columns were prepared as follows: for SPE of one sample two small disks of C18 material (1 mm in diameter) were punched out from CDS Empore™ C18 disks (Thermo Fisher, Waltham, MA, Cat #13-110-018) and transferred to the bottom of a 200 μl Axygen pipette tip (Thermo Fisher, Waltham, MA, Cat #12639535). Columns were washed once with 100 μl 80% ACN/0.1% TFA and equilibrated with 3 times 100 μl 1% TFA. All fluids were run through the column by centrifugation in mini table top centrifuges and eluates were collected in Eppendorf tubes. Then, dried samples were resuspended in 100 μl 1% TFA and loaded onto the columns, washed twice with 100 μl 1% TFA, ran dry and eluted with 50 μl 80% ACN/0.1% TFA. Again, sample volume was reduced by vacuum centrifugation.

LC-MS/MS analysis of HLA ligands. Samples were analyzed by high resolution/high accuracy LC-MS/MS (Lumos Fusion, Thermo Fisher). Peptides were desalted using ZipTips (Millipore®; Sigma Cat. #ZTC18S008) according to the manufacturer's instructions and concentrated using vacuum centrifugation prior to being separated. Peptides were separated using direct loading onto a Packed Emitter C18 column (75 um ID/12 cm, 3 μm particles, Nikkyo Technos Co., Ltd. Japan). The gradient was delivered at 300nl/min increasing linearly from 2% Buffer B (0.1% formic acid in 80% acetonitrile)/98% Buffer A (0.1% formic acid) to 30% Buffer B/70% Buffer A, over 70 minutes. MS and MS/MS were operated at resolutions of 60,000 and 30,000, respectively. Only charge states 1, 2 and 3 were allowed. 1.6 Th was chosen as isolation window and collision energy was set at 30%. For MS/MS, maximum injection time was 100 ms with an AGC of 50,000.

Mass spectrometry data processing. Mass spectrometry data were processed using Byonic™ software (version 2.7.84, Protein Metrics, Palo Alto, CA) through a custom-built computer server equipped with 4 Intel Xeon E5-4620 8-core CPUs operating at 2.2 GHz, and 512 GB physical memory (Exxact Corporation, Freemont, CA). Mass accuracy for MS1 was set to 6 ppm and to 20 ppm for MS2, respectively. Digestion specificity was defined as unspecific and only precursors with charges 1, 2, and 3 and up to 2 kDa were allowed. Protein FDR was disabled to allow complete assessment of potential peptide identifications. Oxidization of methionine, phosphorylation of serine, threonine and tyrosine as well as N-terminal acetylation were set as variable modifications for all samples. Samples were searched against UniProt Human Reviewed Database (20,349 entries, http://www.uniprot.org, downloaded June 2017) with common contaminants added. Peptides were selected with a minimal log probability value of 2 corresponding to p-values<0.01 for PSM in the given database and were HLA assigned by netMHC 4.0 with a 2% rank cutoff. Duplicates were removed.

For network analyses corresponding source proteins of the identified HLA ligands were selected and processed through the GENEMania plug-in at Cytoscape (version 3.8.2). 20 related genes and attributes were enabled and data were weighted based on GO biological process. Processes with a q-value <0.05 were considered for further analysis. Relative quantitation for ALNEQIARL (SEQ ID NO: 1) and potential off-targets was assessed by Skyline (MacCoss Lab, version 20.2) using the retention times identified in the Byonic™ analysis for the MS/MS spectra. Peak intensities were calculated for the precursor and isotopes +1, +2, and +3.

Assignment of peptide sequences to HLA alleles. To assign peptides which passed the MS quality filters described above to their HLA complexes which they most likely bind to, the netMHCpan 4.0 algorithm was used with default settings. No binding affinity predictions were enabled. Therefore, all peptides with affinity % ranks below 2 were considered binder.

Transduction Methods. Human T cells were cultured in RPMI1640 supplemented with 10% FBS and activated with CD3/CD28 Dynabeads™ (Thermo Fisher, Waltham, MA). 24 hours after activation, human T cells were transduced with concentrated lentivirus in RetroNectin (Takara) coated plates. Transduced T cells were then expanded in the presence of 100 U/mL IL2 (Sigma). Transduction efficiency was assessed by flow cytometry using anti-myc-PE antibody.

Flow Cytometry. T2 cells were pulsed overnight with 50 μg/ml of one of the indicated peptides. Pulsed T2 cells were then stained with either BB7.2-PE antibody specific for HLA-A*02 to ensure stabilization of T2 cells with the pulsed peptides or with the labeled TCR mimic antibodies for 30 min on ice as described herein. Cells were washed after incubation with FACS buffer, resuspended in FACS buffer supplemented with DAPI and analyzed using a BD Fortessa™ flow cytometer.

For cell-surface staining, cells were blocked using FcR Blocking Reagent (Miltenyi Biotec, Bergisch Gladbach, Germany, Cat. #130-059-901) at the manufacturer's recommended dilution for 10 minutes on room temperature, then incubated with appropriate fluorophore-conjugated mAbs for 30 minutes on ice and washed twice before resuspension in a viability dye (DAPI). Abs used include anti-HLA-A2 clone BB7.2-PE (Biolegend, San Diego, CA, 343306), anti-CD3-APC-Cy7 clone SK7 (Biolegend, San Diego, CA, 344818), anti-CD19-FITC clone MB19-1 (Biolegend, San Diego, CA, Cat. #101506), anti-CD33-BV711 clone WM53 (Biolegend, San Diego, CA, Cat. #303423), anti-myc clone 71D10-A1647 (Cell Signaling Technology (CST), Danvers, MA, Cat. #63730), CD14-PE clone 61D3 (eBioscience, Cat. #12-0149-42) and anti-mouse-CD45-A1700 clone 30F11 (Biolegend, San Diego, CA, Cat. #103128). Clones 1, 7, 11, 14, 18 and 19 or their murine IgG1 isotype control were conjugated to PE using the lightning-link kit (Novus Biologicals, Cat. #703-0010), and staining was performed at 2 μg/ml, which was determined to be a saturating concentration. For flow cytometry-based quantitation experiments BD Quantibrite-PE™ kits were used (BD Biosciences, San Jose, CA, Cat. #340495) according to the manufacturers protocol. Flow cytometry data were collected on an LSRfortessa™ (BD) and analyzed with FlowJo™ V10 software.

LDH release assay. T2 cells were prepared as described for flow cytometry assays. Then 5000 T2 cells were seeded in 96-well round bottom plates and incubated with the CAR T cells described herein at the indicated E:T ratios for 16-18h. LDH release assay was performed according to the manufacturer's instructions (Promega, Madison, WI) and for analysis purposes, killing of the wild-type peptide ALNEQIARL (SEQ ID NO: 1) was set to 100% and data for the other peptide were depicted as fraction of the 100% killing of the wild-type peptide.

Enhanced killing assays using drug pre-treatment of target cells. Cells were incubated with either 10, 100 or 1000 pM of docetaxel (Sellekchem, Houston, Tx, Cat. #No. S1148) or DMSO at the same concentrations as control for 48h. Then antibody staining was performed as described herein with PE-labeled TCR mimic antibodies.

scFv clones specific for peptide/HLA-A0201 complexes. A human scFv antibody E-ALPHA® phage display library was used for the selection of mAb clones specific to ALNEQIARL (SEQ ID NO: 1):HLA-A*02. In brief, T2 cells pulsed with 50 ug/ml irrelevant control peptides were used to remove any clones that potentially bind to HLA-A*02:01 in the library. Remaining clones were screened for T2 cells pulsed with 50 ug/ml ALNEQIARL (SEQ ID NO: 1) peptides. The selected clones were enriched by 3 rounds of panning. Positive clones were determined by their binding to T2 cells pulsed with ALNEQIARL (SEQ ID NO: 1) peptides but not to T2 cells pulsed with control peptides by flow cytometry. The cells were first stained with purified scFv phage clones and followed by staining with a mouse anti-M13 (bacteriophage) mAb, and finally the goat anti-mouse IgG conjugated to PE. Each step of the staining was done between 30-60 minutes on ice and the cells were washed twice between each step of the staining.

Engineering full-length mAb using the selected ScFv fragments. Full-length human IgG1 of the selected phage clones were produced in HEK293 cell lines. In brief, antibody variable regions were subcloned into mammalian expression vectors, with matching Lambda or Kappa light chain constant sequences and IgG1 subclass Fc. Molecular weight of the purified full-length IgG antibodies were measured under both reducing and non-reducing conditions by electrophoresis.

Peptide stimulation and ELISpot assay. CD14+ monocytes were isolated from cell separation medium-purified (Thermo Fisher, Waltham, MA, Cat. #25072C1) PBMCs from HLA-A*02:01 healthy donors according to Memorial Sloan Kettering Cancer Center (MSK) IRB-approved protocols by positive selection using mAb to human CD14 coupled with magnetic beads (Miltenyi Biotec, Bergisch Gladbach, Germany, Cat. #130-050-201) and used for the first stimulation of T cells. The CD14-fraction of PBMCs was used for isolation of CD3 positive cells by negative immunomagnetic cell separation using a pan T cell isolation kit (Miltenyi Biotec, Bergisch Gladbach, Germany, Cat. Nr. 130-096-535). In vitro T cell stimulation and generation of monocyte-derived dendritic cells (DCs) from CD14+ cells was performed). A week after final stimulation, the peptide-specific T cell response was examined by an IFN-γ enzyme-linked immunospot (ELISpot) assay.

Peptide synthesis. All peptides used in this study were purchased and synthesized by Genemed Synthesis, Inc. (San Antonio, TX). Control peptides used for HLA-A*02:01 was Ewing sarcoma-derived peptide EW (QLQNPSYDK; SEQ ID NO: 178).

Generation of NDC80-CAR T cells. scFvs were grafted onto a second-generation CAR with CD28 and CD3ζ signaling domains engineered in cis to provide intracellular T-cell stimulation signals. The CAR sequence was cloned into a pCDH lentiviral vector (Systems Biosciences) for delivery into T cells. Human T cells were cultured in RPMI1640 supplemented with 10% FBS and activated with CD3/CD28 Dynabeads™ (Thermo Fisher, Waltham, MA, Cat. #11-161-D). One day after activation, human T cells were transduced with concentrated lentivirus in RetroNectin (Takara) coated plates. Transduced T cells were then expanded in the presence of 100 U/mL IL2 (Sigma-Aldrich, St. Louis, MO) for 8-12 days. Transduction efficiency was assessed by direct staining using anti-myc clone 71D10-A1647 (Cell Signaling Technology (CST), Danvers, MA, #63730).

LDH killing assay for cell lines, healthy leukocytes and HSCs. For killing assays 5,000 to 10,000 target cells were incubated for 16-18 hours with either MOCK transduced T cells, 4H11 CAR T cells, NDC80-clone 1 or NDC80-clone 11 cells. The assay was used according to the manufacturer's protocol. For healthy leukocyte killing leukocyte fractions were isolated using MACS® (magnetic activated cell sorting) with CD3, CD4 and CD19 beads. After confirming purity of >90% via flow cytometry CD3 positive cells were stimulated with CD3/CD28 Dynabeads™ (Thermo Fisher, Waltham, MA, Cat. #11-161-D) for 48h, CD19 cells were stimulated with CD40L (1 μg/ml) and IL-4 (20 ng/ml) for 48 hours. For cord blood HSPCs, cells were isolated as described in the colony forming unit assay and used in parallel for killing assays.

NDC80 siRNA knockdown. 0.5 million JMN cells were plated per well in 6-well plates in Gibco Opti-MEM medium (Thermo Fisher, Waltham, MA, Cat. #31985062) and 5, 25 or 50 nM of NDC80 siRNA (Thermo Fisher, Waltham, MA, Cat. #4392420) administered using Lipofectamine RNAiMAX (Thermo Fisher, Waltham, MA, Cat. #LMRNA015). Silencer® Select Negative Control No. 1 siRNA was used as a negative control (Thermo Fisher, Waltham, MA, Cat. #4390843). After 72h cells were harvested, lysed for Western Blot confirmation and were used for killing assays in parallel.

Western Blot. Total cell lysate was extracted using RIPA buffer and quantified using the DC protein assay (Bio-Rad). 15-30 μg of protein was loaded and run on 4%-12% SDS PAGE gels. After a 1 hour block with 5% milk at room temperature, immunoblotting was performed using the anti-NDC80 clone 9G3 (Abcam, Cambridge, United Kingdom, Cat. #ab3613). Abs were probed at the manufacturer's recommended dilution overnight at 4° C. before a secondary Ab conjugated to HRP was used for imaging. Replicate samples were probed using the indicated Abs when noted, or blots were stripped with Restore Western Blot Stripping Buffer (Thermo Fisher, Waltham, MA, 21063), reblocked with 5% milk, and reprobed with an anti-GAPDH-HRP direct conjugated Ab (Cell Signaling Technology, Danvers, MA, Cat. #3683) as a loading control.

Purification of cord blood derived HSPC-CD34+ cells. CD34+ HSPCs were purified from cord blood (CB) units (each unit from one healthy donor) in each purification. Mononuclear cells were first isolated from CB using Hespan® and Ficoll-Paque® Plus density centrifugation, followed by positive selection using the Auto MACS® Pro Seperator and isolation Kit (Miltenyi Biotec, Cat. #130-092-545). CD34+ cells were cultured in Iscove's modified Dulbecco's medium (IMDM, Cellgro) 20% BIT 9500 medium (Stem Cell Technologies, Vancouver, Canada) supplemented with SCF (100 ng/ml), FLT-3 ligand (10 ng/ml), IL-6 (20 ng/ml) and TPO (100 ng/ml) as the basic culture.

Colony forming unit (CFU) assay. 10⁴ CD34+ cells or primary patient samples that were cocultured with 1044H11 CAR T cells or NDC80-clone 1 CAR T cells for 16 hrs were plated (in triplicate) in methylcellulose (MethoCult™ H4434 Classic-Stem Cell Technologies, Vancouver, Canada, Cat. #H4434). CFU colonies were scored 4-14 days after seeding. Total cell number in CFU colonies were counted and flow cytometry analysis were performed on CFU colonies from the CD34+ cells using the following lineage markers: Myeloid panel (PE-CD13, FITC-CD14, APC-CD33, Pacific Blue-Mac1) and erythroid panel (FITC-CD71, PE-GlycophorinA).

Animals and in vivo models. Eight- to ten-week-old NOD.Cg-Prkdc^{scid} IL2rg^{tm1Wjl}/SzJ mice (NSG) were purchased from The Jackson Laboratory or obtained from the MSKCC animal breeding facility. Female mice were used for the BV173 and JMN models. For the BV173 leukemia model, 15 NSG mice were injected intravenously with one million BV173 cells and after 5 days, 5 mice each were either injected with PBS, 2 million 4H11 CAR T cells or 2 million NDC80-clone 1 CAR T cells (8 days after transduction) via tail vein. If transduction efficiency was different between groups, total T cell numbers were equalized with unspecific MOCK T cells. Starting with day 14 mice were cheek bled, and after ACK (Ammonium-Chloride-Potassium) lysis, the samples were stained for HLA-A*02 as well as murine CD45 to determine the fraction of blast cells in the peripheral blood as a marker for tumor burden. For the JMN model, 15 NSG mice were injected intraperitoneally with 300,000 GFP-Luc transduced JMN cells. Tumor burden was assessed by bioluminescence imaging (BLI) twice per week before treatment and then after injection of 150,000 NDC80-clone 1 CAR T cells, 4H11 CAR T cells or PBS.

### Example 2: LC-MS/MS Analysis Defines the NDC80 Derived Peptide ALNEQIARL (SEQ ID NO: 1) as Highly Tumor-Associated HLA-A*02 Ligand

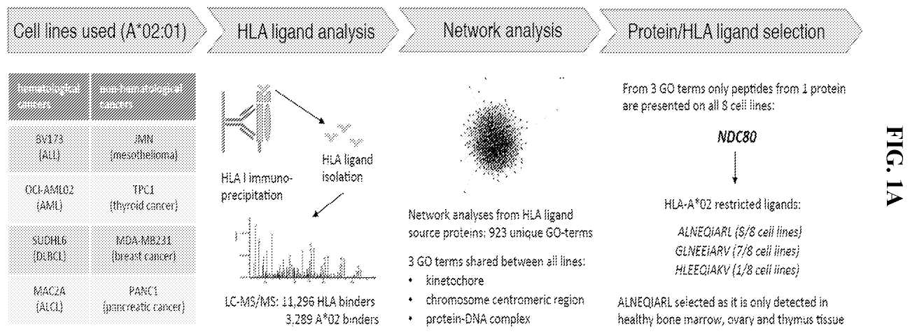

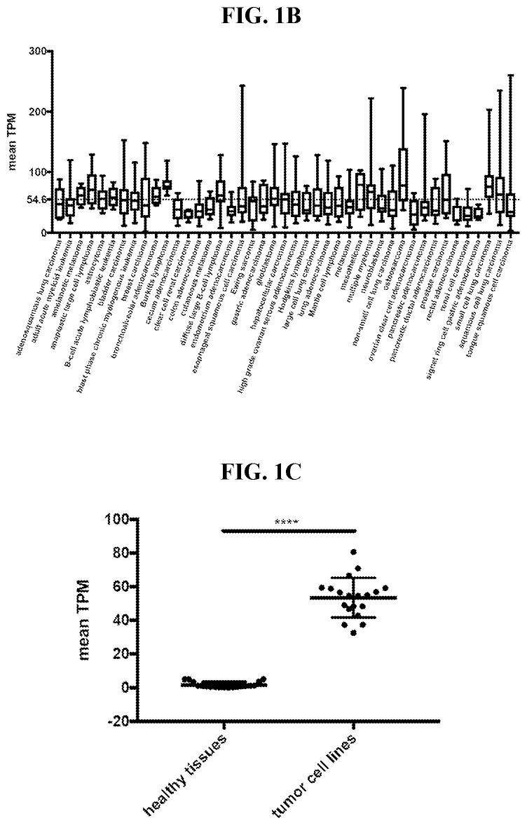

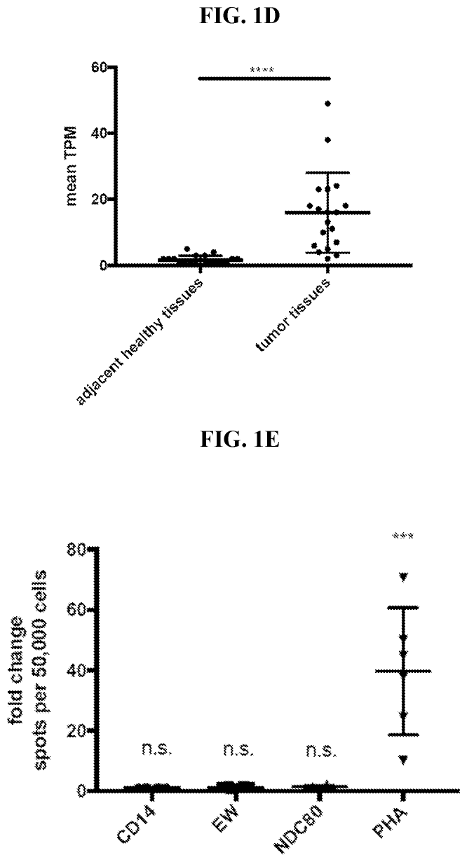

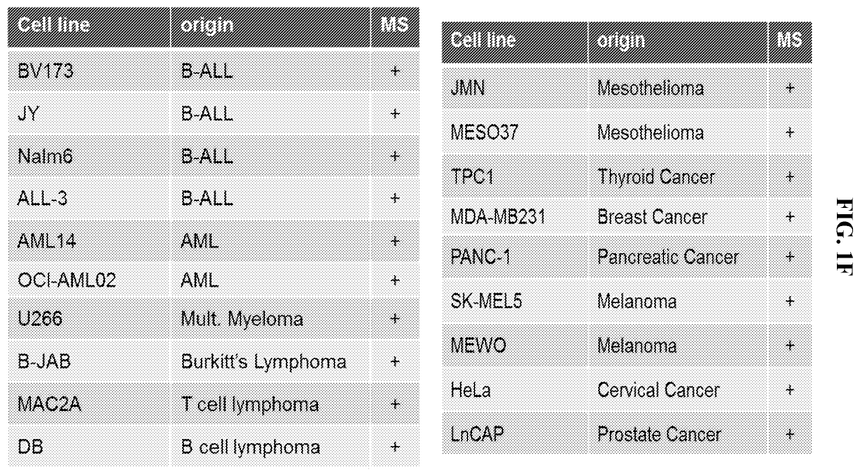

To discover a tumor-associated HLA-A*02 restricted HLA ligand, HLA complexes were immunopurified from four hematological cancer cell lines (BV173, OCI-AML02, SUDHL6, MAC2A) and four non-hematological cancer cell lines (JMN, TPC-1, MDA-MB231, PANC-1). The immunopeptidome of the complexes were analyzed via liquid-tandem mass spectrometry (LC-MS/MS) (FIG. 1A). Over 11,000 unique HLA class I-assigned peptides, of which 3,289 were considered HLA-A*02:01 binders were identified.

Utilizing source proteins from which the HLA ligands were derived, network analyses was performed via the GeneMANIA algorithm through a Cytoscape plugin. Of 923 GO-terms (q-values<0.05) resulting from the analyses of the eight different cell lines, only three processes were shared among all lines: kinetochore (average q-value 1.2×10⁻⁴), chromosome centromeric region (average q-value 1.6×10⁻³), protein DNA complex (average q-value 2.7×10⁻⁴) (FIG. 1A). Finally, from all the proteins involved in these three GO-terms, only peptides from the NDC80 protein were presented in all eight cell lines. Similarly, three HLA-A*02 restricted HLA ligands from NDC80 were detected at the cell surface, i.e., ALNEQIARL (SEQ ID NO: 1), GLNEEIARV (SEQ ID NO: 171), and HLEEQIAKV (SEQ ID NO: 172). Of these, only the ALNEQIARL (SEQ ID NO: 1) peptide ligand was shared between all the tested cancer lines. Of note, though the HLA ligand GLNEEIARV (SEQ ID NO: 171) also showed high presentation frequency (in 7 of 8 cell lines), it was considered to be a less suitable candidate because MS data from healthy human tissues demonstrated that the GLNEEIARV (SEQ ID NO: 171) ligand was presented in multiple essential healthy tissues, including lung, esophagus and colon. In the same study, the ALNEQIARL (SEQ ID NO: 1) peptide was only detected in ovary, thymus and bone marrow, which were initially considered to be potentially acceptable off-tumor targets.

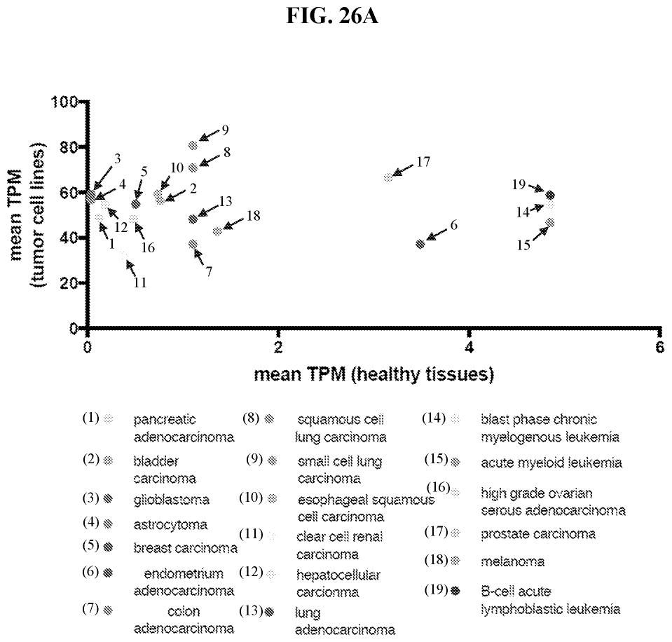

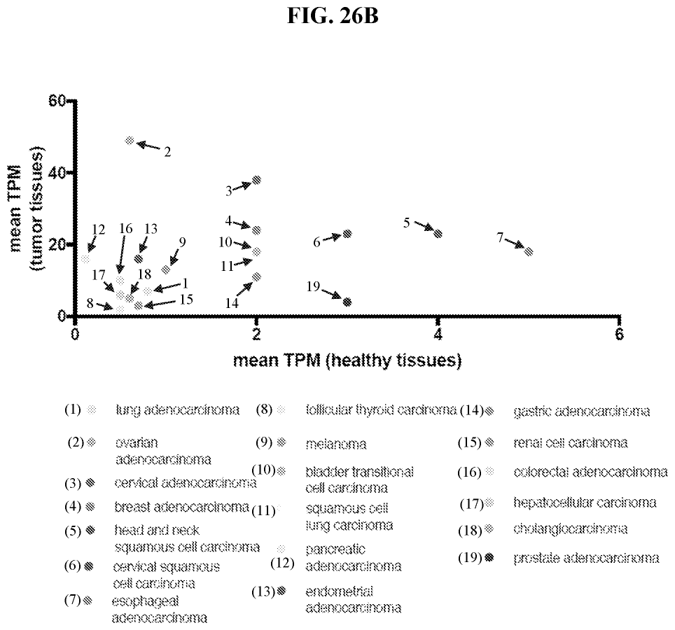

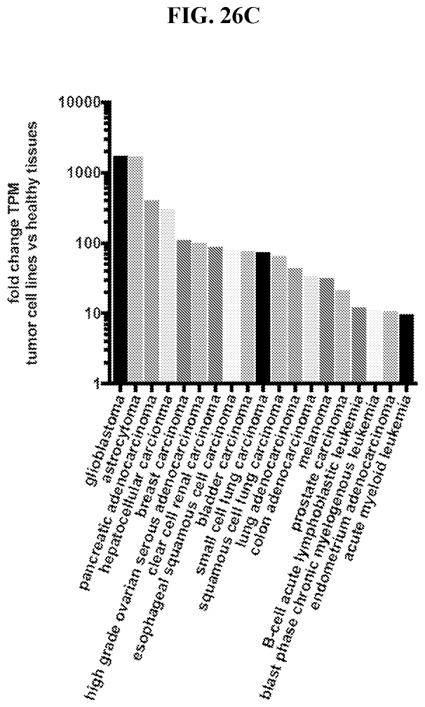

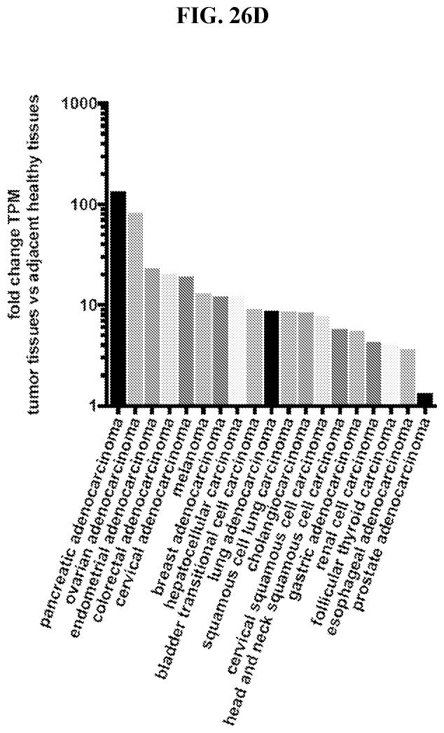

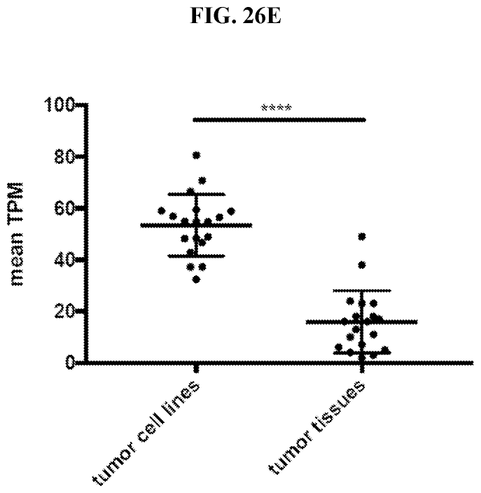

To underline the importance of NDC80 in cancer and its strong tumor-association, expression data was retrieved from 934 cancer cell lines from the Cancer Cell Line Encyclopedia, and mean transcripts per million (TPMs) was calculated for all cancer types where five or more cell lines were available (FIG. 1B). These mean TPMs were significantly higher compared to the NDC80 expression levels of the corresponding healthy tissues published by the GTEX consortium (Aguet et al., Science 369, 1318-1330 (2020)) (FIG. 1C; FIG. 26A). However, as cancer cell lines are already preselected for high proliferation capacity, to ensure a better biological comparison, data from cancer tissues and matched adjacent healthy tissues from the PCAWG project was additionally analyzed (Goldman et al., Nat Commun 11: 3400 (2020)), and a similar, highly significant overexpression of NDC80 (FIG. 1D; FIG. 26B) was found. Of note, NDC80 overexpression reached up to 1,300-fold in glioblastoma and astrocytoma cell lines versus healthy brain tissue and over 100-fold for pancreatic adenocarcinoma versus adjacent healthy tissue (FIGS. 26C-26D). Nevertheless, the mean TPM of NDC80 in cancer cell lines was significantly higher than compared to cancer tissues suggesting a further artificial enhancement in the cancer cell lines (FIG. 26E). A literature search for MS identification of NDC80 peptides and testing of additional cell lines via MS (>90% positivity rate for A*02 positive cell lines) confirmed the presentation of the ALNEQIARL (SEQ ID NO: 1) peptide on the surface of additional cancer cell lines as well as primary cancer tissues (Table 5).

TABLE 5
Mass Spectrometry evidence of HLA-A*02:ALNEQIARL (SEQ
ID NO: 1) in cell lines and primary tissue samples

| | | | |
|---|---|---|---|
| Tumor type | Cell line | Primary tissue | References |

Hematological malignancies

| | | | |
|---|---|---|---|
| AML | AML14, OCI-AML02 | n.t. | disclosed herein |
| B-ALL | JY, BV173, Nalm6, | yes | Kemps et al., 2019 Front Immunol 10, |
| | ALL-3 | | 3045; Lanoix et al., 2018 Proteomics |
| | | | 18, e1700251; Pearson et al., 2016 J |
| | | | Clin Invest 126, 4690-4701. |
| Multiple Myeloma | U266 | n.t. | disclosed herein |
| DLBCL | DB, SUDHL4 | n.t. | disclosed herein |
| T cell lymphoma | MAC2A | n.t. | disclosed herein |
| MCL | — | yes | Khodadoust et al., 2017 Nature 543, |
| | | | 723-727. |

Non-hematological malignancies

| | | | |
|---|---|---|---|
| Melanoma | A375, SKMEL5 | yes | Bassani-Sternberg et al., 2016 Nat |
| | MEWO, MEL624 | | Commun 7, 13404; Gloger et al., 2016 |
| | | | Cancer Immunol Immunother 65, |
| | | | 1377-1393; Koumantou et al., 2019 |
| | | | Cancer Immunol Immunother 68, |
| | | | 1245-1261; Stopfer et al., 2020 Nat |
| | | | Commun 11, 2760. |
| Breast Cancer | MDA-MB231 | yes | Ternette et al., 2018 Proteomics 18, |
| | | | e1700465; disclosed herein |
| Colon Cancer | — | yes | Loffler et al., 2018 Cancer Res 78, |
| | | | 4627-4641 |
| Prostate Cancer | LnCAP | n.t. | disclosed herein |
| Pancreatic Cancer | PANC-1 | n.t. | disclosed herein |
| Thyroid Cancer | TPC1 | n.t. | disclosed herein |
| Mesothelioma | JMN, MESO37 | n.t. | disclosed herein |
| Glioblastoma | — | yes | Shraibman et al., 2019 Mol Cell |
| | | | Proteomics 18, 1255-1268. |

n.t. = not tested

Finally, to investigate whether the ALNEQIARL (SEQ ID NO: 1) ligand could also be targeted by a human TCR-directed T cell, T cell reactivity was tested in healthy A*02 positive blood donors that had been stimulated by the peptide. No reactivity was detected after multiple stimulations with consistent results between donors (FIG. 1E). Overall, these data suggest that the human immune system is tolerant to ALNEQIARL (SEQ ID NO: 1) and that this peptide is a highly tumor-associated HLA ligand, which ideally could be targeted by a TCR mimic-based strategy.

### Example 3: Selection and Characterization of scFv Specific for NDC80/MHC I Complexes (ALNEQIARL (SEQ ID NO: 1)/HLA-A*02:01 Complex)

As described in Example 2, mass spectrometry-based analysis of the presented HLA ligands of several cancer cell lines was checked for highly prevalent HLA ligand and matches with HLA ligands of healthy tissues to prevent off-target toxicity. NDC80 is widely overexpressed in different cancer types, and major solid tumors of many origins. See FIGS. 1A-1F. NDC80 is an essential part of NDC80 complex, which is involved in kinetochore organization and cell division. The ALNEQIARL (SEQ ID NO: 1) epitope was found to be absent in 100 samples of healthy tissue as determined by Mass-Spectrometry.

A collection of 34 human scFv antibody phage display libraries (diversity=10×10¹⁰) constructed by Eureka Therapeutics, Inc. (E-ALPHA® phage libraries) were used for the selection of human mAbs specific to NDC80/HLA-A*02:01. 34 human phage scFv libraries were used to pan against NDC80/HLA-A*02:01 complex. In order to avoid the conformational change of MHC I complex introduced by immobilizing the protein complex onto plastic surfaces, cell panning was used in place of conventional plate panning. In cell panning, the human scFv phage libraries were first mixed with T2 cells loaded with control peptides to remove scFv phage clones that bind to T2 cells alone or MHC alone. The resulting pre-cleared phages (those not bound to T2 cells alone or MHC alone) were then mixed with T2 cells loaded with target NDC80 Peptide A, which has the amino acid sequence of ALNEQIARL (SEQ ID NO: 1). T2 is a TAP-deficient, HLA-A*02:01⁺ lymphoblast cell line that express low amounts of HLA-A*02 on the cell surface and can only present exogenous peptides. To load peptide, T2 cells were pulsed with peptides (50 μg/ml) in serum-free IMDM medium overnight. After extensive washing with PBS, peptide-loaded T2 cells with bound scFv antibody phage were spun down. The bound clones were then eluted and used to infect E. coli XL1-Blue cells. The phage clones expressed in bacteria were then purified. The panning was performed for 3 rounds to enrich scFv phage clones that bound NDC80/HLA-A*02:01 specifically.

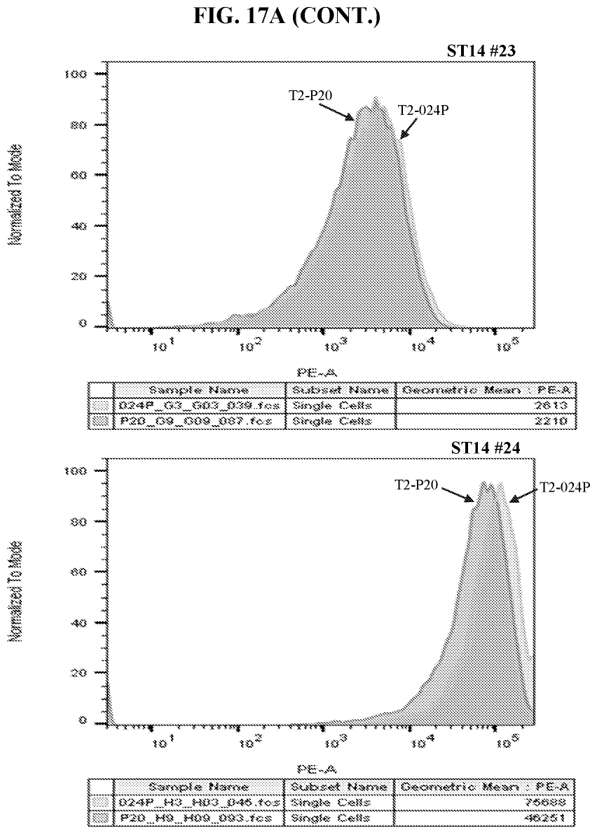

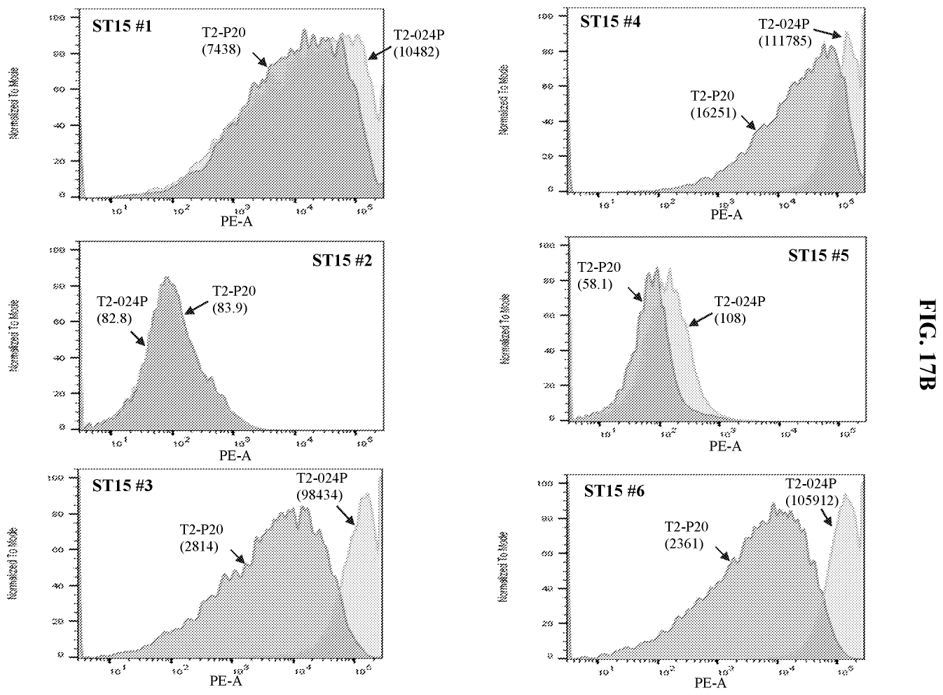

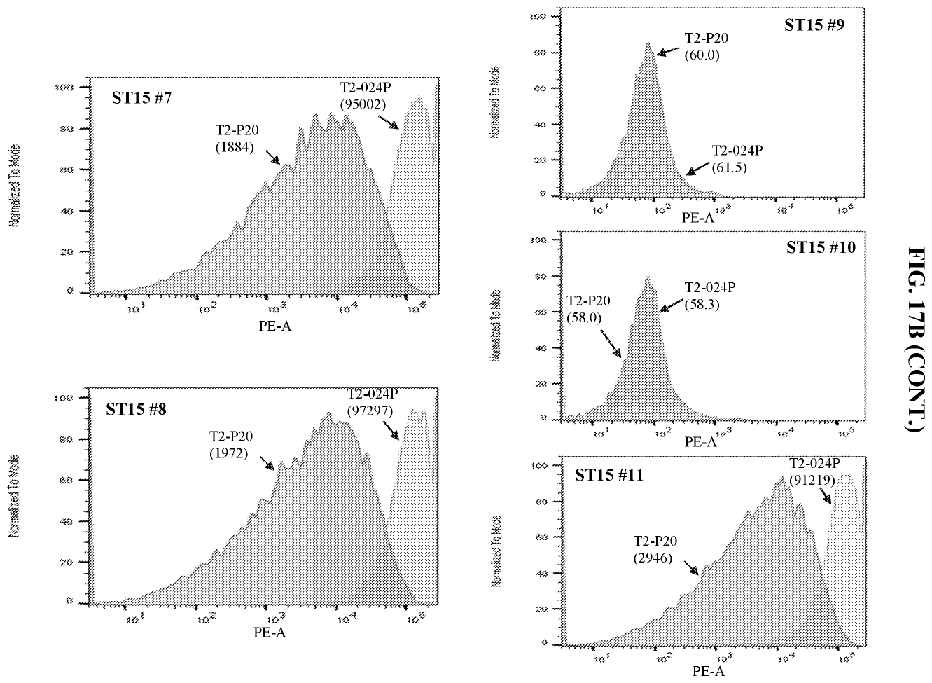

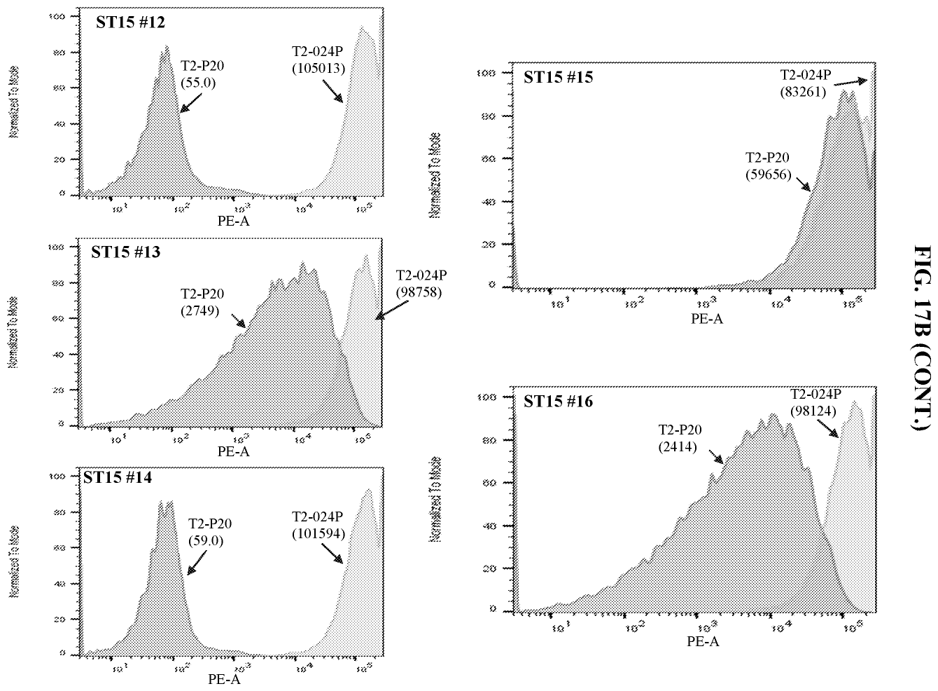

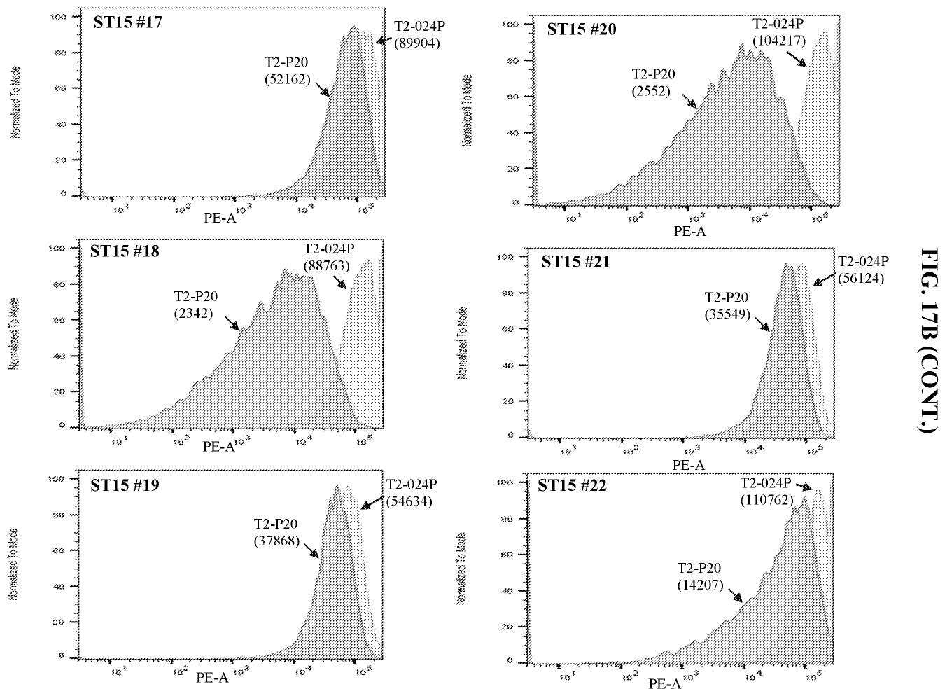

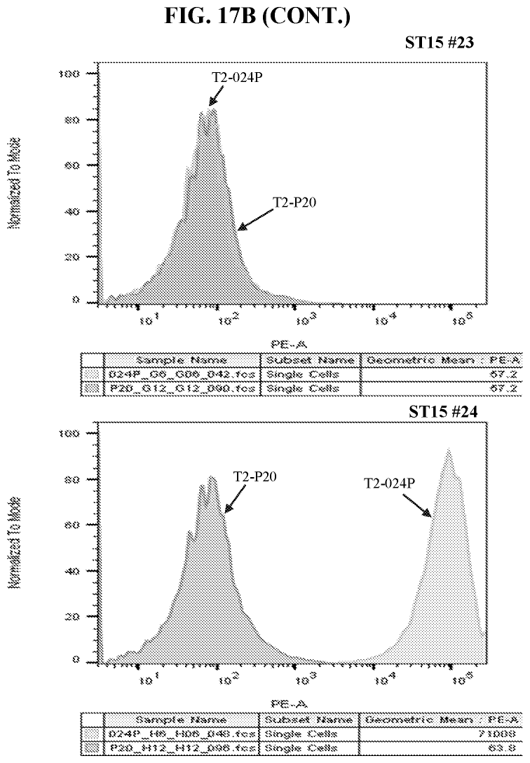

Individual phage clones from enriched phage display panning pools against NDC80/HLA-A*02:01 complex were incubated with T2 cells loaded with the NDC80 peptides (T2 cell loaded with NDC80 peptides, 50 μg/million cells) or T2 cells loaded with control peptides (T2 cell loaded with a mixture of 20 control peptides, 50 μg/million cells), respectively. Binding of the phage clones was detected by staining cells with a biotinylated mouse anti-M13 mAb (Sino Biological, Cat. #11973-MM05T) and a PE conjugated streptavidin (Vector Laboratories Cat. #SA-5207) followed by FACS analysis. See FIGS. 17A-17B. Among the 816 clones screened, 347 recognized NDC80 peptide-loaded T2 cells specifically. After sequence analysis, 60 clones were identified as unique clones. See FIG. 22.

These screening results were unexpected in view of prior studies that reported no T cell reactivity with the ALNEQIARL (SEQ ID NO: 1) peptide in both healthy individuals and AML patients. Dissertation of Anne Claudia Berlin, Kartierung des HLA-Ligandoms der akuten myeloischen Leukämie zur Entwicklung einer therapeutischen Multipeptidvakzine (2018). Initial peptide stimulation studies against the target Peptide A (SEQ ID NO: 1) were also found to be negative for T cell reactivity.

### Example 4: Characterization of FACS-Positive NDC80/MHCI-Specific Phage Clones

Cross-reactivity to NDC80 homologous peptides. Phage clones (K07-D3 phage) selected from FACS binding analysis against NDC80 Peptide A-loaded T2 cells were characterized further for cross-reactivity towards homologous peptide/HLA-A*02:01 complexes on the surface of live T2 cells loaded with peptides homologous to the NDC80 Peptide A, again using FACS analysis. Four highly abundant, potentially cross-reactive peptides were found in normal and malignant cells, as identified by Mass Spectrometry, that share either the amino terminal or carboxyl terminal four amino acids.

| | |
|---|---|
| NDC80 Peptide A | ALNEQIARL |
| | (SEQ ID NO: 1) |

Homologous Peptides

| | |
|---|---|
| NC1 | ALNEKLVNL |
| | (SEQ ID NO: 166) |
| NC2 | ALNELLQHV |
| | (SEQ ID NO: 167) |
| NC3 | MLANDIARL |
| | (SEQ ID NO: 168) |
| NC4 | TLADIIARL |
| | (SEQ ID NO: 169) |

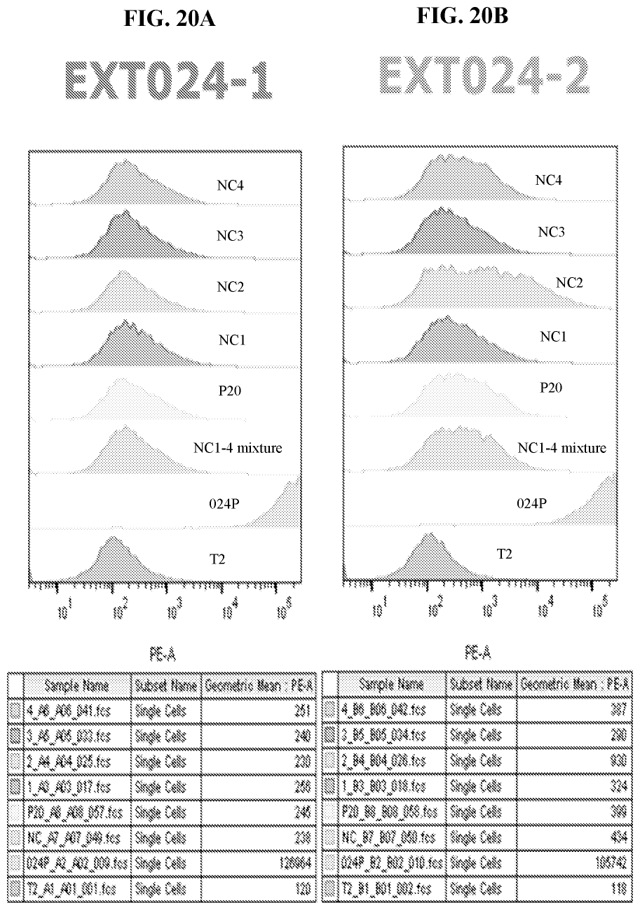

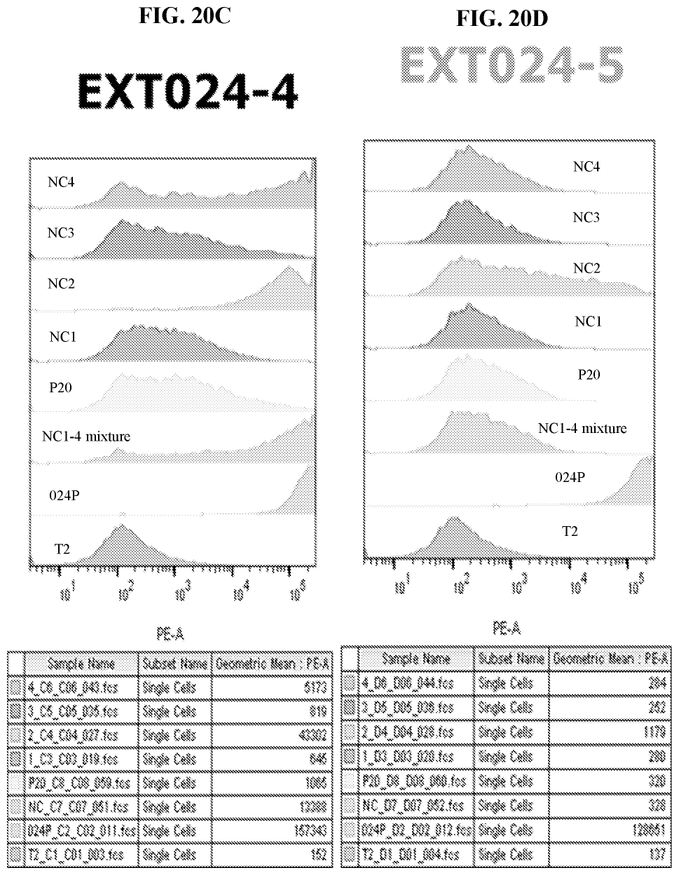

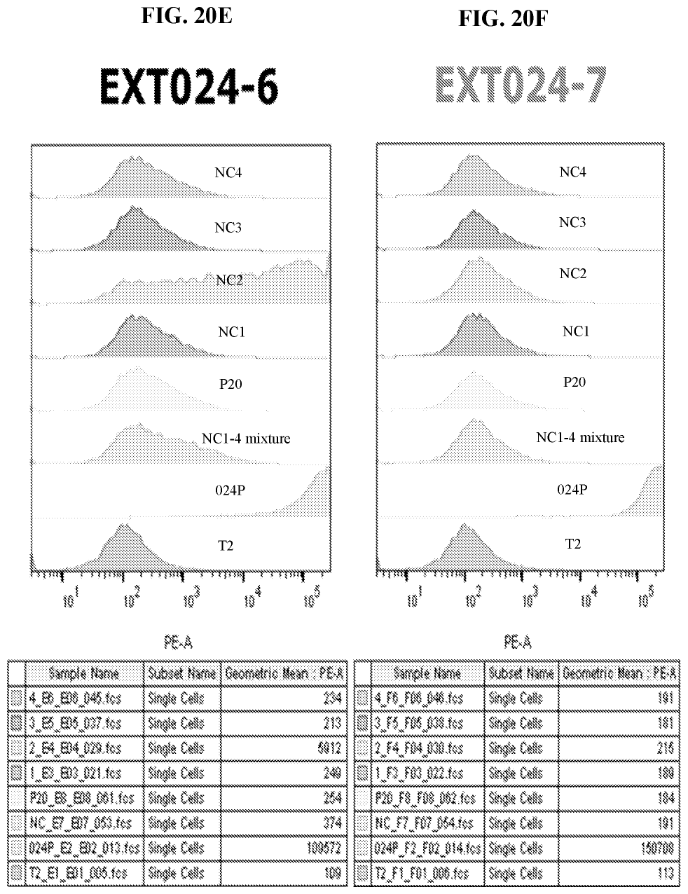

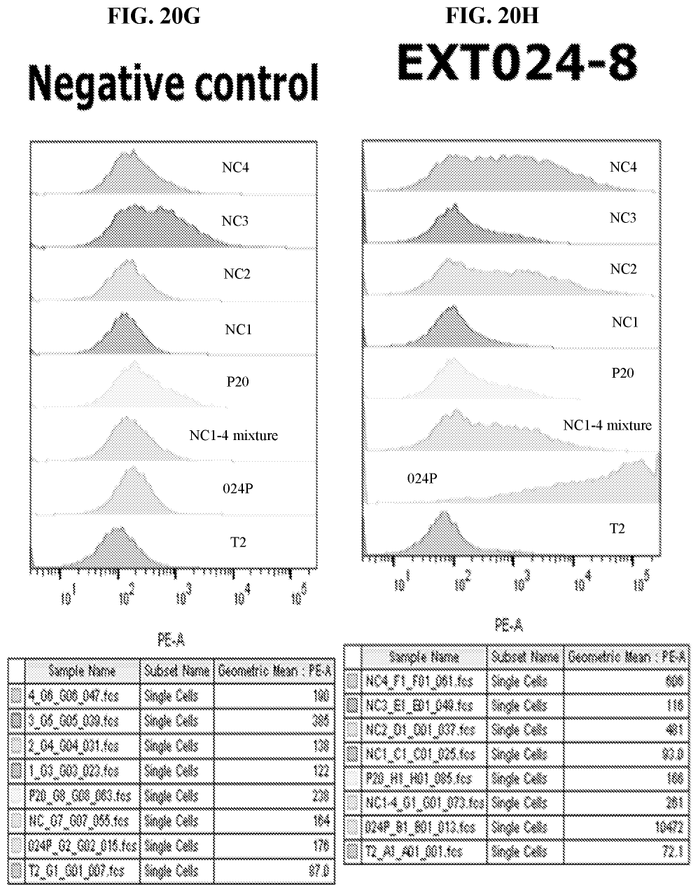

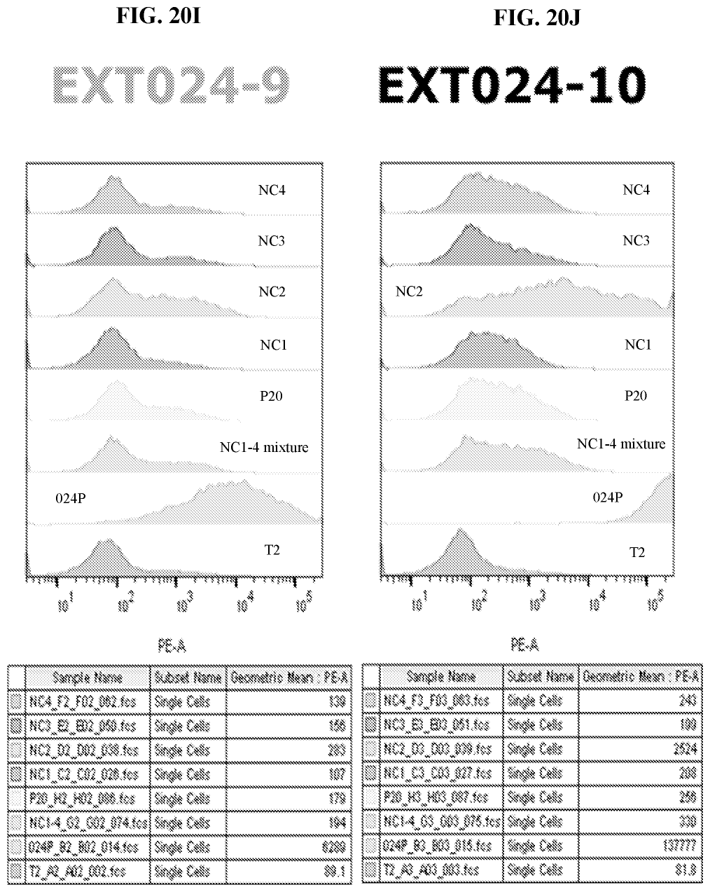

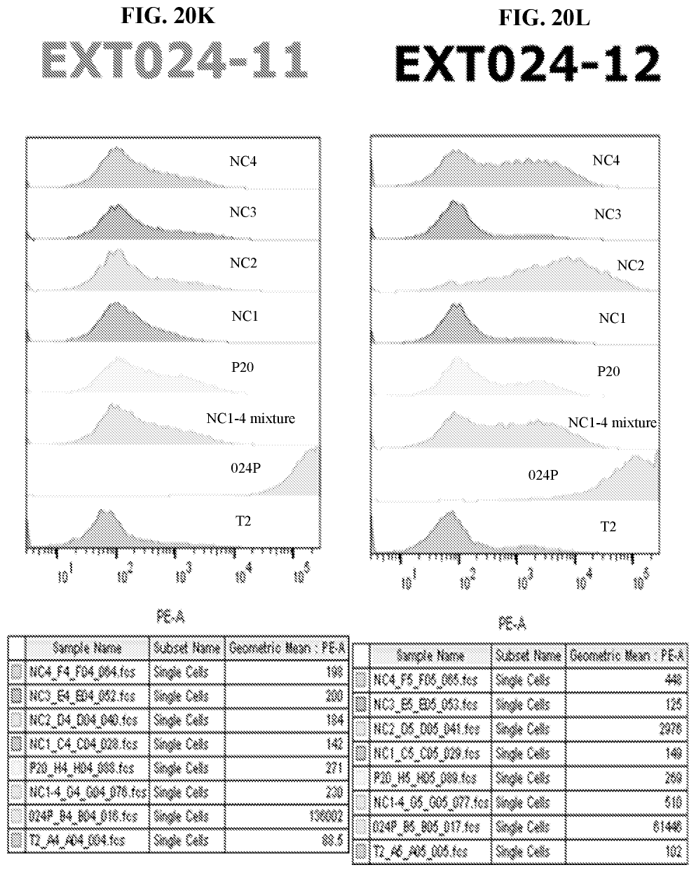

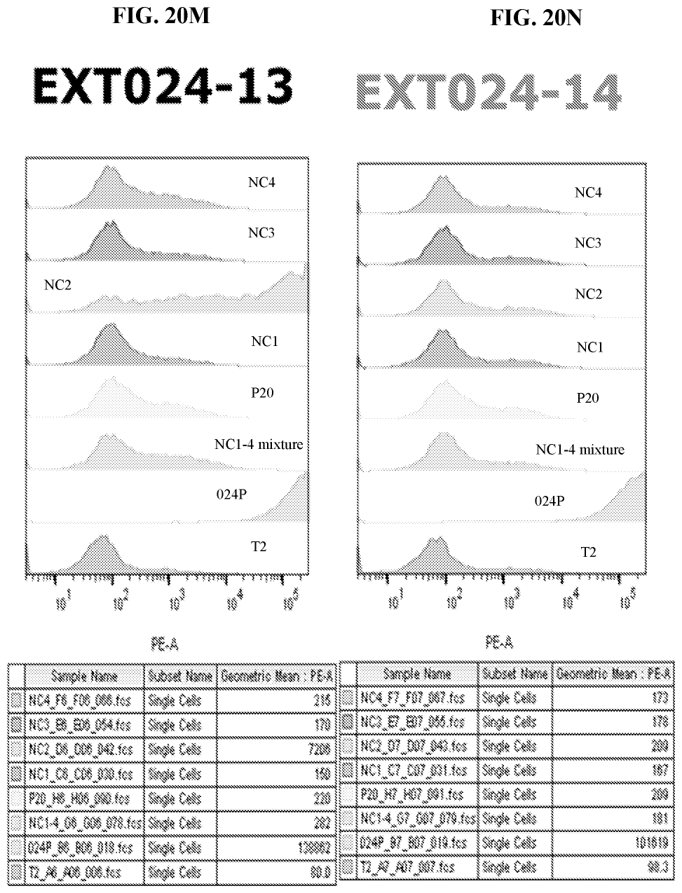

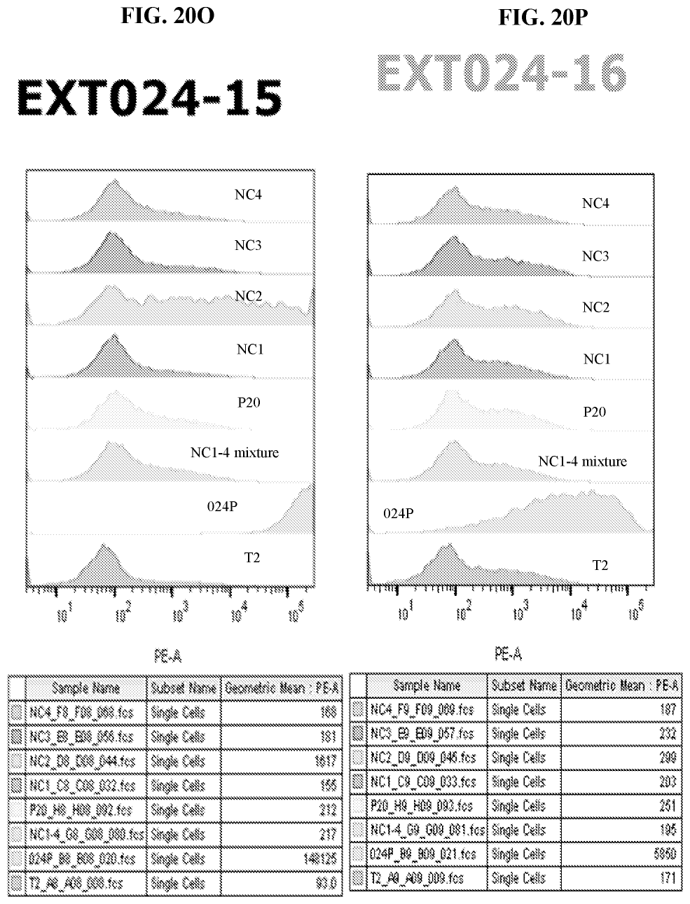

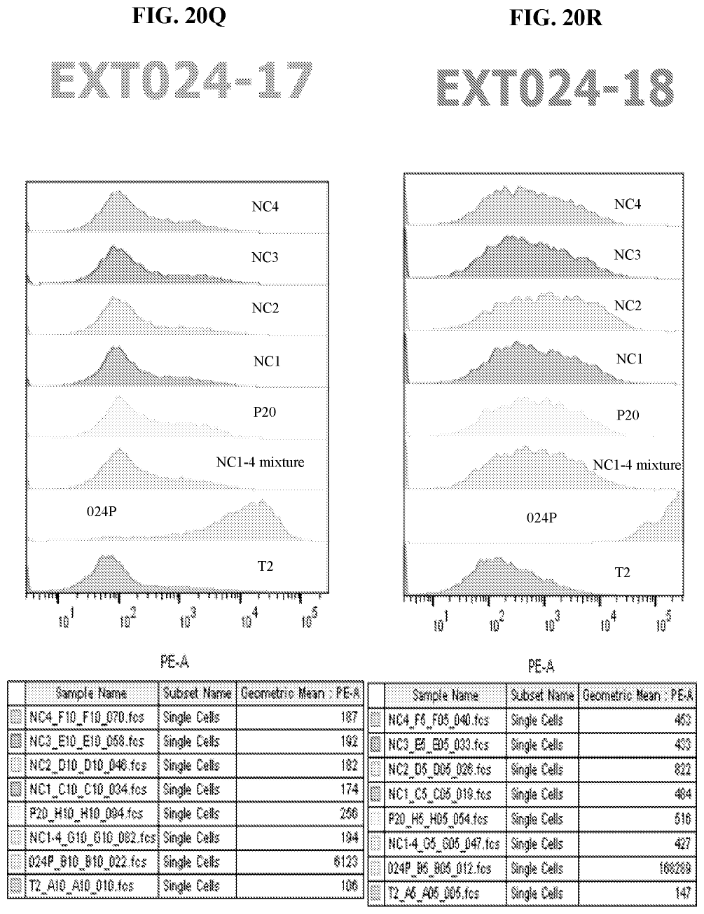

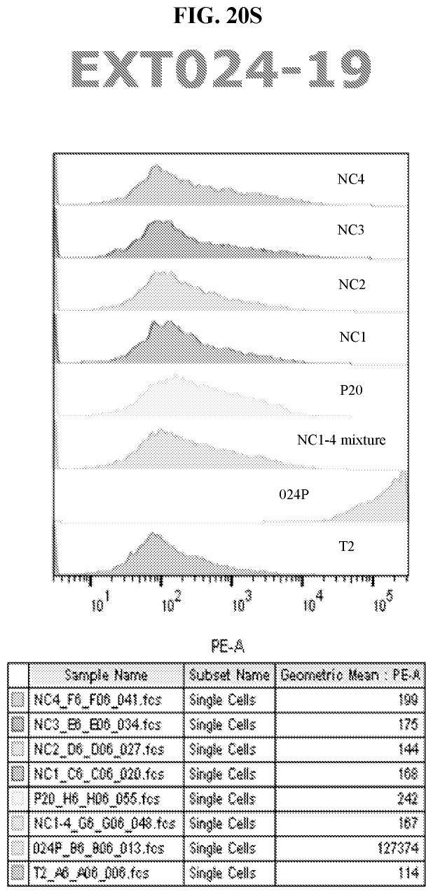

A mixture of these four homologous peptides (NC1-NC4) were loaded on T2 cells to test the specificity of positive phage clones. Among the 60 NDC80/MHC I-positive clones (also called NDC80-positive clones), 18 clones were identified with specific binding to NDC80/HLA-A*02:01 complex but with no or low binding to T2 cells loaded with the mixture of NC1-NC4 peptides. See FIGS. 20A-20S.

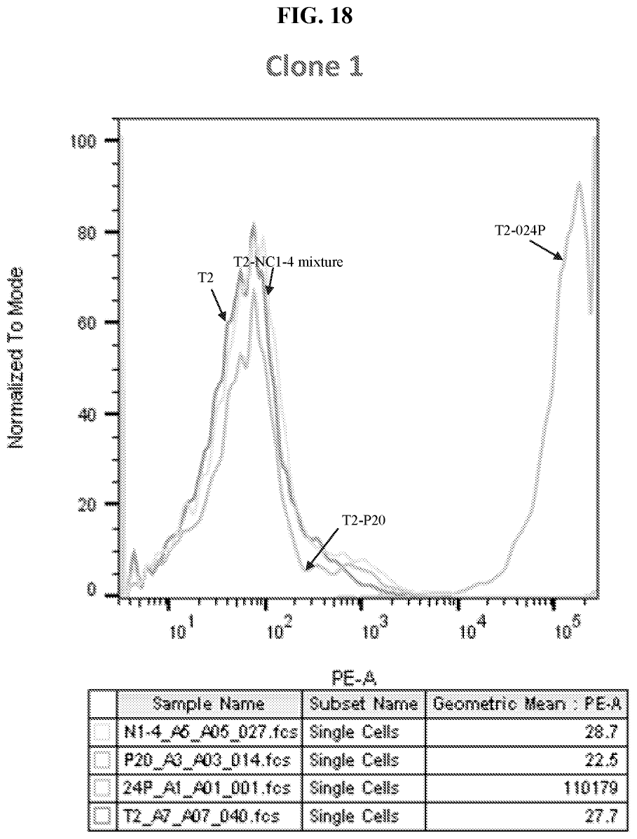

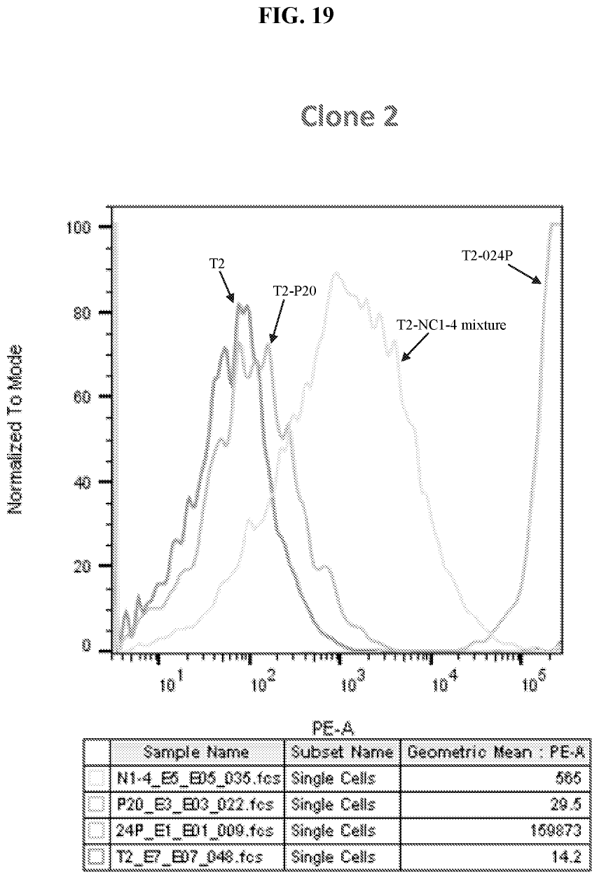

FIG. 18 demonstrates that phage clone 1 had high specificity of NDC80/MHC I binding (with no cross-reactivity to any of the four homologous peptides), while FIG. 19 demonstrates that phage clone 2 had medium specificity of NDC80/MHC I binding (with some low-level cross-reactivity to at least one of the four homologous peptides). The amino acid sequences of the CDR regions of the 18 NDC80/MHC I-specific phage/antibody clones are shown in FIG. 23. The complete V_{H} and V_{L} fragments of the 18 NDC80/MHC I-specific phage/antibody clones are disclosed herein.

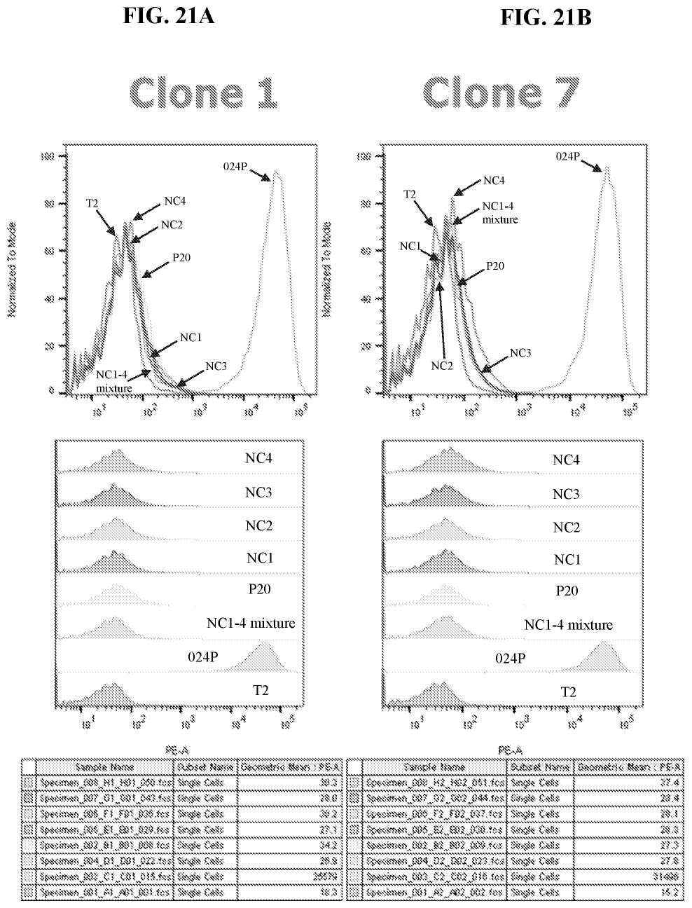

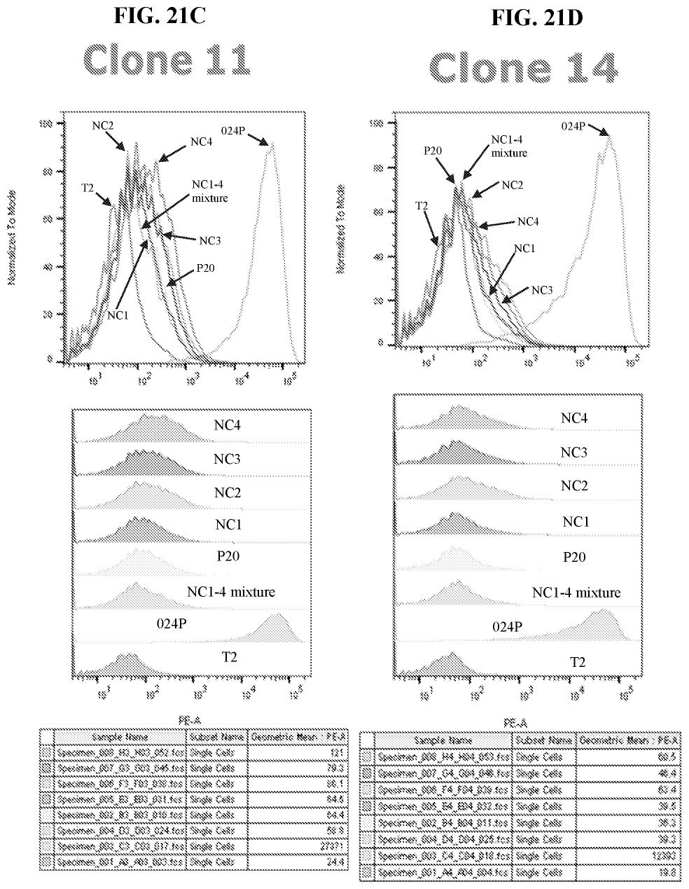

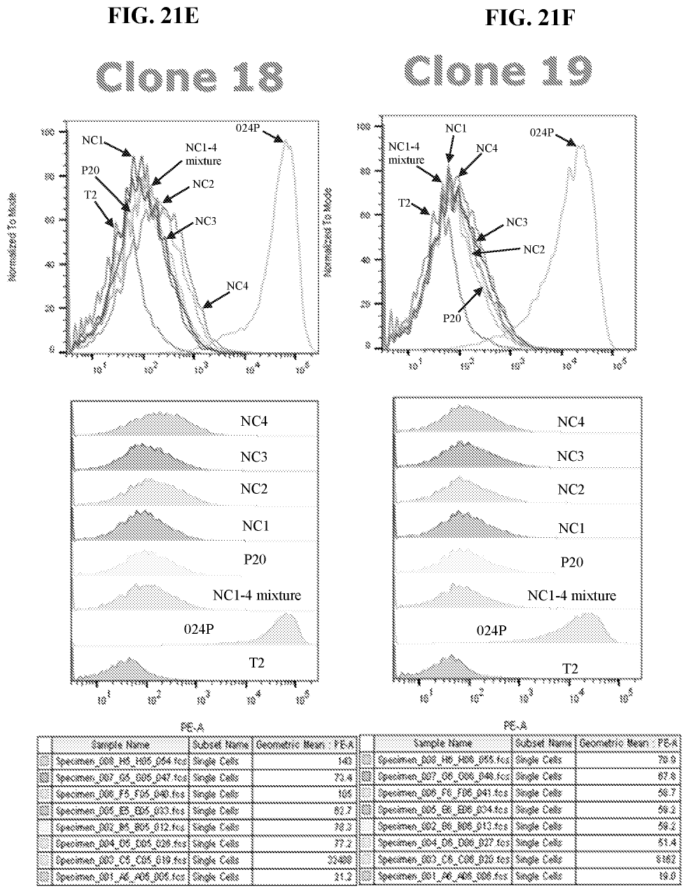

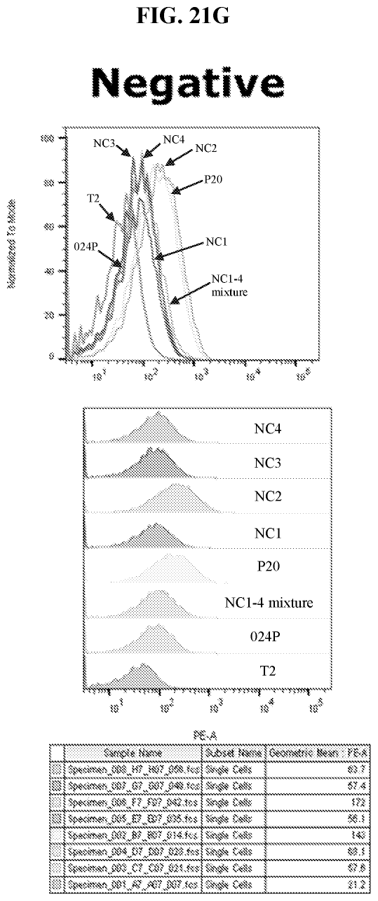

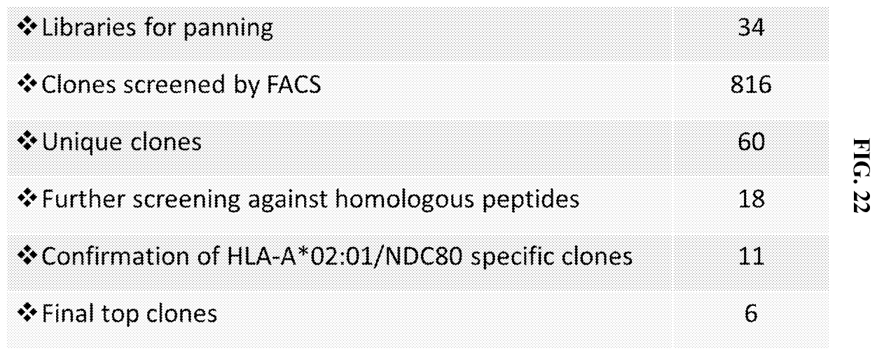

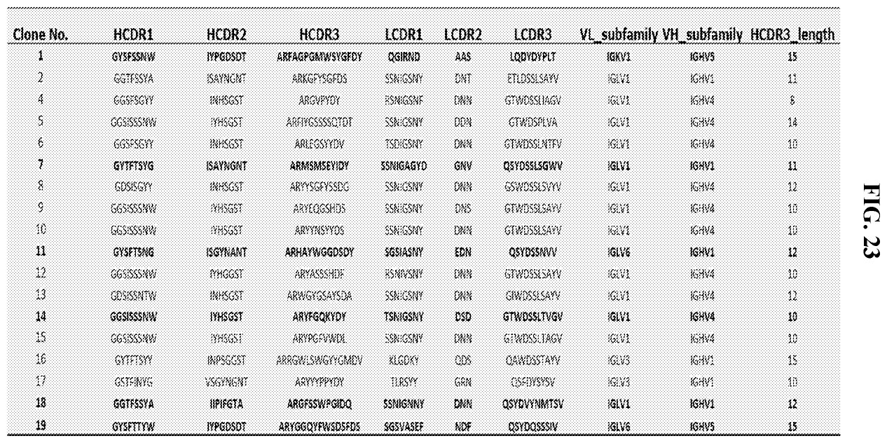

To further investigate potential cross-reactivity of the anti-NDC80/MHC antibody clones towards the four homologous peptides, NC1-NC4 peptides were loaded on T2 cells separately and tested against each of the 18 NDC80/MHC I-specific (also called NDC80-specific) phage/antibody clones. The binding of phage clones to the NDC80 Peptide A and each of the four homologous peptides complexed with MHC I was investigated by FACS analysis. Six top phage clones were identified with highly specific binding to NDC80 peptide and no binding to any of the four homologous peptides: clones 1, 7, 11, 14, 18, and 19. See FIGS. 21A-21G. The amino acid sequences of the complete scFv fragments of the six top NDC80/MHCI-specific phage/antibody clones (SEQ ID NOs: 38-43) are shown below. The V_{H} and V_{L} CDR 1-3 sequences are underlined.

Clone 01 scFv
(SEQ ID NO: 38)
DIQLTQSPSSLSASVGDRVTITCRASQGIRNDLGWYQQKP
GKAPKLMIYAASSLQSGVPSRFSGSGSGTDFTLTISSLQP
EDFATYYCLQDYDYPLTFGGGTKLEIKRSRGGGGSGGGGS
GGGGSLEMAEVQLVQSGAEVKKPGESLKISCEASGYSFSS
NWIAWVRQRPGKGLEWMGIIYPGDSDTRYSPSFQGQVTMS
ADMSISTAYLQWSSLKASDTAMYYCARFAGPGMWSYGFDY
WGQGTLVTVSS
Clone 07 scFv
(SEQ ID NO: 39)
QSVVTQPPSVSGAPGQRITISCTGSSSNIGAGYDVHWYQQ
LPGTAPKLLIFGNVNRPSGVPDRFSGSKSGTSASLAITGL
QAEDEADYYCQSYDSSLSGWVFGGGTKLTVLGSRGGGGSG
GGGSGGGGSLEMAQVQLQQSGAEVKKPGATVKVSCKASGY
TFTSYGISWVRQAPGQGLEWMGWISAYNGNTNYAQKLQGR
VTMTTDTSTSTAYMELRSLRSDDTAVYYCARMSMSEYIDY
WGQGTLVTVSS
Clone 11 scFv
(SEQ ID NO: 40)
NFMLTQPHSVSESPGKTVTISCTRSSGSIASNYVQWYQQR
PGSAPTTVIYEDNQRPSGVPDRFSGSIDSSSNSASLTISG
LKTEDEADYYCQSYDSSNVVFGGGTKLTVLGSRGGGGSGG
GGSGGGGSLEMAQVQLVQSGAEVRKPGASVKVSCKASGYS
FTSNGITWVRQAPGQGLEWMGWISGYNANTRYAQEFQARV
TMTTDTSASTAYMELRSLRSDDTAVYYCARHAYWGGDSDY
WGQGTLVTVSS
Clone 14 scFv
(SEQ ID NO: 41)
QSVLTQPPSVSAAPGQRVTISCSGSTSNIGSNYVSWYQQF
PGTAPKLLIYDSDKRISGIPDRFSGSKSGTSATLGITGLQ
TGDEADYYCGTWDSSLTVGVFGGGTKVTVLGSRGGGGSGG
GGSGGGGSLEMAQLQLQESGPGLVKPSGTLSLTCAVSGGS
ISSSNWWSWVRQPPGKGLEWIGEIYHSGSTNYNPSLKSRV
TISVDKSKNQFSLKLSSVTAADTAVYYCARYFGQKYDYWG
QGTLVTVSS
Clone 18 scFv
(SEQ ID NO: 42)
QSVLTQPPSVSVAPGQKVTISCSGSSSNIGNNYVSWYQQL
PGTAPKLLIYDNNKRPSGIPDRFSGSKSGTSATLGITGLQ
TGDEADYYCQSYDVYNMTSVFGGGTKLTVLGSRGGGGSGG
GGSGGGGSLEMAQMQLVQSGAEVKKPGSSVKVSCKASGGT
FSSYAISWVRQAPGQGLEWMGGIIPIFGTANYAQKFQGRV
TITADESTSTAYMELSSLRSDDTAVYYCARGFSSWPGIDQ
WGQGTLVTVSS
Clone 19 scFv
(SEQ ID NO: 43)
NFMLTQPHSVSESPGKTVTISCVRSSGSVASEFVQWYQQR
PGHAPTLVIYNDFQRPSGVPDRFSGSIDKSSNSASLTISG
LKAEDEADYYCQSYDQSSSIVFGGGTKLTVLGSRGGGGSG
GGGSGGGGSLEMAEVQLVQSGAEVKKPGESLKISCKGSGY
SFTTYWIGWVRQMPGKGLEWMGIIYPGDSDTTYSPSFQGQ
VTISADKSLSIAYLQWSSLKASDTAMYYCARYGGQYFWSD
SFDSWGQGTLVTVSS

### Example 5: In Vitro Cytotoxic Activities of the TCR Mimic Antibodies of the Present Technology

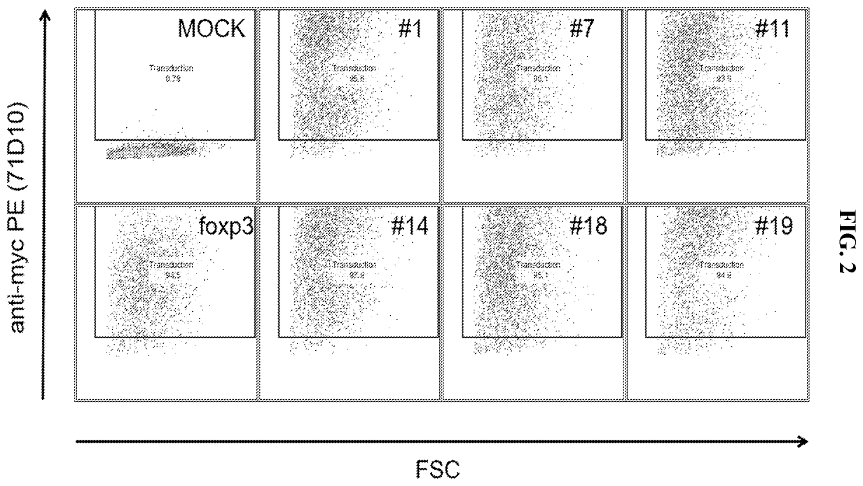

CAR T cells were generated with high and comparable efficiency for clones #1, #7, #11, #14, #18 and #19. The transduction efficiency of 28z-myc-tagged lentiviral CAR T cell constructs for Clones #1, #7, #11, #14, #18, and #19, are shown in FIG. 2.

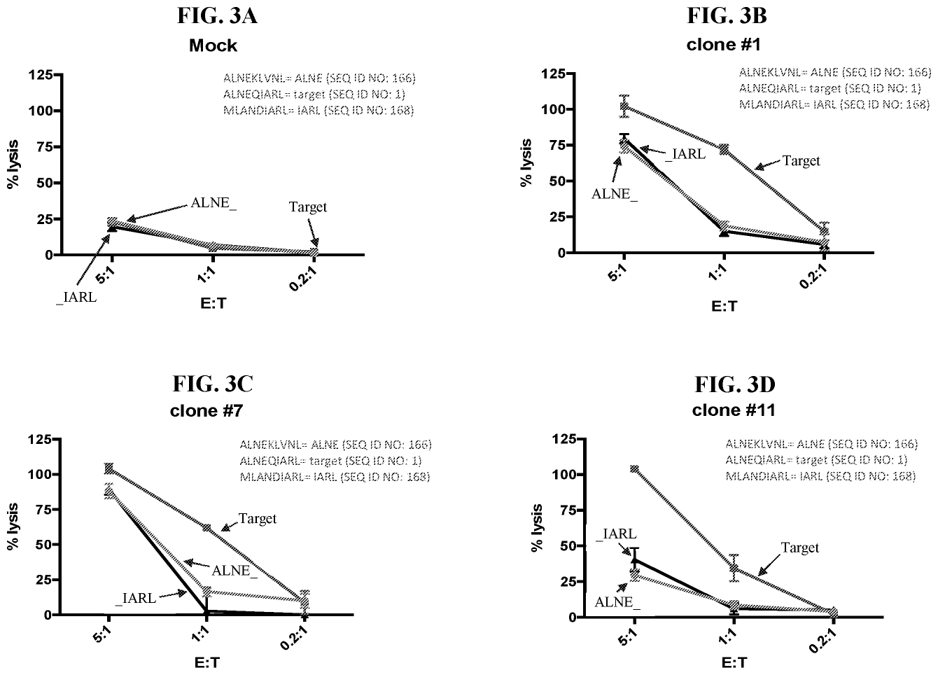

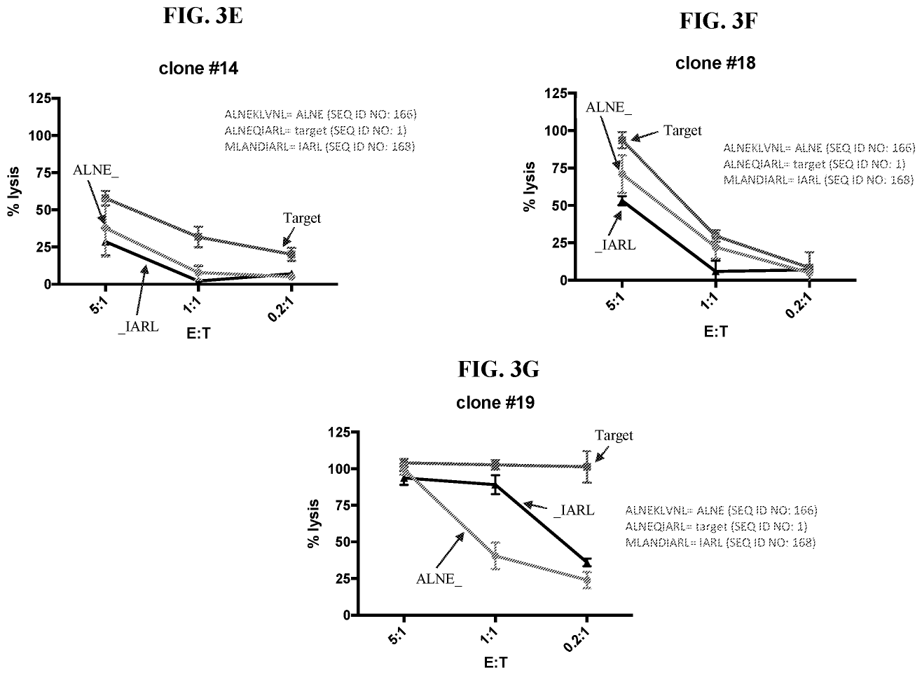

TAP-deficient T2 cells were incubated overnight with 50 μg/ml of the investigated peptides and then 5000 T2 cells were incubated with the CAR T cells described herein at the indicated E:T ratios. LDH release assay was performed according to the manufacturer's instructions (Promega, Madison, WI) after 16-18 h incubation with CAR T-cells. The peptides ALNEQIARL (SEQ ID NO: 1), ALNEKLVNL (SEQ ID NO: 166), and MLANDIARL (SEQ ID NO: 168) were used in the phage library screen for negative selection. FIGS. 3A-3G show the results of peptide-pulsed T2 cells evaluated in a LDH release assay with CAR T cells including Clones #1, #7, #11, #14, #18, and #19.

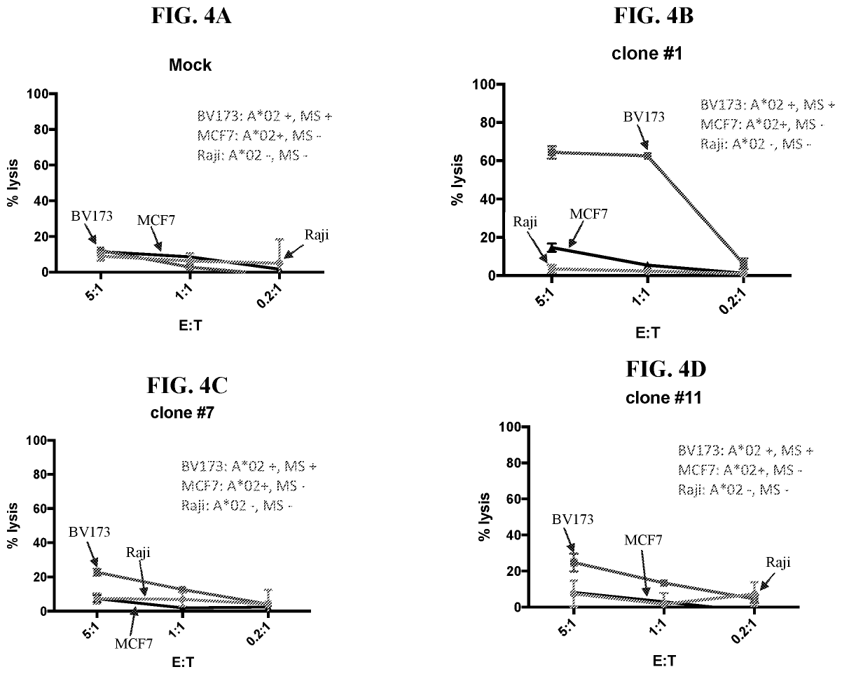

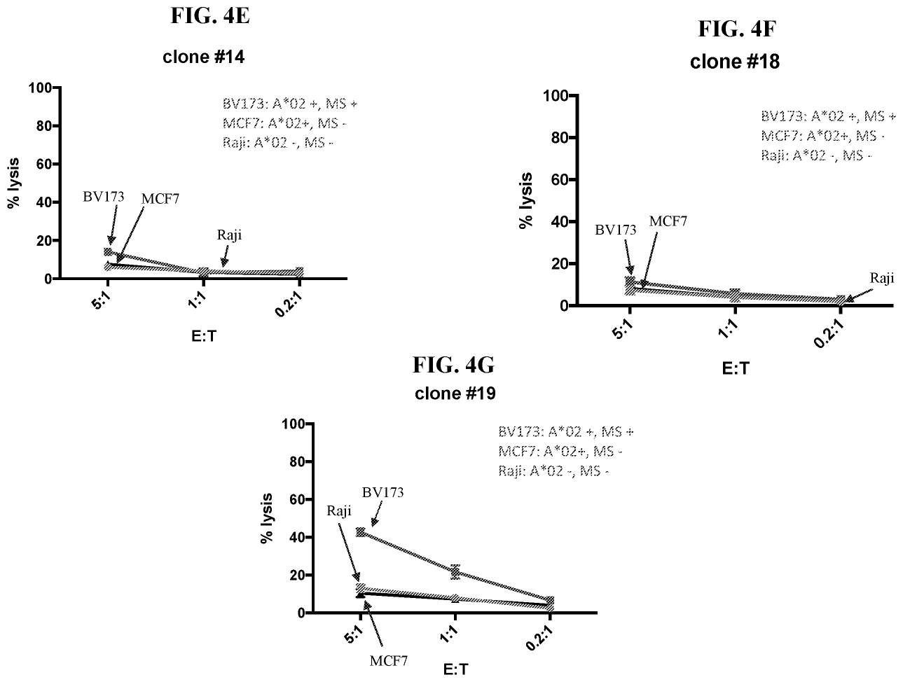

For non-T2 cell lines, 5000-10,000 cells were used per experiment and cancer cells were incubated for 16-18h with the CAR T cells described herein indicated at respective E:T ratios. The % cell lysis was assessed by using an LDH release assay. FIGS. 4A-4G show the 18 hour LDH release assay results with CAR T cells including Clones #1, #7, #11, #14, #18, and #19 in BV173, MCF7 and Raji cell lines.

Seven cell lines were selected each from hematological and non-hematological origin based on HLA typing, as well as evidence for presentation of the ALNEQIARL (SEQ ID NO: 1) target by mass spectrometry (Table 6). Of note, five of the cell lines in each group were ALNEQIARL (SEQ ID NO: 1):HLA-A*02 positive, one in each group was HLA-A*02 positive, but negative by mass spectrometry for ALNEQIARL (SEQ ID NO: 1) (SUDHL4 and HCT116) and one cell line each was negative for both HLA-A*02 and the target peptide (HL60 and T47D).

TABLE 6
Cell line killing based on mass spectrometry
evidence of HLA-A*02:ALNEQIARL (SEQ ID NO: 1)

| | | | |
|---|---|---|---|
| | | NDC80-clone1 | NDC80-clone11 |
| Cell line | HLA-A*02/MS | killing | killing |

Hematological malignancies

| | | | |
|---|---|---|---|
| AML14 | +/+ | +++ | + |
| OCI-AML02 | +/+ | +++ | + |
| BV173 | +/+ | +++ | + |
| SUDHL6 | +/+ | + | − |
| MAC2A | +/+ | +++ | + |
| SUDHL4 | +/− | − | − |
| HL60 | −/− | − | − |

Non-hematological malignancies

| | | | |
|---|---|---|---|
| JMN | +/+ | +++ | + |
| TPC1 | +/+ | ++ | − |
| MDA-MB231 | +/+ | ++ | − |
| PANC1 | +/+ | + | − |
| SKMEL-5 | +/+ | + | − |
| HCT116 | +/− | − | − |
| T47D | −/− | − | − |

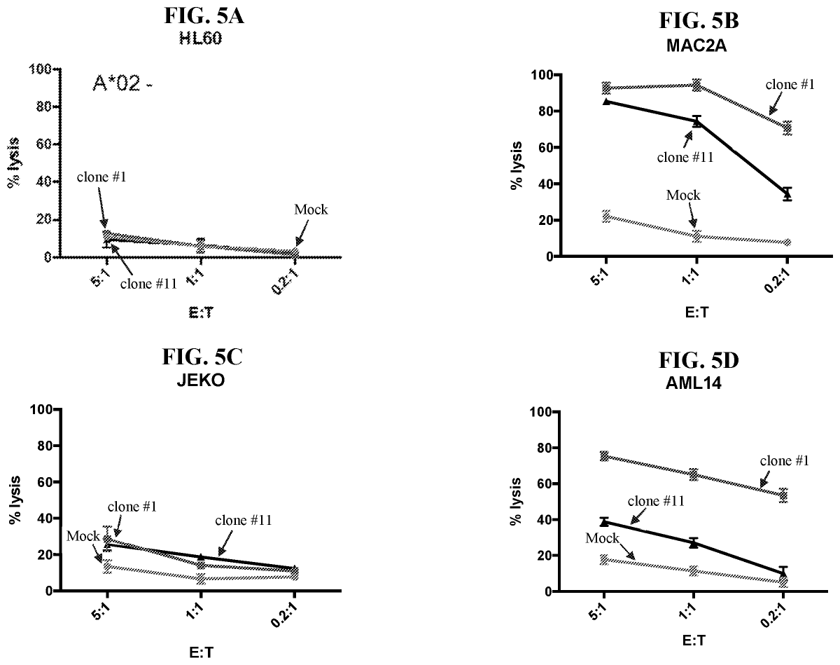

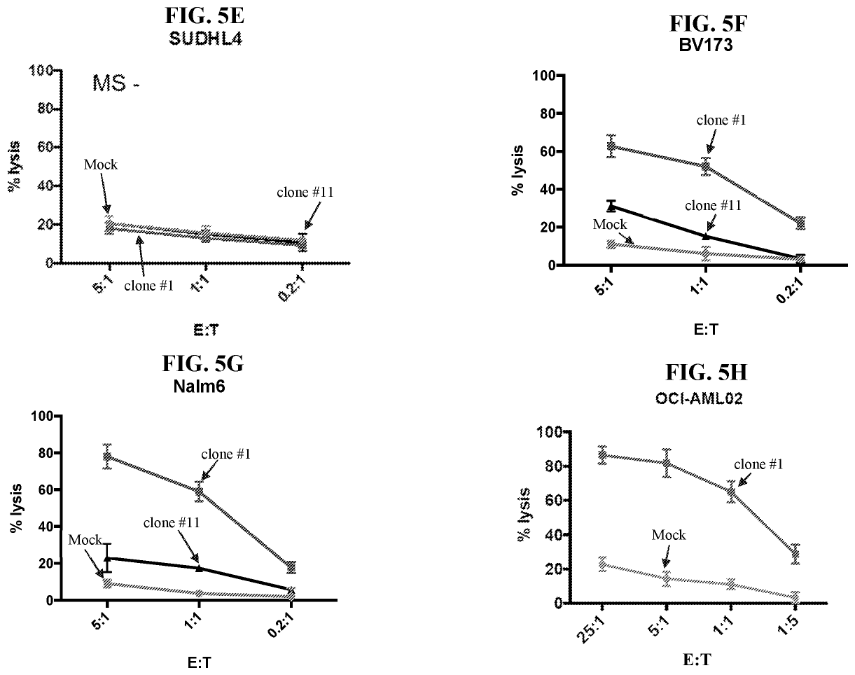

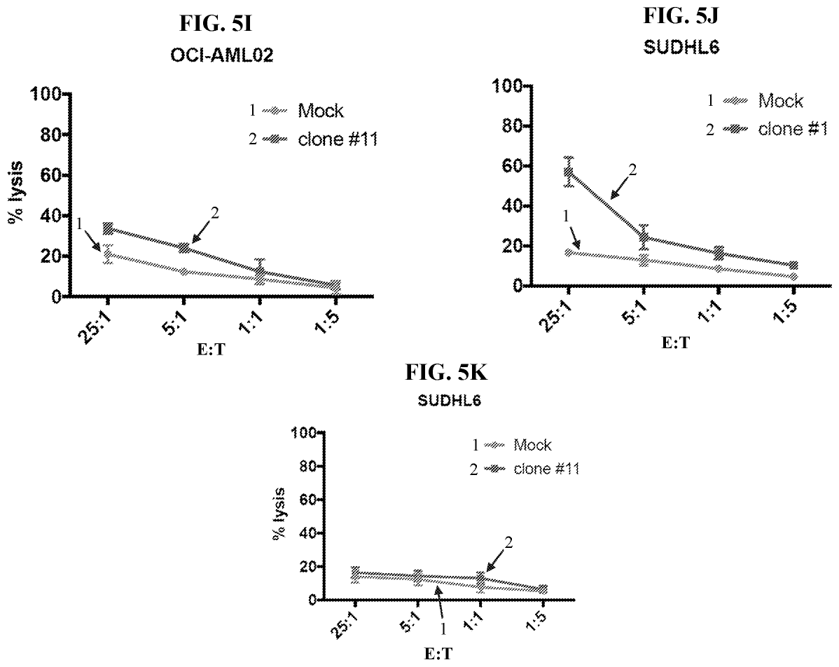

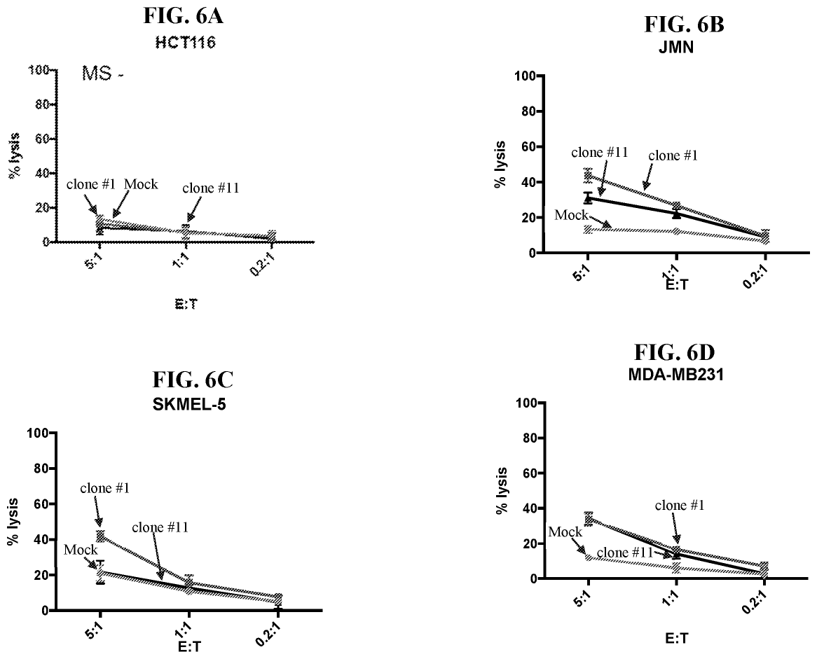

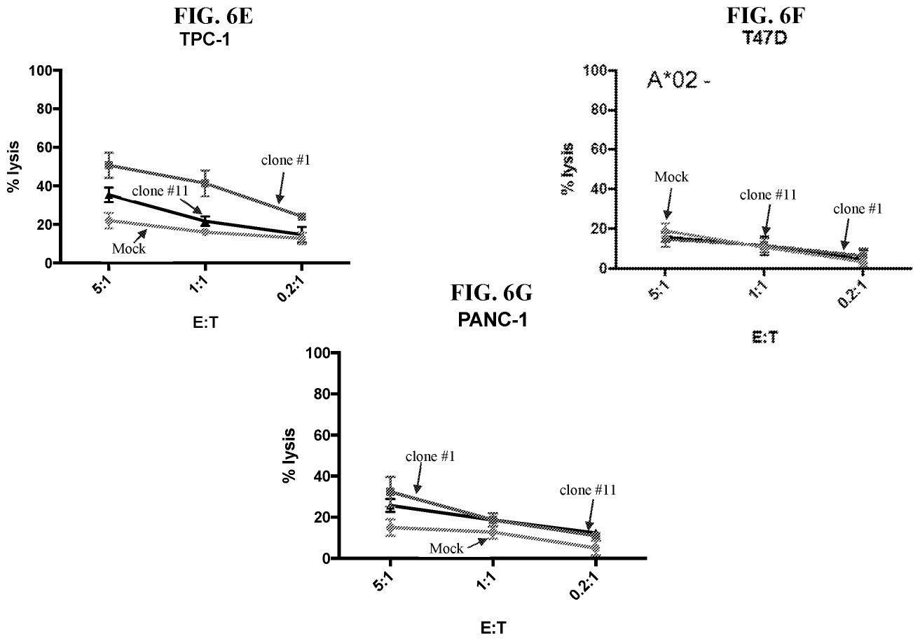

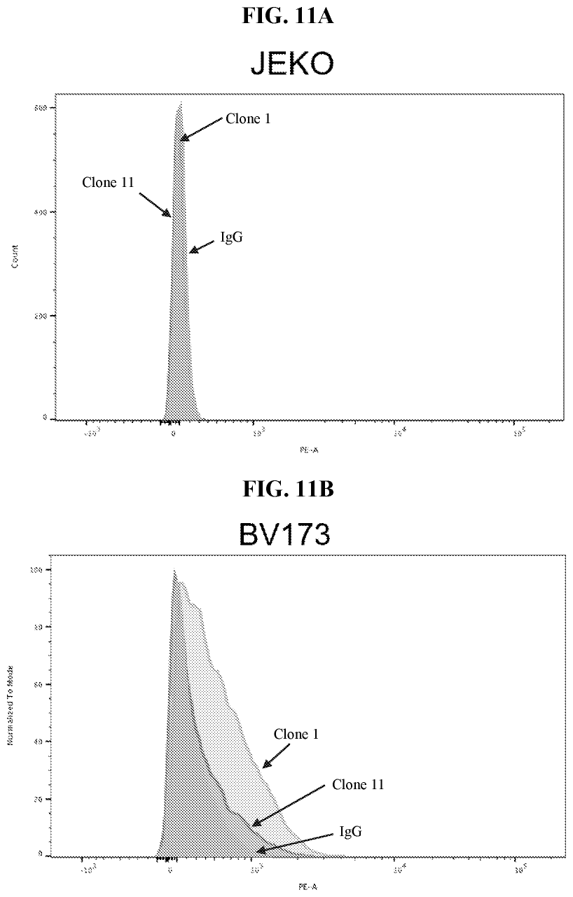

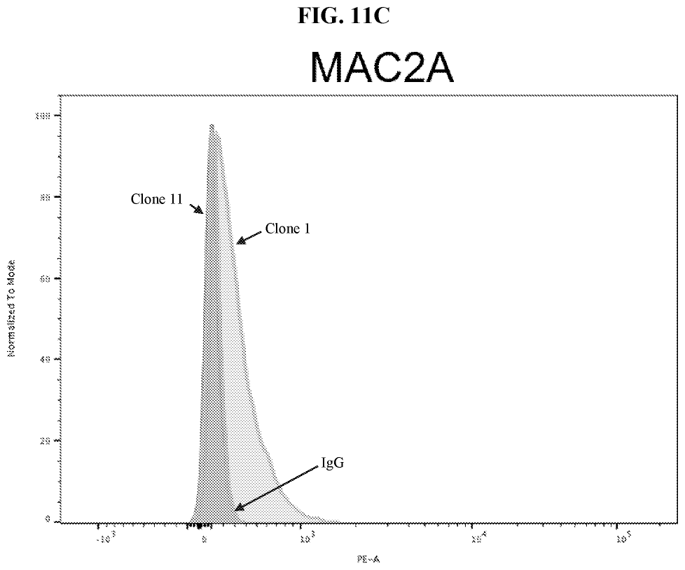

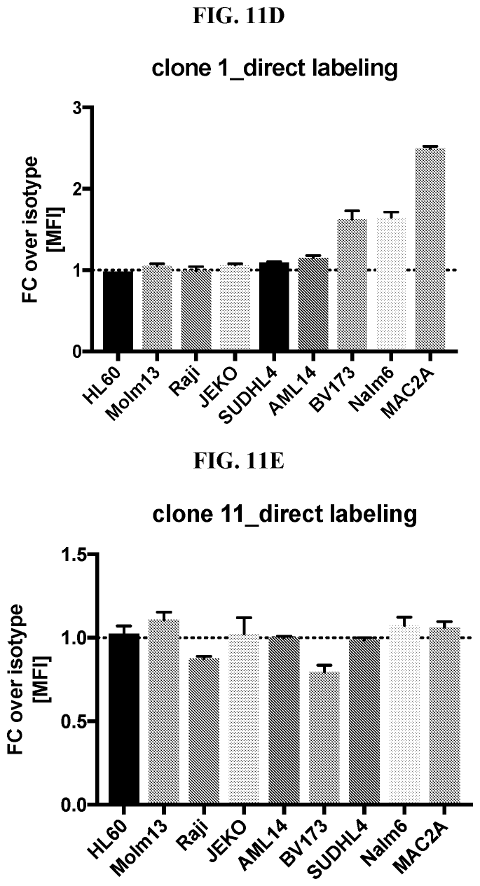

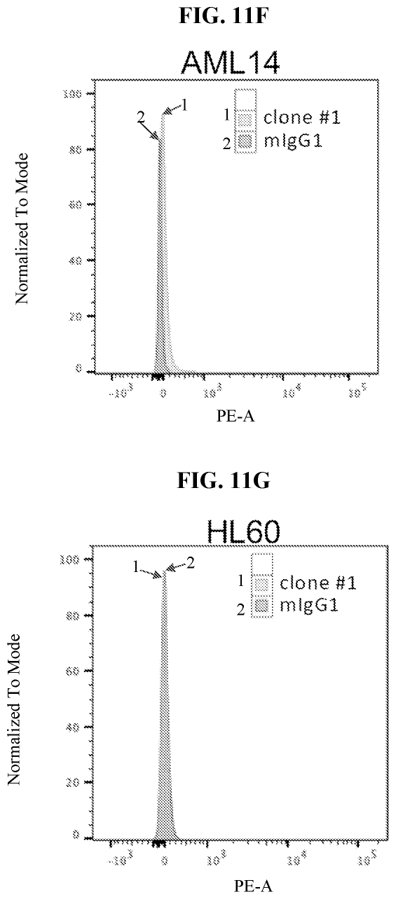

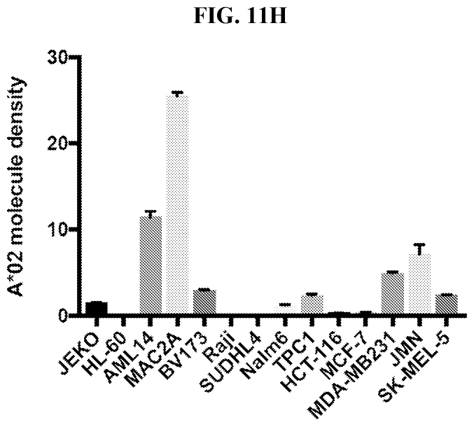

FIGS. 5A-5K and FIGS. 6A-6G show the 18 hour LDH release assay results with CAR T cells including Clones #1 and #11 in various indicated hematological cancer cell lines, and adherent cell lines from solid cancers. Clone 1 kills better than clone 11, but overall different cell lines were lysed: Melanoma (SKMEL5), Thyroid cancer (TPC1), Mesothelioma (JMN), breast cancer (MDA-MB231), and pancreatic cancer (PANC-1). Both clones demonstrated more effective killing against cell lines of hematological origin, which can be explained by their greater abundance of HLA-A*02 molecules on the cell surface relative to their size creating a higher antigen density. FIG. 11H.

These results demonstrate that the immunoglobulin-related compositions of the present technology specifically kill multiple cancer types including hematological malignancies.

### Example 6: Epitope Identification and Characterization of Functional Activity of the TCR Mimic Antibodies of the Present Technology

Alanine scanning was performed to determine the critical residues on NDC80 Peptide A that were required for functional activity of the TCR mimic antibody clones of the present technology.

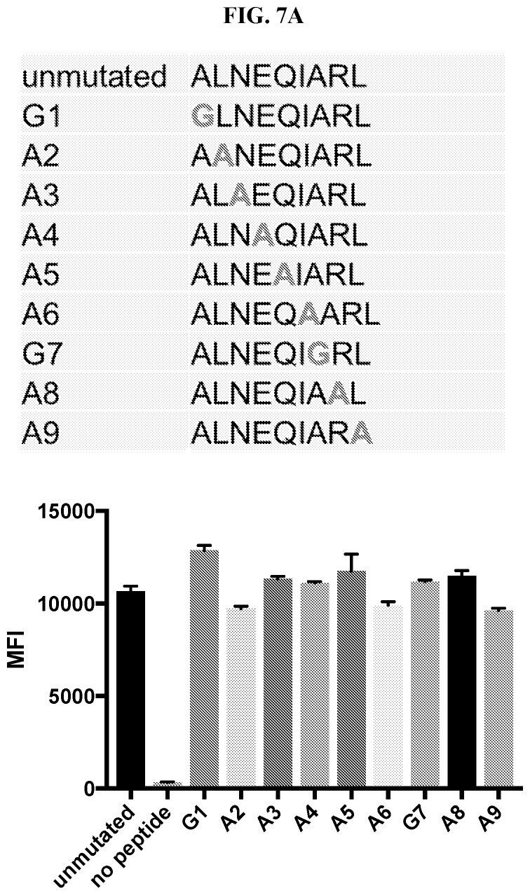

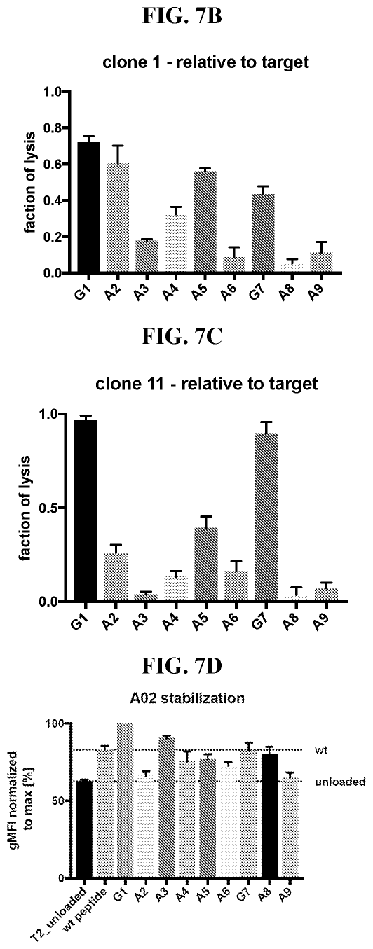

For alanine screening assays, alanine or glycine substituted peptides were purchased from Genemed Synthesis, Inc. (San Antonio, Texas, USA). Besides the unsubstituted sequence of ALNEQIARL (SEQ ID NO: 1), purchased peptides included peptides with single amino acid substitutions from position 1 to 9. If in the original peptide sequence the amino acid present was an alanine, the amino acid was changed to glycine. All other amino acids were changed into alanines. FIG. 7A shows a complete list of the peptides used for the alanine screening assays as well as stabilization of NDC80 peptide variants on T2 cells pulsed with the peptide variants as measured by A02 specific antibody BB7.2. Alanine substitutions at the anchor positions 2 and 9 of the peptide only allowed moderate stabilization of the HLA-A*02 complexes on T2 cells, though all other peptides stabilized the A*02 protein to similar levels as the unmodified peptide (FIG. 7D).

As shown in FIG. 7B, residues 3, 6, and 8 of NDC80 Peptide A are the most critical residues for facilitating cytotoxic activity of CAR T cells including clone 1 sequences compared with residues 4 and 5 as determined by LDH release assay. As shown in FIG. 7C, residues 3, 4, 6, and 8 of NDC80 Peptide A are the most critical residues for facilitating cytotoxic activity of CAR T cells including clone 11 sequences as determined by LDH release assay.

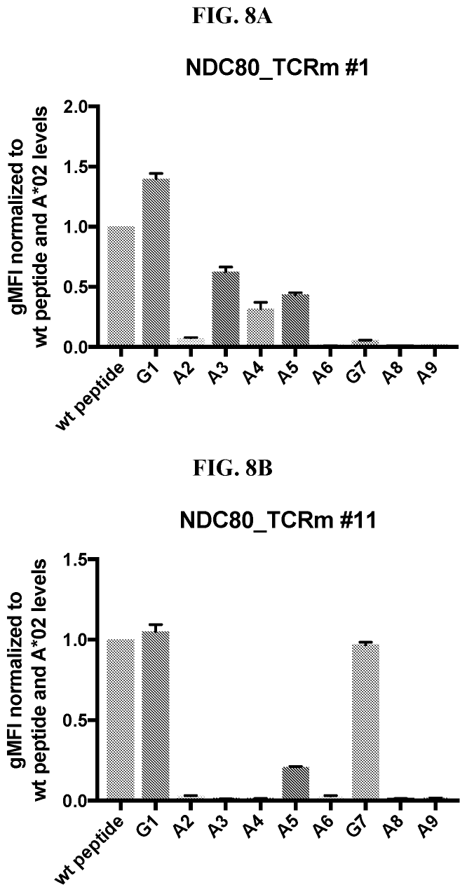

Alanine scanning experiments were performed on T2 cells that were pulsed with NDC80 peptide variants overnight. Binding to NDC80 peptide variants was measured by flow cytometry using direct staining with the given TCR mimic antibody. As evident from FIG. 8A, residues 6, 7, and 8 of NDC80 Peptide A are the most critical residues for binding to TCR mimic antibody including clone 1 sequences compared with residues 3, 4 and 5. As shown in FIG. 8B, residues 3, 4, 6, and 8 of NDC80 Peptide A are the most critical residues for binding to TCR mimic antibody including clone 11 sequences compared with residue 5. Overall results using direct antibody staining were comparable to CAR T cell experiments.

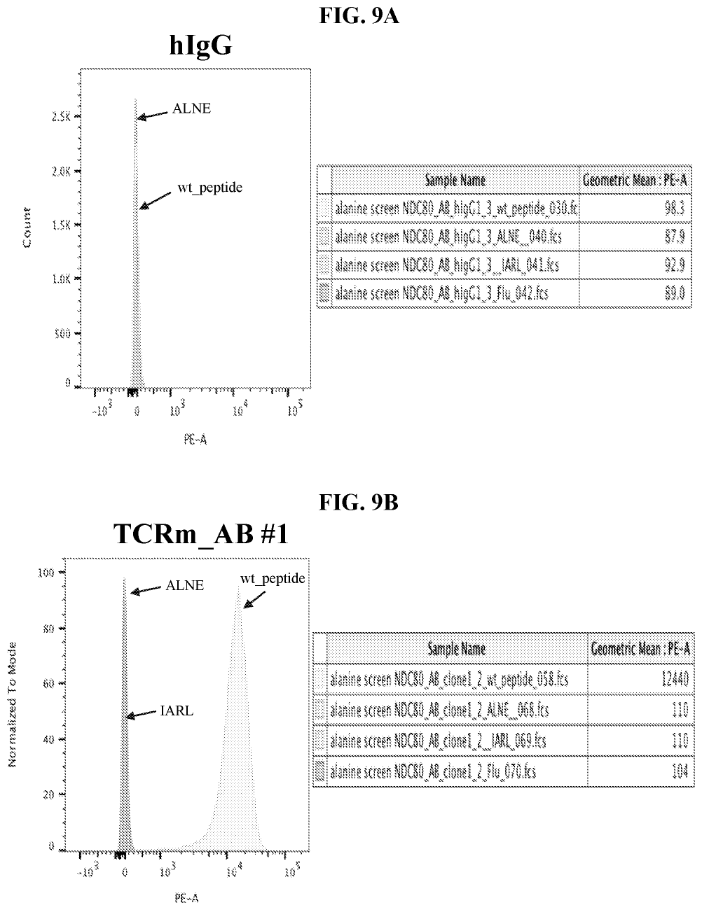

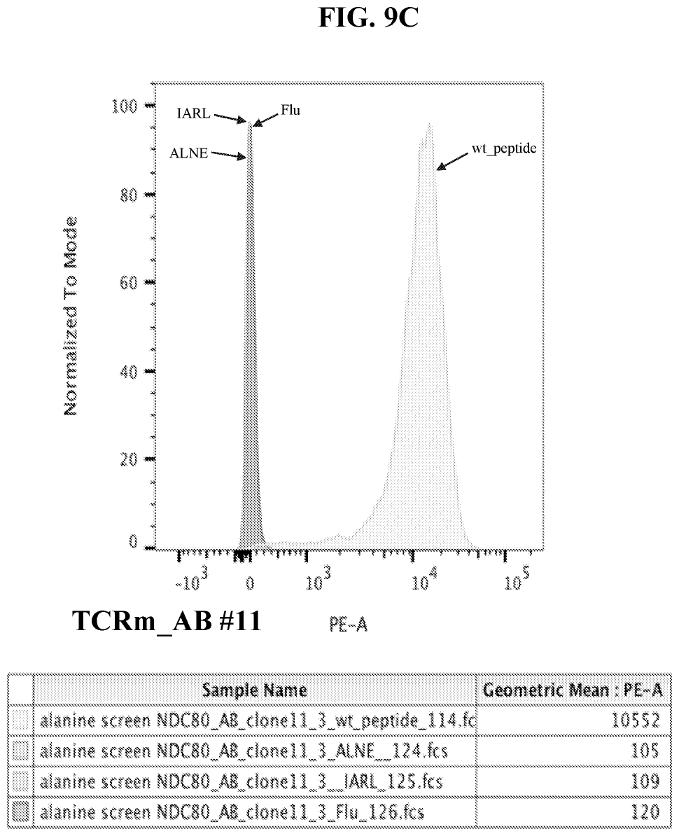

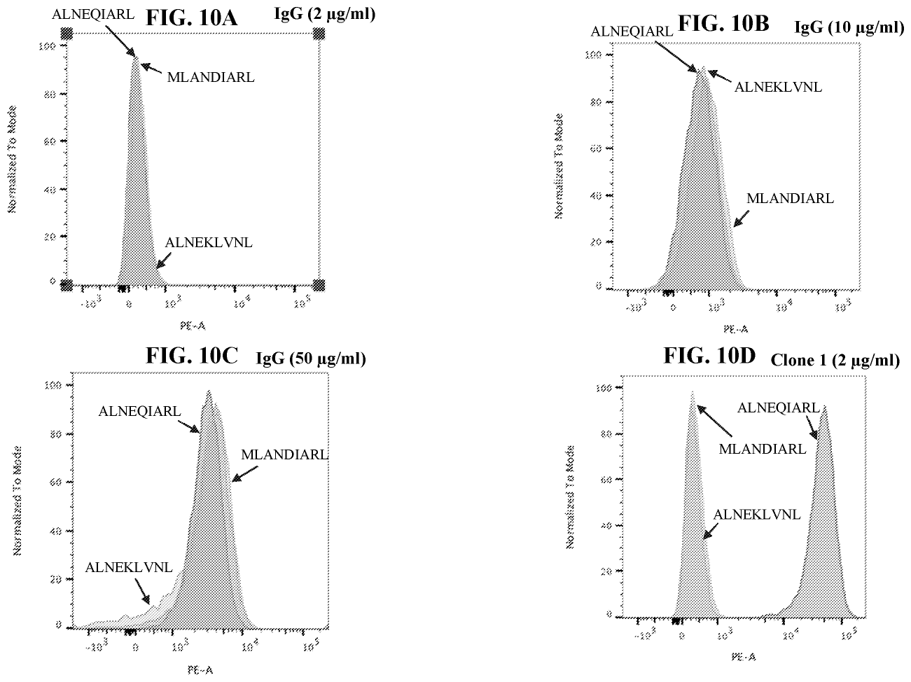

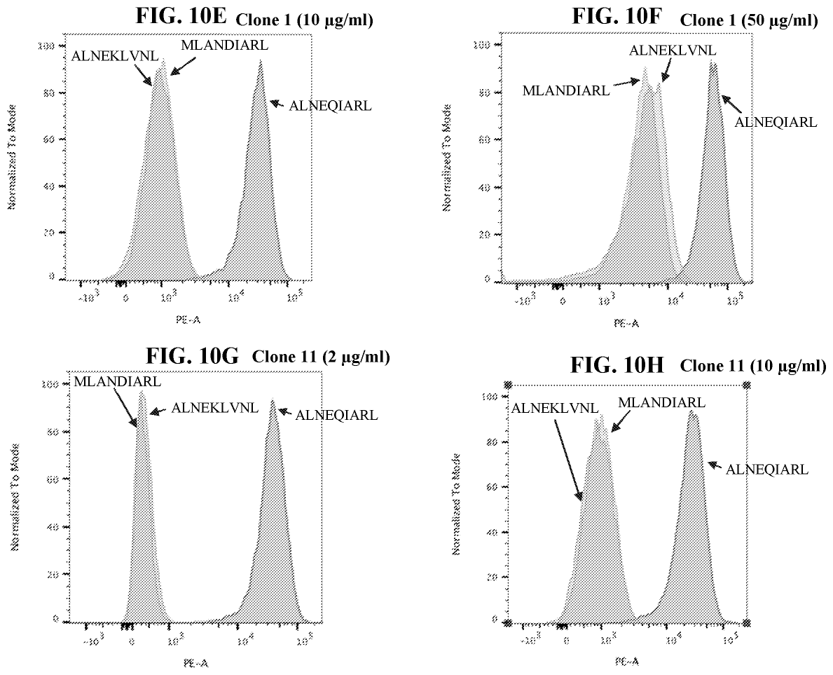

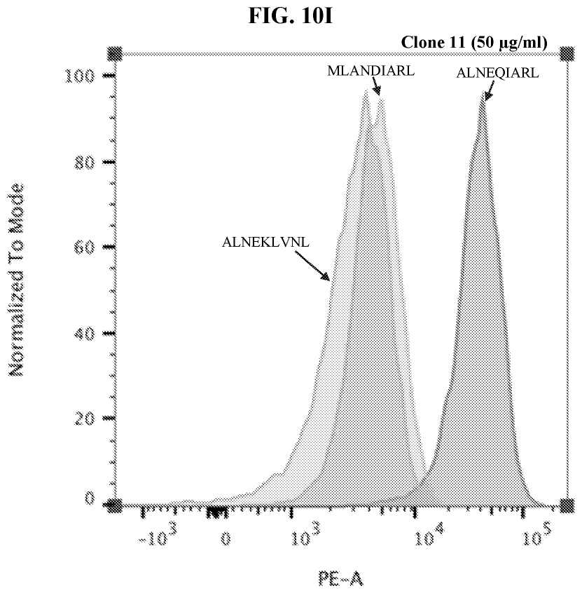

For the experiments described in FIGS. 9A-9C, T2 cells were cultured in RPMI media and pulsed over night with 50 μg/ml of one of the indicated peptides (target NDC80 Peptide A or negative control Flu peptide or NC1 peptide or NC3 peptide). TCR mimic antibodies were coupled to PE using the Lightning-Link® R-PE Antibody Labeling Kit (Novusbio, Cat. #703-0010) according to the manufacturer's instructions. Pulsed T2 cells were harvested, washed with FACS buffer (PBS, 2% FBS and 0.01% sodium azide) and incubated for 30 min, light-protected, on ice with 2 μg/ml of the labeled antibodies. After incubation, cells were washed again with FACS buffer and resuspended in FACS buffer supplemented with DAPI. For the experiments described in FIGS. 10A-10I, incubation with the TCR mimic antibodies was performed at 2, 10 and 50 μg/ml. Cells were analyzed on an LSRFortessa™ (BD) flow cytometer; the data were analyzed using FlowJo™ v9 software.

As shown in FIGS. 9A-9C, clone 1 and clone 11 both recognize the target Peptide A with high specificity in the TCR mimic antibody format at 2 μg/ml using flow cytometry. The specificity of TCR mimic antibody clones 1 and 11 was maintained even at concentrations as high as 50 μg/ml. See FIGS. 10A-10I. No relevant cross-reactivity of the mIgG1 antibodies was observed with T2 cells pulsed with the target ALNEQIARL (SEQ ID NO: 1) or the potential off-target peptides ALNEKLVNL (SEQ ID NO: 166) or MLANDIARL (SEQ ID NO: 168) (which lack homology in the underlined center amino acids, while sharing complete homology at the N-terminal and C-terminals respectively). These potential off-target HLA ligands were also identified by MS on the cell surface of cancerous and healthy cells and therefore represent highly relevant off-targets in contrast to solely predicted off-targets.

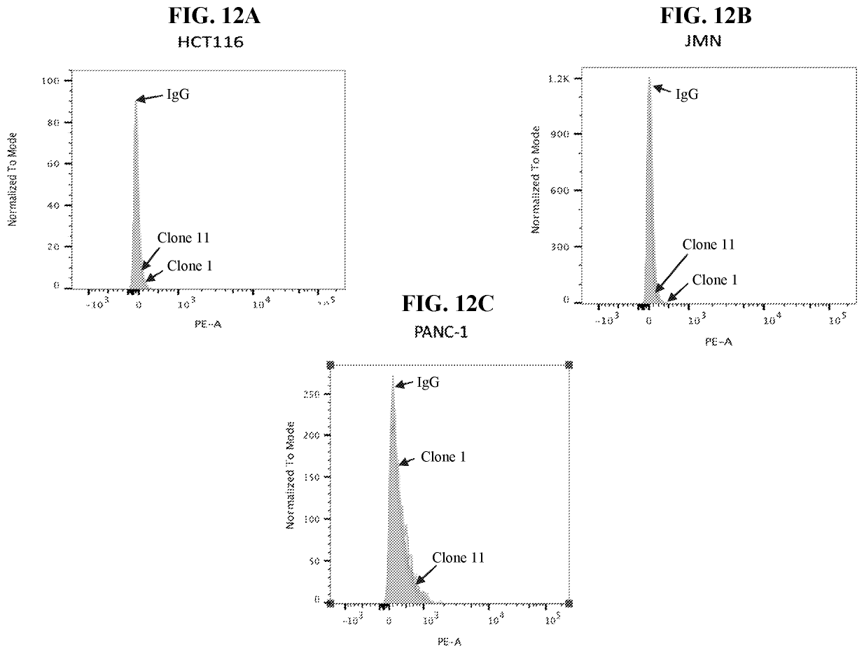

For the experiments described in FIGS. 11A-11G and FIGS. 12A-12C, suspension cell lines were harvested directly and adherent cell lines after 10 min incubation with Cellstripper™ reagent (Corning, Cat.-Nr. 25-056-C1). Cells were washed with FACS buffer (PBS, 2% FBS and 0.01% sodium azide) and incubated for 30 min, light-protected, on ice with 2 μg/ml of the labeled antibodies. After incubation, cells were washed again with FACS buffer and resuspended in FACS buffer supplemented with DAPI. Cells were analyzed on a LSRFortessa™ (BD) flow cytometer; the data were analyzed using FlowJo™ v9 software.

TCR mimic antibodies Clones 1 and 11 bound to various hematological cancer cell lines at 2 μg/ml as determined by direct staining. See FIGS. 11A-11G. Population shifts were observed for AML14, BV173, Nalm6 and MAC2A cell lines (cell lines of very high antigen density) for clone 1, but not for clone 11. All antigen-positive cell lines were killed successfully. IgG binding to a few cell lines allowed an estimate of target complexes on a per cell level using quantitation beads. There is an estimated 503 sites/cell on MAC2A cells, 876 sites/cell on AML14 cells, and 1,356 sites on BV173 cells. These data illustrated high sensitivity of the NDC80-clone 1 CAR T cells towards multiple cancer cell lines independent of their cancer type.

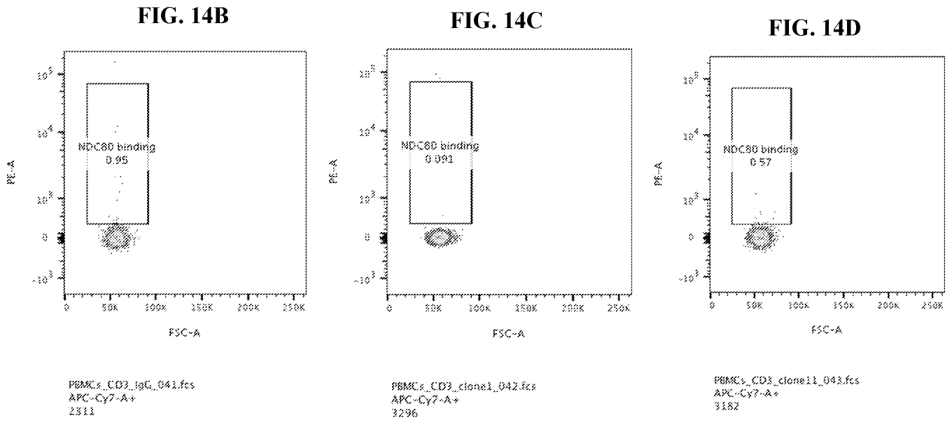

No binding was observed in adherent cell lines with either clone 1 or 11 in the TCR mimic antibody format. See FIGS. 12A-12C. Further, as shown in FIGS. 14A-14D, no binding was observed in mononuclear blood cells (i.e., T cells (CD3), Monocytes (CD14), B cells (CD19), Myeloid cells (CD33) or NK cells (CD56)) with either clone 1 or 11 in the TCR mimic antibody format using flow cytometry.

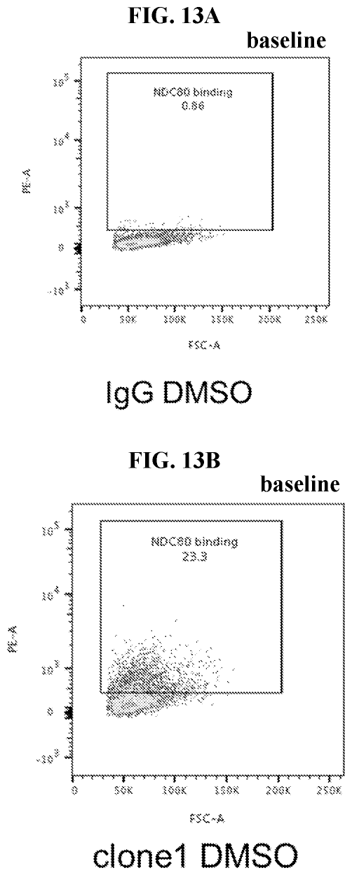

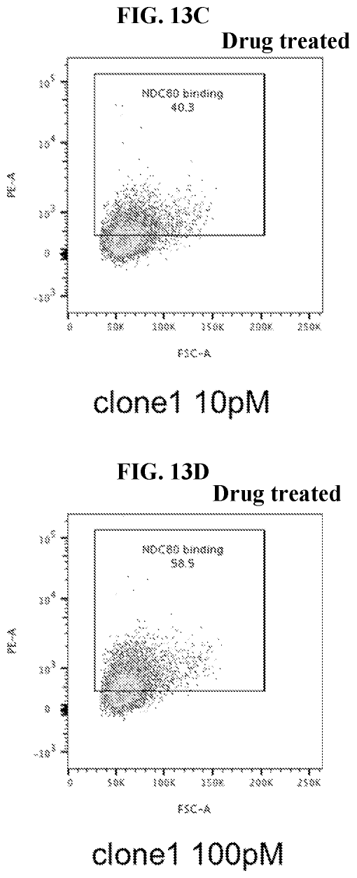

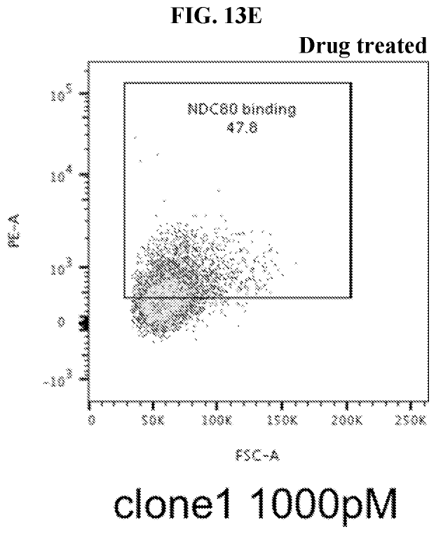

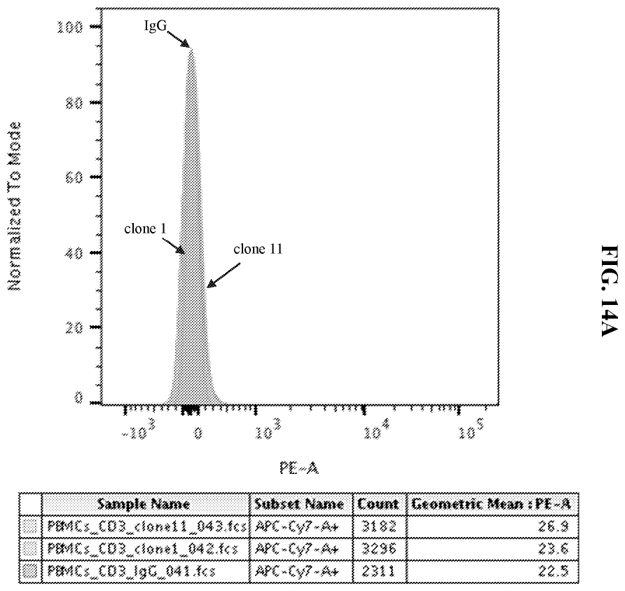

FIGS. 13A-13E show that binding activity of TCR mimic antibodies of the present technology can be enhanced through pre-treatment of cancer cells with docetaxel, a microtubule-stabilizing drug which causes cell cycle arrest in S phase, resulting in a higher presentation of the target Peptide A in HLA complexes.

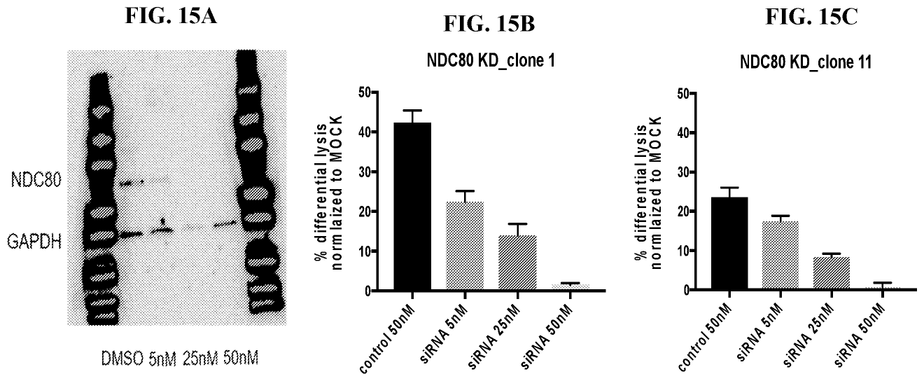

Target sequence specificity was also demonstrated by NDC80 knock down experiments. Unfortunately, as NDC80 plays an essential role in chromosome segregation and cell division stable knockout cell lines cannot be maintained. Accordingly, siRNA-based knock down experiments were used to target NDC80. JMN mesothelioma cells were treated with NDC80 siRNA (Thermo Fisher, Waltham, MA, Cat. #4392420) over 48 hours to knockdown expression of the NDC80 protein and reduce the presentation of the HLA ligand on the cell surface. Knockdown of NDC80 protein was confirmed by Western blotting. See FIG. 15A. CAR T cell killing experiments with Clones 1 and 11 demonstrated that NDC80 knockdown by siRNA abrogates CAR T cell killing in a dose-dependent manner. See FIGS. 15B-15C. Importantly, based on the JMN cell mass-spectrometry experiments, it is evident that that peptides with potential for off-target binding by NDC80-clone 1 CAR T cells (ALNEKLVNL (SEQ ID NO: 166), ALNELLQHV (SEQ ID NO: 167), ALNEEAGRLLL (SEQ ID NO: 175), YLDEYIARM (SEQ ID NO: 176) and LLFEGIARI (SEQ ID NO: 177)) were also presented at the cell surface of these target cells, but did not mediate killing after knockdown of NDC80.

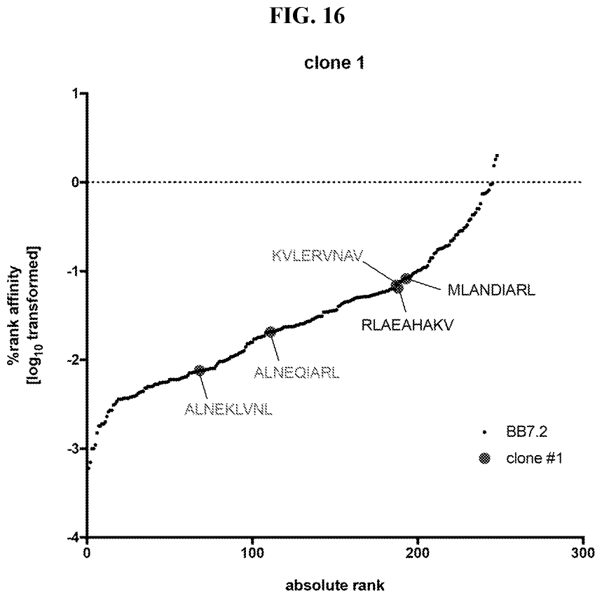

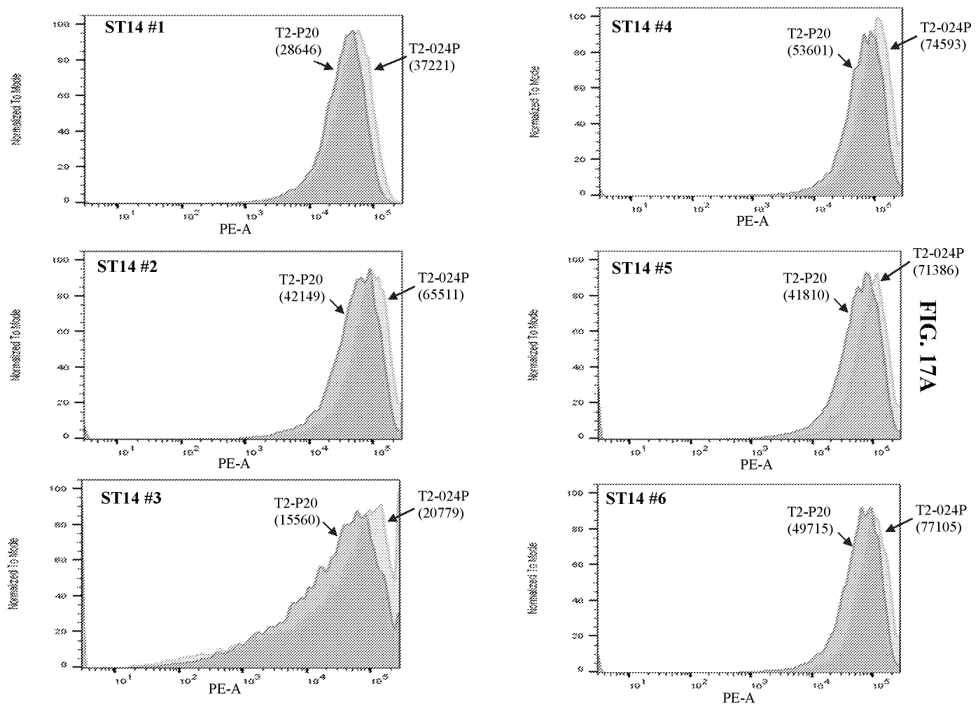

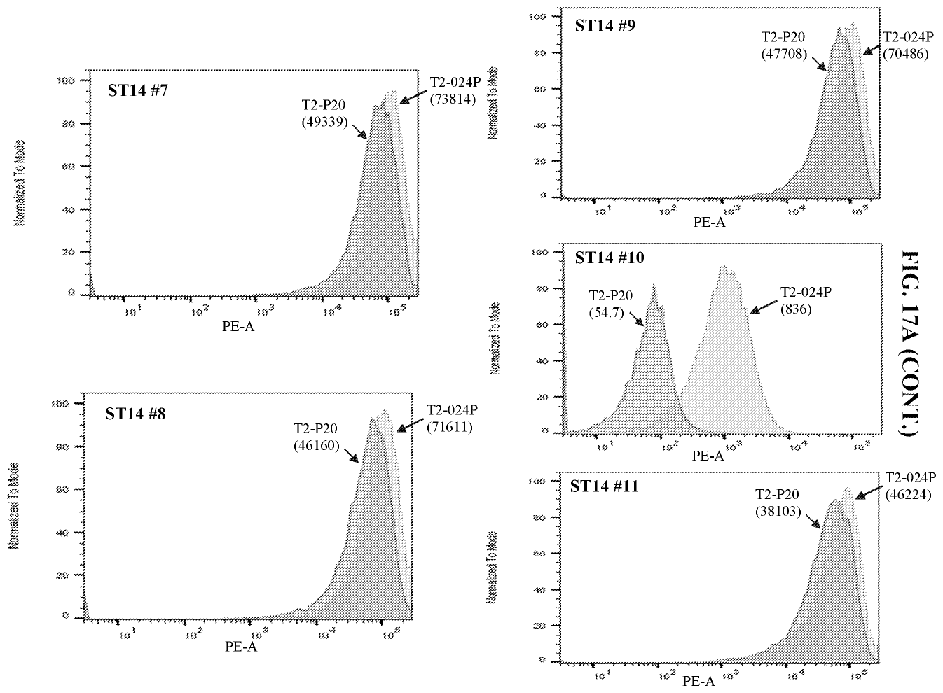

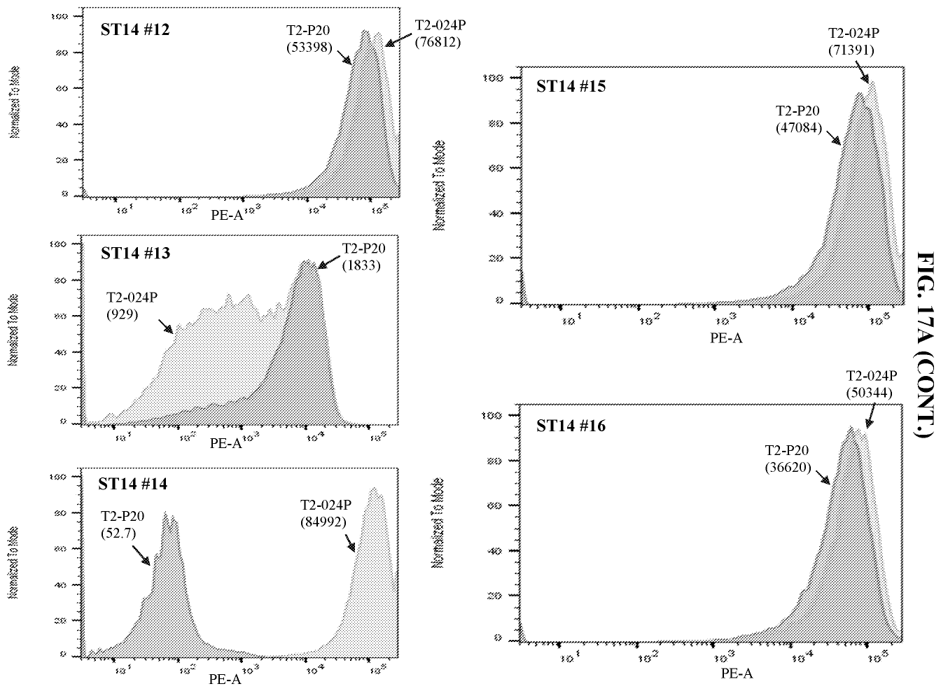

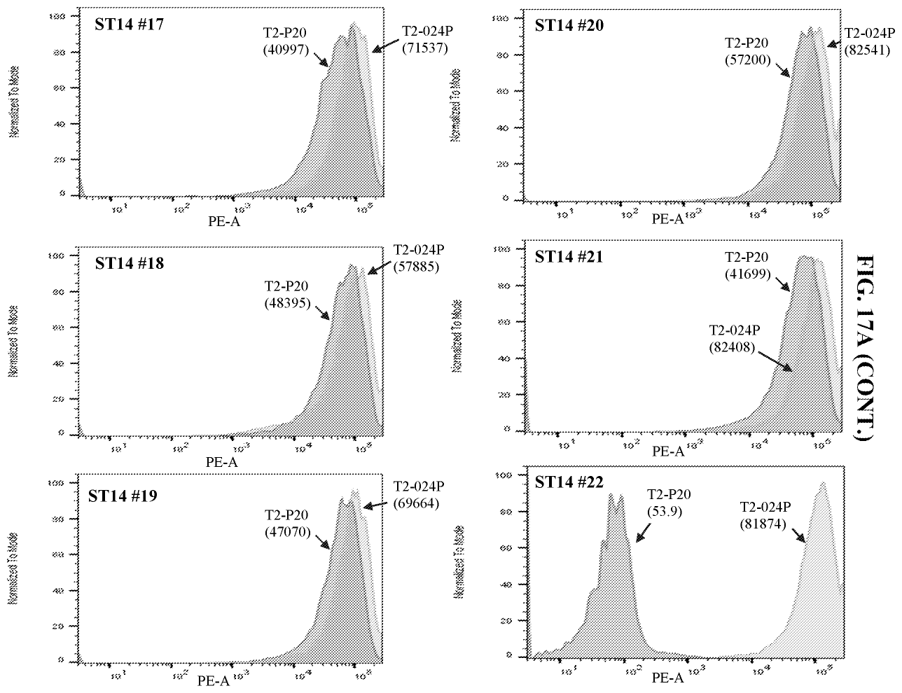

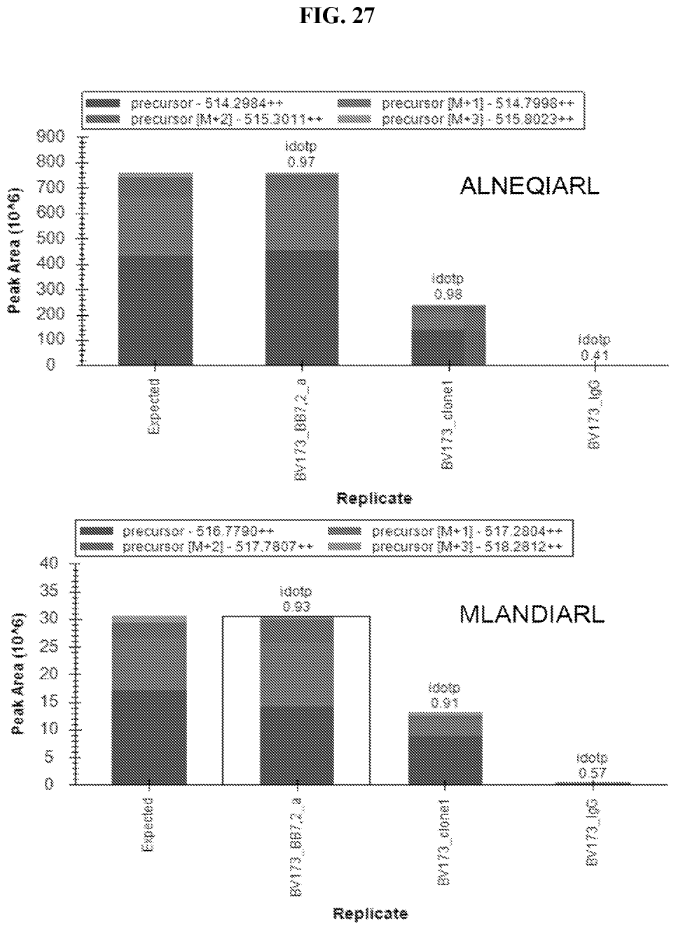

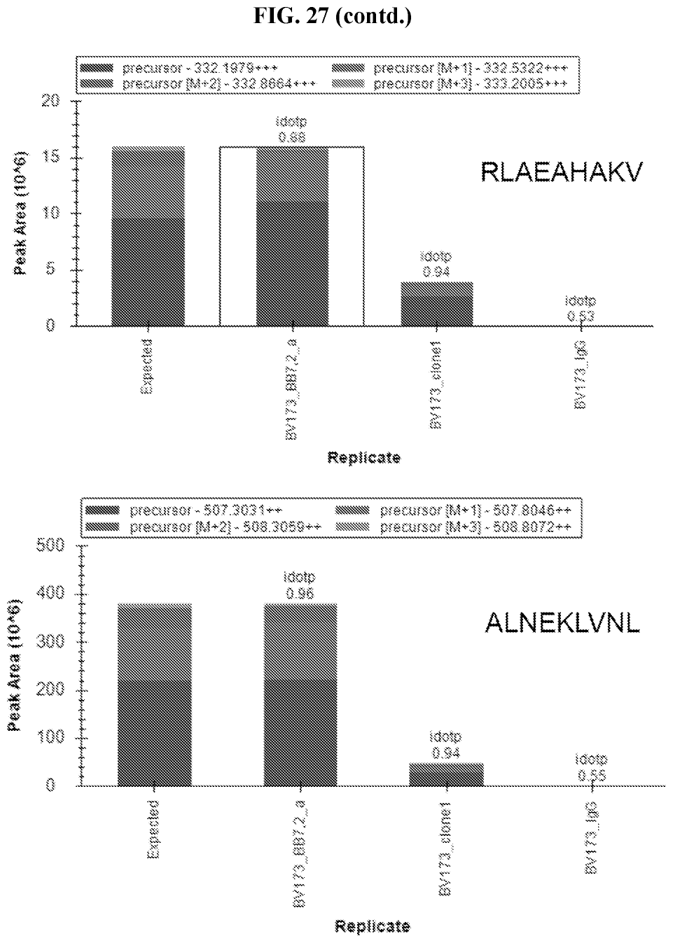

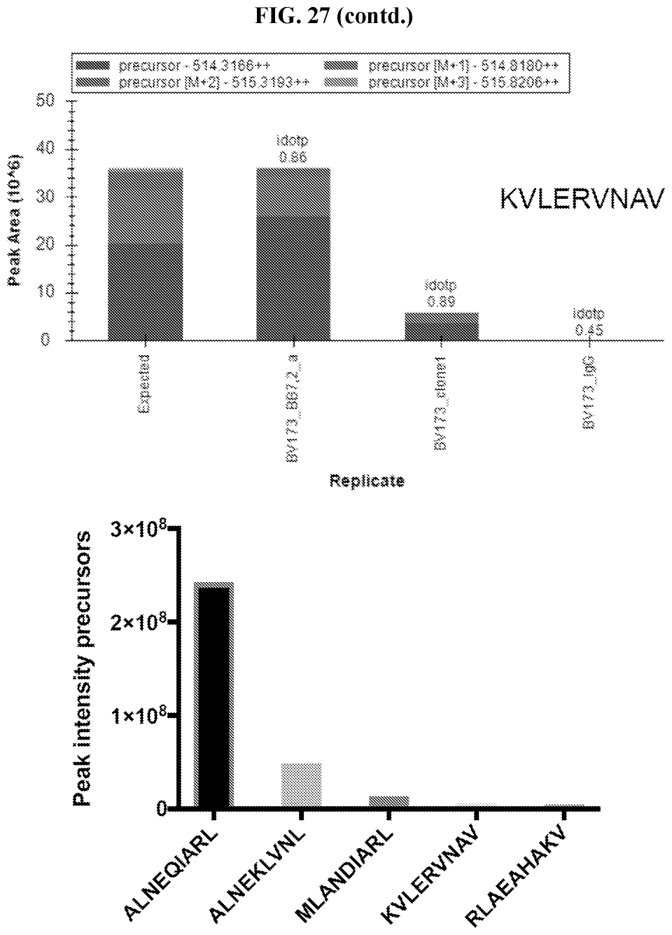

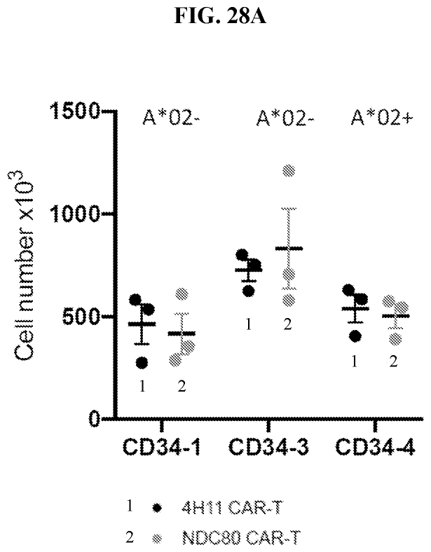

Mass spectrometry pulldown experiments were performed with TCR mimic antibodies clones 1 and 11 as described above. 200M BV173 cells were lysed with 1% CHAPS, incubated for 1h in the cold room and spun down at 20,000 g for 50 min. The lysate was split into 4 parts for IgG, BB7.2, clone 1 and clone 11 TCR mimic antibody pulldowns. For BB7.2 antibody 0.5 mg was used to provide sufficient background information of HLA-A*02 binders. For clone 1, clone 11 and IgG samples 30 μg were used. Clone 11 did not identify any peptides. IgG pulldown pulled 2 peptides that were also found with clone 1, which were filtered out as these were unspecific binders. As shown in FIG. 16, black dots represent NDC80 peptides identified by the A02-specific antibody BB7.2 (theoretically all the A02 ligands on the surface of BV173 cells). The indicated 5 sequences and red dots represent peptides identified in the clone 1 pulldown including the target sequence ALNEQIARL (SEQ ID NO: 1), but also potential off-targets such as ALNEKLVNL (SEQ ID NO: 166), KVLERVNAV (SEQ ID NO: 173), RLAEAHAKV (SEQ ID NO: 174), and MLANDIARL (SEQ ID NO: 168). Two of these potential proteomic off-targets, i.e., ALNEKLVNL (SEQ ID NO: 166) and MLANDIARL (SEQ ID NO: 168), were used in the original counter-screening for the phage library display. However, the risk of these 4 potential off-targets being functionally relevant seems limited as the signal intensity for the off-targets compared to ALNEQIARL (SEQ ID NO: 1) was at least 3-fold lower in the MS experiment (FIG. 27). After pulsing T2 cells with these 5 peptides, i.e., ALNEQIARL (SEQ ID NO: 1) plus 4 off-targets (SEQ ID NOs: 166, 168, 173, and 174), and the other NDC80 derived epitopes, i.e., GLNEEIARV (SEQ ID NO: 171) and HLEEQIAKV (SEQ ID NO: 172), as internal controls, binding was only observed for T2 cells pulsed with the ALNEQIARL (SEQ ID NO: 1) peptide (FIG. 9B), which further confirms the high specificity of the TCR mimic antibody of the present technology toward ALNEQIARL (SEQ ID NO: 1) observed in the previous experiment (FIGS. 10A-10I).

These results demonstrate that the immunoglobulin-related compositions of the present technology specifically kill multiple cancer types.

### Example 7: NDC80-clone 1 CAR T Cells and the TCR Mimic Antibodies of the Present Technology are Non-Toxic to Healthy Leukocytes and Hematopoietic Stem Cells

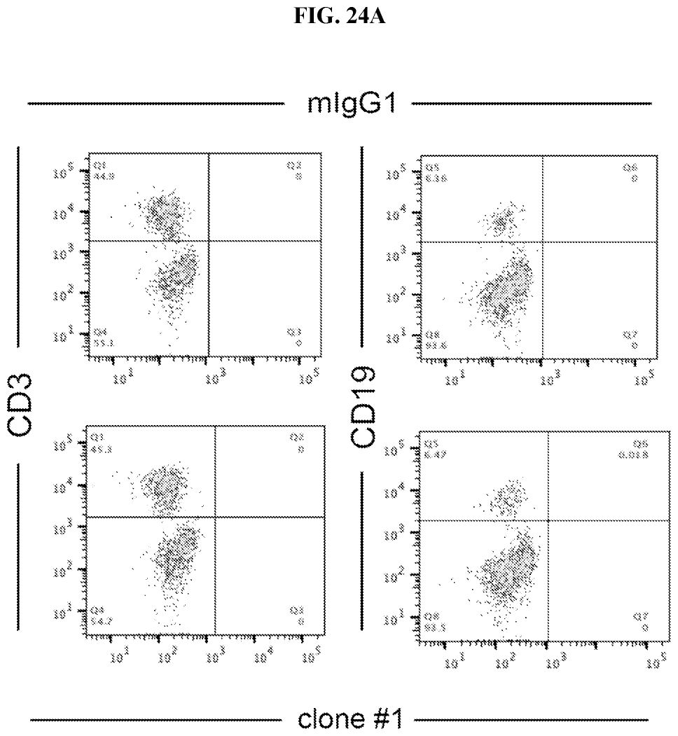

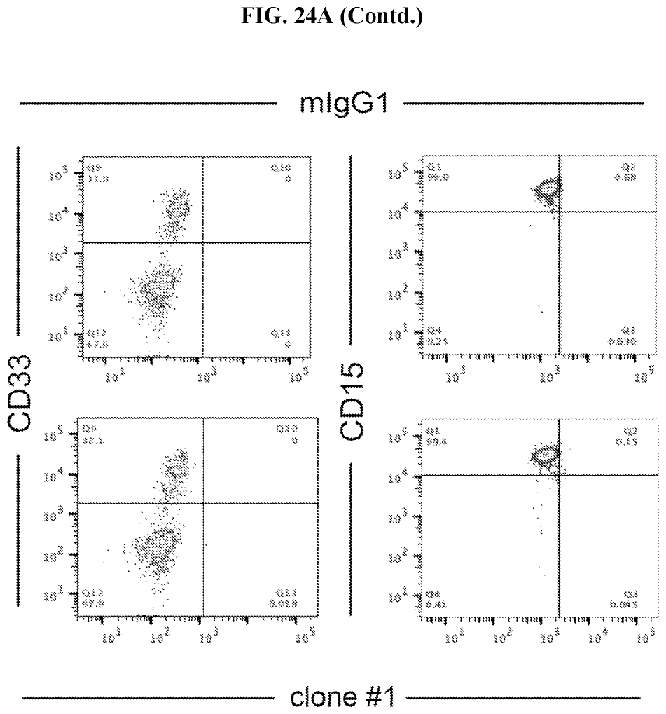

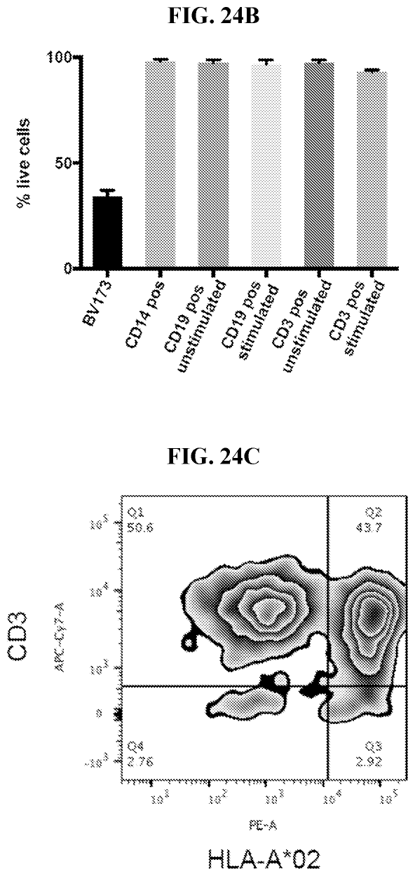

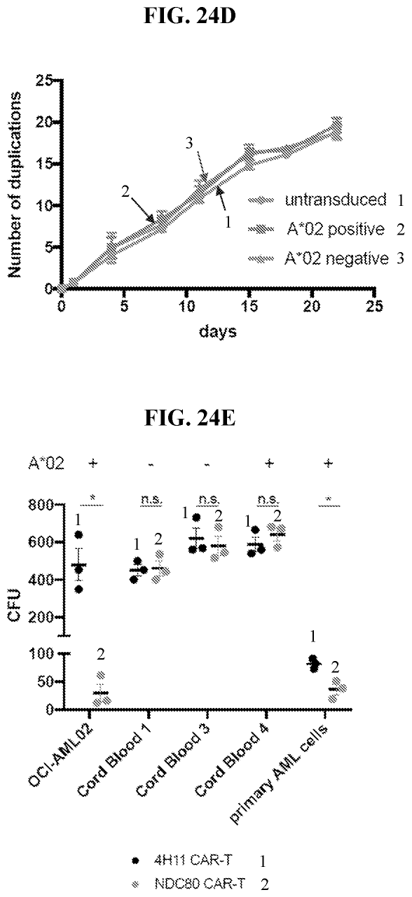

Next, additional remaining risks for on-target off-tumor toxicity to normal leukocytes and hematopoietic cells were evaluated. First, as leukocytes express the highest levels of HLA class I within the different cell types in the body (Boegel, S., et al., BMC Med Genomics 11: 36 (2018)), if these cells would stain positive with the mIgG1 clone 1 antibody was tested. For all tested subsets, T cells (CD3+), B cells (CD19+), myelomonocytic cells (CD33+ and CD15+), no positive staining over IgG background was observed (FIG. 24A). In addition, the potential of the NDC80-clone 1 CAR T cell to kill healthy A*02 positive PBMCs was investigated. Mitogen-stimulated T and B cells were also included in this assay as their increased proliferation could lead to greater expression of NDC80 as well as processing and presentation of the ALNEQIARL (SEQ ID NO: 1) HLA ligand. Little to no killing of sorted hematopoietic cells was observed (FIG. 24B).

As recognition and killing of activated T cells could strongly impact the production and efficacy of A*02 positive NDC80 specific CAR T cells, these NDC80-clone 1 CAR T cells were tested for their potential to mitigate fratricide. In an overnight 1:1 mixed lymphocyte culture of A*02 positive and A*02 negative NDC80 NDC80-clone 1 CAR T cells, only a slight reduction of A*02 positive CAR T cells was observed relative to the A02 negative cells (FIG. 24C). To address the potential long-term fratricide effects of HLA-A*02 positive NDC80 clone 1 CAR T cells, HLA-A*02 positive and HLA-A*02 negative CAR T cells were cultivated over 3 weeks, and their viability as well as cell numbers were monitored. Again, no signs of significant difference in cell proliferation or viability were observed between these two groups indicating no relevant fratricidal effects (FIG. 24D).

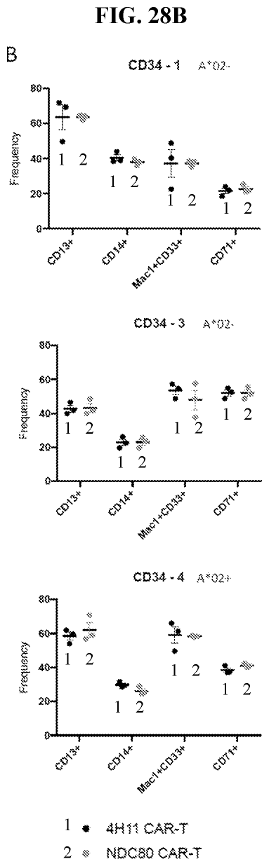

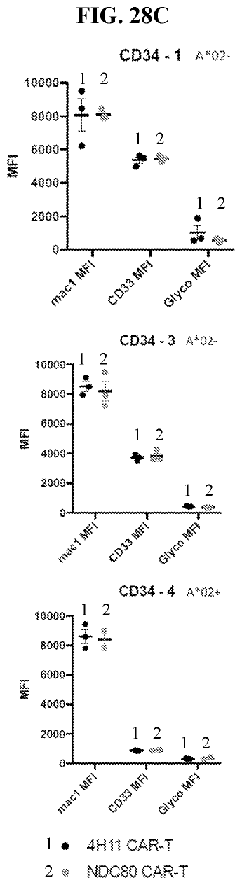

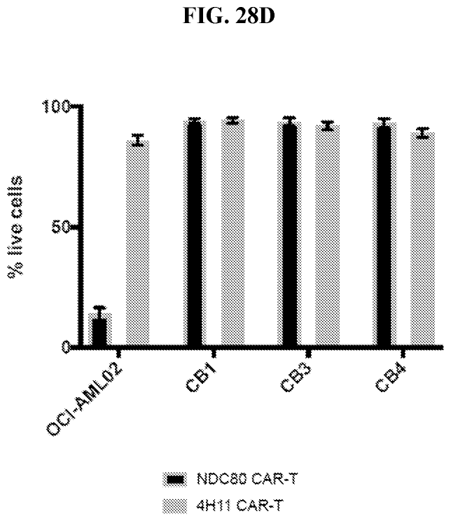

Although no toxicities were seen in our experiments with mature hematopoietic cells, the question remained if other important proliferative cells like hematopoetic stem cells would be affected by the NDC80-clone 1 CAR T cells. This is especially important as the MS ligandome results from healthy donors have shown positivity of a bone marrow sample for ALNEQIARL (SEQ ID NO: 1) (Marcu, A., et al., BioRxiv, doi: https://doi.org/10.1101/778944 (2019)). Therefore, colony forming unit (CFU) assays were used for cord blood-isolated CD34 positive HSCs (HLA-A*02 positive or negative), with OCI-AML02 cancer cells as positive control and one primary HLA-A*02 AML sample as a more physiological positive control. OCI-AML02 cancer cells as well as the primary AML cells formed significantly fewer colonies when treated with the NDC80-clone 1 CAR T cells as compared to a control CAR T cell treatment (FIG. 24E). In contrast, no impact on the ability to form colonies was detected in cord blood isolated HSCs independent of their HLA-A*02 status. Furthermore, cell numbers as well as lineage development were likewise not affected by the NDC80-clone 1 CAR T cells as determined by flow cytometry (FIGS. 28A-28C). The results from colony forming unit assays were corroborated using LDH assays, which showed no toxicity for either HLA-A*02 positive or negative cord blood derived HSCs (FIG. 28D). These data demonstrate that NDC80-clone 1 CAR T cells of the present technology do not cause toxicity towards the CAR T cells themselves, healthy leukocytes or hematopoietic stem cells.

Normal human cardiomyocytes (HCM), cardiac fibroblasts (HCF) and thymic fibroblasts (HTyF) were defrosted and cultured overnight in the medium provided by the vendor. Cells were subsequently detached using trypsin and washed twice before flow cytometric analysis. Cells were blocked with FcR blocker for 30 minutes (20% blocker) and were then stained with the mouse version of NDC80 clone 1 mAb, mouse isotype at 3 μg/ml, or BB7.2 clone (anti-HLA-A2). The AML-14 leukemia cell line was used as a positive control. BB7 mAb was used to determine HLA-A2 expression on targets. As shown in FIG. 30, HCF and HTyF are HLA-A2 positive, whereas HCM cells are HLA-A2 negative. However, none of the normal HCF, HCM and HTyF cells were positive for NDC80 clone 1 mAb staining.

These data demonstrate that NDC80-clone 1 antibodies of the present technology do not recognize and thus do not cause toxicity to healthy cardiomyocytes, cardiac fibroblasts or thymic fibroblasts.

### Example 8: NDC80-clone 1 CAR T Cells of the Present Technology Control Human Leukemia and Mesothelioma Tumor Growth in Mouse Models Leading to Prolonged Survival

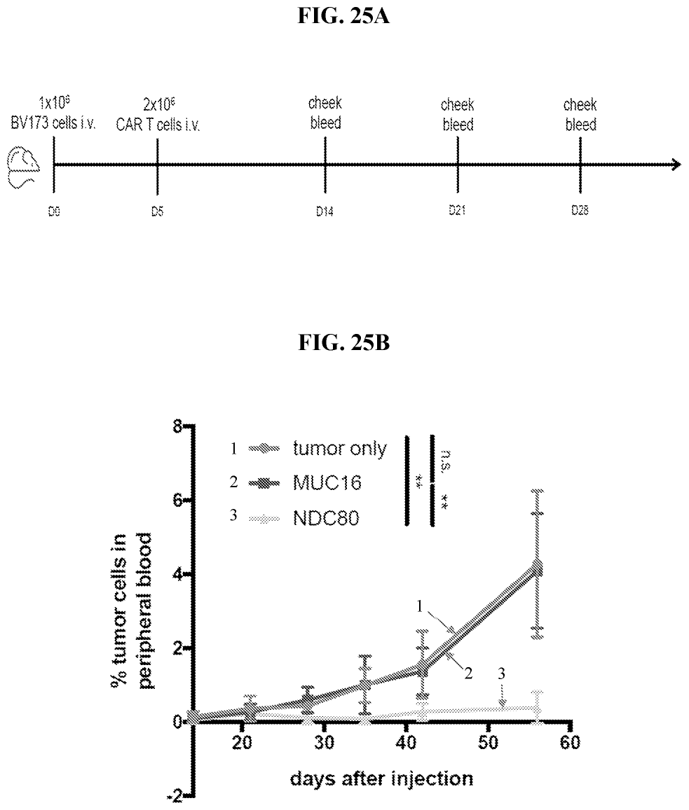

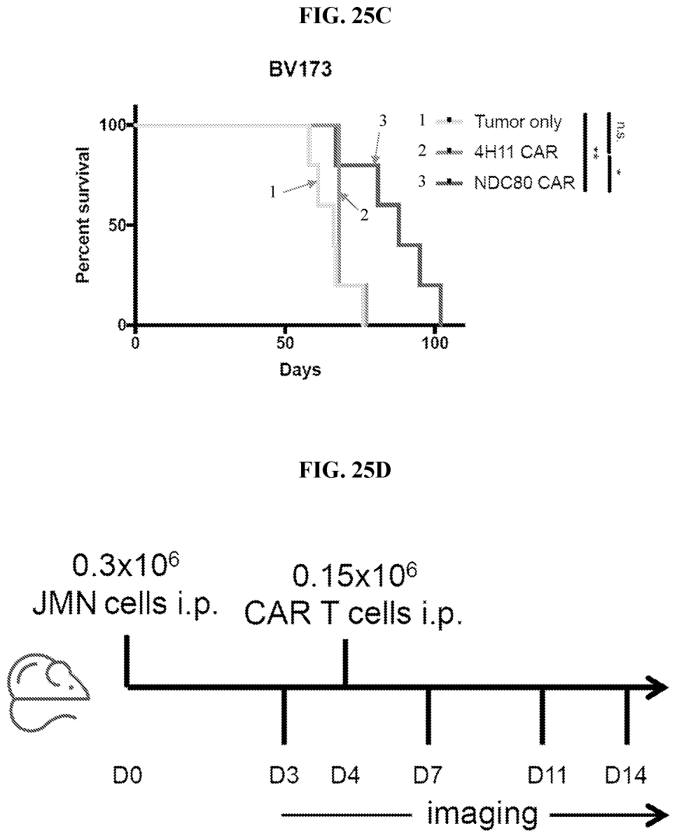

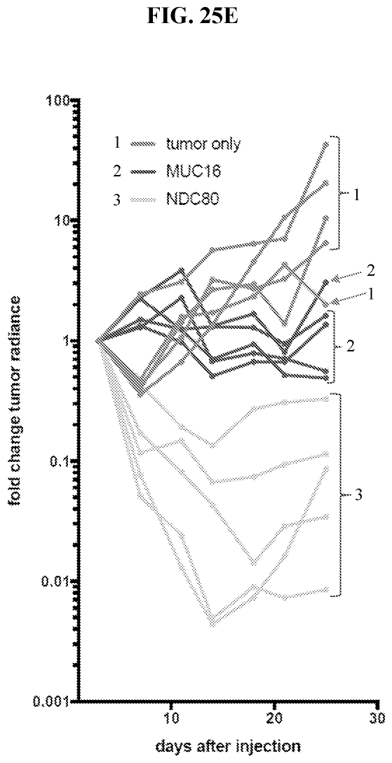

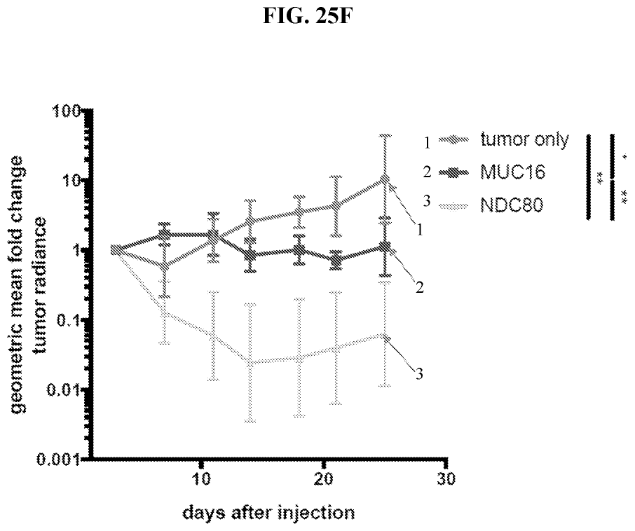

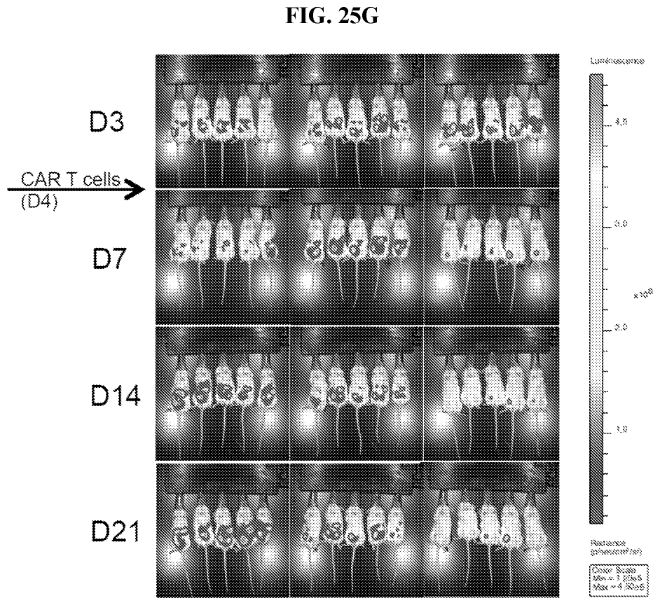

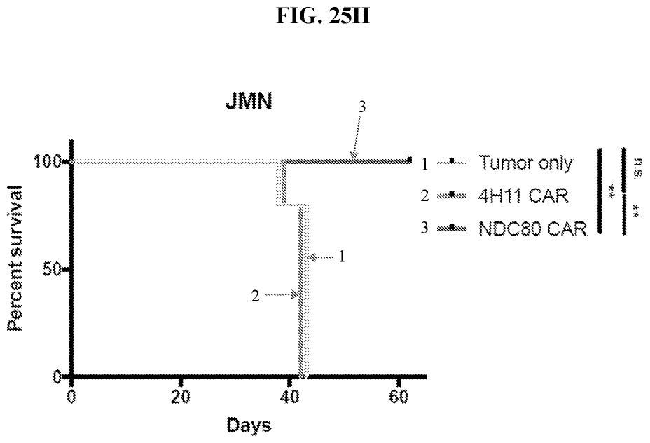

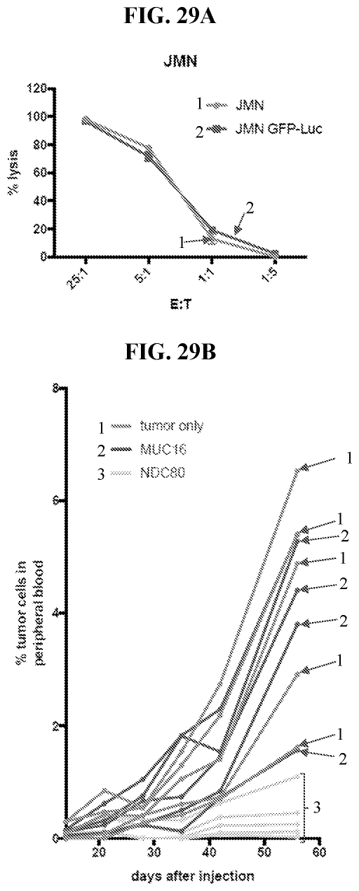

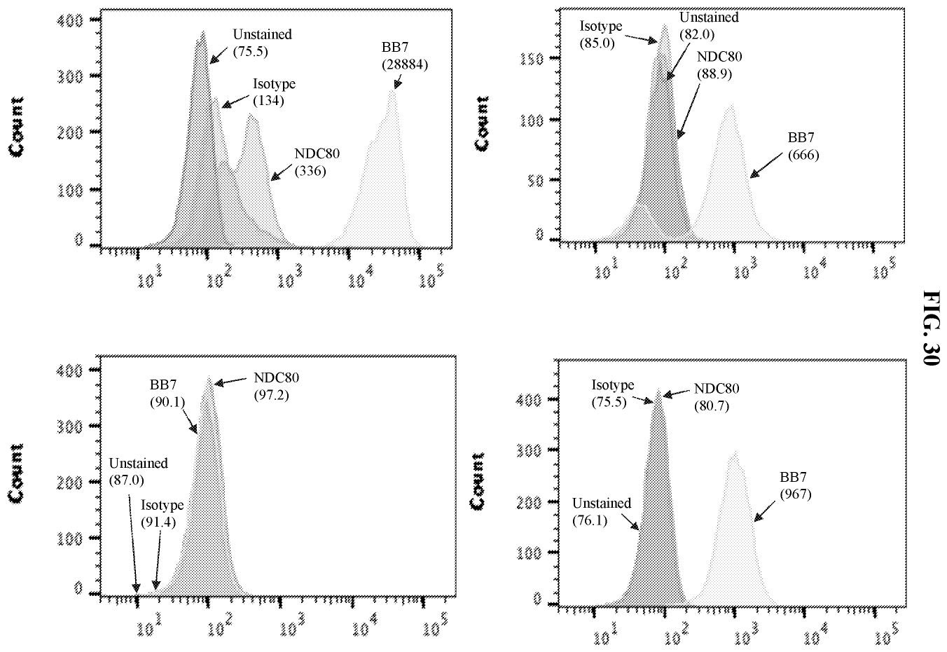

The antitumor potency of the NDC80-clone 1 CAR T cells of the present technology were tested in two mouse models: an intravenous BV173 leukemia model and an intraperitoneal JMN mesothelioma solid tumor model. One million BV173 cells per mouse were injected i.v. followed by injection of 2 million CAR T cells per mouse on day 5; disease burden was monitored through weekly cheek bleeds and subsequently by flow cytometry to determine the fraction of leukemia cells versus CD45 positive mouse leukocytes as described in Gopalakrishnapillai, A., et al., Front Oncol 6: 162 (2016), which is incorporated by reference herein in its entirety (FIG. 25A). MUC16 specific CAR T cells as well as tumor cell injection alone served as controls. Significant reduction of peripheral blood tumor cell count in the group treated with the NDC80-clone 1 CAR T cells was observed over controls (FIG. 25B and FIG. 29A) over the 56 days monitoring. Followed-up observation of overall survival also showed superiority of the NDC80-clone 1 CAR over the control groups (FIG. 25C).

Untransduced JMN cells and luciferase-expressing JMN cells were used in the i.p. solid tumor model experiment. Notably, luciferase transduction of JMN cells did not affect killing by NDC80-c11 CAR T cells compared to untransduced JMN cells in vitro (FIG. 29B). 0.3 million JMN cells were injected i.p., imaged on day 3, followed by the injection of 0.15 million NDC80-clone 1 CAR T cells or MUC16-specific CAR T cells on day 4 (FIG. 25D). Bioluminescence imaging showed profound tumor control with a 10-200-fold decrease in tumor signal in individual NDC80-clone 1 CAR T cell treated mice and an average 10-fold decrease in tumor signal from day 14 onwards in the entire group (FIGS. 25E-25G). This significant tumor control also translated into a survival benefit with 100% survival of mice to day 60; in contrast, no survivors remained in the control CAR T treated group after 42 days (FIG. 25H).

These data demonstrate that NDC80-clone 1 CAR T cells of the present technology are useful in treating NDC80-associated diseases including but not limited to cancers.

### Example 9: Characterization of Functional Activity of the TCR Mimic Antibodies of the Present Technology

Several reports have demonstrated that NDC80 Peptide A (SEQ ID NO: 1) can be presented on several HLA-A2 subtypes including HLA-A*02:02, A*02:03, A*02:04, A*02:07 and A*02:11 subtypes. Abelin et al. Immunity 46(2): 315-326 (2017); Sarkizova et al., Nature Biotechnology 38:199-209(2020). Since other TCR mimic antibodies are known to bind different HLA-A*02 subtypes with similar high affinity (see Ataie et al., J Mol Biol. 428(1):194-205 (2016)), the affinity of the anti-NDC80/MHC TCR mimic antibodies described herein towards different HLA-A*02 subtypes will also be assessed.

HLA subtype construction, purification, and analysis. HLA-A*02:01, 02:02, 02:03, 02:05, 02:06, 02:07, 02:11 sequences will be obtained from The HLA Factsbook (Marsh S G E, Parham P, Barber L D. The HLA Facts Book. Academic Press; 1999). HLA subtype MHC-peptide complexes are produced following standard protocols based on refolding of E. coli inclusion bodies (Altman J D, Davis M M. Curr Protoc Immunol. John Wiley & Sons, Inc; 2001). In brief, each HLA-A*02 subunit as well as the β2-microglubulin (B2M) subunit are overexpressed in E. coli in inclusion bodies and then solubilized in 8M urea. NDC80 Peptide A (SEQ ID NO: 1), HLA-A*02 solution, and β2M urea solutions are mixed together in the refolding buffer at 4° C. for 2 days. The refolding solution is further concentrated and buffer exchanged into PBS. The MHC complex is purified by size exclusion chromatography (SEC) on a GE Healthcare AKTA™ FPLC system.

HLA subtype binding assays. Binding affinity is determined by measuring surface plasmon resonance on a Biacore™ X100 (GE Healthcare). 50 μg/mL of modified streptavidin is immobilized onto a Sensor Chip CAP by applying Biotin CAPture Reagent (GE Healthcare Cat. #28-9202-33) through the flow cells at 2 μL/min for 5 minutes. 10 μg/mL biotinylated MHC complex carrying the ALNEQIARL (SEQ ID NO: 1) peptide is loaded onto the flow cell at a rate of 30 μL/min for 3 minutes. Following the standard protocol for single-cycle kinetics, a series of injections of NDC80 are performed at 0.313, 0.625, 1.25, 2.5, and 5 μg/mL, with each step consisting of a 3 minute injection at 30 μL/min and a 3 minute disassociation. Afterwards, the surface is regenerated for 2 minutes with a solution consisting of 75% v/v 8M guanidine-HCl and 25% v/v 1M NaOH. Kinetic constants are derived by global fitting to a 1:1 Langmuir binding model using the BIAcore™ X100 Evaluation Software v. 2.0.1.

It is anticipated that the anti-NDC80/MHC TCR mimic antibodies of the present technology will bind different HLA-A*02 subtypes with comparable high affinity.

### Exemplary Embodiments

The present disclosure may be described in terms of the following non-limiting embodiments:

Embodiment 1: A composition that comprises an antibody moiety comprising: (a) a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 2, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 20; or (b) a V_{H} comprising a V_{H}-CDR1 sequence, a V_{H}-CDR2 sequence, and a V_{H}-CDR3 sequence of the V_{H} sequence of SEQ ID NO: 3, and a V_{L} comprising a V_{L}-CDR1 sequence, a V_{L}-CDR2 sequence, and a V_{L}-CDR3 sequence of the V_{L} sequence SEQ ID NO: 21; or (c) a V_{H} comprising a V_{H}-CDR1 sequence, a V_{H}-CDR2 sequence, and a V_{H}-CDR3 sequence of the V_{H} sequence of SEQ ID NO: 4, and a V_{L} comprising a V_{L}-CDR1 sequence, a V_{L}-CDR2 sequence, and a V_{L}-CDR3 sequence of the V_{L} sequence SEQ ID NO: 22; or (d) a V_{H} comprising a V_{H}-CDR1 sequence, a V_{H}-CDR2 sequence, and a V_{H}-CDR3 sequence of the V_{H} sequence of SEQ ID NO: 5, and a V_{L} comprising a V_{L}-CDR1 sequence, a V_{L}-CDR2 sequence, and a V_{L}-CDR3 sequence of the V_{L} sequence SEQ ID NO: 23; or (e) a V_{H} comprising a V_{H}-CDR1 sequence, a V_{H}-CDR2 sequence, and a V_{H}-CDR3 sequence of the V_{H} sequence of SEQ ID NO: 6, and a V_{L} comprising a V_{L}-CDR1 sequence, a V_{L}-CDR2 sequence, and a V_{L}-CDR3 sequence of the V_{L} sequence SEQ ID NO: 24; or (f) a V_{H} comprising a V_{H}-CDR1 sequence, a V_{H}-CDR2 sequence, and a V_{H}-CDR3 sequence of the V_{H} sequence of SEQ ID NO: 7, and a V_{L} comprising a V_{L}-CDR1 sequence, a V_{L}-CDR2 sequence, and a V_{L}-CDR3 sequence of the V_{L} sequence SEQ ID NO: 25; or (g) a V_{H} comprising a V_{H}-CDR1 sequence, a V_{H}-CDR2 sequence, and a V_{H}-CDR3 sequence of the V_{H} sequence of SEQ ID NO: 8, and a V_{L} comprising a V_{L}-CDR1 sequence, a V_{L}-CDR2 sequence, and a V_{L}-CDR3 sequence of the V_{L} sequence SEQ ID NO: 26; or (h) a V_{H} comprising a V_{H}-CDR1 sequence, a V_{H}-CDR2 sequence, and a V_{H}-CDR3 sequence of the V_{H} sequence of SEQ ID NO: 9, and a V_{L} comprising a V_{L}-CDR1 sequence, a V_{L}-CDR2 sequence, and a V_{L}-CDR3 sequence of the V_{L} sequence SEQ ID NO: 27; or (i) a V_{H} comprising a V_{H}-CDR1 sequence, a V_{H}-CDR2 sequence, and a V_{H}-CDR3 sequence of the V_{H} sequence of SEQ ID NO: 10, and a V_{L} comprising a V_{L}-CDR1 sequence, a V_{L}-CDR2 sequence, and a V_{L}-CDR3 sequence of the V_{L} sequence SEQ ID NO: 28; or (j) a V_{H} comprising a V_{H}-CDR1 sequence, a V_{H}-CDR2 sequence, and a V_{H}-CDR3 sequence of the V_{H} sequence of SEQ ID NO: 11, and a V_{L} comprising a V_{L}-CDR1 sequence, a V_{L}-CDR2 sequence, and a V_{L}-CDR3 sequence of the V_{L} sequence SEQ ID NO: 29; or (k) a V_{H} comprising a V_{H}-CDR1 sequence, a V_{H}-CDR2 sequence, and a V_{H}-CDR3 sequence of the V_{H} sequence of SEQ ID NO: 12, and a V_{L} comprising a V_{L}-CDR1 sequence, a V_{L}-CDR2 sequence, and a V_{L}-CDR3 sequence of the V_{L} sequence SEQ ID NO: 30; or (1) a V_{H} comprising a V_{H}-CDR1 sequence, a V_{H}-CDR2 sequence, and a V_{H}-CDR3 sequence of the V_{H} sequence of SEQ ID NO: 13, and a V_{L} comprising a V_{L}-CDR1 sequence, a V_{L}-CDR2 sequence, and a V_{L}-CDR3 sequence of the V_{L} sequence SEQ ID NO: 31; or (m) a V_{H} comprising a V_{H}-CDR1 sequence, a V_{H}-CDR2 sequence, and a V_{H}-CDR3 sequence of the V_{H} sequence of SEQ ID NO: 14, and a V_{L} comprising a V_{L}-CDR1 sequence, a V_{L}-CDR2 sequence, and a V_{L}-CDR3 sequence of the V_{L} sequence SEQ ID NO: 32; or (n) a V_{H} comprising a V_{H}-CDR1 sequence, a V_{H}-CDR2 sequence, and a V_{H}-CDR3 sequence of the V_{H} sequence of SEQ ID NO: 15, and a V_{L} comprising a V_{L}-CDR1 sequence, a V_{L}-CDR2 sequence, and a V_{L}-CDR3 sequence of the V_{L} sequence SEQ ID NO: 33; or (o) a V_{H} comprising a V_{H}-CDR1 sequence, a V_{H}-CDR2 sequence, and a V_{H}-CDR3 sequence of the V_{H} sequence of SEQ ID NO: 16, and a V_{L} comprising a V_{L}-CDR1 sequence, a V_{L}-CDR2 sequence, and a V_{L}-CDR3 sequence of the V_{L} sequence SEQ ID NO: 34; or (p) a V_{H} comprising a V_{H}-CDR1 sequence, a V_{H}-CDR2 sequence, and a V_{H}-CDR3 sequence of the V_{H} sequence of SEQ ID NO: 17, and a V_{L} comprising a V_{L}-CDR1 sequence, a V_{L}-CDR2 sequence, and a V_{L}-CDR3 sequence of the V_{L} sequence SEQ ID NO: 35; or (q) a V_{H} comprising a V_{H}-CDR1 sequence, a V_{H}-CDR2 sequence, and a V_{H}-CDR3 sequence of the V_{H} sequence of SEQ ID NO: 18, and a V_{L} comprising a V_{L}-CDR1 sequence, a V_{L}-CDR2 sequence, and a V_{L}-CDR3 sequence of the V_{L} sequence SEQ ID NO: 36, or (r) a V_{H} comprising a V_{H}-CDR1 sequence, a V_{H}-CDR2 sequence, and a V_{H}-CDR3 sequence of the V_{H} sequence of SEQ ID NO: 19, and a V_{L} comprising a V_{L}-CDR1 sequence, a V_{L}-CDR2 sequence, and a V_{L}-CDR3 sequence of the V_{L} sequence SEQ ID NO: 37.

Embodiment 2: The composition of Embodiment 1, wherein (a) the V_{H}-CDR1 sequence comprises the sequence of SEQ ID NO: 44, the V_{H}-CDR2 sequence comprises the sequence of SEQ ID NO: 45, the V_{H}-CDR3 sequence comprises the sequence of SEQ ID NO: 46, the V_{L}-CDR1 sequence comprises the sequence of SEQ ID NO: 98, the V_{L}-CDR2 sequence comprises the sequence of SEQ ID NO: 99, and/or the V_{L}-CDR3 sequence comprises the sequence of SEQ ID NO: 100; or (b) the V_{H}-CDR1 sequence comprises the sequence of SEQ ID NO: 47, the V_{H}-CDR2 sequence comprises the sequence of SEQ ID NO: 48, the V_{H}-CDR3 sequence comprises the sequence of SEQ ID NO: 49, the V_{L}-CDR1 sequence comprises the sequence of SEQ ID NO: 101, the V_{L}-CDR2 sequence comprises the sequence of SEQ ID NO: 102, and/or the V_{L}-CDR3 sequence comprises the sequence of SEQ ID NO: 103; or (c) the V_{H}-CDR1 sequence comprises the sequence of SEQ ID NO: 50, the V_{H}-CDR2 sequence comprises the sequence of SEQ ID NO: 51, the V_{H}-CDR3 sequence comprises the sequence of SEQ ID NO: 52, the V_{L}-CDR1 sequence comprises the sequence of SEQ ID NO: 104, the V_{L}-CDR2 sequence comprises the sequence of SEQ ID NO: 105, and/or the V_{L}-CDR3 sequence comprises the sequence of SEQ ID NO: 106; or (d) the V_{H}-CDR1 sequence comprises the sequence of SEQ ID NO: 53, the V_{H}-CDR2 sequence comprises the sequence of SEQ ID NO: 54, the V_{H}-CDR3 sequence comprises the sequence of SEQ ID NO: 55, the V_{L}-CDR1 sequence comprises the sequence of SEQ ID NO: 107, the V_{L}-CDR2 sequence comprises the sequence of SEQ ID NO: 108, and/or the V_{L}-CDR3 sequence comprises the sequence of SEQ ID NO: 109; or (e) the V_{H}-CDR1 sequence comprises the sequence of SEQ ID NO: 56, the V_{H}-CDR2 sequence comprises the sequence of SEQ ID NO: 57, the V_{H}-CDR3 sequence comprises the sequence of SEQ ID NO: 58, the V_{L}-CDR1 sequence comprises the sequence of SEQ ID NO: 110, the V_{L}-CDR2 sequence comprises the sequence of SEQ ID NO: 111, and/or the V_{L}-CDR3 sequence comprises the sequence of SEQ ID NO: 112; or (f) the V_{H}-CDR1 sequence comprises the sequence of SEQ ID NO: 59, the V_{H}-CDR2 sequence comprises the sequence of SEQ ID NO: 60, the V_{H}-CDR3 sequence comprises the sequence of SEQ ID NO: 61, the V_{L}-CDR1 sequence comprises the sequence of SEQ ID NO: 113, the V_{L}-CDR2 sequence comprises the sequence of SEQ ID NO: 114, and/or the V_{L}-CDR3 sequence comprises the sequence of SEQ ID NO: 115; or (g) the V_{H}-CDR1 sequence comprises the sequence of SEQ ID NO: 62, the V_{H}-CDR2 sequence comprises the sequence of SEQ ID NO: 63, the V_{H}-CDR3 sequence comprises the sequence of SEQ ID NO: 64, the V_{L}-CDR1 sequence comprises the sequence of SEQ ID NO: 116, the V_{L}-CDR2 sequence comprises the sequence of SEQ ID NO: 117, and/or the V_{L}-CDR3 sequence comprises the sequence of SEQ ID NO: 118; or (h) the V_{H}-CDR1 sequence comprises the sequence of SEQ ID NO: 65, the V_{H}-CDR2 sequence comprises the sequence of SEQ ID NO: 66, the V_{H}-CDR3 sequence comprises the sequence of SEQ ID NO: 67, the V_{L}-CDR1 sequence comprises the sequence of SEQ ID NO: 119, the V_{L}-CDR2 sequence comprises the sequence of SEQ ID NO: 120, and/or the V_{L}-CDR3 sequence comprises the sequence of SEQ ID NO: 121; or (i) the V_{H}-CDR1 sequence comprises the sequence of SEQ ID NO: 68, the V_{H}-CDR2 sequence comprises the sequence of SEQ ID NO: 69, the V_{H}-CDR3 sequence comprises the sequence of SEQ ID NO: 70, the V_{L}-CDR1 sequence comprises the sequence of SEQ ID NO: 122, the V_{L}-CDR2 sequence comprises the sequence of SEQ ID NO: 123, and/or the V_{L}-CDR3 sequence comprises the sequence of SEQ ID NO: 124; or (j) the V_{H}-CDR1 sequence comprises the sequence of SEQ ID NO: 71, the V_{H}-CDR2 sequence comprises the sequence of SEQ ID NO: 72, the V_{H}-CDR3 sequence comprises the sequence of SEQ ID NO: 73, the V_{L}-CDR1 sequence comprises the sequence of SEQ ID NO: 125, the V_{L}-CDR2 sequence comprises the sequence of SEQ ID NO: 126, and/or the V_{L}-CDR3 sequence comprises the sequence of SEQ ID NO: 127; or (k) the V_{H}-CDR1 sequence comprises the sequence of SEQ ID NO: 74, the V_{H}-CDR2 sequence comprises the sequence of SEQ ID NO: 75, the V_{H}-CDR3 sequence comprises the sequence of SEQ ID NO: 76, the V_{L}-CDR1 sequence comprises the sequence of SEQ ID NO: 128, the V_{L}-CDR2 sequence comprises the sequence of SEQ ID NO: 129, and/or the V_{L}-CDR3 sequence comprises the sequence of SEQ ID NO: 130; or (1) the V_{H}-CDR1 sequence comprises the sequence of SEQ ID NO: 77, the V_{H}-CDR2 sequence comprises the sequence of SEQ ID NO: 78, the V_{H}-CDR3 sequence comprises the sequence of SEQ ID NO: 79, the V_{L}-CDR1 sequence comprises the sequence of SEQ ID NO: 131, the V_{L}-CDR2 sequence comprises the sequence of SEQ ID NO: 132, and/or the V_{L}-CDR3 sequence comprises the sequence of SEQ ID NO: 133; or (m) the V_{H}-CDR1 sequence comprises the sequence of SEQ ID NO: 80, the V_{H}-CDR2 sequence comprises the sequence of SEQ ID NO: 81, the V_{H}-CDR3 sequence comprises the sequence of SEQ ID NO: 82, the V_{L}-CDR1 sequence comprises the sequence of SEQ ID NO: 134, the V_{L}-CDR2 sequence comprises the sequence of SEQ ID NO: 135, and/or the V_{L}-CDR3 sequence comprises the sequence of SEQ ID NO: 136; or (n) the V_{H}-CDR1 sequence comprises the sequence of SEQ ID NO: 83, the V_{H}-CDR2 sequence comprises the sequence of SEQ ID NO: 84, the V_{H}-CDR3 sequence comprises the sequence of SEQ ID NO: 85, the V_{L}-CDR1 sequence comprises the sequence of SEQ ID NO: 137, the V_{L}-CDR2 sequence comprises the sequence of SEQ ID NO: 138, and/or the V_{L}-CDR3 sequence comprises the sequence of SEQ ID NO: 139; or (o) the V_{H}-CDR1 sequence comprises the sequence of SEQ ID NO: 86, the V_{H}-CDR2 sequence comprises the sequence of SEQ ID NO: 87, the V_{H}-CDR3 sequence comprises the sequence of SEQ ID NO: 88, the V_{L}-CDR1 sequence comprises the sequence of SEQ ID NO: 140, the V_{L}-CDR2 sequence comprises the sequence of SEQ ID NO: 141, and/or the V_{L}-CDR3 sequence comprises the sequence of SEQ ID NO: 142; or (p) the V_{H}-CDR1 sequence comprises the sequence of SEQ ID NO: 89, the V_{H}-CDR2 sequence comprises the sequence of SEQ ID NO: 90, the V_{H}-CDR3 sequence comprises the sequence of SEQ ID NO: 91, the V_{L}-CDR1 sequence comprises the sequence of SEQ ID NO: 143, the V_{L}-CDR2 sequence comprises the sequence of SEQ ID NO: 144, and/or the V_{L}-CDR3 sequence comprises the sequence of SEQ ID NO: 145; or (q) the V_{H}-CDR1 sequence comprises the sequence of SEQ ID NO: 92, the V_{H}-CDR2 sequence comprises the sequence of SEQ ID NO: 93, the V_{H}-CDR3 sequence comprises the sequence of SEQ ID NO: 94, the V_{L}-CDR1 sequence comprises the sequence of SEQ ID NO: 146, the V_{L}-CDR2 sequence comprises the sequence of SEQ ID NO: 147, and/or the V_{L}-CDR3 sequence comprises the sequence of SEQ ID NO: 148; or (r) the V_{H}-CDR1 sequence comprises the sequence of SEQ ID NO: 95, the V_{H}-CDR2 sequence comprises the sequence of SEQ ID NO: 96, the V_{H}-CDR3 sequence comprises the sequence of SEQ ID NO: 97, the V_{L}-CDR1 sequence comprises the sequence of SEQ ID NO: 149, the V_{L}-CDR2 sequence comprises the sequence of SEQ ID NO: 150, and/or the V_{L}-CDR3 sequence comprises the sequence of SEQ ID NO: 151.

Embodiment 3: The composition of Embodiment 1 or 2, wherein (a) the V_{H} comprises an amino acid sequence having at least 90% identity to a sequence selected from the group consisting of: SEQ ID NOs: 2-19, and/or (b) the V_{L} comprises an amino acid sequence having at least 90% identity to a sequence selected from the group consisting of: SEQ ID NOs: 20-37.

Embodiment 4: The composition of any one of Embodiments 1-3, wherein (a) the V_{H} comprises an amino acid sequence selected from the group consisting of: SEQ ID NOs: 2-19 or a variant thereof having one or more conservative amino acid substitutions; and/or (b) the V_{L} comprises an amino acid sequence selected from the group consisting of: SEQ ID NOs: 20-37 or a variant thereof having one or more conservative amino acid substitutions.

Embodiment 5: The composition of any one of Embodiments 1-4, wherein the V_{H} amino acid sequence and the V_{L} amino acid sequence selected from the group consisting of: SEQ ID NO: 2 and SEQ ID NO: 20; SEQ ID NO: 3 and SEQ ID NO: 21; SEQ ID NO: 4 and SEQ ID NO: 22; SEQ ID NO: 5 and SEQ ID NO: 23; SEQ ID NO: 6 and SEQ ID NO: 24; SEQ ID NO: 7 and SEQ ID NO: 25; SEQ ID NO: 8 and SEQ ID NO: 26; SEQ ID NO: 9 and SEQ ID NO: 27; SEQ ID NO: 10 and SEQ ID NO: 28; SEQ ID NO: 11 and SEQ ID NO: 29; SEQ ID NO: 12 and SEQ ID NO: 30; SEQ ID NO: 13 and SEQ ID NO: 31; SEQ ID NO: 14 and SEQ ID NO: 32; SEQ ID NO: 15 and SEQ ID NO: 33; SEQ ID NO: 16 and SEQ ID NO: 34; SEQ ID NO: 17 and SEQ ID NO: 35; SEQ ID NO: 18 and SEQ ID NO: 36; and SEQ ID NO: 19 and SEQ ID NO: 37.

Embodiment 6: The composition of any one of Embodiments 1-5, comprising an amino acid sequence having at least 90% identity to a sequence selected from the group consisting of: SEQ ID NOs: 38-43.

Embodiment 7: The composition of Embodiment 6, comprising an amino acid sequence selected from the group consisting of: SEQ ID NOs: 38-43.

Embodiment 8: An anti-NDC80/MHC composition comprising an antibody moiety that competes with the composition of Embodiment 5 for specific binding to a NDC80 peptide/MHC complex.

Embodiment 9: The composition of any one of Embodiments 1-7, further comprising a Fc domain of an isotype selected from the group consisting of IgG1, IgG2, IgG3, IgG4, IgA1, IgA2, IgM, IgD, and IgE.

Embodiment 10: The composition of any one of Embodiments 1-7, wherein the antibody moiety is a full-length antibody, a Fab, a F(ab′)2, a Fab′, a Fv, or a single chain Fv (scFv).

Embodiment 11: The composition of any one of Embodiments 1-10, wherein the composition is a chimeric antibody-T cell receptor (caTCR).

Embodiment 12: The composition of any one of Embodiments 1-11, wherein the composition comprises at least a fragment of a T cell receptor (TCR) chain.

Embodiment 13: The composition of Embodiment 12, wherein the fragment of TCR chain comprises the transmembrane domain of the TCR chain.

Embodiment 14: The composition of any one of Embodiments 12-13, wherein the fragment of TCR chain does not comprise any CDR sequence of the TCR chain.

Embodiment 15: The composition of any one of Embodiments 1-10, wherein the composition is a chimeric antigen receptor (CAR).

Embodiment 16: The composition of any one of Embodiments 1-15, wherein the composition is monospecific.

Embodiment 17: The composition of any one of Embodiments 1-15, wherein the composition is multispecific.

Embodiment 18: The composition of Embodiment 17, wherein the composition is bispecific.

Embodiment 19: The composition of any one of Embodiments 1-18, wherein the composition comprises a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody.

Embodiment 20: The composition of any one of Embodiments 17-19, wherein the composition comprises a tandem scFv with at least one peptide linker between two scFvs.

Embodiment 21: The composition of any one of Embodiments 17-20, wherein the composition comprises a second antibody moiety that specifically binds to a second antigen.

Embodiment 22: The composition of Embodiment 21, wherein the second antigen is an antigen on the surface of a T cell, a natural killer cell, a neutrophil, a monocyte, a macrophage, or a dendritic cell.

Embodiment 23: The composition of Embodiment 21, wherein the second antigen is a disease-specific antigen that is not NDC80/MHC.

Embodiment 24: The composition of any one of Embodiments 1-23, wherein the composition is a chimeric antibody, a humanized antibody, or a human antibody.

Embodiment 25: The composition of Embodiment 24, wherein the composition is a fully human antibody.

Embodiment 26: The composition of any one of Embodiments 1-25, wherein the composition is a monoclonal antibody.

Embodiment 27: The composition of any one of Embodiments 1-26, wherein the composition is an immunoglobulin-related composition, an immunoglobulin polypeptide, or an immunoglobulin-like polypeptide.

Embodiment 28: The composition of any one of Embodiments 1-27, wherein the composition specifically binds to a NDC80 peptide/HLA-A*02 complex.

Embodiment 29: The composition of Embodiment 28, wherein said NDC80 peptide comprises the amino acid sequence ALNEQIARL (SEQ ID NO: 1).

Embodiment 30: The composition of Embodiment 28 or 29, wherein said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Embodiment 31: A recombinant nucleic acid or a set of recombinant nucleic acids encoding the composition of any one of Embodiments 1-30, with all components of the composition encoded by one nucleic acid or by the set of nucleic acids.

Embodiment 32: A vector comprising the recombinant nucleic acid of Embodiment 31.

Embodiment 33: A set of vectors comprising the set of recombinant nucleic acids of Embodiment 31.

Embodiment 34: A cell comprising the recombinant nucleic acid or the set of recombinant nucleic acids of Embodiment 31, the vector of Embodiment 32, or the set of vectors of Embodiment 33.

Embodiment 35: A cell that displays on its surface or secretes the composition of any one of Embodiments 1-30.

Embodiment 36: The cell of Embodiment 34 or 35, wherein the cell is a T cell, a NK cell, a B cell, or a monocyte/macrophage.

Embodiment 37: A pharmaceutical composition comprising the composition of any one of Embodiments 1-30, the recombinant nucleic acid or the set of recombinant nucleic acids of Embodiment 31, the vector of Embodiment 32, the set of vectors of Embodiment 33, or the cell of any one of Embodiments 34-36, and a pharmaceutically-acceptable carrier.

Embodiment 38: The composition of any one of Embodiments 1-30 or the pharmaceutical composition of Embodiment 37, wherein the composition is conjugated to an agent selected from the group consisting of detectable label, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

Embodiment 39: A method for treating a NDC80-associated disease in a subject in need thereof, comprising administering to the subject an effective amount of the composition of any one of Embodiments 1-30 or 38.

Embodiment 40: A method for treating a NDC80-associated disease in a subject in need thereof, comprising administering to the subject an effective amount of the recombinant nucleic acid or the set of recombinant nucleic acids of Embodiment 31, the vector of Embodiment 32, or the set of vectors of Embodiment 33.

Embodiment 41: A method for treating a NDC80-associated disease in a subject in need thereof, comprising administering to the subject an effective amount of the cell of any one of Embodiments 34-36.

Embodiment 42: A method for treating a NDC80-associated disease in a subject in need thereof, comprising administering to the subject an effective amount of the pharmaceutical composition of Embodiment 37 or 38.

Embodiment 43: A method for treating a NDC80-associated disease in a subject in need thereof, comprising administering to the subject an effective amount of a composition comprising an antibody moiety that specifically binds to a NDC80 peptide/HLA-A*02 complex.

Embodiment 44: A method for treating a NDC80-associated disease in a subject in need thereof, comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

Embodiment 45: A method for treating a NDC80-associated disease in a subject in need thereof, comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a).

Embodiment 46: The method of Embodiment 45, wherein the cell of (c) or (d) is a T cell, a NK cell, a B cell, or a monocyte/macrophage.

Embodiment 47: The method of any one of Embodiments 43-46, wherein the NDC80 peptide comprises the amino acid sequence ALNEQIARL (SEQ ID NO: 1).

Embodiment 48: The method of any one of Embodiments 43-47, wherein said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Embodiment 49: The method of any one of Embodiments 39-48, wherein the NDC80-associated disease is a cancer.

Embodiment 50: The method of Embodiment 49, wherein the cancer is acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02.

Embodiment 51: The method of any one of Embodiments 39-50, wherein the composition is administered to the subject separately, sequentially or simultaneously with an additional therapeutic agent.

Embodiment 52: The method of Embodiment 51, wherein the additional therapeutic agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, and bisphosphonate therapy agents.

Embodiment 53: The method of Embodiment 51 or 52, wherein the additional therapeutic agent is one or more of immune checkpoint inhibitors, monoclonal antibodies that specifically target tumor antigens, T-cell therapy, immune activating agents, oncolytic virus therapy and cancer vaccines.

Embodiment 54: A method for detecting NDC80 expression levels in a biological sample comprising (a) contacting the biological sample with the composition of any one of Embodiments 1-30 or 38; and (b) detecting binding to a NDC80 peptide-HLA-A*02 complex in the biological sample.

Embodiment 55: The method of Embodiment 54, wherein the NDC80 peptide comprises the amino acid sequence ALNEQIARL (SEQ ID NO: 1).

Embodiment 56: A kit comprising the composition of any one of Embodiments 1-30 or 38 and instructions for use.

Embodiment 57: The kit of Embodiment 56, wherein the composition of any one of Embodiments 1-30 or 38 is coupled to at least one detectable label selected from the group consisting of a radioactive label, a fluorescent label, and a chromogenic label.

Embodiment 58: The kit of Embodiment 56 or 57, further comprising a secondary antibody that specifically binds to the composition of any one of Embodiments 1-30 or 38.

Embodiment 59: A method for mitigating immunotherapy-related toxicity in a subject in need thereof comprising administering to the subject an effective amount of the composition of any one of Embodiments 1-30 or 38.

Embodiment 60: A method for mitigating immunotherapy-related toxicity in a subject in need thereof comprising administering to the subject an effective amount of the recombinant nucleic acid or the set of recombinant nucleic acids of Embodiment 31, the vector of Embodiment 32, or the set of vectors of Embodiment 33.

Embodiment 61: A method for mitigating immunotherapy-related toxicity in a subject in need thereof comprising administering to the subject comprising administering to the subject an effective amount of the cell of any one of Embodiments 34-36.

Embodiment 62: A method for mitigating immunotherapy-related toxicity in a subject in need thereof comprising administering to the subject an effective amount of the pharmaceutical composition of Embodiment 37 or 38.

Embodiment 63: A method for mitigating immunotherapy-related toxicity in a subject in need thereof comprising administering to the subject an effective amount of a composition comprising an antibody moiety that specifically binds to a NDC80 peptide/HLA-A*02 complex.

Embodiment 64: A method for mitigating immunotherapy-related toxicity in a subject in need thereof comprising administering to the subject an effective amount of a recombinant nucleic acid, a set of recombinant nucleic acids, a vector, or a set of vectors that encode a composition comprising an antibody moiety that specifically binds to a NDC80 peptide/HLA-A*02 complex.

Embodiment 65: A method for mitigating immunotherapy-related toxicity in a subject in need thereof comprising administering to the subject an effective amount of a pharmaceutical composition comprising a pharmaceutically-acceptable carrier and (a) a composition comprising an antibody moiety that specifically binds to a NDC80 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a).

Embodiment 66: The method of Embodiment 65, wherein the cell of (c) or (d) is a T cell, a NK cell, a B cell, or a monocyte/macrophage.

Embodiment 67: The method of any one of Embodiments 63-66, wherein the NDC80 peptide comprises the amino acid sequence ALNEQIARL (SEQ ID NO: 1).

Embodiment 68: The method of any one of Embodiments 63-67, wherein said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

Embodiment 69: The method of any one of Embodiments 59-68, wherein the subject has been diagnosed with or is suffering from a NDC80-associated disease.

Embodiment 70: The method of Embodiment 69, wherein the NDC80-associated disease is cancer.

Embodiment 71: The method of Embodiment 70, wherein the cancer is acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02.

Embodiment 72: The method of any one of Embodiments 59-71, wherein the immunotherapy-related toxicity is selected from the group consisting of T-cell fratricide, hematopoietic stem cell toxicity, peripheral blood mononuclear cell (PBMC) toxicity, cardiomyocyte toxicity, cardiac fibroblast toxicity and thymic fibroblast toxicity.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A composition that comprises an antibody moiety comprising:
(a) a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 2, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 20,
(b) a V_{H} comprising a V_{H}-CDR1 sequence, a V_{H}-CDR2 sequence, and a V_{H}-CDR3 sequence of the V_{H} sequence of SEQ ID NO: 3, and a V_{L} comprising a V_{L}-CDR1 sequence, a V_{L}-CDR2 sequence, and a V_{L}-CDR3 sequence of the V_{L} sequence SEQ ID NO: 21,
(c) a V_{H} comprising a V_{H}-CDR1 sequence, a V_{H}-CDR2 sequence, and a V_{H}-CDR3 sequence of the V_{H} sequence of SEQ ID NO: 4, and a V_{L} comprising a V_{L}-CDR1 sequence, a V_{L}-CDR2 sequence, and a V_{L}-CDR3 sequence of the V_{L} sequence SEQ ID NO: 22,
(d) a V_{H} comprising a V_{H}-CDR1 sequence, a V_{H}-CDR2 sequence, and a V_{H}-CDR3 sequence of the V_{H} sequence of SEQ ID NO: 5, and a V_{L} comprising a V_{L}-CDR1 sequence, a V_{L}-CDR2 sequence, and a V_{L}-CDR3 sequence of the V_{L} sequence SEQ ID NO: 23,
(e) a V_{H} comprising a V_{H}-CDR1 sequence, a V_{H}-CDR2 sequence, and a V_{H}-CDR3 sequence of the V_{H} sequence of SEQ ID NO: 6, and a V_{L} comprising a V_{L}-CDR1 sequence, a V_{L}-CDR2 sequence, and a V_{L}-CDR3 sequence of the V_{L} sequence SEQ ID NO: 24,
(f) a V_{H} comprising a V_{H}-CDR1 sequence, a V_{H}-CDR2 sequence, and a V_{H}-CDR3 sequence of the V_{H} sequence of SEQ ID NO: 7, and a V_{L} comprising a V_{L}-CDR1 sequence, a V_{L}-CDR2 sequence, and a V_{L}-CDR3 sequence of the V_{L} sequence SEQ ID NO: 25,
(g) a V_{H} comprising a V_{H}-CDR1 sequence, a V_{H}-CDR2 sequence, and a V_{H}-CDR3 sequence of the V_{H} sequence of SEQ ID NO: 8, and a V_{L}, comprising a V_{L}-CDR1 sequence, a V_{L}-CDR2 sequence, and a V_{L}-CDR3 sequence of the V_{L} sequence SEQ ID NO: 26,
(h) a V_{H} comprising a V_{H}-CDR1 sequence, a V_{H}-CDR2 sequence, and a V_{H}-CDR3 sequence of the V_{H} sequence of SEQ ID NO: 9, and a V_{L} comprising a V_{L}-CDR1 sequence, a V_{L}-CDR2 sequence, and a V_{L}-CDR3 sequence of the V_{L} sequence SEQ ID NO: 27,
(i) a V_{H} comprising a V_{H}-CDR1 sequence, a V_{H}-CDR2 sequence, and a V_{H}-CDR3 sequence of the V_{H} sequence of SEQ ID NO: 10, and a V_{L} comprising a V_{L}-CDR1 sequence, a V_{L}-CDR2 sequence, and a V_{L}-CDR3 sequence of the V_{L} sequence SEQ ID NO: 28,
(j) a V_{H} comprising a V_{H}-CDR1 sequence, a V_{H}-CDR2 sequence, and a V_{H}-CDR3 sequence of the V_{H} sequence of SEQ ID NO: 11, and a V_{L} comprising a V_{L}-CDR1 sequence, a V_{L}-CDR2 sequence, and a V_{L}-CDR3 sequence of the V_{L} sequence SEQ ID NO: 29,
(k) a V_{H} comprising a V_{H}-CDR1 sequence, a V_{H}-CDR2 sequence, and a V_{H}-CDR3 sequence of the V_{H} sequence of SEQ ID NO: 12, and a V_{L} comprising a V_{L}-CDR1 sequence, a V_{L}-CDR2 sequence, and a V_{L}-CDR3 sequence of the V_{L} sequence SEQ ID NO: 30,
(l) a V_{H} comprising a V_{H}-CDR1 sequence, a V_{H}-CDR2 sequence, and a V_{H}-CDR3 sequence of the V_{H} sequence of SEQ ID NO: 13, and a V_{L} comprising a V_{L}-CDR1 sequence, a V_{L}-CDR2 sequence, and a V_{L}-CDR3 sequence of the V_{L} sequence SEQ ID NO: 31,
(m) a V_{H} comprising a V_{H}-CDR1 sequence, a V_{H}-CDR2 sequence, and a V_{H}-CDR3 sequence of the V_{H} sequence of SEQ ID NO: 14, and a V_{L} comprising a V_{L}-CDR1 sequence, a V_{L}-CDR2 sequence, and a V_{L}-CDR3 sequence of the V_{L} sequence SEQ ID NO: 32,
(n) a V_{H} comprising a V_{H}-CDR1 sequence, a V_{H}-CDR2 sequence, and a V_{H}-CDR3 sequence of the V_{H} sequence of SEQ ID NO: 15, and a V_{L} comprising a V_{L}-CDR1 sequence, a V_{L}-CDR2 sequence, and a V_{L}-CDR3 sequence of the V_{L} sequence SEQ ID NO: 33,
(o) a V_{H} comprising a V_{H}-CDR1 sequence, a V_{H}-CDR2 sequence, and a V_{H}-CDR3 sequence of the V_{H} sequence of SEQ ID NO: 16, and a V_{L} comprising a V_{L}-CDR1 sequence, a V_{L}-CDR2 sequence, and a V_{L}-CDR3 sequence of the V_{L} sequence SEQ ID NO: 34,
(p) a V_{H} comprising a V_{H}-CDR1 sequence, a V_{H}-CDR2 sequence, and a V_{H}-CDR3 sequence of the V_{H} sequence of SEQ ID NO: 17, and a V_{L} comprising a V_{L}-CDR1 sequence, a V_{L}-CDR2 sequence, and a V_{L}-CDR3 sequence of the V_{L} sequence SEQ ID NO: 35,
(q) a V_{H} comprising a V_{H}-CDR1 sequence, a V_{H}-CDR2 sequence, and a V_{H}-CDR3 sequence of the V_{H} sequence of SEQ ID NO: 18, and a V_{L} comprising a V_{L}-CDR1 sequence, a V_{L}-CDR2 sequence, and a V_{L}-CDR3 sequence of the V_{L} sequence SEQ ID NO: 36, or
(r) a V_{H} comprising a V_{H}-CDR1 sequence, a V_{H}-CDR2 sequence, and a V_{H}-CDR3 sequence of the V_{H} sequence of SEQ ID NO: 19, and a V_{L} comprising a V_{L}-CDR1 sequence, a V_{L}-CDR2 sequence, and a V_{L}-CDR3 sequence of the V_{L} sequence SEQ ID NO: 37.

2. The composition of claim 1, wherein
(a) the V_{H}-CDR1 sequence comprises the sequence of SEQ ID NO: 44, the V_{H}-CDR2 sequence comprises the sequence of SEQ ID NO: 45, the V_{H}-CDR3 sequence comprises the sequence of SEQ ID NO: 46, the V_{L}-CDR1 sequence comprises the sequence of SEQ ID NO: 98, the V_{L}-CDR2 sequence comprises the sequence of SEQ ID NO: 99, and/or the V_{L}-CDR3 sequence comprises the sequence of SEQ ID NO: 100;
(b) the V_{H}-CDR1 sequence comprises the sequence of SEQ ID NO: 47, the V_{H}-CDR2 sequence comprises the sequence of SEQ ID NO: 48, the V_{H}-CDR3 sequence comprises the sequence of SEQ ID NO: 49, the V_{L}-CDR1 sequence comprises the sequence of SEQ ID NO: 101, the V_{L}-CDR2 sequence comprises the sequence of SEQ ID NO: 102, and/or the V_{L}-CDR3 sequence comprises the sequence of SEQ ID NO: 103;
(c) the V_{H}-CDR1 sequence comprises the sequence of SEQ ID NO: 50, the V_{H}-CDR2 sequence comprises the sequence of SEQ ID NO: 51, the V_{H}-CDR3 sequence comprises the sequence of SEQ ID NO: 52, the V_{L}-CDR1 sequence comprises the sequence of SEQ ID NO: 104, the V_{L}-CDR2 sequence comprises the sequence of SEQ ID NO: 105, and/or the V_{L}-CDR3 sequence comprises the sequence of SEQ ID NO: 106;
(d) the V_{H}-CDR1 sequence comprises the sequence of SEQ ID NO: 53, the V_{H}-CDR2 sequence comprises the sequence of SEQ ID NO: 54, the V_{H}-CDR3 sequence comprises the sequence of SEQ ID NO: 55, the V_{L}-CDR1 sequence comprises the sequence of SEQ ID NO: 107, the V_{L}-CDR2 sequence comprises the sequence of SEQ ID NO: 108, and/or the V_{L}-CDR3 sequence comprises the sequence of SEQ ID NO: 109;
(e) the V_{H}-CDR1 sequence comprises the sequence of SEQ ID NO: 56, the V_{H}-CDR2 sequence comprises the sequence of SEQ ID NO: 57, the V_{H}-CDR3 sequence comprises the sequence of SEQ ID NO: 58, the V_{L}-CDR1 sequence comprises the sequence of SEQ ID NO: 110, the V_{L}-CDR2 sequence comprises the sequence of SEQ ID NO: 111, and/or the V_{L}-CDR3 sequence comprises the sequence of SEQ ID NO: 112;
(f) the V_{H}-CDR1 sequence comprises the sequence of SEQ ID NO: 59, the V_{H}-CDR2 sequence comprises the sequence of SEQ ID NO: 60, the V_{H}-CDR3 sequence comprises the sequence of SEQ ID NO: 61, the V_{L}-CDR1 sequence comprises the sequence of SEQ ID NO: 113, the V_{L}-CDR2 sequence comprises the sequence of SEQ ID NO: 114, and/or the V_{L}-CDR3 sequence comprises the sequence of SEQ ID NO: 115;
(g) the V_{H}-CDR1 sequence comprises the sequence of SEQ ID NO: 62, the V_{H}-CDR2 sequence comprises the sequence of SEQ ID NO: 63, the V_{H}-CDR3 sequence comprises the sequence of SEQ ID NO: 64, the V_{L}-CDR1 sequence comprises the sequence of SEQ ID NO: 116, the V_{L}-CDR2 sequence comprises the sequence of SEQ ID NO: 117, and/or the V_{L}-CDR3 sequence comprises the sequence of SEQ ID NO: 118;
(h) the V_{H}-CDR1 sequence comprises the sequence of SEQ ID NO: 65, the V_{H}-CDR2 sequence comprises the sequence of SEQ ID NO: 66, the V_{H}-CDR3 sequence comprises the sequence of SEQ ID NO: 67, the V_{L}-CDR1 sequence comprises the sequence of SEQ ID NO: 119, the V_{L}-CDR2 sequence comprises the sequence of SEQ ID NO: 120, and/or the V_{L}-CDR3 sequence comprises the sequence of SEQ ID NO: 121;
(i) the V_{H}-CDR1 sequence comprises the sequence of SEQ ID NO: 68, the V_{H}-CDR2 sequence comprises the sequence of SEQ ID NO: 69, the V_{H}-CDR3 sequence comprises the sequence of SEQ ID NO: 70, the V_{L}-CDR1 sequence comprises the sequence of SEQ ID NO: 122, the V_{L}-CDR2 sequence comprises the sequence of SEQ ID NO: 123, and/or the V_{L}-CDR3 sequence comprises the sequence of SEQ ID NO: 124;
(j) the V_{H}-CDR1 sequence comprises the sequence of SEQ ID NO: 71, the V_{H}-CDR2 sequence comprises the sequence of SEQ ID NO: 72, the V_{H}-CDR3 sequence comprises the sequence of SEQ ID NO: 73, the V_{L}-CDR1 sequence comprises the sequence of SEQ ID NO: 125, the V_{L}-CDR2 sequence comprises the sequence of SEQ ID NO: 126, and/or the V_{L}-CDR3 sequence comprises the sequence of SEQ ID NO: 127;
(k) the V_{H}-CDR1 sequence comprises the sequence of SEQ ID NO: 74, the V_{H}-CDR2 sequence comprises the sequence of SEQ ID NO: 75, the V_{H}-CDR3 sequence comprises the sequence of SEQ ID NO: 76, the V_{L}-CDR1 sequence comprises the sequence of SEQ ID NO: 128, the V_{L}-CDR2 sequence comprises the sequence of SEQ ID NO: 129, and/or the V_{L}-CDR3 sequence comprises the sequence of SEQ ID NO: 130;
(l) the V_{H}-CDR1 sequence comprises the sequence of SEQ ID NO: 77, the V_{H}-CDR2 sequence comprises the sequence of SEQ ID NO: 78, the V_{H}-CDR3 sequence comprises the sequence of SEQ ID NO: 79, the V_{L}-CDR1 sequence comprises the sequence of SEQ ID NO: 131, the V_{L}-CDR2 sequence comprises the sequence of SEQ ID NO: 132, and/or the V_{L}-CDR3 sequence comprises the sequence of SEQ ID NO: 133;
(m) the V_{H}-CDR1 sequence comprises the sequence of SEQ ID NO: 80, the V_{H}-CDR2 sequence comprises the sequence of SEQ ID NO: 81, the V_{H}-CDR3 sequence comprises the sequence of SEQ ID NO: 82, the V_{L}-CDR1 sequence comprises the sequence of SEQ ID NO: 134, the V_{L}-CDR2 sequence comprises the sequence of SEQ ID NO: 135, and/or the V_{L}-CDR3 sequence comprises the sequence of SEQ ID NO: 136;
(n) the V_{H}-CDR1 sequence comprises the sequence of SEQ ID NO: 83, the V_{H}-CDR2 sequence comprises the sequence of SEQ ID NO: 84, the V_{H}-CDR3 sequence comprises the sequence of SEQ ID NO: 85, the V_{L}-CDR1 sequence comprises the sequence of SEQ ID NO: 137, the V_{L}-CDR2 sequence comprises the sequence of SEQ ID NO: 138, and/or the V_{L}-CDR3 sequence comprises the sequence of SEQ ID NO: 139;
(o) the V_{H}-CDR1 sequence comprises the sequence of SEQ ID NO: 86, the V_{H}-CDR2 sequence comprises the sequence of SEQ ID NO: 87, the V_{H}-CDR3 sequence comprises the sequence of SEQ ID NO: 88, the V_{L}-CDR1 sequence comprises the sequence of SEQ ID NO: 140, the V_{L}-CDR2 sequence comprises the sequence of SEQ ID NO: 141, and/or the V_{L}-CDR3 sequence comprises the sequence of SEQ ID NO: 142;
(p) the V_{H}-CDR1 sequence comprises the sequence of SEQ ID NO: 89, the V_{H}-CDR2 sequence comprises the sequence of SEQ ID NO: 90, the V_{H}-CDR3 sequence comprises the sequence of SEQ ID NO: 91, the V_{L}-CDR1 sequence comprises the sequence of SEQ ID NO: 143, the V_{L}-CDR2 sequence comprises the sequence of SEQ ID NO: 144, and/or the V_{L}-CDR3 sequence comprises the sequence of SEQ ID NO: 145;
(q) the V_{H}-CDR1 sequence comprises the sequence of SEQ ID NO: 92, the V_{H}-CDR2 sequence comprises the sequence of SEQ ID NO: 93, the V_{H}-CDR3 sequence comprises the sequence of SEQ ID NO: 94, the V_{L}-CDR1 sequence comprises the sequence of SEQ ID NO: 146, the V_{L}-CDR2 sequence comprises the sequence of SEQ ID NO: 147, and/or the V_{L}-CDR3 sequence comprises the sequence of SEQ ID NO: 148; or
(r) the V_{H}-CDR1 sequence comprises the sequence of SEQ ID NO: 95, the V_{H}-CDR2 sequence comprises the sequence of SEQ ID NO: 96, the V_{H}-CDR3 sequence comprises the sequence of SEQ ID NO: 97, the V_{L}-CDR1 sequence comprises the sequence of SEQ ID NO: 149, the V_{L}-CDR2 sequence comprises the sequence of SEQ ID NO: 150, and/or the V_{L}-CDR3 sequence comprises the sequence of SEQ ID NO: 151.

3. The composition of claim 1, wherein
(a) the V_{H} comprises an amino acid sequence having at least 90% identity to a sequence selected from the group consisting of: SEQ ID NOs: 2-19, and/or
(b) the V_{L} comprises an amino acid sequence having at least 90% identity to a sequence selected from the group consisting of: SEQ ID NOs: 20-37; and/or
(c) the V_{H} comprises an amino acid sequence selected from the group consisting of: SEQ ID NOs: 2-19 or a variant thereof having one or more conservative amino acid substitutions; and/or
(d) the V_{L} comprises an amino acid sequence selected from the group consisting of: SEQ ID NOs: 20-37 or a variant thereof having one or more conservative amino acid substitutions.

4. (canceled)

5. The composition of claim 1, wherein the V_{H} amino acid sequence and the V_{L} amino acid sequence selected from the group consisting of:
SEQ ID NO: 2 and SEQ ID NO: 20;
SEQ ID NO: 3 and SEQ ID NO: 21;
SEQ ID NO: 4 and SEQ ID NO: 22;
SEQ ID NO: 5 and SEQ ID NO: 23;
SEQ ID NO: 6 and SEQ ID NO: 24;
SEQ ID NO: 7 and SEQ ID NO: 25;
SEQ ID NO: 8 and SEQ ID NO: 26;
SEQ ID NO: 9 and SEQ ID NO: 27;
SEQ ID NO: 10 and SEQ ID NO: 28;
SEQ ID NO: 11 and SEQ ID NO: 29;
SEQ ID NO: 12 and SEQ ID NO: 30;
SEQ ID NO: 13 and SEQ ID NO: 31;
SEQ ID NO: 14 and SEQ ID NO: 32;
SEQ ID NO: 15 and SEQ ID NO: 33;
SEQ ID NO: 16 and SEQ ID NO: 34;
SEQ ID NO: 17 and SEQ ID NO: 35;
SEQ ID NO: 18 and SEQ ID NO: 36; and
SEQ ID NO: 19 and SEQ ID NO: 37.

6. The composition of claim 1, comprising an amino acid sequence having at least 90% identity to a sequence selected from the group consisting of: SEQ ID NOs: 38-43, optionally wherein the composition comprises an amino acid sequence selected from the group consisting of: SEQ ID NOs: 38-43.

7. (canceled)

8. An anti-NDC80/MHC composition comprising an antibody moiety that competes with the composition of claim 5 for specific binding to a NDC80 peptide/MHC complex.

9. The composition of claim 1, further comprising a Fc domain of an isotype selected from the group consisting of IgG1, IgG2, IgG3, IgG4, IgA1, IgA2, IgM, IgD, and IgE, or wherein the antibody moiety is a full-length antibody, a Fab, a F(ab′)₂, a Fab′, a Fv, or a single chain Fv (scFv).

10. (canceled)

11. The composition of claim 1, wherein the composition is a chimeric antibody-T cell receptor (caTCR).

12. The composition of claim 1, wherein the composition comprises at least a fragment of a T cell receptor (TCR) chain, optionally wherein the fragment of TCR chain comprises the transmembrane domain of the TCR chain or the fragment of TCR chain does not comprise any CDR sequence of the TCR chain.

13. (canceled)

14. (canceled)

15. The composition of claim 1, wherein the composition is a chimeric antigen receptor (CAR).

16. The composition of claim 1, wherein the composition is monospecific, multispecific, or bispecific, optionally wherein
the composition comprises a tandem scFv with at least one peptide linker between two scFvs; or
the composition comprises a second antibody moiety that specifically binds to a second antigen, optionally wherein the second antigen is a disease-specific antigen that is not NDC80/MHC, optionally wherein the second antigen is an antigen on the surface of a T cell, a natural killer cell, a neutrophil, a monocyte, a macrophage, or a dendritic cell.

17. The composition of claim 1, wherein the composition comprises a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody; or
is a chimeric antibody, a humanized antibody, a human antibody, a fully human antibody, or a monoclonal antibody; or
is an immunoglobulin-related composition, an immunoglobulin polypeptide, or an immunoglobulin-like polypeptide; or
specifically binds to a NDC80 peptide/HLA-A*02 complex, optionally wherein said NDC80 peptide comprises the amino acid sequence ALNEQIARL (SEQ ID NO: 1), optionally wherein said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

18. (canceled)

19. (canceled)

20. (canceled)

21. (canceled)

22. (canceled)

23. (canceled)

24. (canceled)

25. A recombinant nucleic acid or a set of recombinant nucleic acids encoding the composition of claim 1, with all components of the composition encoded by one nucleic acid or by the set of nucleic acids.

26. A vector or cell comprising the recombinant nucleic acid of claim 25.

27. A cell (a) comprising the vector of claim 26; or (b) that displays on its surface or secretes the composition of claim 1, optionally wherein the cell is a T cell, a NK cell, a B cell, or a monocyte/macrophage.

28. (canceled)

29. A pharmaceutical composition comprising the composition of claim 1, and a pharmaceutically-acceptable carrier, optionally wherein the composition is conjugated to an agent selected from the group consisting of detectable label, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

30. (canceled)

31. A method for treating a NDC80-associated disease in a subject in need thereof, comprising administering to the subject an effective amount of the composition of claim 1, optionally wherein
the NDC80-associated disease is a cancer, optionally wherein the cancer is acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02; or
wherein the composition is administered to the subject separately, sequentially or simultaneously with an additional therapeutic agent, optionally wherein the additional therapeutic agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, immune checkpoint inhibitors, monoclonal antibodies that specifically target tumor antigens, T-cell therapy, immune activating agents, oncolytic virus therapy and cancer vaccines.

32. (canceled)

33. (canceled)

34. (canceled)

35. A method for detecting NDC80 expression levels in a biological sample comprising (a) contacting the biological sample with the composition of claim 1; and (b) detecting binding to a NDC80 peptide-HLA-A*02 complex in the biological sample, optionally wherein the NDC80 peptide comprises the amino acid sequence ALNEQIARL (SEQ ID NO: 1).

36. A kit comprising the composition of claim 1 and instructions for use, optionally wherein the composition is coupled to at least one detectable label selected from the group consisting of a radioactive label, a fluorescent label, and a chromogenic label and/or wherein the kit further comprises a secondary antibody that specifically binds to the composition.

37. A method for mitigating immunotherapy-related toxicity in a subject in need thereof comprising administering to the subject an effective amount of the composition of claim 1, optionally wherein
the subject has been diagnosed with or is suffering from a NDC80-associated disease or cancer, optionally wherein the cancer is acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, breast cancer, cervical cancer, prostate cancer, melanoma, mesothelioma, pancreatic cancer, thyroid cancer, or a cancer presenting the peptide of SEQ ID NO: 1 in complex with HLA-A*02; or
the immunotherapy-related toxicity is selected from the group consisting of T-cell fratricide, hematopoietic stem cell toxicity, and peripheral blood mononuclear cell (PBMC) toxicity.

38. (canceled)

39. (canceled)

40. (canceled)
